(19) United States
(12) Patent Application Publication
(10) Pub. No.: US 2022/0306719 A1
(43) Pub. Date: Sep. 29, 2022

(54) ULTRAMODULAR IGG3-BASED SPACER DOMAIN AND MULTI-FUNCTION SITE FOR IMPLEMENTATION IN CHIMERIC ANTIGEN RECEPTOR DESIGN

(71) Applicant: JULIUS-MAXIMILIANS-UNIVERSITÄT WÜRZBURG, Würzburg (DE)

(72) Inventors: Michael HUDECEK, Höchberg (DE); Thomas NERRETER, Würzburg (DE)

(21) Appl. No.: 17/619,569

(22) PCT Filed: Jun. 19, 2020
(86) PCT No.: PCT/EP2020/067124

§ 371 Date: Dec. 15, 2021

(30) Foreign Application Priority Data
Jun. 19, 2019 (EP) 19181237.9

(51) Int. Cl. C07K 14/725; C07K 16/28; A61P 31/00; A61P 35/02; C07K 16/42; A61K 47/68

(52) U.S. Cl. CPC C07K 14/7051; C07K 16/2803; A61P 31/00; A61P 35/02; C07K 16/2848; C07K 16/2863; C07K 16/2878; C07K 16/2887; C07K 16/4283; A61K 47/6809; A61K 47/6849; A61K 47/6873

(57) ABSTRACT

The invention generally relates to immunotherapy using immune cells such as chimeric antigen receptor (CAR)-engineered T cells. In particular, the invention relates to immunotherapy using chimeric antigen receptor (CAR)-engineered T cells that carry a novel, IgG3-Hinge-based spacer domain, allowing a finely modulated response to target antigens. In addition, the invention relates to the introduction of one or more IgG3-Hinge-based multi-function sites (MFs) into CARs and other immunoreceptors, allowing purification, stimulation, expansion and depletion of CAR T cells. The invention includes also the sequence of an antibody targeting this motif, allowing the execution of the before-mentioned functions.

### FIELD OF THE INVENTION

The invention generally relates to immunotherapy using immune cells such as chimeric antigen receptor (CAR)-engineered T cells. In particular, the invention relates to immunotherapy using chimeric antigen receptor (CAR)-engineered T cells that carry a novel, IgG3-Hinge-based spacer domain, allowing a finely modulated response to target antigens. In addition, the invention relates to the introduction of one or more IgG3-Hinge-based multi-function sites (MFS) into CARs and other immunoreceptors, allowing purification, stimulation, expansion and depletion of CAR T cells. The invention includes also the sequence of an antibody targeting this motif, allowing the execution of the before-mentioned functions.

### BACKGROUND OF THE INVENTION

Chimeric antigen receptors (CARs) are synthetic immune receptors that have been developed with the intention to redirect T cells to recognize surface antigens on tumor cells. In their most basic format, CARs comprise the variable heavy and variable light chain (in cis, i.e. as a single chain variable fragment, scFv) of a monoclonal antibody fused to a transmembrane domain and the signaling domain of CD3ζ¹. A step to improving this basic CAR design was the inclusion of a spacer domain located between the scFv and the transmembrane domain to provide reach and flexibility in order to promote antigen binding by the CAR². In the sequel, several spacer domains were used in CAR constructs including Fc regions and immunoglobulin-like domains derived from IgG1 and IgG4, IgD, CD4, CD7, CD8α and CD28³⁻⁶.

The conventional approach in the field is to use a single spacer design for all CAR constructs, even though they may recognize distinct epitopes in a given antigen, or distinct antigens (‘one CAR has to fit all’). However, because CARs bind to surface antigens on tumor cells, the spatial requirements that allow optimal antigen binding, and optimal interaction between CAR-modified T cell and tumor cell may differ depending on the epitope and target antigen. Therefore, the conventional approach of using a single spacer design for all epitopes and antigens seems naïve and suboptimal. If there is suboptimal CAR binding and/or suboptimal interaction between CAR-modified T cell and tumor cell, the ensuing CAR-T cell stimulation and anti-tumor response may also be suboptimal^{3,7}. To increase the chance of achieving a more optimized CAR-target molecule interaction, the inventors have previously investigated variants of IgG4-derived spacers that differ in length and composition. The paradigm that emerged was that there is a correlation between spacer length and efficacy whereby membrane-proximal epitopes on target cells are reached better by CARs containing longer spacer, and membrane-distal epitopes by CARs containing a shorter one⁷. Based on the architecture of the IgG4 molecule, three IgG4-Hinge based spacer variants are available that differ in size in increments>100 aa (IgG4_short: IgG4 Hinge, 12 aa; IgG4_intermediate: IgG4 Hinge+C_{H}3, 119 aa; IgG4_long: IgG4 Hinge+CH₂+CH₃, 228 aa)⁷.

Of all human IgG molecules, IgG3 shows the highest Fab-Fab folding flexibility and Fab Fc folding flexibility. The architecture of IgG3 is unique, as the hinge of IgG3, in contrast to all other immunoglobulins, incorporates 3 copies of a 15 aa motif caused by exon multiplication⁸⁻¹¹. Naturally occurring variants of IgG3 bearing only one or two copies of this motif in their hinge region show a much smaller distance between Fab and Fc (45 Å and 65 Å compared to 105 Å)⁸. These graduated differences and the opportunity of prolonging and shortening a spacer region by addition or removal of one or more copies of this 15 aa motif led in the present invention to the construction of an IgG3 Hinge library, using that, the length of the spacer can be fine-tuned to an optimal setting for every target. In addition, the inventors identified a monoclonal antibody that is specific to the IgG3 middle hinge motifs, allowing exploitation for additional, antigen-independent though CAR-specific functions, including purification, stimulation, expansion and depletion of CART cells.

### DESCRIPTION OF THE INVENTION

The invention generally relates to immunotherapy using immune cells such as chimeric antigen receptor (CAR)-engineered T cells. In particular, the invention relates to immunotherapy using chimeric antigen receptor (CAR)-engineered T cells that carry a novel, IgG3-Hinge-based spacer domain, allowing a finely modulated response to target antigens. In addition, the invention relates to the introduction of one or more IgG3-Hinge-based multi-function sites (MFS) into CARs and other immunoreceptors, allowing purification, stimulation, expansion and depletion of CAR T cells. The invention includes also the sequence of an antibody targeting this motif, allowing the execution of the before-mentioned functions.

The present invention provides and is characterized by, inter alio, the following items.
1. An immunoreceptor, comprising one or more IgG3 middle hinge repeat domain motifs, wherein the immunoreceptor does not comprise an IgG3 CH2 and/or CH3 domain.
2. The immunoreceptor according to item 1, wherein the immunoreceptor comprises an amino acid sequence which has at least 80% sequence identity, preferably at least 90% sequence identity, or most preferably 100% sequence identity with the amino acid sequence of [A-Bₙ],
   wherein
   A is the amino acid sequence of SEQ ID NO: 2;
   B is said IgG3 middle hinge domain repeat motif, wherein said motif has the amino acid sequence of SEQ ID NO: 1; and
   n is selected from the group consisting of 0, 1, 2, 3, 4, 5, 6, 7, 8, 9, 10, 11, 12, 13, 14 and 15 and is preferably an integer between 1 and 15, more preferably an integer between 1 and 10, even more preferably an integer between 1 and 5, most preferably an integer between 3 and 5.
3. The immunoreceptor according to item 2, wherein the immunoreceptor comprises an amino acid sequence which has 100% sequence identity with the amino acid sequence of [A-Bₙ].
4. The immunoreceptor according to item 2 or 3, wherein n is an integer between 1 and 10.
5. The immunoreceptor according to item 2 or 3, wherein n is an integer between 1 and 5.
6. The immunoreceptor according to item 2 or 3, wherein n is an integer between 3 and 5.
7. The immunoreceptor according to any one of the preceding items, comprising:
   an extracellular antigen-binding domain,
   a spacer domain,
   a transmembrane domain, and
   an intracellular signaling domain;
   wherein the spacer domain is located between the extracellular antigen-binding domain and the transmembrane domain,
   and wherein optionally the spacer domain comprises one or more IgG3 middle hinge domain repeat motifs.
8. The immunoreceptor according to item 7, wherein the transmembrane domain and the intracellular domain together consist of a sequence selected from the group consisting of SEQ ID NO: 109, 110, 111, 112, 113, 114, 115 and 174.
9. The immunoreceptor according to any one of the preceding items, comprising an extracellular antigen-binding domain comprising:
   a first domain,
   a linker, and, optionally,
   a second domain;
   optionally wherein the linker is located between the first domain and the second domain,
   and wherein optionally the linker comprises one or more IgG3 middle hinge domain repeat motifs.
10. The immunoreceptor according to items 7, 8 or 9,
   wherein the spacer domain comprises one or more IgG3 middle hinge domain repeat motifs,
   and/or
   wherein the linker comprised in the extracellular antigen-binding domain comprises one or more IgG3 middle hinge domain repeat motifs.
11. The immunoreceptor according to any one of the preceding items, wherein the immunoreceptor is selected from the group consisting of a T-cell receptor (TCR), preferably a recombinant TCR; a B-cell receptor (BCR), preferably a recombinant BCR; and a chimeric antigen receptor (CAR).
12. The immunoreceptor according to any one of items 9 to 11, wherein the immunoreceptor comprises the antigen-binding domain, wherein
   I) the first domain comprises a heavy chain variable domain;
   II) the first domain comprises a light chain variable domain;
   III) the first domain comprises a heavy chain variable domain, and the second domain comprises a light chain variable domain;
   IV) the first domain comprises a heavy chain variable domain, and the second domain comprises a heavy chain variable domain; or
   V) the first domain comprises a light chain variable domain, and the second domain comprises a light chain variable domain.
13. The immunoreceptor according to any one of items 9 to 12, wherein the immunoreceptor comprises the antigen-binding domain, said antigen-binding domain comprising the first domain, linker, and second domain, which are part of a single chain variable fragment (scFv),
   wherein the scFv optionally comprises, as heavy/light chain variable sequences comprised in the first/second domain, heavy/light chain variable sequences of scFvs specific for one of the following antigens:
   A) CD19, optionally wherein
      i) the heavy chain variable domain has an amino acid sequence having at least 80% sequence identity, preferably at least 90% sequence identity, to SEQ ID NO: 27,
         the light chain variable domain has an amino acid sequence having at least 80% sequence identity, preferably at least 90% sequence identity, to SEQ ID NO: 28,
         and the scFv is capable of specifically binding to CD19; or
      ii) the heavy chain variable domain has the amino acid sequence of SEQ ID NO: 27 and the light chain variable domain has the amino acid sequence of SEQ ID NO: 28;
   B) CD20, optionally wherein
      i) the heavy chain variable domain has an amino acid sequence having at least 80% sequence identity, preferably at least 90% sequence identity, to SEQ ID NO: 30,
         the light chain variable domain has an amino acid sequence having at least 80% sequence identity, preferably at least 90% sequence identity, to SEQ ID NO: 29,
         and the scFv is capable of specifically binding to CD20; or
      ii) the heavy chain variable domain has the amino acid sequence of SEQ

ID NO: 30 and the light chain variable domain has the amino acid sequence of SEQ ID NO: 29;
C) ROR1, optionally wherein
   i) the heavy chain variable domain has an amino acid sequence having at least 80% sequence identity, preferably at least 90% sequence identity, to SEQ ID NO: 31, 33, 35, or 37,
      the light chain variable domain has an amino acid sequence having at least 80% sequence identity, preferably at least 90% sequence identity, to SEQ ID NO: 32, 34, 36, or 38, respectively,
      and the scFv is capable of specifically binding to ROR1; or
   ii) the heavy chain variable domain has the amino acid sequence of SEQ ID NO: 31, 33, 35, or 37 and the light chain variable domain has the amino acid sequence of SEQ ID NO: 32, 34, 36, 38, respectively;
D) ROR2, optionally wherein
   i) the heavy chain variable domain has an amino acid sequence having at least 80% sequence identity, preferably at least 90% sequence identity, to SEQ ID NO: 39,
      the light chain variable domain has an amino acid sequence having at least 80% sequence identity, preferably at least 90% sequence identity, to SEQ ID NO: 40,
      and the scFv is capable of specifically binding to ROR2; or
   ii) the heavy chain variable domain has the amino acid sequence of SEQ ID NO: 39 and the light chain variable domain has the amino acid sequence of SEQ ID NO: 40;
E) SLAMF7, optionally wherein
   i) the heavy chain variable domain has an amino acid sequence having at least 80% sequence identity, preferably at least 90% sequence identity, to SEQ ID NO: 41 or 43,
      the light chain variable domain has an amino acid sequence having at least 80% sequence identity, preferably at least 90% sequence identity, to SEQ ID NO: 42 or 44, respectively,
      and the scFv is capable of specifically binding to SLAMF7; or
   ii) the heavy chain variable domain has the amino acid sequence of SEQ ID NO: 41 or 43 and the light chain variable domain has the amino acid sequence of SEQ ID NO: 42 or 44, respectively;
F) FLT3, optionally wherein
   i) the heavy chain variable domain has an amino acid sequence having at least 80% sequence identity, preferably at least 90% sequence identity, to SEQ ID NO: 45 or 47,
      the light chain variable domain has an amino acid sequence having at least 80% sequence identity, preferably at least 90% sequence identity, to SEQ ID NO: 46 or 48, respectively,
      and the scFv is capable of specifically binding to FLT3; or
   ii) the heavy chain variable domain has the amino acid sequence of SEQ ID NO: 45 or 47 and the light chain variable domain has the amino acid sequence of SEQ ID NO: 46 or 48, respectively;
G) Siglec-6, optionally wherein
   i) the heavy chain variable domain has an amino acid sequence having at least 80% sequence identity, preferably at least 90% sequence identity, to SEQ ID NO: 49,
      the light chain variable domain has an amino acid sequence having at least 80% sequence identity, preferably at least 90% sequence identity, to SEQ ID NO: 50,
      and the scFv is capable of specifically binding to Siglec-6; or
   ii) the heavy chain variable domain has the amino acid sequence of SEQ ID NO: 49 and the light chain variable domain has the amino acid sequence of SEQ ID NO: 50;
H) αᵥβ₃ integrin, optionally wherein
   i) the heavy chain variable domain has an amino acid sequence having at least 80% sequence identity, preferably at least 90% sequence identity, to SEQ ID NO: 51 or 53,
      the light chain variable domain has an amino acid sequence having at least 80% sequence identity, preferably at least 90% sequence identity, to SEQ ID NO: 52 or 54, respectively,
      and the scFv is capable of specifically binding to αᵥβ₃integrin; or
   ii) the heavy chain variable domain has the amino acid sequence of SEQ ID NO: 51 or 53 and the light chain variable domain has the amino acid sequence of SEQ ID NO: 52 or 54, respectively;
or
I) BCMA, optionally wherein
   i) the heavy chain variable domain has an amino acid sequence having at least 80% sequence identity, preferably at least 90% sequence identity, to SEQ ID NO: 55 or 57,
      the light chain variable domain has an amino acid sequence having at least 80% sequence identity, preferably at least 90% sequence identity, to SEQ ID NO: 56 or 58, respectively,
      and the scFv is capable of specifically binding to BCMA; or
   ii) the heavy chain variable domain has the amino acid sequence of SEQ ID NO: 55 or 57 and the light chain variable domain has the amino acid sequence of SEQ ID NO: 56 or 58, respectively.
14. The immunoreceptor according to any one of items 9 to 13, wherein the immunoreceptor comprises the antigen-binding domain, said antigen-binding domain comprising an scFv:
   I) specific to CD19, optionally wherein said scFv comprises an amino acid sequence having at least 80% sequence identity, preferably at least 90% sequence identity, to SEQ ID NO: 3 or 71 and is capable of specifically binding to CD19, or wherein said scFv has the amino acid sequence of SEQ ID NO: 3 or 71;
   II) specific to CD20 optionally wherein said scFv comprises an amino acid sequence having at least 80% sequence identity, preferably at least 90% sequence identity, to SEQ ID NO: 4 or 72 and is capable of specifically binding to CD20, or wherein said scFv has the amino acid sequence of SEQ ID NO: 4 or 72;
   III) specific to ROR1, optionally wherein said scFv comprises an amino acid sequence having at least 80% sequence identity, preferably at least 90% sequence identity, to SEQ ID NO: 5, 6, 7, 8, 73, 74, 75, 76, 87, 88, 89, 90, 91, 92, 93, 94, 95, 96, 97, 98, 99 or 100 and is capable of specifically binding to ROR1, or wherein said scFv has the amino acid sequence of SEQ ID NO: 5, 6, 7, 8, 73, 74, 75, 76, 87, 88, 89, 90, 91, 92, 93, 94, 95, 96, 97, 98, 99 or 100;
   IV) specific to ROR2, optionally wherein said scFv comprises an amino acid sequence having at least 80% sequence identity, preferably at least 90% sequence identity, to SEQ ID NO: 9, 77, 101, 102, 103, 104, 105, 106, 107 or 108 and is capable of specifically binding to ROR2, or wherein said scFv has the amino acid sequence of SEQ ID NO: 9, 77, 101, 102, 103, 104, 105, 106, 107 or 108;
   V) specific to SLAMF7, optionally wherein said scFv comprises an amino acid sequence having at least 80% sequence identity, preferably at least 90% sequence identity, to SEQ ID NO: 10, 11, 78 or 79 and is capable of specifically binding to SLAMF7, or wherein said scFv has the amino acid sequence of SEQ ID NO: 10, 11, 78 or 79;
   VI) specific to FLT3, optionally wherein said scFv comprises an amino acid sequence having at least 80% sequence identity, preferably at least 90% sequence identity, to SEQ ID NO: 12, 13, 80 or 81 and is capable of specifically binding to FLT3, or wherein said scFv has the amino acid sequence of SEQ ID NO: 12, 13, 80 or 81;
   VII) specific to Siglec-6, optionally wherein said scFv comprises an amino acid sequence having at least 80% sequence identity, preferably at least 90% sequence identity, to SEQ ID NO: 14 or 82 and is capable of specifically binding to Siglec-6, or wherein said scFv has the amino acid sequence of SEQ ID NO: 14 or 82;
   VIII) specific to αᵥβ₃ integrin, optionally wherein said scFv comprises an amino acid sequence having at least 80% sequence identity, preferably at least 90% sequence identity, to SEQ ID NO: 15, 16, 83 or 84 and is capable of specifically binding to αᵥβ₃integrin, or wherein said scFv has the amino acid sequence of SEQ ID NO: 15, 16, 83 or 84;
   IX) specific to BCMA, optionally wherein said scFv comprises an amino acid sequence having at least 80% sequence identity, preferably at least 90% sequence identity, to SEQ ID NO: 17, 18, 85 or 86 and is capable of specifically binding to BCMA, or wherein said scFv has the amino acid sequence of SEQ ID NO: 17, 18, 85 or 86.
15. The immunoreceptor according to any one items 1 to 14, wherein the immunoreceptor is a chimeric antigen receptor (CAR).
16. The immunoreceptor or CAR according to any one of items 1 to 15, wherein the one or more IgG3 middle hinge domain repeat motifs
   I) Are from a human IgG3 middle hinge; and/or
   II) Consist of the amino acid sequence of SEQ ID NO: 1; and/or
   III) Have reduced immunogenicity compared to repeats of an IgG1 hinge domain and/or an IgG4 hinge domain.
17. The immunoreceptor or CAR according to any one of items 1 to 16, wherein the immunoreceptor or CAR:
   I) Does not comprise all or part of the sequence of the lower hinge domain of a human IgG3 hinge domain;
   II) Comprises an amino acid sequence which has at least 80% sequence identity, preferably at least 90% sequence identity, or most preferably 100% sequence identity
      with the amino acid sequence of [A-Bₙ],
      wherein
      A is the amino acid sequence of SEQ ID NO: 2;
      B is said IgG3 middle hinge domain repeat motif, wherein said motif has the amino acid sequence of SEQ ID NO: 1; and
      n is selected from the group consisting of 0, 1, 2, 3, 4, 5, 6, 7, 8, 9, 10, 11, 12, 13, 14 and 15 and is preferably an integer between 1 and 15, more preferably an integer between 1 and 10, even more preferably an integer between 1 and 5, most preferably an integer between 3 and 5;
   III) Comprises the IgG3 middle hinge domain repeat motif 1, 2, 3, 4, 5, 6, 7, 8, 9 or 10 times; and/or
   IV) has reduced immunogenicity compared to a second CAR which differs from the first CAR in that it does not comprise said one or more IgG3 middle hinge domain repeat motifs.
18. The immunoreceptor or CAR according to any one of items 1 to 17, wherein the immunoreceptor or CAR comprises at least two, preferably at least three of said IgG3 middle hinge domain repeat motifs which are adjacent to each other.
19. A CAR according to any one of the preceding items, comprising an amino acid sequence selected from the group consisting of SEQ ID NO: 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, and 171.
20. A nucleic acid encoding the immunoreceptor or CAR according to any one of items 1 to 19.
21. A cell, comprising the nucleic acid according to item 20.
22. The cell according to item 21, wherein:
   I) The cell is an immune cell, preferably a B cell, macrophage, NK cell or T cell, more preferably T cell, and even more preferably a CD4⁺ and/or CD8⁺ T cell;
   II) The cell expresses the immunoreceptor or CAR according to any one of items 1 to 19;
   III) The cell comprises the nucleic acid stably integrated into the genome; and/or
   IV) The nucleic acid comprised in the cell is comprised in an episomal vector.
23. The nucleic acid, cell comprising the nucleic acid, immunoreceptor, or CAR, according to any one of items 1 to 22 for use in a method of treating a cancer, an autoimmune disease, an infectious disease or a degenerative disease.
24. The immunoreceptor, CAR, nucleic acid or cell comprising the nucleic acid for use of item 23, wherein the disease is a cancer, wherein the cancer is is a hematological cancer or a solid cancer,
   optionally wherein the hematological cancer is leukemia or lymphoma, preferably acute myeloid leukemia, multiple myeloma, non-Hodgkin-lymphoma, Burkitt's lymphoma, mantle cell lymphoma, acute lymphoblastic leukemia, chronic lymphocytic leukemia, or diffuse large B cell lymphoma;
   optionally wherein the solid cancer is breast cancer, colon carcinoma, lung cancer, pancreatic or prostate cancer or glioblastoma.
25. An antigen-binding protein, streptamer or aptamer which is capable of binding to an epitope comprised by a sequence consisting of at least one, preferably at least two, more preferably at least three repeats of the amino acid sequence of SEQ NO: 1, optionally wherein at least two repeats are adjacent to each other.
26. The antigen-binding protein, streptamer or aptamer of item 25, wherein the antigen-binding protein, streptamer or aptamer is capable of binding to the immunoreceptor or CAR according to any one of items 1 to 19.
27. The antigen-binding protein, streptamer or aptamer of item 26, wherein the antigen-binding protein, streptamer or aptamer is capable of stimulating the immunoreceptor or CAR according to any one of items 1 to 19.
28. The antigen-binding protein, streptamer or aptamer according to any one of items 25 to 27, wherein the antigen-binding protein, streptamer or aptamer is an antigen-binding protein which is an antibody or fragment thereof, preferably a monoclonal antibody or fragment thereof.
29. The antigen-binding protein of any one of items 25 to 28, wherein the antigen-binding protein comprises
   a) a heavy chain variable region having at least 80%, preferably at least 90% sequence identity with the amino acid sequence of SEQ ID NO: 19, and wherein the heavy chain variable region preferably contains a CDR1 having the amino acid sequence of SEQ ID NO: 20, a CDR2 having the amino acid sequence of SEQ ID NO: 21, and a CDR3 having the amino acid sequence of SEQ ID NO: 22; and
   b) a light chain variable region having at least 80%, preferably at least 90% sequence identity with the amino acid sequence of SEQ ID NO: 23, wherein the light chain variable region preferably contains a CDR1 having the amino acid sequence of SEQ ID NO: 24, a CDR2 having the amino acid sequence of SEQ ID NO: 25, and a CDR3 having the amino acid sequence of SEQ ID NO: 26.
30. Use of the antigen-binding protein, streptamer or aptamer according to any one of items 25 to 29 for purification, detection, depletion, stimulation, expansion, or enrichment of cells expressing the immunoreceptor or CAR as defined in any one of items 1 to 19.
31. A method, comprising the step of:

Binding an antigen-binding protein, streptamer or aptamer to cells expressing the immunoreceptor or CAR as defined in any one of items 1 to 19, preferably wherein the binding is binding specifically to the IgG3 middle hinge repeat domain comprised in said immunoreceptor or CAR, and/or wherein the antigen-binding protein, streptamer or aptamer is an antigen-binding protein, streptamer or aptamer as defined in any one of items 25 to 29.
32. The method of item 31, wherein the method is a method of purification of cells expressing the immunoreceptor or CAR as defined in any one of items 1 to 19, comprising the steps of:
   A) Optionally obtaining the cells expressing the chimeric antigen receptor;
   B) Incubating said cells with a primary antibody, streptamer or aptamer, wherein the primary antibody, streptamer or aptamer is said antigen-binding protein, streptamer or aptamer as defined in any one of items 25 to 29, under conditions which allow the antibody, streptamer or aptamer to bind to the immunoreceptor or CAR expressed by the cells;
   C) Separating the antibody-, streptamer- or aptamer-bound cells from the non-bound cells in order to obtain the purified cells.
33. The purification method of item 32, wherein step C comprises incubating the cells of step B with an entity capable of binding to the antibody, streptamer or aptamer; and wherein
   I) The entity is preferably a secondary antibody, more preferably labelled with a fluorescent marker; or a bead, more preferably a magnetic bead;
   II) The primary antibody, streptamer or aptamer is labelled, wherein the label is preferably a tag or a fluorescent dye;
   III) The separation of step C is carried out by means of MACS or FACS; and/or
   IV) Wherein the separation is carried out using a Streptamer or an Aptamer.
34. The method of item 31, wherein the method is a method of depletion of cells expressing the immunoreceptor or CAR as defined in any one of items 1 to 19, comprising the steps of:
   A) Optionally obtaining the cells expressing the immunoreceptor or CAR; and
   B) Incubating said cells with an antigen-binding protein, streptamer or aptamer as defined in any one of items 25 to 29 coupled to a cytotoxic molecule.
35. The method of item 31, wherein the method is a method of a) stimulation and/or b) expansion of cells expressing the immunoreceptor or CAR as defined in any one of items 1 to 19, comprising the steps of:
   A) Optionally obtaining the cells expressing the immunoreceptor or CAR; and
   B) Incubating said cells with an antigen-binding protein, streptamer or aptamer as defined in any one of items 25 to 29, optionally wherein the antigen-binding protein, streptamer or aptamer is coupled to a solid phase, or wherein the antigen-binding protein, streptamer or aptamer is expressed on the surface of a cell.
36. The stimulation or expansion method of item 35, wherein:
   I) The solid phase is a tissue culture surface or a bead, preferably a magnetic bead; and/or
   II) The solid phase is a scaffold consisting of polymers, preferably starch or sugar.
37. The method of item 31, wherein the method is a method of enrichment of cells expressing the immunoreceptor or CAR as defined in any one of items 1 to 19, comprising the steps of:
   A) Stimulating and/or expanding the cells according to the method of items 35 or 36; and
   B) Purifying the cells of step A according to the method of item 32 or 33.
38. The method or use of any one of items 30 to 37, wherein said method or use is an in vitro method or use.
39. The method or use of any one of items 30 to 38, wherein said method or use does not comprise a method for treatment of the human or animal body by surgery or therapy or a diagnostic method practised on the human or animal body.
40. A pharmaceutical composition, comprising the antigen-binding protein, streptamer or aptamer according to any one of items 25 to 29 or a cell expressing a chimeric antigen receptor comprising all or part of said antigen-binding protein, streptamer or aptamer, the composition optionally further comprising a pharmaceutically acceptable carrier and/or excipient.
41. The antigen-binding protein, streptamer or aptamer according to any one of items 25 to 29 or a cell expressing a chimeric antigen receptor comprising all or part of said antigen-binding protein, streptamer or aptamer, or the pharmaceutical composition of item 40, for use in a therapeutic method of depletion of cells expressing the immunoreceptor or CAR as defined in any one of items 1 to 19, comprising administering to a subject in need thereof said antigen-binding protein, streptamer or aptamer coupled to a cytotoxic molecule or cells expressing said chimeric antigen receptor comprising said all or part of said antigen-binding protein, streptamer or aptamer.
42. A kit, comprising the immunoreceptor or CAR as defined in any one of items 1 to 19 and the antigen-binding protein, streptamer or aptamer as defined in any one of items 25 to 27.
43. A bispecific antibody, comprising one or more IgG3 middle hinge repeat domain motifs.
44. The bispecific antibody according to item 43, wherein the one or more IgG3 middle hinge domain repeat motifs
   I) Are from a human IgG3 middle hinge; and/or
   II) Consist of the amino acid sequence of SEQ ID NO: 1; and/or
   III) Have reduced immunogenicity compared to repeats of an IgG1 hinge domain and/or an IgG4 hinge domain.
45. The bispecific antibody according to items 43 or 44, wherein the bispecific antibody:
   I) Does not comprise all or part of the sequence of the lower hinge domain of a human IgG3 hinge domain;
   II) Comprises an amino acid sequence which has at least 80% sequence identity, preferably at least 90% sequence identity, or most preferably 100% sequence identity
      with the amino acid sequence of [A-Bₙ],
      wherein
      A is the amino acid sequence of SEQ ID NO: 2;
      B is said IgG3 middle hinge domain repeat motif, wherein said motif has the amino acid sequence of SEQ ID NO: 1; and
      n is selected from the group consisting of 0, 1, 2, 3, 4, 5, 6, 7, 8, 9, 10, 11, 12, 13, 14 and 15 and is preferably an integer between 1 and 15, more preferably an integer between 1 and 10, even more preferably an integer between 1 and 5, most preferably an integer between 3 and 5;
   III) Comprises the IgG3 middle hinge domain repeat motif 1, 2, 3, 4, 5, 6, 7, 8, 9 or 10 times; and/or
   IV) has reduced immunogenicity compared to a second bispecific antibody which differs from the first bispecific antibody in that it does not comprise said one or more IgG3 middle hinge domain repeat motifs.
46. The bispecific antibody according to any one of items 43 to 45, wherein the immunoreceptor comprises an amino acid sequence which has 100% sequence identity with the amino acid sequence of [A-Bₙ].
47. The bispecific antibody according to item 45 or 46, wherein n is an integer between 1 and 10.
48. The immunoreceptor according to item 45 or 46, wherein n is an integer between 1 and 5.
49. The immunoreceptor according to item 45 or 46, wherein n is an integer between 3 and 5.
50. The bispecific antibody according to any one of items 43 to 49, comprising at least two, preferably at least three IgG3 middle hinge repeat domain motifs, optionally wherein at least two of said IgG3 middle hinge repeat domain motifs are adjacent to each other.
51. The immunoreceptor, CAR, nucleic acid, cell, method, pharmaceutical composition, kit or bispecific antibody according to any one of items 1 to 24 or 31 to 50, wherein the IgG3 middle hinge repeat domain motif is not a mouse IgG3 middle hinge repeat domain.

### BRIEF DESCRIPTION OF THE DRAWINGS

FIG. 1. CAR design. A. Schematic illustration of CARs carrying IgG3-derived spacers of different lengths.

FIG. 2. CD19 CAR T cells equipped with IgG3-derived spacers are functional in vitro. A. Antigen-specific proliferation. CD8⁺ T cells were labelled with CFSE and stimulated using irradiated K562 tumor cells with or without CD19 expression at a 4:1 E:T ratio. Proliferation as visualized by dilution of CFSE was determined after 72 h. No exogenous cytokines were added. Representative example for n=3 independent experiments. B. Specific cytolytic activity of CD8⁺ CD19-CAR T cells equipped with different spacer domains and untransduced T cells against CD19⁺ Jeko-1 cells in a bioluminescence-based assay (5-hour incubation). Assay was performed in triplicate wells with 5,000 target cells/well. Values are presented as mean±SEM from n=3 independent experiments. C. ELISA to detect IFNγ in supernatant obtained from 24-hour co-cultures of CD8⁺ CD19 CAR T cells with K562 target cells with or without CD19 expression. T cells (50,000/well) and target cells (12,500/well) were seeded in triplicate wells. Values are presented as mean±SEM from n=3 independent experiments.

FIG. 3. ROR1 CAR T cells equipped with IgG3-derived spacers are functional in vitro. Assessing of in vitro functions for ROR1-specific CARs based on the scFv 4-2. A Antigen-specific proliferation. CD8⁺ T cells were labelled with CFSE and stimulated using irradiated K562 tumor cells with or without expression of ROR1 (K562_ROR1) at a 4:1 E:T ratio. Proliferation as visualized by dilution of CFSE was determined after 72 h. No exogenous cytokines were added. Representative example for n=3 independent experiments. B. Specific cytolytic activity of CD8⁺ ROR1-CAR T cells equipped with different spacer domains and untransduced T cells against K562_ROR1 in a bioluminescence-based assay (5-hour incubation). Assay was performed in triplicate wells with 5,000 target cells/well. Values are presented as mean±SEM from n=3 independent experiments. C. ELISA to detect IFNγ in supernatant obtained from 24-hour co-cultures of CD8⁺ ROR1 CAR T cells with K562 cells with or without expression of ROR1. T cells (50,000/well) and target cells (12,500/well) were seeded in triplicate wells. Values are presented as mean±SEM from n=3 independent experiments.

FIG. 4. ROR1 CAR T cells equipped with IgG3-derived spacers are functional in vitro. Assessing of in vitro functions for ROR1-specific CARs based on the scFv R11. A,E. Antigen-specific proliferation. CD8⁺ T cells were labelled with CFSE and stimulated using irradiated K562 tumor cells with or without expression of ROR1 (K562_ROR1) or a modified version of ROR1 with increased distance between R11 epitope and tumor cell membrane (K562_ROR1/E3AK) at a 4:1 E:T ratio. Proliferation as visualized by dilution of CFSE was determined after 72 h. No exogenous cytokines were added. Representative example for n=3 independent experiments. B,F. Specific cytolytic activity of CD8⁺ ROR1-CAR T cells equipped with different spacer domains and untransduced T cells against K562_ROR1 (B) or K562_ROR1/E3AK (F) in a bioluminescence-based assay (5-hour incubation). Assay was performed in triplicate wells with 5,000 target cells/well. Values are presented as mean±SEM from n=3 independent experiments. C,G. ELISA to detect IFNγ in supernatant obtained from 24-hour co-cultures of CD8⁺ ROR1 CAR T cells and K562_ROR1 (C) or K562_ROR1/E3AK (G). T cells (50,000/well) and target cells (12,500/well) were seeded in triplicate wells. Values are presented as mean±SEM from n=3 independent experiments. D. Schematic illustration of how the R11 epitope in ROR1 is moved further away from the tumor cell membrane using the E3AK linker.

FIG. 5. CD20 CAR T cells equipped with IgG3-derived spacers are functional in vitro. A. Antigen-specific proliferation. CD8⁺ T cells were labelled with CFSE and stimulated using irradiated K562 tumor cells with or without CD20 expression at a 4:1 E:T ratio. Proliferation as visualized by dilution of CFSE was determined after 72 h. No exogenous cytokines were added. Representative example for n=3 independent experiments. B. Specific cytolytic activity of CD8⁺ CD20-CAR T cells equipped with different spacer domains and untransduced T cells against CD20⁺ Raji cells in a bioluminescence-based assay (3-hour incubation). Assay was performed in triplicate wells with 5,000 target cells/well. Values are presented as mean±SEM from n=3 independent experiments. C. ELISA to detect IFNγ in supernatant obtained from 24-hour co-cultures of CD8⁺ CD20 CAR T cells with K562 target cells with or without CD20 expression. T cells (50,000/well) and target cells (12,500/well) were seeded in triplicate wells. Values are presented as mean±SEM from n=2 independent experiments.

FIG. 6. SLAMF7 CAR T cells equipped with IgG3-derived spacers are functional in vitro. A. Antigen-specific proliferation. CD4⁺ T cells were labelled with CFSE and stimulated using irradiated K562 tumor cells with or without SLAMF7 expression at a 4:1 E:T ratio. Proliferation as visualized by dilution of CFSE was determined after 72 h. No exogenous cytokines were added. Representative example for n=3 independent experiments. B. Specific cytolytic activity of CD8⁺ SLAMF7-CAR T cells equipped with different spacer domains and untransduced T cells against SLAMF7⁺ MM.1S cells in a bioluminescence-based assay (3-hour incubation). Assay was performed in triplicate wells with 5,000 target cells/well. Values are presented as mean±SEM from n=3 independent experiments. C. ELISA to detect IFNγ in supernatant obtained from 24-hour co-cultures of CD4⁺ SLAMF7 CAR T cells with K562 or MM.1S target cells. T cells (50,000/well) and target cells (12,500/well) were seeded in triplicate wells. Values are presented as mean±SEM from n=2 independent experiments.

FIG. 7. ROR2 CAR T cells equipped with IgG3-derived spacers are functional in vitro. A. Antigen-specific proliferation. CD8⁺ T cells were labelled with CFSE and stimulated using irradiated MDA-MB231 tumor cells with or without ROR2 expression at a 4:1 E:T ratio. Proliferation as visualized by dilution of CFSE was determined after 72 h. No exogenous cytokines were added. Representative example for n=3 independent experiments. B. Specific cytolytic activity of CD8⁺ ROR2-CAR T cells equipped with different spacer domains and untransduced T cells against ROR2⁺ U266 cells in a bioluminescence-based assay (3-hour incubation). Assay was performed in triplicate wells with 5,000 target cells/well. Values are presented as mean±SEM from n=3 independent experiments. C. ELISA to detect IFNγ in supernatant obtained from 24-hour co-cultures of CD8⁺ ROR2 CAR T cells with MDA-MB231 target cells with or without ROR2 expression. T cells (50,000/well) and target cells (12,500/well) were seeded in triplicate wells. Values are presented as mean±SEM from n=2 independent experiments.

FIG. 8. CD19 CAR T cells equipped with IgG3-derived spacers are functional in vivo. NSG mice were inoculated with 1×10⁶ Raji cells (ffluc+GFP+) and were treated on day 7 with 5×10⁶ CD8⁺ CD19-CAR T cells or were left untreated. A. Serial bioluminescence imaging to assess leukemia progression/regression in each treatment group. B. Kaplan-Meier analysis of survival of mice shown in panel A in groups of mice treated with CD19-CAR T cells (n=3) or untransduced T cells (n=2). Statistical analyses were conducted using the log-rank test; * p<0.01. C. Additional 6 mice were inoculated with 1×10⁶ Raji cells (ffluc⁺GFP⁺) and were treated on day 7 with 5×10⁶ CD8⁺ CD19-CAR T cells (IgG3_MiH3 variant or IgG4 control CAR). After 7 days, mice were sacrificed and analyzed for the presence of CAR T cells in peripheral blood, bone marrow and spleen.

FIG. 9. ROR1 CAR T cells equipped with IgG3-derived spacers are functional in vivo. NSG mice were inoculated with 1×10⁶ Jeko-1 cells (ffluc⁺GFP⁺) and were treated on day 7 with 5×10⁶ CD8⁺ ROR1-CAR T cells (R11 scFv), or were left untreated. Kaplan-Meier analysis of survival in groups of mice treated with R11 ROR1-CAR T cells (n=5) or untransduced T cells (n=5).

FIG. 10. Design and Detection of CARs carrying an additional multifunction site. A. Direct staining of CAR surface expression using the anti-MiH antibody #1 in CD8⁺ T cells transduced with CD19 CARs equipped with different IgG3 spacers or the IgG4 control CAR. B. Schematic illustration of the IgG4 reference CAR and 1^{st} generation of IgG3 spacer CARs or the advanced IgG3 format with additional multifunction site.

FIG. 11. The advanced IgG3 CAR format with additional MFS provides potent CD19 CAR T antitumor function in vitro and in vivo. A. Antigen-specific proliferation. CD8⁺ T cells were labelled with CFSE and stimulated using irradiated K562 tumor cells with or without CD19 expression at a 4:1 E:T ratio. Proliferation as visualized by dilution of CFSE was determined after 72 h. No exogenous cytokines were added. Representative example for n=3 independent experiments. B. Specific cytolytic activity of CD8⁺ CD19-CAR T cells equipped with different spacer domains±the additional IgG3-based multifunction site between scFv V_{H} and V_{L} or untransduced T cells against CD19⁺ Jeko-1 cells in a bioluminescence-based assay (3-hour incubation). Assay was performed in triplicate wells with 5,000 target cells/well. Values are presented as mean±SEM from n=3 independent experiments. C. ELISA to detect IFNγ in supernatant obtained from 24-hour co-cultures of CD8+ CD19 CAR T cells with K562 target cells with or without CD19 expression. T cells (50,000/well) and target cells (12,500/well) were seeded in triplicate wells. Values are presented as mean±SEM from n=2 independent experiments. D. NSG mice were inoculated with 1×10⁶ Raji cells (ffluc⁺GFP⁺) and were treated on day 7 with 5×10⁶ CD19-CAR T cells (CD4⁺:CD8⁺ ratio 1:1) or were left untreated. Serial bioluminescence imaging was conducted to assess leukemia progression/regression in each treatment group. E. Kaplan-Meier analysis of survival of mice shown in panel D in groups of mice treated with CD19-CAR T cells (n=5) or untransduced T cells (n=5).

FIG. 12. Enrichment of CAR T cells by targeting the multifunction site(s). CD8⁺ CAR T cells were mixed with Mock T cells at a 1:1 ratio and labeled with either anti-MiH antibody #1 or anti-EGFRt antibody, both in a biotinylated form. Cells were washed, labeled with anti-Biotin-MicroBeads, washed again, isolated using Miltenyi MACS LS columns and analyzed by flow cytometry the next day. A. Purity of positive fractions after sort as percentage of CD8⁺EGFRt⁺ cells for IgG3-spacer CARS vs. the IgG4 format (n=4 independent experiments). B. Efficiency of enrichment in A, shown is percentage as the number of EGFRt⁺ cells in the positive fraction divided by the total number of EGFRt⁺ cells in positive+negative fractions (n=4 independent experiments). Upper Panel shows an example for purity of CD19_IgG3_MiH5 CAR T cells after sort as measured by flow cytometry. C. Purity of positive fractions after sort as percentage of CD8⁺EGFRt⁺ cells for a first generation IgG3-spacer CAR (R11_IgG3_MiH3) or the advanced IgG3 format (R11_IgG3_MiH5/MiH3) (n=3 independent experiments). D. Efficiency of enrichment in C, shown is percentage as the number of EGFRt⁺ cells in the positive fraction divided by the total number of EGFRt⁺ cells in positive+negative fractions (n=3 independent experiments). Values depicted are calculated as foldchange over cells treated with medium only.

FIG. 13. Activation and expansion of CAR T cells by targeting the IgG3 spacer. CD8⁺ CAR T cells equipped with different IgG3-based spacers were plated in triplicates on 96 well plates precoated with 5 μg/mlanti-MiH antibody #1 and cultured either for 24 h (A,B) followed by flowcytometric analysis of CD25 (A) and CD69 (B) expression, or for 7 days for expansion assays (C, upper panel), followed by counting of the cells (C, lower panel). Asterisks indicate statistical significance established by Wilcoxon test, p<0.05.

FIG. 14. Induction of CAR T proliferation by targeting the multi-function site(s). CD8⁺ T cells were labelled with CFSE and stimulated using irradiated K562 tumor cells with or without expression of the anti-IgG3 Hinge CAR (K562_Anti-CAR) at a 4:1 E:T ratio, or Dynabeads® coated with either anti-CD3/anti-CD28, anti-MiH antibody #1, anti-MiH antibody #1/anti-CD28 or anti-MiH antibody #1/anti-4-1BB at a Bead to T cell ratio of 1.6. Proliferation as visualized by dilution of CFSE was determined after 72 h. No exogenous cytokines were added. A. Schematic illustration of agents used to induce proliferation. B. Representative examples from n=3 independent experiments for a first generation IgG3-spacer CAR (CD19_IgG3_MiH1, (left panel) or the advanced IgG3 format (CD19_IgG3_MiH5/MiH1, right panel).

FIG. 15. ADC-mediated depletion of CAR T cells by targeting the IgG3 spacer. 5×10⁴ CD8⁺ CAR T cells equipped with different lengths of IgG3-derived spacers or the IgG4 reference CAR, as well as untransduced T cells were plated in triplicate wells and treated with different concentrations of anti-MiH antibody #1-ADC (anti-MiH antibody #1, conjugated to an anthracycline-based cytotoxic payload). Cells were cultivated in the presence of 50 IU IL-2 for 72 h, washed and subjected to flowcytometric analysis. The percentage of viable cells depicted is the normalized cell count of the treated samples divided by the normalized cell count of the respective cells cultured in medium only (n=5 independent experiments).

FIG. 16. ADC-mediated depletion of CAR T cells by targeting the multi-function site(s) in vitro. 5×10⁴ CD8⁺ CAR T cells equipped with either the optimized IgG3 spacer version, the optimized IgG3 spacer version+additional multi-function site between scFv VH and VL or the IgG4 reference CAR, as well as untransduced T cells were plated in triplicate wells and treated with different concentrations of anti-MiH antibody #1-ADC (anti-MiH antibody #1, conjugated to an anthracycline-based cytotoxic payload). Cells were cultivated in the presence of 50 IU IL-2 for 72 h, washed and subjected to flowcytometric analysis. The percentage of viable cells depicted is the normalized cell count of the treated samples divided by the normalized cell count of the respective cells cultured in medium only. A. ADC assay with CD19 CARs (CD19_IgG3_MiH1 vs. CD19_IgG3_MiH5/MiH1 vs. CD19_IgG4), data from n=3 independent experiments. B. ADC assay with CD20 CARs (CD2O_IgG3_MiH3 vs. C20_IgG3_MiH5/MiH3 vs. CD20_IgG4), data from n=2 independent experiments. C. ADC assay with ROR1 CARs (R11_IgG3_MiH3 vs. R11_IgG3_MiH5/MiH3 vs. R11_IgG4), data from n=2 independent experiments.

FIG. 17. Depletion of CAR expressing cells by targeting with an Anti-IgG3 Hinge CAR in vitro. Specific cytolytic activity of CD8⁺ T cells equipped with an Anti-CAR (anti-MiH antibody #1-based CAR with IgG4 spacer) against K562 cells transduced with the CD19-CAR CD19_MiH5 (A) or K562 cells (B) in a bioluminescence-based assay (5-hour incubation). Assay was performed in triplicate wells with 5,000 target cells/well. Values are presented as mean±SEM from n=3 independent experiments.

FIG. 18. ADC-mediated depletion of CAR T cells in vivo. NSG mice were inoculated with 4.5×10⁶ CD4⁺ T cells transduced with the advanced version of the IgG3-based CD19 CAR (CD19_IgG3_MiH5/MiH1) as well as with a ffluc_GFP fusion protein. At day 8, half of the mice (n=8 animals per group) were treated with 100 μg of anti-MiH antibody #1 ADC (corresponding to 4.5 mg/kg bodyweight). At d11, T cells were restimulated with irradiated K562 cells equipped with an anti-MiH antibody #1-based Anti-CAR (1×10\hat{ }6 irradiated K562_Anti-CAR cells per mice). A. Kinetics of T cell persistence were assessed by serial bioluminescence imaging. B. Endpoint bioluminescence at d18.

FIG. 19. CAR-specific stimulation of CAR T cells in vivo. NSG mice were inoculated with 4.5×10⁶ CD4⁺ T cells transduced with the advanced version of the IgG3-based CD19 CAR (CD19_IgG3_MiH5/MiH1) that have been labeled with the proliferation dye eFluor670. After T cell transfer, groups of n=5 mice were subsequently treated with 3×10\hat{ }6 irradiated K562 or K562_Anti-CAR cells at different time points. One group received K562_Anti-CAR cells at the day of T cell injection (d0), 3 h after T transfer. A second group received an additional dose of irradiated K526_Anti-CAR cells at d3 post T cell injection (d0+d3), two other groups were treated with irradiated K562_Anti-CAR cells at day 1 post T cell transfer (d1) or at d1+d3, respectively. A control group received irradiated K562 cells at d0+d3. At day 4 post T cell transfer, mice were sacrificed, and bone marrow cells were collected. Cells were stained with antibodies against CD4, CD45 and EGFRt and subjected to flow cytometric analysis. CD45⁺/CD4⁺/EGFR⁺ bone-marrow derived T cells were analyzed for eFluor 670 dilution.

FIG. 20. The advanced IgG3 CAR format with additional MFS provides potent ROR1 CAR T antitumor function in vitro. A. Antigen-specific proliferation. CD8⁺ T cells were labelled with CFSE and stimulated using irradiated K562 tumor cells with or without ROR1 expression at a 4:1 E:T ratio. Proliferation as visualized by dilution of CFSE was determined after 72 h. No exogenous cytokines were added. Representative example for n=3 independent experiments. B. Specific cytolytic activity of CD8⁺ ROR1-CAR T cells equipped with different spacer domains±the additional IgG3-based multifunction site between scFv VH and VL or untransduced T cells against ROR1+K562_ROR1 cells in a bioluminescence-based assay (5-hour incubation). Assay was performed in triplicate wells with 5,000 target cells/well. Values are presented as mean±SEM from n=4 independent experiments. C. ELISA to detect IFNγ in supernatant obtained from 24-hour co-cultures of CD8⁺ ROR1 CAR T cells with K562 target cells with or without ROR1 expression. T cells (50,000/well) and target cells (12,500/well) were seeded in triplicate wells. Values are presented as mean±SEM from n=3 independent experiments.

FIG. 21. The advanced IgG3 CAR format with additional MFS provides potent CD19 CAR T antitumor function in vitro. A. Antigen-specific proliferation. CD8⁺ T cells were labelled with CFSE and stimulated using irradiated K562 tumor cells with or without CD19 expression at a 4:1 E:T ratio. Proliferation as visualized by dilution of CFSE was determined after 72 h. No exogenous cytokines were added. Representative example for n=3 independent experiments. B. Specific cytolytic activity of CD8⁺ CD19-CAR T cells equipped with different spacer domains±the additional IgG3-based multifunction site between scFv V_{H} and V_{L} or untransduced T cells against CD19⁺ Jeko-1 cells in a bioluminescence-based assay (3-hour incubation). Assay was performed in triplicate wells with 5,000 target cells/well. Values are presented as mean±SEM from n=3 independent experiments. C. ELISA to detect IFNγ in supernatant obtained from 24-hour co-cultures of CD8⁺ CD19 CAR T cells with K562 target cells with or without CD19 expression. T cells (50,000/well) and target cells (12,500/well) were seeded in triplicate wells. Values are presented as mean±SEM from n=3 independent experiments.

FIG. 22. The advanced IgG3 CAR format with additional MFS provides potent cytotoxic effects in vitro. Specific cytolytic activity of CD8⁺ CAR T cells equipped with different spacer domains±the additional IgG3-based multifunction site between scFv VH and VL or untransduced T cells against Antigen+tumor cells in a bioluminescence-based assay. Assays were performed in triplicate wells with 5,000 target cells/well. Values are presented as mean±SEM from n=2 independent experiments. A. ROR1-specific CAR T cells (4-2 scFv) against ROR1⁺ Jeko-1 cells (3-hour incubation). B. FLT3-specific CAR T cells (4G8 scFv) against FLT3⁺ MOLM-13 cells (5-hour incubation). C. FLT3-specific CAR T cells (BV10 scFv) against FLT3⁺ MOLM-13 cells (5-hour incubation). D. Siglec-6-specific CAR T cells (JML-1 scFv) against Siglec-6⁺ MV4-11 cells (3-hour incubation).

FIG. 23. The advanced IgG3 CAR format with additional MFS provides potent CD19 CAR T antitumor function in vitro. A-B. NSG mice were inoculated with 1×10⁶ Raji cells (ffluc⁺GFP⁺) and were treated on day 7 with 5×10⁶ CD19-CAR T cells (CD4⁺:CD8⁺ ratio 1:1) or were left untreated. Serial bioluminescence imaging was conducted to assess leukemia progression/regression in each treatment group. C. Kaplan-Meier analysis of survival of mice shown in panels A-B in groups of mice treated with CD19-CAR T cells (n=7) or untransduced T cells (n=4).

FIG. 24. Expansion of CAR T cells in vitro by targeting the IgG3 MFS. 5×10⁵ CD4⁺ or CD8⁺ untransduced control T cells or CD4⁺ or CD8⁺ T cells equipped with a CD19-specific CAR in the advanced IgG3 format were expanded in the presence of 5×10⁶ irradiated TM-EBV-LCL (CD19⁺) or K562_Anti-CAR (CD19⁻) feeder cells and 50 IU IL-2 for 14 days, and T cells were counted after 14 days of expansion. For untransduced T cells only, expansion setup contained 30 ng/ml OKT3. Values are presented as x-fold expansion over starting count after 14 days (mean±SEM from n=2 independent experiments).

FIG. 25. Expansion of CAR T cells in vivo by targeting the IgG3 MFS. NSG mice were inoculated with 1×10⁷ ffluc⁺GFP⁺ T cells transduced with CD19-CAR CD19_MiH5/MiH1 (CD4⁺:CD8⁺ ratio 2.7:1) and were treated on d8 with 1×10⁷ K562_Anti-CAR cells or untransduced control K562 cells. Serial bioluminescence imaging was conducted to assess T cell persistence/expansion in the treatment groups.

FIG. 26. Depletion of CAR expressing cells by targeting with an Anti-IgG3 Hinge CAR in vitro and in vivo. A-D. Specific cytolytic activity of CD8⁺ T cells from three different donors equipped with an Anti-CAR (anti-MiH antibody #1-based CAR with IgG4 spacer) against CD4⁺ T cells from the same three donors that were transduced with either firefly-luciferase (A, C) or firefly luciferase and the anti-CD19-CAR CD19_MiH5/MiH1 (B,D) in a bioluminescence-based assay (5-hour incubation). Assay was performed in triplicate wells with 5,000 target cells/well. Values are presented as mean of triplicate wells from n=1 experiment with CD8⁺ Effector Anti-CAR T cells from Donor 1 (A-B) and Donor 2 (C-D), respectively. E-F. 4 NSG mice per group were inoculated with 2.2×10⁶ Target T cells (CD4⁺:CD8⁺ ratio 1:1) from Donor 2 (ffluc⁺GFP⁺+anti-CD19-CAR CD19_MiH5/MiH1) and were treated after 24 h with 4×10⁶ CD8⁺ Anti-CAR-CAR T cells (Donor 2) or untransduced control T cells from the same donor. Serial bioluminescence imaging was conducted to assess T cell persistence/depletion in each treatment group. Note: left mouse depicted in untransduced group at d1 only was not included in the experiment.

### DETAILED DESCRIPTION OF THE INVENTION

During the past decades, the design of chimeric antigen receptors (CARs), previously also termed T-Bodies, evolved from rather simple constructs to more complex molecules assembled from domains of distinct proteins. In their most simple form, nowadays also termed first-generation CARs, they consisted of the scFv of a monoclonal antibody fused to the signaling domains of the CD3ζ subunit¹. Subsequently, it turned out that most CAR constructs require a spacer between scFv and transmembrane domain to induce full T cell effector functions². While from the mid-1990s until now, Fc regions or immunoglobulin-like domains derived from different proteins (including CD4, CD7, CD8α, CD28, IgD, IgG1 and IgG4, with the CD8α Hinge being the most commonly used and best examined one³⁻⁶), most researchers in the field are using only one spacer format for all their CAR constructs. This led to effective results, even though the CAR designs used may not necessarily be the most functional ones; different antibodies bind distinct epitopes on their target molecules and the inventors have shown in previous work that a spacer adjusted in composition and length to optimally fit the target epitope leads to maximum anti-tumor function⁷.

The present invention provides novel variants of the Hinge domain of human IgG3 for incorporation into genetically engineered immunoreceptors, such as incorporation as a spacer domain in CAR constructs.

The inventors generated a library of CARs with IgG3-derived spacers, in which scFv and transmembrane domain are connected by variants of the human IgG3 Hinge domain. This naturally consists of upper hinge (12 aa, ELKTPLGDTTHT, SEQ ID NO: 2), middle hinge (50 aa, CPRCP, SEQ ID NO: 59+3 repeats of the 15 aa motif EPKSCDTPPPCPRCP, SEQ ID NO: 1) and lower hinge (8 aa, APELLGGP, SEQ ID NO: 60), leading to a total spacer size of 70 aa for this wild-type spacer termed IgG3_UMLH (upper, middle and lower hinge). From that the inventors constructed variants consisting of upper hinge (ELKTPLGDTTHT, SEQ ID NO: 2), the n-terminal part of the middle hinge (CPRCP, SEQ ID NO: 59) and 0-10 copies of the EPKSCDTPPPCPRCP motif (SEQ ID NO: 1) leading to spacer domains spanning 17 to 167 aa in 15 aa steps named IgG3_MiHO to IgG3_MiH10.

Other investigators have previously implemented CH2-CH3-Hinge versions of IgG3 as spacer domains in CAR design^{12, 13}. In contrast to the present invention, these researchers have used two variants in length where they removed the upper hinge (ELKTPLGDTTHT, SEQ ID NO: 2) and the start of the middle hinge (CPRCP, SEQ ID NO: 59), but instead additionally used IgG3 CH2 and CH3 domains. These two versions, termed CH2-CH3-Hinge and CH2-CH3-Hinge-Hinge, are both much longer (232 aa or 247 aa), carry FC-binding motifs potentially causing immunogenicity and are due to the inclusion of the relatively stiff CH2 and CH3 regions much less flexible than any of the variants the inventors have included in their present IgG3 spacer library^{12, 13}.

The inventor's data show that an optimal IgG3 spacer configuration can be generated for every target investigated, with the sweet spot depending on the location of the scFv epitope within the target molecule. A general principle is that for epitopes that are located tumor-membrane distally, a shorter variant leads to most potent T cell effector functions, for epitopes that are located membrane-proximally, a longer variant leads to better function. In general, the inventors show that IgG3-based spacers inherit a great flexibility, surpassing that of other formats. In particular, CARs carrying a relatively short IgG3-based spacer of only 62 aa (IgG3_MiH3) outperform IgG4 variants that show best functionality with a very long spacer of 228 aa, thereby reducing the size of the CAR and the genetic cargo that has to be delivered. The reduction of the genetic cargo is associated with several advantageous effects, such as an increase of transfection or transduction efficiency, improved genetic safety, as well as enablement of the use of vectors which are limited to a particular maximum size. For potent scFvs like the CD19-specific scFv FMC63, the inventors show that most variants are functional in principle and that an optimized version of the IgG3 spacer is equally effective in inducing proliferation, cytokine secretion and cytotoxicity. For most other targets, an optimal configuration can be identified that leads to best antitumor efficacy in vitro as well as in vivo.

The inventor identified an antibody, termed anti-MiH antibody #1, that is capable of specifically binding the IgG3 middle Hinge region, though their data suggest that proper binding requires 3 or more IgG3_MiH repeats. Using this antibody and its derivatives, the inventors could show, that CAR T cells can be targeted antigen-independently but CAR-specifically. The inventors reveal that additional functions that can be exploited include stimulation, expansion and depletion as well as enrichment of CAR T cells directly via the CAR itself instead via a CAR-independent transduction marker.

Since the inventors' data suggest that a majority of CAR scFvs shows best function with a rather short spacer undercutting 3 IgG3_MiH repeats, they included a second multi-function site comprising 5 IgG3_MiH repeats between the first and the second domain of a scFv, thereby replacing the commonly used (G₄S)₃ linker.

The inventors show that this alternative linker between scFv VH and VL does not impair the target recognition of the CAR construct, allowing its exploitation for additional functions. The inventors' data demonstrate that targeting the multifunction site leads to efficient antigen-independent but CAR-specific stimulation and proliferation, as well as specific enrichment and depletion of CAR T cells.

In previous attempts, a StrepTag II was used as part of the spacer as well as a tag¹⁴, or a myc-tag was used as part of the spacer domain as well as a tag¹⁵. In contrast to that, the concept that the inventors provide originates from a fully human protein in an unmodified form, making the occurrence of immunogenicity much less likely as for such artificial proteins. Moreover, for these tags, it has not been shown that it is possible to arrange several copies of the motif one after another in order to optimize spacer length and flexibility.

In summary, the inventors' data encourage the use of IgG3-Hinge-derived spacer domains for implementation in CAR design. Their good functionality, in association with the unique exploitation of antigen-independent though CAR-specific functions using a spacer-targeting antibody, accompanied with a low immunogenicity of the CAR construct make this approach an attractive option for pre-clinical, clinical and commercial exploitation.

Definitions and Embodiments

Unless otherwise defined below, the terms used in the present invention shall be understood in accordance with the common meaning known to the person skilled in the art.

Each publication, patent application, patent, and other reference cited herein is incorporated by reference in its entirety to the extent that it is not inconsistent with the present invention. References are indicated by their reference numbers and their corresponding reference details which are provided in the “references” section.

An IgG3 middle hinge domain repeat motif in accordance with the invention is a motif located in the middle hinge of an antibody of an IgG3 class, which can occur more than once in the hinge region. In a preferred embodiment, the IgG3 middle hinge domain repeat motif consists the amino acid sequence of SEQ ID NO: 1.

An immunoreceptor according to the invention is a transmembrane receptor, which, when expressed by an immune cell, is capable of mediating an immune response. The immunoreceptor can be an endogenous immunoreceptor or a non-natural immunoreceptor, i.e. genetically engineered. Exemplary immunoreceptors in accordance with the invention are B-cell receptors (BCRs), T-cell receptors (TCRs), and chimeric antigen receptor (CARS). The immunoreceptor in its monomeric form may either consist of a single molecule comprising all of its domains or consist of a heterodimer that comprises all of its domains. The immunoreceptor can bind to its antigen either directly, or it can bind indirectly through an adapter.

The immunoreceptor according to the invention can comprise an antigen-binding domain which comprises a first domain, linker, and optionally a second domain. The first and second domain are not limited to a specific molecular orientation, i.e. both first and second domain can be located N-terminal or C-terminal to each other. Optionally, the second domain can be absent, i.e. the antigen-binding domain can be comprised of the first domain and the linker, in any orientation in respect of N-terminal or C-terminal orientation. An exemplary embodiment of an antigen-binding domain is a single chain variable fragment (scFv). In this case, the first domain can comprise a light chain variable domain or a heavy chain variable domain, and the second domain can comprise a light chain variable domain or a heavy chain variable domain, which are connected by a peptide linker. The first and second domain can both either be located at the N-terminus of the scFv, or at the C-terminus of the scFv.

In one embodiment, the immunoreceptor is capable of binding to an antigen, preferably a cancer antigen, more preferably a cancer cell surface antigen. In a preferred embodiment, the immunoreceptor is capable of binding to extracellular domain of a cancer antigen. In a preferred embodiment, the immunoreceptor is a chimeric antigen receptor. In a preferred embodiment, the immunoreceptor is a genetically engineered T-cell receptor.

In a preferred embodiment, the immunoreceptor is expressed in T cells. In a preferred embodiment of the invention, the immunoreceptor is expressed in T cells and allows said T cells to bind specifically to antigen-expressing cancer cells with high specificity to exert a growth inhibiting effect, preferably a cytotoxic effect, on said cancer cells.

In a preferred embodiment in accordance with the invention, immune cells are isolated from a healthy donor or a patient having cancer, transduced with a gene transfer vector encoding an immunoreceptor comprising one or more IgG3 middle hinge repeat domain motifs, which is capable of binding to an antigen expressed by said cancer, and administered to the patient to treat said cancer. In a preferred embodiment, the immune cells are B cells, NK cells, macrophages or T cells. In a more preferred embodiment, the T cells are CD8+ T cells or CD4+ T cells.

The term antibody as used herein refers to any functional antibody that is capable of specific binding to the antigen of interest. Without particular limitation, the term antibody encompasses antibodies from any appropriate source species, including avian such as chicken and mammalian such as mouse, goat, rabbit, non-human primate and human. Preferably, the antibody is a humanized antibody. Humanized antibodies are antibodies which contain human sequences and a minor portion of non-human sequences which confer binding specificity to an antigen of interest (e.g. human FLT3). The antibody is preferably a monoclonal antibody which can be prepared by methods well-known in the art. The term antibody encompasses an IgG-1, -2, -3, or -4, IgE, IgA, IgM, or IgD isotype antibody. The term antibody encompasses monomeric antibodies (such as IgD, IgE, IgG) or oligomeric antibodies (such as IgA or IgM). The term antibody also encompasses—without particular limitations—isolated antibodies and modified antibodies such as genetically engineered antibodies, e.g. chimeric antibodies or bispecific antibodies.

An antibody fragment or fragment of an antibody as used herein refers to a portion of an antibody that retains the capability of the antibody to specifically bind to the antigen (e.g. the IgG3 middle hinge repeat domain). This capability can, for instance, be determined by determining the capability of the antigen-binding portion to compete with the antibody for specific binding to the antigen by methods known in the art. Without particular limitation, the antibody fragment can be produced by any suitable method known in the art, including recombinant DNA methods and preparation by chemical or enzymatic fragmentation of antibodies. Antibody fragments may be Fab fragments, F(ab′) fragments, F(ab′)2 fragments, single chain antibodies (scFv), single-domain antibodies, diabodies or any other portion(s) of the antibody that retain the capability of the antibody to specifically bind to the antigen.

An “antibody” (e.g. a monoclonal antibody) or “a fragment thereof” as described herein may have been derivatized or be linked to a different molecule. For example, molecules that may be linked to the antibody are other proteins (e.g. other antibodies), a molecular label (e.g. a fluorescent, luminescent, colored or radioactive molecule), a pharmaceutical and/or a toxic agent. The antibody or antigen-binding portion may be linked directly (e.g. in form of a fusion between two proteins), or via a linker molecule (e.g. any suitable type of chemical linker known in the art).

A “bispecific antibody” is an antibody or fragment thereof as described herein which is capable of specifically binding to two antigens which are different from each other. An exemplary embodiment of a bispecific antibody is an antibody which is capable of specifically binding to a cancer cell surface antigen (e.g. CD19 or CD20) and an immune cell surface antigen (e.g. CD3). The bispecific antibody is preferably capable of recruiting immune cells to target cells, such as cancer cells, and thereby mediate antibody-dependent cell-mediated cytotoxicity (ADCC). The bispecific antibody may comprise a portion which interacts with Fc receptors.

Terms such as “treatment of cancer” or “treating cancer” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). An assessment of whether or not a therapeutic treatment works can be made based on known clinical indicators of cancer progression.

A treatment of cancer according to the present invention does not exclude that additional or secondary therapeutic benefits also occur in patients. For example, an additional or secondary benefit may be an enhancement of engraftment of transplanted hematopoietic stem cells that is carried out prior to, concurrently to, or after the treatment of cancer. However, it is understood that the primary treatment for which protection is sought is for treating the cancer itself, and any secondary or additional effects only reflect optional, additional advantages of the treatment of cancer growth.

The treatment of cancer according to the invention can be a first-line therapy, a second-line therapy, a third-line therapy, or a fourth-line therapy. The treatment can also be a therapy that is beyond fourth-line therapy. The meaning of these terms is known in the art and in accordance with the terminology that is commonly used by the US National Cancer Institute.

The treatment of infectious, automimmune and degenerative diseases, respectively, can be a first-line therapy, a second-line therapy, a third-line therapy, or a fourth-line therapy. The treatment can also be a therapy that is beyond fourth-line therapy. The meaning of these terms is known in the art.

The term “capable of binding” as used herein refers to the capability to form a complex with a molecule that is to be bound (e.g. the IgG3 middle hinge repeat domain). Binding typically occurs non-covalently by intermolecular forces, such as ionic bonds, hydrogen bonds and Van der Waals forces and is typically reversible. Various methods and assays to determine binding capability are known in the art. Binding is usually a binding with high affinity, wherein the affinity as measured in KD values is preferably is less than 1 μM, more preferably less than 100 nM, even more preferably less than 10 nM, even more preferably less than 1 nM, even more preferably less than 100 pM, even more preferably less than 10 pM, even more preferably less than 1 pM.

As used herein, each occurrence of terms such as “comprising” or “comprises” may optionally be substituted with “consisting of” or “consists of”.

As used herein, terms such as a “linker” which “comprises one or more IgG3 middle hinge domain repeat motifs” or a “spacer domain” which “comprises one or more IgG3 middle hinge domain repeat motifs” can refer to a linker or spacer domain where said one or more IgG3 middle hinge domain repeat motifs are present in addition to said one or more IgG3 middle hinge domain repeat motifs of the immunoreceptor of the invention. Alternatively, terms such as a “linker” which “comprises one or more IgG3 middle hinge domain repeat motifs” or a “spacer domain” which “comprises one or more IgG3 middle hinge domain repeat motifs” can refer to a linker or spacer domain where said one or more IgG3 middle hinge domain repeat motifs are identical to said one or more IgG3 middle hinge domain repeat motifs of the immunoreceptor of the invention.

The term “reduced immunogenicity” in connection with an immunoreceptor or CAR or bispecific antibody is to be understood in accordance with its general meaning in the art. In a preferred embodiment in accordance with all other embodiments of the invention, “reduced immunogenicity” in connection with an immunoreceptor or CAR means that the immunoreceptor or CAR has reduced immunogenicity in comparison to a second immunoreceptor or CAR in an assay wherein said immunoreceptor or CAR is expressed in a HLA/A2-positive tumor cell line, followed by co-incubation of the cell line with PBMCs of a HLA/A2-positive donor, and followed by an enzyme-linked immunosorbent assay (ELISA)-based determination of whether the immunoreceptor or CAR causes reduced cytokine production by the PBMCs. In a preferred embodiment in accordance with all other embodiments of the invention, “reduced immunogenicity” in connection with a bispecific antibody means that the bispecific antibody causes reduced anti-drug antibody levels in human patients in comparison to a second bispecific antibody. Anti-drug antibody levels can be determined by methods known in the art including ELISA-based methods.

A pharmaceutically acceptable carrier, including any suitable diluent or, can be used herein as known in the art. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. Pharmaceutically acceptable carriers include, but are not limited to, saline, buffered saline, dextrose, water, glycerol, sterile isotonic aqueous buffer, and combinations thereof. It will be understood that the formulation will be appropriately adapted to suit the mode of administration.

Compositions and formulations in accordance with the present invention are prepared in accordance with known standards for the preparation of pharmaceutical compositions and formulations. For instance, the compositions and formulations are prepared in a way that they can be stored and administered appropriately, e.g. by using pharmaceutically acceptable components such as carriers, excipients or stabilizers. Such pharmaceutically acceptable components are not toxic in the amounts used when administering the pharmaceutical composition or formulation to a patient. The pharmaceutical acceptable components added to the pharmaceutical compositions or formulations may depend on the chemical nature of the inhibitor and targeting agent present in the composition or formulation (depend on whether the targeting agent is e.g. an antibody or fragment thereof or a cell expressing a chimeric antigen receptor), the particular intended use of the pharmaceutical compositions and the route of administration.

In a preferred embodiment in accordance with the invention, the composition or formulation is suitable for administration to humans, preferably the formulation is sterile and/or non-pyrogenic.

In a preferred embodiment, the invention provides an immunoreceptor, comprising one or more IgG3 hinge repeat domain motifs, wherein the immunoreceptor does not comprise an IgG3 CH2 and/or CH3 domain.

In an alternative embodiment, the immunoreceptor comprises an IgG3 CH2 domain. In a further alternative embodiment, the immunoreceptor comprises an IgG3 CH3 domain. In a further alternative embodiment, the immunoreceptor comprises an IgG3 CH2 and CH3 domain. In a further alternative embodiment, the immunoreceptor comprises an IgG3 CH1 domain. In a further alternative embodiment, the immunoreceptor comprises an IgG3 CH1, CH2 and CH3 domain.

The terms “IgG3 CH2 domain” and “IgG3 CH3 domain” are to be understood in accordance with their meaning known in the art. In a preferred embodiment in accordance with all other embodiments of the invention, the IgG3 CH2 domain is the CH2 domain of human IgG3 consisting of the sequence of SEQ ID NO: 172, and the IgG3 CH3 domain is the CH3 domain of human IgG3 consisting of the sequence of SEQ ID NO: 173.

In a preferred embodiment, the immunoreceptor in accordance with the invention comprises an extracellular antigen-binding domain, a spacer domain, and a transmembrane domain, wherein the spacer domain is located between the antigen-binding domain and the transmembrane domain. In a preferred embodiment, the spacer domain comprises one or more IgG3 middle hinge domain repeat motifs. In a preferred embodiment, the transmembrane domain and the intracellular domain of the immunoreceptor together consist of a sequence selected from the group consisting of SEQ ID NO: 109, 110, 111, 112, 113, 114, 115 and 174. In a preferred embodiment, the immunoreceptor is a chimeric antigen receptor, and the antigen-binding domain is a single chain variable fragment, which is linked to the chimeric antigen receptor by said spacer, which comprises one or more IgG3 middle hinge domain repeat motifs, preferably two or more IgG3 middle hinge domain repeat motifs, more preferably three or more IgG3 middle hinge domain repeat motifs. In this embodiment, the antigen-binding protein (e.g. an antibody or fragment thereof) is capable of binding to the immunoreceptor by specifically binding to the one or more, preferably two or more, more preferably three or more IgG3 middle hinge domain repeat motifs comprised in the immunoreceptor. Binding of said antigen-binding protein to said immunoreceptor by means of recognition of said IgG3 middle hinge domain repeat motifs may affect the immunoreceptor's effector function, such as its downstream signaling that modulates the properties of the cell which express said immunoreceptor, e.g. its proliferation or interaction with other immune cells. In this embodiment, the number of repeats of said IgG3 middle hinge domain repeat motifs comprised in said spacer domain comprised in said immunoreceptor can affect the capability of said immunoreceptor to selectively and efficiently bind to a particular target antigen present on a target cell's surface (e.g. CD19, CD20, ROR1, ROR2, SLAMF7, FLT3, Siglec-6, αᵥβ₃ integrin, or BCMA). Preferably, the target cell is a cancer cell, and the target antigen is a cancer antigen, i.e. a cell surface marker expressed to a higher degree in cancer cells than in non-disease cells. In an exemplary embodiment, the immunoreceptor of the invention is a chimeric antigen receptor which comprises, as the transmembrane domain, the amino acid sequence of SEQ ID NO: 65. In this embodiment, the chimeric antigen receptor may further comprise a 4-1BB domain having an amino acid sequence as set forth in SEQ ID NO: 66, and a CD3 zeta domain having an amino acid sequence as set forth in SEQ ID NO: 67. In an exemplary embodiment, the immunoreceptor is a CD19 chimeric antigen receptor having an amino acid sequence as set forth in SEQ ID NO: 68.

In another preferred embodiment, the immunoreceptor is a chimeric antigen receptor, and the antigen-binding domain is a single chain variable fragment, wherein the single chain variable fragment comprises a first domain, a linker, and a second domain, and the linker comprises one or more IgG3 middle hinge domain repeat motifs, preferably two or more IgG3 middle hinge domain repeat motifs, more preferably three or more IgG3 middle hinge domain repeat motifs. In this embodiment, the antigen-binding protein (e.g. an antibody or fragment thereof) is capable of binding to the immunoreceptor by specifically binding to the one or more, preferably two or more, more preferably three or more IgG3 middle hinge domain repeat motifs comprised in the immunoreceptor. In this embodiment, the antigen-binding protein (e.g. an antibody or fragment thereof) is capable of binding to the immunoreceptor by specifically binding to the one or more, preferably two or more, more preferably three or more IgG3 middle hinge domain repeat motifs comprised in the immunoreceptor. Binding of said antigen-binding protein to said immunoreceptor by means of recognition of said IgG3 middle hinge domain repeat motifs may affect the immunoreceptor's effector function, such as its downstream signaling that modulates the properties of the cell which express said immunoreceptor, e.g. its proliferation or interaction with other immune cells.

In a preferred embodiment, the presence of the one or more, preferably two or more, more preferably three or more IgG3 middle hinge domain repeat motifs in said immunoreceptor does not cause unspecific or otherwise undesired immunogenic reactions against the cell expressing said immunoreceptor.

The antigen-binding domain in accordance with the invention is generally capable of specifically binding to a given target antigen. In a preferred embodiment, the antigen-binding domain is capable of specifically binding to a cell surface antigen, preferably a cancer antigen i.e. a cell surface marker expressed to a higher degree in cancer cells than in non-disease cells. The antigen-binding domain, when incorporated into an immunoreceptor in accordance with the invention, enables said immunoreceptor to specifically recognize and bind the target antigen which the antigen-binding domain is able to specifically bind to. In this embodiment, when said immunoreceptor comprising said antigen-binding domain is expressed by a cell, said cell acquires the capability of specifically recognizing a target cell which expresses said target antigen. In a preferred embodiment, the immunoreceptor is a chimeric antigen receptor, and the antigen-binding domain is a single chain variable fragment which is part of said chimeric antigen receptor.

In an embodiment, the immunoreceptor in accordance with the invention is a chimeric antigen receptor which comprises a spacer domain located between an extracellular antigen-binding domain and a transmembrane domain, wherein the spacer domain comprises one or more, preferably two or more, more preferably three or more IgG3 middle hinge domain repeat motifs, and wherein the extracellular antigen-binding domain is an scFv specific for CD19, CD20, ROR1, ROR2, SLAMF7, FLT3, Siglec-6, αᵥβ₃ integrin, or BCMA, wherein the scFv does not comprise IgG3 middle hinge domain repeat motif.

In an embodiment, the immunoreceptor in accordance with the invention is a chimeric antigen receptor which comprises a spacer domain located between an extracellular antigen-binding domain and a transmembrane domain, wherein the spacer domain does comprise an IgG3 middle hinge domain repeat motifs, and wherein the extracellular antigen-binding domain is an scFv specific for CD19, CD20, ROR1, ROR2, SLAMF7, FLT3, Siglec-6, αᵥβ₃ integrin, or BCMA, wherein the scFv comprises a first domain, linker, and second domain, and said linker comprises one or more, preferably two or more, more preferably three or more IgG3 middle hinge domain repeat motifs.

In one embodiment, the immunoreceptor in accordance with the invention is a chimeric antigen receptor specific for CD19, wherein said chimeric antigen receptor comprises a heavy chain variable domain which has an amino acid sequence having at least 80% sequence identity, preferably at least 90% sequence identity, to SEQ ID NO: 27,and the light chain variable domain has an amino acid sequence having at least 80% sequence identity, preferably at least 90% sequence identity, to SEQ ID NO: 28. In one embodiment, the heavy chain variable domain has the amino acid sequence of SEQ ID NO: 27 and the light chain variable domain has the amino acid sequence of SEQ ID NO: 28. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for CD20, wherein said chimeric antigen receptor comprises a heavy chain variable domain which has an amino acid sequence having at least 80% sequence identity, preferably at least 90% sequence identity, to SEQ ID NO: 30,and the light chain variable domain has an amino acid sequence having at least 80% sequence identity, preferably at least 90% sequence identity, to SEQ ID NO: 29. In one embodiment, the heavy chain variable domain has the amino acid sequence of SEQ ID NO: 30 and the light chain variable domain has the amino acid sequence of SEQ ID NO: 29. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for ROR1, wherein said chimeric antigen receptor comprises a heavy chain variable domain which has an amino acid sequence having at least 80% sequence identity, preferably at least 90% sequence identity, to SEQ ID NO: 31, 33, 35, or 37, and the light chain variable domain has an amino acid sequence having at least 80% sequence identity, preferably at least 90% sequence identity, to SEQ ID NO: 32, 34, 36, or 38, respectively. In one embodiment, the heavy chain variable domain has the amino acid sequence of SEQ ID NO: 31, 33, 35, or 37, and the light chain variable domain has the amino acid sequence of SEQ ID NO: 32, 34, 36, or 38, respectively. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for ROR2, wherein said chimeric antigen receptor comprises a heavy chain variable domain which has an amino acid sequence having at least 80% sequence identity, preferably at least 90% sequence identity, to SEQ ID NO: 39,and the light chain variable domain has an amino acid sequence having at least 80% sequence identity, preferably at least 90% sequence identity, to SEQ ID NO: 40. In one embodiment, the heavy chain variable domain has the amino acid sequence of SEQ ID NO: 39 and the light chain variable domain has the amino acid sequence of SEQ ID NO: 40. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for SLAMF7, wherein said chimeric antigen receptor comprises a heavy chain variable domain which has an amino acid sequence having at least 80% sequence identity, preferably at least 90% sequence identity, to SEQ ID NO: 41 or 43, and the light chain variable domain has an amino acid sequence having at least 80% sequence identity, preferably at least 90% sequence identity, to SEQ ID NO: 42 or 44, respectively. In one embodiment, the heavy chain variable domain has the amino acid sequence of SEQ ID NO: 41 or 43, and the light chain variable domain has the amino acid sequence of SEQ ID NO: 42 or 44, respectively. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for FLT3, wherein said chimeric antigen receptor comprises a heavy chain variable domain which has an amino acid sequence having at least 80% sequence identity, preferably at least 90% sequence identity, to SEQ ID NO: 45 or 47, and the light chain variable domain has an amino acid sequence having at least 80% sequence identity, preferably at least 90% sequence identity, to SEQ ID NO: 46 or 48, respectively. In one embodiment, the heavy chain variable domain has the amino acid sequence of SEQ ID NO: 45 or 47, and the light chain variable domain has the amino acid sequence of SEQ ID NO: 46 or 48, respectively. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for Siglec-6, wherein said chimeric antigen receptor comprises a heavy chain variable domain which has an amino acid sequence having at least 80% sequence identity, preferably at least 90% sequence identity, to SEQ ID NO: 49,and the light chain variable domain has an amino acid sequence having at least 80% sequence identity, preferably at least 90% sequence identity, to SEQ ID NO: 50. In one embodiment, the heavy chain variable domain has the amino acid sequence of SEQ ID NO: 49 and the light chain variable domain has the amino acid sequence of SEQ ID NO: 50. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for αᵥβ₃ integrin, wherein said chimeric antigen receptor comprises a heavy chain variable domain which has an amino acid sequence having at least 80% sequence identity, preferably at least 90% sequence identity, to SEQ ID NO: 51 or 53, and the light chain variable domain has an amino acid sequence having at least 80% sequence identity, preferably at least 90% sequence identity, to SEQ ID NO: 52 or 54, respectively. In one embodiment, the heavy chain variable domain has the amino acid sequence of SEQ ID NO: 51 or 53, and the light chain variable domain has the amino acid sequence of SEQ ID NO: 52 or 54, respectively. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for BCMA, wherein said chimeric antigen receptor comprises a heavy chain variable domain which has an amino acid sequence having at least 80% sequence identity, preferably at least 90% sequence identity, to SEQ ID NO: 55 or 57, and the light chain variable domain has an amino acid sequence having at least 80% sequence identity, preferably at least 90% sequence identity, to SEQ ID NO: 56 or 58, respectively. In one embodiment, the heavy chain variable domain has the amino acid sequence of SEQ ID NO: 55 or 57, and the light chain variable domain has the amino acid sequence of SEQ ID NO: 56 or 58, respectively. In this embodiment, said chimeric antigen receptor comprises one or more, preferably two or more, more preferably three or more IgG3 middle hinge domain repeat motifs. In one embodiment, said one or more, preferably two or more, more preferably three or more IgG3 middle hinge domain repeat motifs is/are located within the spacer domain of said chimeric antigen receptor. In another embodiment, said one or more, preferably two or more, more preferably three or more IgG3 middle hinge domain repeat motifs is/are located within the linker comprised in the extracellular antigen-binding domain comprised in said chimeric antigen receptor, wherein the extracellular antigen-binding domain is an scFv. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for CD19, wherein said chimeric antigen receptor comprises an scFv which has an amino acid sequence having at least 80% sequence identity, preferably at least 90% sequence identity, optionally 100% sequence identity, to SEQ ID NO: 3 or 71. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for CD20, wherein said chimeric antigen receptor comprises an scFv which has an amino acid sequence having at least 80% sequence identity, preferably at least 90% sequence identity, optionally 100% sequence identity, to SEQ ID NO: 4 or 72. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for ROR1, wherein said chimeric antigen receptor comprises an scFv which has an amino acid sequence having at least 80% sequence identity, preferably at least 90% sequence identity, optionally 100% sequence identity, to SEQ ID NO: 5, 6, 7, 8, 73, 74, 75, 76, 87, 88, 89, 90, 91, 92, 93, 94, 95, 96, 97, 98, 99 or 100. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for ROR2, wherein said chimeric antigen receptor comprises an scFv which has an amino acid sequence having at least 80% sequence identity, preferably at least 90% sequence identity, optionally 100% sequence identity, to SEQ ID NO: 9, 77, 101, 102, 103, 104, 105, 106, 107 or 108. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for SLAMF7, wherein said chimeric antigen receptor comprises an scFv which has an amino acid sequence having at least 80% sequence identity, preferably at least 90% sequence identity, optionally 100% sequence identity, to SEQ ID NO: 10, 11, 78 or 79. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for FLT3, wherein said chimeric antigen receptor comprises an scFv which has an amino acid sequence having at least 80% sequence identity, preferably at least 90% sequence identity, optionally 100% sequence identity, to SEQ ID NO: 12, 13, 80 or 81. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for Siglec-6, wherein said chimeric antigen receptor comprises an scFv which has an amino acid sequence having at least 80% sequence identity, preferably at least 90% sequence identity, optionally 100% sequence identity, to SEQ ID NO: 14 or 82. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for αᵥβ₃ integrin, wherein said chimeric antigen receptor comprises an scFv which has an amino acid sequence having at least 80% sequence identity, preferably at least 90% sequence identity, optionally 100% sequence identity, to SEQ ID NO: 15, 16, 83 or 84. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In one embodiment, the immunoreceptor in accordance with the invention is a chimeric antigen receptor specific for BCMA, wherein said chimeric antigen receptor comprises an scFv which has an amino acid sequence having at least 80% sequence identity, preferably at least 90% sequence identity, optionally 100% sequence identity, to SEQ ID NO: 17, 18, 85 or 86. In this embodiment, said chimeric antigen receptor comprises one or more, preferably two or more, more preferably three or more IgG3 middle hinge domain repeat motifs which are located within the spacer domain of said chimeric antigen receptor. In this embodiment, the linker comprised in the extracellular antigen-binding domain comprised in said scFv comprised in said chimeric antigen receptor does not comprise an IgG3 middle hinge domain repeat motif. In a preferred embodiment, said chimeric antigen receptor does not cause unspecific or undesired immune reactions compared to a chimeric antigen receptor which does not comprise said one or more, preferably two or more, more preferably three or more IgG3 middle hinge domain repeat motifs but is otherwise similar, i.e. comprises the same intracellular, extracellular, and transmembrane domains, and only differs from said chimeric antigen receptor of the invention in the spacer domain and/or the linker comprised in the scFv comprised in said chimeric antigen receptor.

In another aspect, the invention also provides a chimeric antigen receptor comprising
an extracellular antigen-binding domain,
a spacer domain,
a transmembrane domain, and
an intracellular signaling domain;

wherein the spacer domain is located between the extracellular antigen-binding domain and the transmembrane domain, and wherein the spacer domain comprises an amino acid sequence which has 100% sequence identity with the amino acid sequence of [A-Bₙ], wherein
A is the amino acid sequence of SEQ ID NO: 2;
B has the amino acid sequence of SEQ ID NO: 1; and
n is selected from the group consisting of 0, 1, 2, 3, 4, 5, 6, 7, 8, 9, 10, 11, 12, 13, 14 and 15 and is preferably an integer between 1 and 15, more preferably an integer between 1 and 10, even more preferably an integer between 1 and 5, most preferably an integer between 3 and 5.

In a preferred embodiment of this aspect, the transmembrane domain and the intracellular domain together consist of a sequence selected from the group consisting of SEQ ID NO: 109, 110, 111, 112, 113, 114, 115 and 174.

All embodiments of the invention which are defined above for the immunoreceptor or CAR of the invention also apply to the chimeric antigen receptor of this aspect of the invention.

In preferred embodiments, the chimeric antigen receptor of the invention including this aspect of the invention can be specific for CD19, wherein said extracellular antigen-binding domain comprises an scFv which has an amino acid sequence having 100% sequence identity to SEQ ID NO: 3 or 71, or the chimeric antigen receptor can be specific for CD20, wherein said extracellular antigen-binding domain comprises an scFv which has an amino acid sequence having 100% sequence identity to SEQ ID NO: 4 or 72, or the chimeric antigen receptor can be specific for ROR1, wherein said extracellular antigen-binding domain comprises an scFv which has an amino acid sequence having 100% sequence identity to SEQ ID NO: 5, 6, 7, 8, 73, 74, 75, 76, 87, 88, 89, 90, 91, 92, 93, 94, 95, 96, 97, 98, 99 or 100, or the chimeric antigen receptor can be specific for ROR2, wherein said extracellular antigen-binding domain comprises an scFv which has an amino acid sequence having 100% sequence identity to SEQ ID NO: 9, 77, 101, 102, 103, 104, 105, 106, 107 or 108, or the chimeric antigen receptor can be specific for SLAMF7, wherein said extracellular antigen-binding domain comprises an scFv which has an amino acid sequence having 100% sequence identity to SEQ ID NO: 10, 11, 78 or 79, or the chimeric antigen receptor can be specific for FLT3, wherein said extracellular antigen-binding domain comprises an scFv which has an amino acid sequence having 100% sequence identity to SEQ ID NO: 12, 13, 80 or 81, or the chimeric antigen receptor can be specific for Siglec-6, wherein said extracellular antigen-binding domain comprises an scFv which has an amino acid sequence having 100% sequence identity to SEQ ID NO: 14 or 82, or the chimeric antigen receptor can be specific for αᵥβ₃ integrin, wherein said extracellular antigen-binding domain comprises an scFv which has an amino acid sequence having 100% sequence identity to SEQ ID NO: 15, 16, 83 or 84, or the chimeric antigen receptor can be specific for BCMA, wherein said extracellular antigen-binding domain comprises an scFv which has an amino acid sequence having 100% sequence identity to SEQ ID NO: 17, 18, 85 or 86.

In a very preferred embodiment of the invention, the chimeric antigen receptor of the invention comprises an amino acid sequence selected from the group consisting of SEQ ID NO: 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, and 171.

In one embodiment, the immunoreceptor in accordance with the invention comprising one or more, preferably two or more, more preferably three or more IgG3 middle hinge domain repeat motifs is/are used in therapy. In an embodiment, the invention provides a medicine, comprising, as one active ingredient, cells, e.g. immune cells such as T cells, expressing an immunoreceptor in accordance with the invention.

In a preferred embodiment, the CD19-specific chimeric antigen receptor comprising one or more, preferably two or more, more preferably three or more IgG3 middle hinge domain repeat motifs in accordance with the invention is used in the treatment of cancer, wherein in the method, cells expressing said CD19-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the CD19-specific chimeric antigen receptor is used in the treatment of Non-Hodgkin lymphoma, Multiple Myeloma, Burkitt's lymphoma, Mantle cell lymphoma, Acute lymphoblastic leukemia, Chronic lymphocytic leukemia and Diffuse large B-cell lymphoma.

In a preferred embodiment, the CD20-specific chimeric antigen receptor comprising one or more, preferably two or more, more preferably three or more IgG3 middle hinge domain repeat motifs in accordance with the invention is used in the treatment of cancer, wherein in the method, cells expressing said CD20-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the CD20-specific chimeric antigen receptor is used in the treatment of Non-Hodgkin lymphoma, Multiple Myeloma, Burkitt's lymphoma, Mantle cell lymphoma, Acute lymphoblastic leukemia, Chronic lymphocytic leukemia and Diffuse large B-cell lymphoma.

In a preferred embodiment, the ROR1-specific chimeric antigen receptor comprising one or more, preferably two or more, more preferably three or more IgG3 middle hinge domain repeat motifs in accordance with the invention is used in the treatment of cancer, wherein in the method, cells expressing said ROR1-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the ROR1-specific chimeric antigen receptor is used in the treatment of breast cancer, lung cancer, Mantle cell lymphoma, Chronic lymphocytic leukemia and Diffuse large B-cell lymphoma.

In a preferred embodiment, the ROR2-specific chimeric antigen receptor comprising one or more, preferably two or more, more preferably three or more IgG3 middle hinge domain repeat motifs in accordance with the invention is used in the treatment of cancer, wherein in the method, cells expressing said ROR2-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the ROR2-specific chimeric antigen receptor is used in the treatment of breast cancer, colon cancer prostate cancer, osteosarcoma and Multiple Myeloma.

In a preferred embodiment, the SLAMF7-specific chimeric antigen receptor comprising one or more, preferably two or more, more preferably three or more IgG3 middle hinge domain repeat motifs in accordance with the invention is used in the treatment of cancer, wherein in the method, cells expressing said SLAMF7-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the SLAMF7-specific chimeric antigen receptor is used in the treatment of Multiple Myeloma, T cell and B cell leukemia or lymphoma.

In a preferred embodiment, the FLT3-specific chimeric antigen receptor comprising one or more, preferably two or more, more preferably three or more IgG3 middle hinge domain repeat motifs in accordance with the invention is used in the treatment of cancer, wherein in the method, cells expressing said FLT3-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the FLT3-specific chimeric antigen receptor is used in the treatment of Acute Myeloid leukemia, Acute lymphoblastic leukemia and Myelodysplastic Syndromes.

In a preferred embodiment, the Siglec-6-specific chimeric antigen receptor comprising one or more, preferably two or more, more preferably three or more IgG3 middle hinge domain repeat motifs in accordance with the invention is used in the treatment of cancer, wherein in the method, cells expressing said Siglec-6-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the Siglec-6-specific chimeric antigen receptor is used in the treatment of Acute Myeloid leukemia.

In a preferred embodiment, the αᵥβ₃ integrin-specific chimeric antigen receptor comprising one or more, preferably two or more, more preferably three or more IgG3 middle hinge domain repeat motifs in accordance with the invention is used in the treatment of cancer, wherein in the method, cells expressing said αᵥβ₃ integrin-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the αᵥβ₃ integrin-specific chimeric antigen receptor is used in the treatment of breast cancer, pancreatic cancer, prostate cancer, melanoma and glioblastoma.

In a preferred embodiment, the BCMA-specific chimeric antigen receptor comprising one or more, preferably two or more, more preferably three or more IgG3 middle hinge domain repeat motifs in accordance with the invention is used in the treatment of cancer, wherein in the method, cells expressing said BCMA-specific chimeric antigen receptor are administered to a patient in need thereof, thereby treating said cancer. In one embodiment, the cells are autologous, i.e. are obtained from the same patient that is to be treated. In one embodiment, the cells are allogeneic (because they are obtained from a source other than the patient that is to be treated). In a preferred embodiment, the BCR-specific chimeric antigen receptor is used in the treatment of Multiple Myeloma and amyloidosis.

In one embodiment, the immunoreceptor is a T-cell receptor (TCR), preferably a recombinant TCR; a B-cell receptor (BCR), preferably a recombinant BCR; or a chimeric antigen receptor (CAR). In one embodiment, the immunoreceptor is a recombinant, i.e. non-natural, genetically engineered T-cell receptor (TCR). In one embodiment, the immunoreceptor is a recombinant, i.e. non-natural, genetically engineered B-cell receptor (BCR). In a preferred embodiment, the immunoreceptor is a chimeric antigen receptor (CAR).

In a preferred embodiment, the IgG3 middle hinge repeat domain motif in accordance with the invention is from a human IgG3 middle hinge. In a preferred embodiment, said IgG3 middle hinge repeat domain motif comprises at least 10, 11, 12, 13, or 14 contiguous amino acids of SEQ ID NO: 1. In one embodiment, said IgG3 middle hinge repeat domain motif comprises at least 10 contiguous amino acids of SEQ ID NO: 1. In one embodiment, said IgG3 middle hinge repeat domain motif comprises at least 11 contiguous amino acids of SEQ ID NO: 1. In one embodiment, said IgG3 middle hinge repeat domain motif comprises at least 12 contiguous amino acids of SEQ ID NO: 1. In one embodiment, said IgG3 middle hinge repeat domain motif comprises at least 13 contiguous amino acids of SEQ ID NO: 1. In one embodiment, said IgG3 middle hinge repeat domain motif comprises at least 14 contiguous amino acids of SEQ ID NO: 1. In one embodiment, said IgG3 middle hinge repeat domain motif comprises at least 15 contiguous amino acids of SEQ ID NO: 1. In a preferred embodiment, said IgG3 middle hinge repeat domain motif consists of the amino acid sequence of SEQ ID NO: 1 having not more than 5, 4, 3, 2, or 1 conservative amino acid substitutions. In one embodiment, said IgG3 middle hinge repeat domain motif consists of the amino acid sequence of SEQ ID NO: 1 having not more than 5 conservative amino acid substitutions. In one embodiment, said IgG3 middle hinge repeat domain motif consists of the amino acid sequence of SEQ ID NO: 1 having not more than 4 conservative amino acid substitutions. In one embodiment, said IgG3 middle hinge repeat domain motif consists of the amino acid sequence of SEQ ID NO: 1 having not more than 3 conservative amino acid substitutions. In one embodiment, said IgG3 middle hinge repeat domain motif consists of the amino acid sequence of SEQ ID NO: 1 having not more than 2 conservative amino acid substitutions. In one embodiment, said IgG3 middle hinge repeat domain motif consists of the amino acid sequence of SEQ ID NO: 1 having not more than 1 conservative amino acid substitutions. In a preferred embodiment, said IgG3 middle hinge repeat domain motif has the amino acid sequence of SEQ ID NO: 1.

In a preferred embodiment, the immunoreceptor in accordance with the invention does not comprise all or part of the sequence of the lower hinge domain of an IgG3 hinge domain, preferably said IgG3 hinge domain being human.

In an embodiment, the immunoreceptor in accordance with the invention comprises the IgG3 middle hinge domain repeat motif 1, 2, 3, 4, 5, 6, 7, 8, 9 or 10 times. In an embodiment, the immunoreceptor in accordance with the invention comprises the IgG3 middle hinge domain repeat motif once. In an embodiment, the immunoreceptor in accordance with the invention comprises the IgG3 middle hinge domain repeat motif twice. In an embodiment, the immunoreceptor in accordance with the invention comprises the IgG3 middle hinge domain repeat motif three times. In an embodiment, the immunoreceptor in accordance with the invention comprises the IgG3 middle hinge domain repeat motif four times. In an embodiment, the immunoreceptor in accordance with the invention comprises the IgG3 middle hinge domain repeat motif five times. In a preferred embodiment, the immunoreceptor in accordance with the invention comprises the IgG3 middle hinge domain repeat motif at least three times. In a preferred embodiment, the immunoreceptor in accordance with the invention comprises the IgG3 middle hinge domain repeat motif not more than five times.

In a preferred embodiment, the immunoreceptor in accordance with the invention comprises an amino acid sequence which has at least 80% sequence identity, preferably at least 90% sequence identity, or optionally 100% sequence identity with the amino acid sequence of [A-Bn], wherein A is the amino acid sequence of SEQ ID NO: 2; B is said IgG3 middle hinge domain repeat motif, wherein said motif has the amino acid sequence of SEQ ID NO: 1; and n is an integer between 1 and 15, preferably between 1 and 10, more preferably between 1 and 5, most preferably between 3 and 5. In one embodiment, n is 1. In one embodiment, n is 2. In one embodiment, n is 3. In one embodiment, n is 4. In one embodiment, n is 5. In a preferred embodiment, n is between 3 and 5. In a preferred embodiment, the immunoreceptor in accordance with the invention comprises at least two IgG3 middle hinge domain repeat motifs which are adjacent to each other. In a preferred embodiment, the immunoreceptor in accordance with the invention comprises at least three IgG3 middle hinge domain repeat motifs which are adjacent to each other.

The present invention provides a nucleic acid which encodes the immunoreceptor in accordance with the invention. There are no particular limitations to the nucleic acids of the invention and to how it can be expressed. For example, the nucleic acid which encodes the immunoreceptor in accordance with the invention can be expressed stably or transiently. In a preferred embodiment, the nucleic acid is a viral vector. In one embodiment, the viral vector is a retroviral vector. In a preferred embodiment, the retroviral vector is a lentiviral vector. The lentiviral vector can be a first, second, third, or fourth generation lentiviral vector. Preferably, the lentiviral vector is a third or fourth generation lentiviral vector. In an exemplary embodiment, the lentiviral vector encoding the immunoreceptor comprises the nucleic acid sequence of SEQ ID NO: 61 and SEQ ID NO: 62, wherein the nucleic acid sequence of SEQ ID NO: 61 is located 5′ to the sequence encoding the immunoreceptor, and the nucleic acid sequence of SEQ ID NO: 62 is located 3′ relative to the sequence encoding the immunoreceptor, and the vector is circularized. In one embodiment, the viral vector is an episomal vector. In one embodiment, the viral vector is an adenoviral vector. In one embodiment, the viral vector is an adeno-associated viral vector. In one embodiment, the nucleic acid comprises nucleic acid sequences which enable stable integration into a host cell's genome via transpositions, such as inverted repeats. In an exemplary embodiment, the nucleic acid is a vector encoding the immunoreceptor which comprises the nucleic acid sequence of SEQ ID NO: 63 and SEQ ID NO: 64, wherein the nucleic acid sequence of SEQ ID NO: 63 is located 5′ to the sequence encoding the immunoreceptor, and the nucleic acid sequence of SEQ ID NO: 64 is located 3′ relative to the sequence encoding the immunoreceptor, and the vector is circularized.

In a preferred embodiment, the nucleic acid can be integrated into a host cell's genome via site-directed genome engineering techniques such as CRISPR/Cas9, Zinc finger nucleases or TALEN. In an embodiment, the nucleic acid is a DNA. In one embodiment, the nucleic acid is RNA. In one embodiment, the nucleic acid comprises non-natural nucleotides. In one embodiment, the nucleic acid does not comprise non-natural nucleotides.

The present invention provides a cell comprising the nucleic acid encoding the immunoreceptor in accordance with the invention. In a preferred embodiment, the cell expresses the immunoreceptor. In one embodiment, the cell can be induced to express the immunoreceptor. In a preferred embodiment, the cell is an immune cell. In a more preferred embodiment, the cell is a T cell. In a preferred embodiment, the T cell is a CD4+ T cell. In a preferred embodiment, the T cell is a CD8+ T cell. In a preferred embodiment, the T cell is a cytotoxic T cell (CTL). In one embodiment, the cell comprises all or part of the nucleic acid encoding the immunoreceptor in accordance with the invention stably integrated into its genome. In a preferred embodiment, the cell comprises the entire sequence encoding the immunoreceptor of the invention stably integrated into its genome. In one embodiment, the cell comprises all or part of the nucleic acid encoding the immunoreceptor in accordance with the invention as an episome. In a preferred embodiment, the cell comprises the entire sequence encoding the immunoreceptor of the invention stably as an episome.

In a preferred embodiment, the nucleic acid and cell comprising the immunoreceptor in accordance with the invention are provided for use in the treatment of cancer or autoimmune diseases, infectious diseases or degenerative diseases.

In a preferred embodiment, the cancer is a hematological cancer. In a preferred embodiment, the hematological cancer is leukemia or lymphoma, preferably acute myeloid leukemia, multiple myeloma, non-Hodgkin-lymphoma, Burkitt's lymphoma, mantle cell lymphoma, acute lymphoblastic leukemia, chronic lymphocytic leukemia, or diffuse large B cell lymphoma. In one embodiment, the cancer is a solid cancer. In an embodiment, the solid cancer is breast cancer, colon carcinoma, lung cancer, or prostate cancer.

The present invention provides an antigen-binding protein, which is capable of specifically binding to an epitope comprised of a sequence consisting of at least one, preferably at least two, more preferably at least three repeats of the IgG3 middle hinge repeat domain motifs. In a preferred embodiment, the antigen-binding protein in accordance with the invention is capable of specifically binding to an epitope comprised of the junction of two adjacent IgG3 middle hinge repeat domain motifs. In a preferred embodiment, the antigen-binding protein is an antibody or fragment thereof.

In a preferred embodiment, the antigen-binding protein is an antibody or fragment thereof comprising, as complementarity determining regions (CDRs) comprised in the heavy chain variable region a CDR1 having the amino acid sequence of SEQ ID NO: 20, a CDR2 having the amino acid sequence of SEQ ID NO: 21, and a CDR3 having the amino acid sequence of SEQ ID NO: 22; and as complementarity determining regions (CDRs) comprised in the light chain variable region a CDR1 having the amino acid sequence of SEQ ID NO: 24, a CDR2 having the amino acid sequence of SEQ ID NO: 25, and a CDR3 having the amino acid sequence of SEQ ID NO: 26.

In a preferred embodiment, antigen-binding protein is an antibody or fragment thereof and comprises a heavy chain variable domain having at least 80%, preferably at least 90%, optionally 100% sequence identity with the amino acid sequence of SEQ ID NO: 19, and a light chain variable region having at least 80%, preferably at least 90%, optionally 100% sequence identity with the amino acid sequence of SEQ ID NO: 23, capable of specifically binding to one or more, preferably two or more, more preferably three or more IgG3 middle hinge domain repeat motifs. In a preferred embodiment, the antibody or fragment thereof maintains 100% sequence identity in its CDRs to SEQ ID NO: 20, 21, 22, 24, 25, and 26.

In one embodiment, the antigen-binding protein capable of binding to an epitope comprised of at least one, preferably at least two, more preferably at least three IgG3 middle hinge repeat domain motifs is an antigen-binding protein which does not comprise SEQ ID NO: 19 and/or SEQ ID NO: 23.

The antigen-binding protein in accordance with the invention can be used for purification, detection, depletion, stimulation, expansion, or enrichment of cells expressing the immunoreceptor of the invention.

The present invention provides a method, comprising a step of binding the antigen-binding protein of the invention to cells expressing the immunoreceptor in accordance with the invention.

In one embodiment, the method of the invention is used for purification of cells expressing the immunoreceptor of the invention. In this embodiment said cells are incubated with a primary antibody which is an antigen-binding protein in accordance with the invention, under conditions which allow the primary antibody to bind to the immunoreceptor expressed on the cells' surface, and subsequently said cells are purified by means of separating antibody-bound cells from non-antibody bound cells. In one embodiment, incubation further comprises incubating said cells with an entity capable of binding to the antibody. In a preferred embodiment, the entity is a secondary antibody, preferably labelled with a fluorescent marker; or a bead, preferably a magnetic bead. In one embodiment, the primary antibody is labelled, preferably with a tag or a fluorescent dye. In a preferred embodiment, the separation is carried out by means of MACS or FACS.

In one embodiment, the method of the invention is used for depletion of cells expressing the immunoreceptor of the invention. In this embodiment, said cells are incubated with an antigen-binding protein in accordance with the invention which is coupled to a cytotoxic molecule. In one embodiment, the antigen-binding protein is comprised in a chimeric antigen receptor expressed by another cell, preferably a T cell.

In one embodiment, the method of the invention is used for stimulation of cells expressing the immunoreceptor of the invention. In this embodiment, said cells are incubated with an antigen-binding protein in accordance with the invention, thereby stimulating said cells. In a preferred embodiment, the antigen-binding protein is coupled to a solid phase. In a preferred embodiment, the solid phase is a tissue culture surface. In a preferred embodiment, the solid phase is a bead, preferably a magnetic bead. In one embodiment, the antigen-binding protein is expressed on the surface of another cell.

In one embodiment, the method of the invention is used for expansion of cells expressing the immunoreceptor of the invention. In this embodiment, said cells are incubated with an antigen-binding protein in accordance with the invention, thereby increasing proliferation and thus expanding said cells. In a preferred embodiment, the antigen-binding protein is coupled to a solid phase. In a preferred embodiment, the solid phase is a tissue culture surface. In a preferred embodiment, the solid phase is a bead, preferably a magnetic bead. In one embodiment, the antigen-binding protein is expressed on the surface of another cell.

The invention provides a method of enrichment of cells expressing the immunoreceptor in accordance with the invention, comprising the steps of stimulating or expanding the cells using the stimulation method of the invention and subsequently purifying said cells using the purification method of the invention.

In one embodiment, the method or use of the invention is an in vitro use or method. In one embodiment, the method or use of the invention is an in vivo use or method. In one embodiment, the method or use of the invention does not comprise a method for treatment of the human or animal body by surgery or therapy or a diagnostic method practised on the human or animal body.

The present invention provides a pharmaceutical composition, comprising the antigen-binding protein of the invention.

The present invention provides a pharmaceutical composition, comprising the nucleic acid of the invention.

The present invention provides a pharmaceutical composition, comprising the cells expressing the immunoreceptor of the invention.

The pharmaceutical composition of the invention can further comprise a pharmaceutically acceptable carrier, and/or excipient. The pharmaceutical composition can further comprise additional active ingredients. In a preferred embodiment, the pharmaceutical composition is useful for therapy.

The present invention provides the antigen-binding protein or pharmaceutical composition comprising same in accordance with the invention for use in a therapeutic method of depletion of cells expressing the immunoreceptor of the invention. In the method, the antigen-binding protein coupled to a cytotoxic molecule, or cells expressing the antigen-binding protein as part of a chimeric antigen receptor, optionally comprised in a pharmaceutical composition, are administered to a patient which has been administered the cells expressing the immunoreceptor of the invention, in order to deplete said cells.

The present invention provides a kit, comprising the immunoreceptor of the invention and the antigen-binding protein of the invention. The present invention provides a kit, comprising cells comprising a nucleic acid encoding the immunoreceptor of the invention and the antigen-binding protein of the invention.

Sequences

SEQ ID NO: 1 (15 aa MiH repeat sequence)
Glu Pro Lys Ser Cys Asp Thr Pro Pro Pro Cys Pro Arg Cys Pro
SEQ ID NO:2 (Upper Hinge)
Glu Leu Lys Thr Pro Leu Gly Asp Thr Thr His Thr Cys Pro Arg Cys Pro
SEQ ID NO: 3 (scFv CD19_FMC63 VH_Linker_VL)
Asp Ile Gln Met Thr Gln Thr Thr Ser Ser Leu Ser Ala Ser Leu Gly Asp Arg Val Thr Ile Ser Cys Arg Ala Ser
Gln Asp Ile Ser Lys Tyr Leu Asn Trp Tyr Gln Gln Lys Pro Asp Gly Thr Val Lys Leu Leu Ile Tyr His Thr Ser
Arg Leu His Ser Gly Val Pro Ser Arg Phe Ser Gly Ser Gly Ser Gly Thr Asp Tyr Ser Leu Thr Ile Ser Asn Leu
Glu Gln Glu Asp Ile Ala Thr Tyr Phe Cys Gln Gln Gly Asn Thr Leu Pro Tyr Thr Phe Gly Gly Gly Thr Lys Leu
Glu Ile Thr
Gly Ser Thr Ser Gly Ser Gly Lys Pro Gly Ser Gly Glu Gly Ser Thr Lys Gly
Glu Val Lys Leu Gln Glu Ser Gly Pro Gly Leu Val Ala Pro Ser Gln Ser Leu Ser Val Thr Cys Thr Val Ser Gly
Val Ser Leu Pro Asp Tyr Gly Val Ser Trp Ile Arg Gln Pro Pro Arg Lys Gly Leu Glu Trp Leu Gly Val Ile Trp
Gly Ser Glu Thr Thr Tyr Tyr Asn Ser Ala Leu Lys Ser Arg Leu Thr Ile Ile Lys Asp Asn Ser Lys Ser Gln Val
Phe Leu Lys Met Asn Ser Leu Gln Thr Asp Asp Thr Ala Ile Tyr Tyr Cys Ala Lys His Tyr Tyr Tyr Gly Gly Ser
Tyr Ala Met Asp Tyr Trp Gly Gln Gly Thr Ser Val Thr Val Ser Ser
SEQ ID NO: 27 (scFv CD19_FMC63 VH)
Asp Ile Gln Met Thr Gln Thr Thr Ser Ser Leu Ser Ala Ser Leu Gly Asp Arg Val Thr Ile Ser Cys Arg Ala Ser
Gln Asp Ile Ser Lys Tyr Leu Asn Trp Tyr Gln Gln Lys Pro Asp Gly Thr Val Lys Leu Leu Ile Tyr His Thr Ser
Arg Leu His Ser Gly Val Pro Ser Arg Phe Ser Gly Ser Gly Ser Gly Thr Asp Tyr Ser Leu Thr Ile Ser Asn Leu
Glu Gln Glu Asp Ile Ala Thr Tyr Phe Cys Gln Gln Gly Asn Thr Leu Pro Tyr Thr Phe Gly Gly Gly Thr Lys Leu
Glu Ile Thr
SEQ ID NO: 28 (scFv CD19_FMC63 VL)
Glu Val Lys Leu Gln Glu Ser Gly Pro Gly Leu Val Ala Pro Ser Gln Ser Leu Ser Val Thr Cys Thr Val Ser Gly
Val Ser Leu Pro Asp Tyr Gly Val Ser Trp Ile Arg Gln Pro Pro Arg Lys Gly Leu Glu Trp Leu Gly Val Ile Trp
Gly Ser Glu Thr Thr Tyr Tyr Asn Ser Ala Leu Lys Ser Arg Leu Thr Ile Ile Lys Asp Asn Ser Lys Ser Gln Val
Phe Leu Lys Met Asn Ser Leu Gln Thr Asp Asp Thr Ala Ile Tyr Tyr Cys Ala Lys His Tyr Tyr Tyr Gly Gly Ser
Tyr Ala Met Asp Tyr Trp Gly Gln Gly Thr Ser Val Thr Val Ser Ser
SEQ ID NO: 4 (scFv CD20_Leu16 VL_Linker_VH)
Asp Ile Val Leu Thr Gln Ser Pro Ala Ile Leu Ser Ala Ser Pro Gly Glu Lys Val Thr Met Thr Cys Arg Ala Ser
Ser Ser Val Asn Tyr Met Asp Trp Tyr Gln Lys Lys Pro Gly Ser Ser Pro Lys Pro Trp Ile Tyr Ala Thr Ser Asn
Leu Ala Ser Gly Val Pro Ala Arg Phe Ser Gly Ser Gly Ser Gly Thr Ser Tyr Ser Leu Thr Ile Ser Arg Val Glu
Ala Glu Asp Ala Ala Thr Tyr Tyr Cys Gln Gln Trp Ser Phe Asn Pro Pro Thr Phe Gly Gly Gly Thr Lys Leu
Glu Ile Lys
Gly Ser Thr Ser Gly Gly Gly Ser Gly Gly Gly Ser Gly Gly Gly Gly Ser Ser
Glu Val Gln Leu Gln Gln Ser Gly Ala Glu Leu Val Lys Pro Gly Ala Ser Val Lys Met Ser Cys Lys Ala Ser Gly
Tyr Thr Phe Thr Ser Tyr Asn Met His Trp Val Lys Gln Thr Pro Gly Gln Gly Leu Glu Trp Ile Gly Ala Ile Tyr
Pro Gly Asn Gly Asp Thr Ser Tyr Asn Gln Lys Phe Lys Gly Lys Ala Thr Leu Thr Ala Asp Lys Ser Ser Ser Thr
Ala Tyr Met Gln Leu Ser Ser Leu Thr Ser Glu Asp Ser Ala Asp Tyr Tyr Cys Ala Arg Ser Asn Tyr Tyr Gly Ser
Ser Tyr Trp Phe Phe Asp Val Trp Gly Ala Gly Thr Thr Val Thr Val Ser Ser
SEQ ID NO: 29 (scFv CD20_Leu16 VL)
Asp Ile Val Leu Thr Gln Ser Pro Ala Ile Leu Ser Ala Ser Pro Gly Glu Lys Val Thr Met Thr Cys Arg Ala Ser
Ser Ser Val Asn Tyr Met Asp Trp Tyr Gln Lys Lys Pro Gly Ser Ser Pro Lys Pro Trp Ile Tyr Ala Thr Ser Asn
Leu Ala Ser Gly Val Pro Ala Arg Phe Ser Gly Ser Gly Ser Gly Thr Ser Tyr Ser Leu Thr Ile Ser Arg Val Glu
Ala Glu Asp Ala Ala Thr Tyr Tyr Cys Gln Gln Trp Ser Phe Asn Pro Pro Thr Phe Gly Gly Gly Thr Lys Leu
Glu Ile Lys
SEQ ID NO: 30 (scFv CD20_Leu16 VH)
Glu Val Gln Leu Gln Gln Ser Gly Ala Glu Leu Val Lys Pro Gly Ala Ser Val Lys Met Ser Cys Lys Ala Ser Gly
Tyr Thr Phe Thr Ser Tyr Asn Met His Trp Val Lys Gln Thr Pro Gly Gln Gly Leu Glu Trp Ile Gly Ala Ile Tyr
Pro Gly Asn Gly Asp Thr Ser Tyr Asn Gln Lys Phe Lys Gly Lys Ala Thr Leu Thr Ala Asp Lys Ser Ser Ser Thr
Ala Tyr Met Gln Leu Ser Ser Leu Thr Ser Glu Asp Ser Ala Asp Tyr Tyr Cys Ala Arg Ser Asn Tyr Tyr Gly Ser
Ser Tyr Trp Phe Phe Asp Val Trp Gly Ala Gly Thr Thr Val Thr Val Ser Ser
SEQ ID NO: 5 (scFv ROR1_2A2 VH_4GS3_VL)
Gln Val Gln Leu Gln Gln Ser Gly Ala Glu Leu Val Arg Pro Gly Ala Ser Val Thr Leu Ser Cys Lys Ala Ser Gly
Tyr Thr Phe Ser Asp Tyr Glu Met His Trp Val Ile Gln Thr Pro Val His Gly Leu Glu Trp Ile Gly Ala Ile Asp
Pro Glu Thr Gly Gly Thr Ala Tyr Asn Gln Lys Phe Lys Gly Lys Ala Ile Leu Thr Ala Asp Lys Ser Ser Ser Thr
Ala Tyr Met Glu Leu Arg Ser Leu Thr Ser Glu Asp Ser Ala Val Tyr Tyr Cys Thr Gly Tyr Tyr Asp Tyr Asp Ser
Phe Thr Tyr Trp Gly Gln Gly Thr Leu Val Thr Val Ser Ala
Gly Gly Gly Gly Ser Gly Gly Gly Gly Ser Gly Gly Gly Gly Ser
Asp Ile Val Met Thr Gln Ser Gln Lys Ile Met Ser Thr Thr Val Gly Asp Arg Val Ser Ile Thr Cys Lys Ala Ser
Gln Asn Val Asp Ala Ala Val Ala Trp Tyr Gln Gln Lys Pro Gly Gln Ser Pro Lys Leu Leu Ile Tyr Ser Ala Ser
Asn Arg Tyr Thr Gly Val Pro Asp Arg Phe Thr Gly Ser Gly Ser Gly Thr Asp Phe Thr Leu Thr Ile Ser Asn
Met Gln Ser Glu Asp Leu Ala Asp Tyr Phe Cys Gln Gln Tyr Asp Ile Tyr Pro Tyr Thr Phe Gly Gly Gly Thr
Lys Leu Glu Ile Lys
SEQ ID NO: 31 (scFv ROR1_2A2 VH)
Gln Val Gln Leu Gln Gln Ser Gly Ala Glu Leu Val Arg Pro Gly Ala Ser Val Thr Leu Ser Cys Lys Ala Ser Gly
Tyr Thr Phe Ser Asp Tyr Glu Met His Trp Val Ile Gln Thr Pro Val His Gly Leu Glu Trp Ile Gly Ala Ile Asp
Pro Glu Thr Gly Gly Thr Ala Tyr Asn Gln Lys Phe Lys Gly Lys Ala Ile Leu Thr Ala Asp Lys Ser Ser Ser Thr
Ala Tyr Met Glu Leu Arg Ser Leu Thr Ser Glu Asp Ser Ala Val Tyr Tyr Cys Thr Gly Tyr Tyr Asp Tyr Asp Ser
Phe Thr Tyr Trp Gly Gln Gly Thr Leu Val Thr Val Ser Ala
SEQ ID NO: 32 (scFv ROR1_2A2 VL)
Asp Ile Val Met Thr Gln Ser Gln Lys Ile Met Ser Thr Thr Val Gly Asp Arg Val Ser Ile Thr Cys Lys Ala Ser
Gln Asn Val Asp Ala Ala Val Ala Trp Tyr Gln Gln Lys Pro Gly Gln Ser Pro Lys Leu Leu Ile Tyr Ser Ala Ser
Asn Arg Tyr Thr Gly Val Pro Asp Arg Phe Thr Gly Ser Gly Ser Gly Thr Asp Phe Thr Leu Thr Ile Ser Asn
Met Gln Ser Glu Asp Leu Ala Asp Tyr Phe Cys Gln Gln Tyr Asp Ile Tyr Pro Tyr Thr Phe Gly Gly Gly Thr
Lys Leu Glu Ile Lys
SEQ ID NO: 6 (scFv ROR1_4-2 VH_4GS3_VL)
Gln Glu Gln Gln Lys Glu Ser Gly Gly Gly Leu Phe Lys Pro Thr Asp Thr Leu Thr Leu Thr Cys Thr Ala Ser Gly
Phe Asp Ile Ser Ser Tyr Tyr Met Ser Trp Val Arg Gln Ala Pro Gly Asn Gly Leu Glu Trp Ile Gly Ala Ile Gly
Ile Ser Gly Asn Ala Tyr Tyr Ala Ser Trp Ala Lys Ser Arg Ser Thr Ile Thr Arg Asn Thr Asn Leu Asn Thr Val
Thr Leu Lys Met Thr Ser Leu Thr Ala Ala Asp Thr Ala Thr Tyr Phe Cys Ala Arg Asp His Pro Thr Tyr Gly
Met Asp Leu Trp Gly Pro Gly Thr Leu Val Thr Val Ser Ser
Gly Gly Gly Gly Ser Gly Gly Gly Gly Ser Gly Gly Gly Gly Ser
Ser Tyr Glu Leu Thr Gln Leu Pro Ser Val Ser Val Ser Leu Gly Gln Thr Ala Arg Ile Thr Cys Glu Gly Asn Asn
Ile Gly Ser Lys Ala Val His Trp Tyr Gln Gln Lys Pro Gly Leu Ala Pro Gly Leu Leu Ile Tyr Asp Asp Asp Glu
Arg Pro Ser Gly Val Pro Asp Arg Phe Ser Gly Ser Asn Ser Gly Asp Thr Ala Thr Leu Thr Ile Ser Gly Ala Gln
Ala Gly Asp Glu Ala Asp Tyr Tyr Cys Gln Val Trp Asp Ser Ser Ala Tyr Val Phe Gly Gly Gly Thr Gln Leu Thr
Val Thr Gly
SEQ ID NO: 33 (scFv ROR1_4-2 VH)
Gln Glu Gln Gln Lys Glu Ser Gly Gly Gly Leu Phe Lys Pro Thr Asp Thr Leu Thr Leu Thr Cys Thr Ala Ser Gly
Phe Asp Ile Ser Ser Tyr Tyr Met Ser Trp Val Arg Gln Ala Pro Gly Asn Gly Leu Glu Trp Ile Gly Ala Ile Gly
Ile Ser Gly Asn Ala Tyr Tyr Ala Ser Trp Ala Lys Ser Arg Ser Thr Ile Thr Arg Asn Thr Asn Leu Asn Thr Val
Thr Leu Lys Met Thr Ser Leu Thr Ala Ala Asp Thr Ala Thr Tyr Phe Cys Ala Arg Asp His Pro Thr Tyr Gly
Met Asp Leu Trp Gly Pro Gly Thr Leu Val Thr Val Ser Ser
SEQ ID NO: 34 (scFv ROR1_4-2 VL)
Ser Tyr Glu Leu Thr Gln Leu Pro Ser Val Ser Val Ser Leu Gly Gln Thr Ala Arg Ile Thr Cys Glu Gly Asn Asn
Ile Gly Ser Lys Ala Val His Trp Tyr Gln Gln Lys Pro Gly Leu Ala Pro Gly Leu Leu Ile Tyr Asp Asp Asp Glu
Arg Pro Ser Gly Val Pro Asp Arg Phe Ser Gly Ser Asn Ser Gly Asp Thr Ala Thr Leu Thr Ile Ser Gly Ala Gln
Ala Gly Asp Glu Ala Asp Tyr Tyr Cys Gln Val Trp Asp Ser Ser Ala Tyr Val Phe Gly Gly Gly Thr Gln Leu Thr
Val Thr Gly
SEQ ID NO: 7 (scFv ROR1_R11 VH_4GS3_VL)
Gln Ser Val Lys Glu Ser Glu Gly Asp Leu Val Thr Pro Ala Gly Asn Leu Thr Leu Thr Cys Thr Ala Ser Gly Ser
Asp Ile Asn Asp Tyr Pro Ile Ser Trp Val Arg Gln Ala Pro Gly Lys Gly Leu Glu Trp Ile Gly Phe Ile Asn Ser
Gly Gly Ser Thr Trp Tyr Ala Ser Trp Val Lys Gly Arg Phe Thr Ile Ser Arg Thr Ser Thr Thr Val Asp Leu Lys
Met Thr Ser Leu Thr Thr Asp Asp Thr Ala Thr Tyr Phe Cys Ala Arg Gly Tyr Ser Thr Tyr Tyr Gly Asp Phe
Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Met Thr Gln Thr Pro Ser Ser Thr Ser Gly Ala Val Gly Gly Thr Val Thr Ile Asn Cys Gln Ala Ser
Gln Ser Ile Asp Ser Asn Leu Ala Trp Phe Gln Gln Lys Pro Gly Gln Pro Pro Thr Leu Leu Ile Tyr Arg Ala Ser
Asn Leu Ala Ser Gly Val Pro Ser Arg Phe Ser Gly Ser Arg Ser Gly Thr Glu Tyr Thr Leu Thr Ile Ser Gly Val
Gln Arg Glu Asp Ala Ala Thr Tyr Tyr Cys Leu Gly Gly Val Gly Asn Val Ser Tyr Arg Thr Ser Phe Gly Gly Gly
Thr Glu Val Val Val Lys
SEQ ID NO: 35 (scFv ROR1_R11 VH)
Gln Ser Val Lys Glu Ser Glu Gly Asp Leu Val Thr Pro Ala Gly Asn Leu Thr Leu Thr Cys Thr Ala Ser Gly Ser
Asp Ile Asn Asp Tyr Pro Ile Ser Trp Val Arg Gln Ala Pro Gly Lys Gly Leu Glu Trp Ile Gly Phe Ile Asn Ser
Gly Gly Ser Thr Trp Tyr Ala Ser Trp Val Lys Gly Arg Phe Thr Ile Ser Arg Thr Ser Thr Thr Val Asp Leu Lys
Met Thr Ser Leu Thr Thr Asp Asp Thr Ala Thr Tyr Phe Cys Ala Arg Gly Tyr Ser Thr Tyr Tyr Gly Asp Phe
Asn Ile Trp Gly Pro Gly Thr Leu Val Thr Ile Ser Ser
SEQ ID NO: 36 (scFv ROR1_R11 VL)
Glu Leu Val Met Thr Gln Thr Pro Ser Ser Thr Ser Gly Ala Val Gly Gly Thr Val Thr Ile Asn Cys Gln Ala Ser
Gln Ser Ile Asp Ser Asn Leu Ala Trp Phe Gln Gln Lys Pro Gly Gln Pro Pro Thr Leu Leu Ile Tyr Arg Ala Ser
Asn Leu Ala Ser Gly Val Pro Ser Arg Phe Ser Gly Ser Arg Ser Gly Thr Glu Tyr Thr Leu Thr Ile Ser Gly Val
Gln Arg Glu Asp Ala Ala Thr Tyr Tyr Cys Leu Gly Gly Val Gly Asn Val Ser Tyr Arg Thr Ser Phe Gly Gly Gly
Thr Glu Val Val Val Lys
SEQ ID NO: 8 (scFv ROR1_R12 VH_Linker_VL)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
SEQ ID NO: 37 (scFv ROR1_R12 VH)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
SEQ ID NO: 38 (scFv ROR1_R12 VL)
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
SEQ ID NO: 9 (scFv ROR2_4-1 VH_4GS3_VL)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
Gly Gly Gly Gly Ser Gly Gly Gly Gly Ser Gly Gly Gly Gly Ser
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 39 (scFv ROR2_4-1 VH)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
SEQ ID NO: 40 (scFv ROR2_4-1 VL)
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 10 (scFv SLAM F7_ERCS409 VH_4GS3_VL)
Gln Ser Val Lys Glu Ser Glu Gly Gly Leu Phe Lys Pro Thr Asp Thr Leu Thr Leu Thr Cys Thr Val Ser Gly Phe
Ser Leu Asn Ser Tyr Gly Val Ile Trp Val Arg Gln Ala Pro Gly Asn Gly Leu Glu Tyr Ile Gly Ile Ile Gly Ser Ser
Gly Asn Thr Tyr Tyr Ala Ser Trp Ala Lys Ser Arg Ser Thr Ile Thr Arg Asn Thr Arg Leu Asn Thr Val Thr Leu
Lys Met Thr Ser Leu Thr Ala Ala Asp Thr Ala Thr Tyr Phe Cys Ala Arg Tyr Tyr Gly Asp Ser Gly Phe Asp
Ser Trp Gly Pro Gly Thr Leu Val Thr Val Ser Ser
Gly Gly Gly Gly Ser Gly Gly Gly Gly Ser Gly Gly Gly Gly Ser
Ala Gln Val Leu Thr Gln Thr Pro Ser Ser Thr Ser Val Ala Val Gly Gly Thr Val Thr Ile Lys Cys Gln Ala Ser
Gln Ser Ile Gly Ser Trp Leu Ser Trp Tyr Gln Gln Lys Pro Gly Gln Pro Pro Lys Leu Leu Ile Tyr Gly Ala Ser
Asn Leu Ala Ser Gly Val Pro Ser Arg Phe Lys Gly Gly Arg Ser Gly Thr Glu Tyr Ser Leu Thr Ile Ser Gly Val
Gln Arg Glu Asp Ala Ala Thr Tyr Tyr Cys Leu Gly Ala Ser Pro Asn Gly Trp Ala Phe Gly Ala Gly Thr Asn Val
Glu Ile Lys
SEQ ID NO: 41 (scFv SLAM F7_ERCS409 VH)
Gln Ser Val Lys Glu Ser Glu Gly Gly Leu Phe Lys Pro Thr Asp Thr Leu Thr Leu Thr Cys Thr Val Ser Gly Phe
Ser Leu Asn Ser Tyr Gly Val Ile Trp Val Arg Gln Ala Pro Gly Asn Gly Leu Glu Tyr Ile Gly Ile Ile Gly Ser Ser
Gly Asn Thr Tyr Tyr Ala Ser Trp Ala Lys Ser Arg Ser Thr Ile Thr Arg Asn Thr Arg Leu Asn Thr Val Thr Leu
Lys Met Thr Ser Leu Thr Ala Ala Asp Thr Ala Thr Tyr Phe Cys Ala Arg Tyr Tyr Gly Asp Ser Gly Phe Asp
Ser Trp Gly Pro Gly Thr Leu Val Thr Val Ser Ser
SEQ ID NO: 42 (scFv SLAM F7_ERCS409 VL)
Ala Gln Val Leu Thr Gln Thr Pro Ser Ser Thr Ser Val Ala Val Gly Gly Thr Val Thr Ile Lys Cys Gln Ala Ser
Gln Ser Ile Gly Ser Trp Leu Ser Trp Tyr Gln Gln Lys Pro Gly Gln Pro Pro Lys Leu Leu Ile Tyr Gly Ala Ser
Asn Leu Ala Ser Gly Val Pro Ser Arg Phe Lys Gly Gly Arg Ser Gly Thr Glu Tyr Ser Leu Thr Ile Ser Gly Val
Gln Arg Glu Asp Ala Ala Thr Tyr Tyr Cys Leu Gly Ala Ser Pro Asn Gly Trp Ala Phe Gly Ala Gly Thr Asn Val
Glu Ile Lys
SEQ ID NO: 11 (scFv SLAM F7_huLuc63 VH_4GS3_VL)
Glu Val Gln Leu Val Glu Ser Gly Gly Gly Leu Val Gln Pro Gly Gly Ser Leu Arg Leu Ser Cys Ala Ala Ser Gly
Phe Asp Phe Ser Arg Tyr Trp Met Ser Trp Val Arg Gln Ala Pro Gly Lys Gly Leu Glu Trp Ile Gly Glu Ile Asn
Pro Asp Ser Ser Thr Ile Asn Tyr Ala Pro Ser Leu Lys Asp Lys Phe Ile Ile Ser Arg Asp Asn Ala Lys Asn Ser
Leu Tyr Leu Gln Met Asn Ser Leu Arg Ala Glu Asp Thr Ala Val Tyr Tyr Cys Ala Arg Pro Asp Gly Asn Tyr
Trp Tyr Phe Asp Val Trp Gly Gln Gly Thr Leu Val Thr Val Ser Ser
Gly Gly Gly Gly Ser Gly Gly Gly Gly Ser Gly Gly Gly Gly Ser
Asp Ile Gln Met Thr Gln Ser Pro Ser Ser Leu Ser Ala Ser Val Gly Asp Arg Val Thr Ile Thr Cys Lys Ala Ser
Gln Asp Val Gly Ile Ala Val Ala Trp Tyr Gln Gln Lys Pro Gly Lys Val Pro Lys Leu Leu Ile Tyr Trp Ala Ser
Thr Arg His Thr Gly Val Pro Asp Arg Phe Ser Gly Ser Gly Ser Gly Thr Asp Phe Thr Leu Thr Ile Ser Ser Leu
Gln Pro Glu Asp Val Ala Thr Tyr Tyr Cys Gln Gln Tyr Ser Ser Tyr Pro Tyr Thr Phe Gly Gln Gly Thr Lys Val
Glu Ile Lys
SEQ ID NO: 43 (scFv SLAM F7_huLuc63 VH)
Glu Val Gln Leu Val Glu Ser Gly Gly Gly Leu Val Gln Pro Gly Gly Ser Leu Arg Leu Ser Cys Ala Ala Ser Gly
Phe Asp Phe Ser Arg Tyr Trp Met Ser Trp Val Arg Gln Ala Pro Gly Lys Gly Leu Glu Trp Ile Gly Glu Ile Asn
Pro Asp Ser Ser Thr Ile Asn Tyr Ala Pro Ser Leu Lys Asp Lys Phe Ile Ile Ser Arg Asp Asn Ala Lys Asn Ser
Leu Tyr Leu Gln Met Asn Ser Leu Arg Ala Glu Asp Thr Ala Val Tyr Tyr Cys Ala Arg Pro Asp Gly Asn Tyr
Trp Tyr Phe Asp Val Trp Gly Gln Gly Thr Leu Val Thr Val Ser Ser
SEQ ID NO: 44 (scFv SLAM F7_huLuc63 VL)
Asp Ile Gln Met Thr Gln Ser Pro Ser Ser Leu Ser Ala Ser Val Gly Asp Arg Val Thr Ile Thr Cys Lys Ala Ser
Gln Asp Val Gly Ile Ala Val Ala Trp Tyr Gln Gln Lys Pro Gly Lys Val Pro Lys Leu Leu Ile Tyr Trp Ala Ser
Thr Arg His Thr Gly Val Pro Asp Arg Phe Ser Gly Ser Gly Ser Gly Thr Asp Phe Thr Leu Thr Ile Ser Ser Leu
Gln Pro Glu Asp Val Ala Thr Tyr Tyr Cys Gln Gln Tyr Ser Ser Tyr Pro Tyr Thr Phe Gly Gln Gly Thr Lys Val
Glu Ile Lys
SEQ ID NO: 12 (scFv FLT3_BV10 VH_4GS3_VL)
Gln Val Gln Leu Lys Gln Ser Gly Pro Gly Leu Val Gln Pro Ser Gln Ser Leu Ser Ile Thr Cys Thr Val Ser Gly
Phe Ser Leu Thr Asn Tyr Gly Leu His Trp Val Arg Gln Ser Pro Gly Lys Gly Leu Glu Trp Leu Gly Val Ile Trp
Ser Gly Gly Ser Thr Asp Tyr Asn Ala Ala Phe Ile Ser Arg Leu Ser Ile Ser Lys Asp Asn Ser Lys Ser Gln Val
Phe Phe Lys Met Asn Ser Leu Gln Ala Asp Asp Thr Ala Ile Tyr Tyr Cys Ala Arg Lys Gly Gly Ile Tyr Tyr Ala
Asn His Tyr Tyr Ala Met Asp Tyr Trp Gly Gln Gly Thr Ser Val Thr Val Ser Ser
Gly Gly Gly Gly Ser Gly Gly Gly Gly Ser Gly Gly Gly Gly Ser
Asp Ile Val Met Thr Gln Ser Pro Ser Ser Leu Ser Val Ser Ala Gly Glu Lys Val Thr Met Ser Cys Lys Ser Ser
Gln Ser Leu Leu Asn Ser Gly Asn Gln Lys Asn Tyr Met Ala Trp Tyr Gln Gln Lys Pro Gly Gln Pro Pro Lys
Leu Leu Ile Tyr Gly Ala Ser Thr Arg Glu Ser Gly Val Pro Asp Arg Phe Thr Gly Ser Gly Ser Gly Thr Asp Phe
Thr Leu Thr Ile Ser Ser Val Gln Ala Glu Asp Leu Ala Val Tyr Tyr Cys Gln Asn Asp His Ser Tyr Pro Leu Thr
Phe Gly Ala Gly Thr Lys Leu Glu Leu Lys Arg
SEQ ID NO: 45 (scFv FLT3_BV10 VH)
Gln Val Gln Leu Lys Gln Ser Gly Pro Gly Leu Val Gln Pro Ser Gln Ser Leu Ser Ile Thr Cys Thr Val Ser Gly
Phe Ser Leu Thr Asn Tyr Gly Leu His Trp Val Arg Gln Ser Pro Gly Lys Gly Leu Glu Trp Leu Gly Val Ile Trp
Ser Gly Gly Ser Thr Asp Tyr Asn Ala Ala Phe Ile Ser Arg Leu Ser Ile Ser Lys Asp Asn Ser Lys Ser Gln Val
Phe Phe Lys Met Asn Ser Leu Gln Ala Asp Asp Thr Ala Ile Tyr Tyr Cys Ala Arg Lys Gly Gly Ile Tyr Tyr Ala
Asn His Tyr Tyr Ala Met Asp Tyr Trp Gly Gln Gly Thr Ser Val Thr Val Ser Ser
SEQ ID NO: 46 (scFv FLT3_BV10 VL)
Asp Ile Val Met Thr Gln Ser Pro Ser Ser Leu Ser Val Ser Ala Gly Glu Lys Val Thr Met Ser Cys Lys Ser Ser
Gln Ser Leu Leu Asn Ser Gly Asn Gln Lys Asn Tyr Met Ala Trp Tyr Gln Gln Lys Pro Gly Gln Pro Pro Lys
Leu Leu Ile Tyr Gly Ala Ser Thr Arg Glu Ser Gly Val Pro Asp Arg Phe Thr Gly Ser Gly Ser Gly Thr Asp Phe
Thr Leu Thr Ile Ser Ser Val Gln Ala Glu Asp Leu Ala Val Tyr Tyr Cys Gln Asn Asp His Ser Tyr Pro Leu Thr
Phe Gly Ala Gly Thr Lys Leu Glu Leu Lys Arg
SEQ ID NO: 13 (scFv FLT3_4G8 VH_4GS3_VL)
Gln Val Gln Leu Gln Gln Pro Gly Ala Glu Leu Val Lys Pro Gly Ala Ser Leu Lys Leu Ser Cys Lys Ser Ser Gly
Tyr Thr Phe Thr Ser Tyr Trp Met His Trp Val Arg Gln Arg Pro Gly His Gly Leu Glu Trp Ile Gly Glu Ile Asp
Pro Ser Asp Ser Tyr Lys Asp Tyr Asn Gln Lys Phe Lys Asp Lys Ala Thr Leu Thr Val Asp Arg Ser Ser Asn
Thr Ala Tyr Met His Leu Ser Ser Leu Thr Ser Asp Asp Ser Ala Val Tyr Tyr Cys Ala Arg Ala Ile Thr Thr Thr
Pro Phe Asp Phe Trp Gly Gln Gly Thr Thr Leu Thr Val Ser Ser
Gly Gly Gly Gly Ser Gly Gly Gly Gly Ser Gly Gly Gly Gly Ser
Asp Ile Val Leu Thr Gln Ser Pro Ala Thr Leu Ser Val Thr Pro Gly Asp Ser Val Ser Leu Ser Cys Arg Ala Ser
Gln Ser Ile Ser Asn Asn Leu His Trp Tyr Gln Gln Lys Ser His Glu Ser Pro Arg Leu Leu Ile Lys Tyr Ala Ser
Gln Ser Ile Ser Gly Ile Pro Ser Arg Phe Ser Gly Ser Gly Ser Gly Thr Asp Phe Thr Leu Ser Ile Asn Ser Val
Glu Thr Glu Asp Phe Gly Val Tyr Phe Cys Gln Gln Ser Asn Thr Trp Pro Tyr Thr Phe Gly Gly Gly Thr Lys
Leu Glu Ile Lys Arg
SEQ ID NO: 47 (scFv FLT3_4G8 VH)
Gln Val Gln Leu Gln Gln Pro Gly Ala Glu Leu Val Lys Pro Gly Ala Ser Leu Lys Leu Ser Cys Lys Ser Ser Gly
Tyr Thr Phe Thr Ser Tyr Trp Met His Trp Val Arg Gln Arg Pro Gly His Gly Leu Glu Trp Ile Gly Glu Ile Asp
Pro Ser Asp Ser Tyr Lys Asp Tyr Asn Gln Lys Phe Lys Asp Lys Ala Thr Leu Thr Val Asp Arg Ser Ser Asn
Thr Ala Tyr Met His Leu Ser Ser Leu Thr Ser Asp Asp Ser Ala Val Tyr Tyr Cys Ala Arg Ala Ile Thr Thr Thr
Pro Phe Asp Phe Trp Gly Gln Gly Thr Thr Leu Thr Val Ser Ser
SEQ ID NO: 48 (scFv FLT3_4G8 VL)
Asp Ile Val Leu Thr Gln Ser Pro Ala Thr Leu Ser Val Thr Pro Gly Asp Ser Val Ser Leu Ser Cys Arg Ala Ser
Gln Ser Ile Ser Asn Asn Leu His Trp Tyr Gln Gln Lys Ser His Glu Ser Pro Arg Leu Leu Ile Lys Tyr Ala Ser
Gln Ser Ile Ser Gly Ile Pro Ser Arg Phe Ser Gly Ser Gly Ser Gly Thr Asp Phe Thr Leu Ser Ile Asn Ser Val
Glu Thr Glu Asp Phe Gly Val Tyr Phe Cys Gln Gln Ser Asn Thr Trp Pro Tyr Thr Phe Gly Gly Gly Thr Lys
Leu Glu Ile Lys Arg
SEQ ID NO: 14 (scFv Siglec-6_JML-1 VH_4GS3_VL)
Lys Val Gln Leu Leu Glu Ser Gly Gly Gly Leu Val Gln Pro Gly Arg Ser Leu Arg Leu Ser Cys Ala Ala Ser Gly
Phe Thr Phe Asp Asp Tyr Gly Met His Trp Val Arg Gln Ala Pro Gly Lys Gly Leu Glu Trp Val Ser Gly Ile Ser
Trp Asn Ser Gly Ser Ile Gly Tyr Ala Asp Ser Val Lys Gly Arg Phe Thr Ile Ser Arg Asp Asn Ser Lys Asn Thr
Leu Tyr Leu Gln Met Asn Ser Leu Arg Ala Glu Asp Thr Ala Val Tyr Tyr Cys Ala Arg Gly Gly Gln Thr Ile Asp
Ile Trp Gly Gln Gly Thr Met Val Thr Val Ser Ser
Gly Gly Gly Gly Ser Gly Gly Gly Gly Ser Gly Gly Gly Gly Ser
Asp Ile Gln Met Thr Gln Ser Pro Ser Ser Leu Ser Ala Ser Val Gly Asp Arg Val Thr Ile Thr Cys Arg Ala Ser
Gln Ser Ile Ser Ser Tyr Leu Asn Trp Tyr Gln Gln Lys Pro Gly Lys Ala Pro Lys Leu Leu Ile Tyr Ala Ala Ser
Ser Leu Gln Ser Gly Val Pro Ser Arg Phe Ser Gly Ser Gly Ser Gly Thr Asp Phe Thr Leu Thr Ile Ser Ser Leu
Gln Pro Glu Asp Phe Ala Thr Tyr Tyr Cys Gln Gln Ser Tyr Ser Thr Pro Phe Thr Phe Gly Pro Gly Thr Lys
Val Asp Ile Lys
SEQ ID NO: 49 (scFv Siglec-6_JML-1 VH)
Lys Val Gln Leu Leu Glu Ser Gly Gly Gly Leu Val Gln Pro Gly Arg Ser Leu Arg Leu Ser Cys Ala Ala Ser Gly
Phe Thr Phe Asp Asp Tyr Gly Met His Trp Val Arg Gln Ala Pro Gly Lys Gly Leu Glu Trp Val Ser Gly Ile Ser
Trp Asn Ser Gly Ser Ile Gly Tyr Ala Asp Ser Val Lys Gly Arg Phe Thr Ile Ser Arg Asp Asn Ser Lys Asn Thr
Leu Tyr Leu Gln Met Asn Ser Leu Arg Ala Glu Asp Thr Ala Val Tyr Tyr Cys Ala Arg Gly Gly Gln Thr Ile Asp
Ile Trp Gly Gln Gly Thr Met Val Thr Val Ser Ser
SEQ ID NO: 50 (scFv Siglec-6_JML-1 VL)
Asp Ile Gln Met Thr Gln Ser Pro Ser Ser Leu Ser Ala Ser Val Gly Asp Arg Val Thr Ile Thr Cys Arg Ala Ser
Gln Ser Ile Ser Ser Tyr Leu Asn Trp Tyr Gln Gln Lys Pro Gly Lys Ala Pro Lys Leu Leu Ile Tyr Ala Ala Ser
Ser Leu Gln Ser Gly Val Pro Ser Arg Phe Ser Gly Ser Gly Ser Gly Thr Asp Phe Thr Leu Thr Ile Ser Ser Leu
Gln Pro Glu Asp Phe Ala Thr Tyr Tyr Cys Gln Gln Ser Tyr Ser Thr Pro Phe Thr Phe Gly Pro Gly Thr Lys
Val Asp Ile Lys
SEQ ID NO: 15 (scFv avb3_LM609v7 VH_4GS3_VL)
Gln Val Gln Leu Gln Glu Ser Gly Pro Gly Leu Val Lys Pro Ser Gln Thr Leu Ser Leu Thr Cys Thr Val Ser Gly
Ala Ser Ile Ser Arg Gly Gly Tyr Tyr Trp Ser Trp Ile Arg Gln Tyr Pro Gly Lys Gly Leu Glu Trp Ile Gly Tyr Ile
His His Ser Gly Ser Thr Tyr Tyr Asn Pro Ser Leu Lys Ser Arg Val Thr Ile Ala Ile Asp Thr Ser Lys Asn Gln
Leu Ser Leu Arg Leu Thr Ser Val Thr Ala Ala Asp Thr Ala Val Tyr Tyr Cys Ala Arg His Asn Tyr Gly Ser Phe
Ala Tyr Trp Gly Gln Gly Thr Leu Val Thr Val Ser Ser
Gly Gly Gly Gly Ser Gly Gly Gly Gly Ser Gly Gly Gly Gly Ser
Glu Leu Val Met Thr Gln Ser Pro Glu Phe Gln Ser Val Thr Pro Lys Glu Thr Val Thr Ile Thr Cys Arg Ala Ser
Gln Asp Ile Gly Asn Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Ser Arg Leu
Glu Pro Glu Asp Phe Ala Val Tyr Tyr Cys Gln Gln Ser Asn Ser Trp Pro His Thr Phe Gly Gln Gly Thr Lys Leu
Glu Ile Lys
SEQ ID NO: 51 (scFv avb3_LM609v7 VH)
Gln Val Gln Leu Gln Glu Ser Gly Pro Gly Leu Val Lys Pro Ser Gln Thr Leu Ser Leu Thr Cys Thr Val Ser Gly
Ala Ser Ile Ser Arg Gly Gly Tyr Tyr Trp Ser Trp Ile Arg Gln Tyr Pro Gly Lys Gly Leu Glu Trp Ile Gly Tyr Ile
His His Ser Gly Ser Thr Tyr Tyr Asn Pro Ser Leu Lys Ser Arg Val Thr Ile Ala Ile Asp Thr Ser Lys Asn Gln
Leu Ser Leu Arg Leu Thr Ser Val Thr Ala Ala Asp Thr Ala Val Tyr Tyr Cys Ala Arg His Asn Tyr Gly Ser Phe
Ala Tyr Trp Gly Gln Gly Thr Leu Val Thr Val Ser Ser
SEQ ID NO: 52 (scFv avb3_LM609v7 VL)
Glu Leu Val Met Thr Gln Ser Pro Glu Phe Gln Ser Val Thr Pro Lys Glu Thr Val Thr Ile Thr Cys Arg Ala Ser
Gln Asp Ile Gly Asn Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Ser Arg Leu
Glu Pro Glu Asp Phe Ala Val Tyr Tyr Cys Gln Gln Ser Asn Ser Trp Pro His Thr Phe Gly Gln Gly Thr Lys Leu
Glu Ile Lys
SEQ ID NO: 16 (scFv avb3_LM609v11 VH_4GS3_VL)
Gln Val Gln Leu Val Gln Ser Gly Ala Glu Val Arg Lys Pro Gly Ser Ser Val Arg Val Ser Cys Lys Ala Ser Gly
Gly Thr Phe Ser Gly Phe Ala Val Ser Trp Val Arg Gln Ala Pro Gly Gln Arg Phe Glu Trp Leu Gly Gly Ile Val
Ala Ser Leu Gly Ser Thr Asp Tyr Ala Gln Lys Phe Gln Asp Lys Leu Thr Ile Thr Val Asp Glu Ser Thr Ala Thr
Val Tyr Met Glu Met Arg Asn Leu Arg Ser Asp Asp Thr Ala Val Tyr Tyr Cys Ala Arg His Asn Tyr Gly Ser
Phe Ala Tyr Trp Gly Gln Gly Thr Leu Val Thr Val Ser Ser
Gly Gly Gly Gly Ser Gly Gly Gly Gly Ser Gly Gly Gly Gly Ser
Glu Leu Val Met Thr Gln Ser Pro Glu Phe Gln Ser Val Thr Pro Lys Glu Thr Val Thr Ile Thr Cys Arg Ala Ser
Gln Asp Ile Gly Thr Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Tyr Ser Leu
Glu Ala Glu Asp Phe Ala Val Tyr Tyr Cys Gln Gln Ser Asn Ser Trp Pro His Thr Phe Gly Gln Gly Thr Lys Leu
Glu Ile Lys
SEQ ID NO: 53 (scFv avb3_LM609v11 VH)
Gln Val Gln Leu Val Gln Ser Gly Ala Glu Val Arg Lys Pro Gly Ser Ser Val Arg Val Ser Cys Lys Ala Ser Gly
Gly Thr Phe Ser Gly Phe Ala Val Ser Trp Val Arg Gln Ala Pro Gly Gln Arg Phe Glu Trp Leu Gly Gly Ile Val
Ala Ser Leu Gly Ser Thr Asp Tyr Ala Gln Lys Phe Gln Asp Lys Leu Thr Ile Thr Val Asp Glu Ser Thr Ala Thr
Val Tyr Met Glu Met Arg Asn Leu Arg Ser Asp Asp Thr Ala Val Tyr Tyr Cys Ala Arg His Asn Tyr Gly Ser
Phe Ala Tyr Trp Gly Gln Gly Thr Leu Val Thr Val Ser Ser
SEQ ID NO: 54 (scFv avb3_LM609v11 VL)
Glu Leu Val Met Thr Gln Ser Pro Glu Phe Gln Ser Val Thr Pro Lys Glu Thr Val Thr Ile Thr Cys Arg Ala Ser
Gln Asp Ile Gly Thr Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Tyr Ser Leu
Glu Ala Glu Asp Phe Ala Val Tyr Tyr Cys Gln Gln Ser Asn Ser Trp Pro His Thr Phe Gly Gln Gly Thr Lys Leu
Glu Ile Lys
SEQ ID NO: 17 (scFv BCMA_BCMA30 VH_4GS3_VL)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ser Ser
Thr Ala Tyr Met Glu Leu Ser Ser Leu Arg Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
Gly Gly Gly Gly Ser Gly Gly Gly Gly Ser Gly Gly Gly Gly Ser
Asp Ile Val Met Thr Gln Thr Pro Leu Ser Leu Ser Val Thr Pro Gly Glu Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Ala Asp Phe Thr Leu
Lys Ile Ser Arg Val Glu Ala Glu Asp Val Gly Val Tyr Tyr Cys Ala Glu Thr Ser His Val Pro Trp Thr Phe Gly
Gln Gly Thr Lys Leu Glu Ile Lys
SEQ ID NO: 55 (scFv BCMA_BCMA30 VH)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ser Ser
Thr Ala Tyr Met Glu Leu Ser Ser Leu Arg Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
SEQ ID NO: 56 (scFv BCMA_BCMA30 VL)
Asp Ile Val Met Thr Gln Thr Pro Leu Ser Leu Ser Val Thr Pro Gly Glu Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Ala Asp Phe Thr Leu
Lys Ile Ser Arg Val Glu Ala Glu Asp Val Gly Val Tyr Tyr Cys Ala Glu Thr Ser His Val Pro Trp Thr Phe Gly
Gln Gly Thr Lys Leu Glu Ile Lys
SEQ ID NO: 18 (scFv BCMA_BCMA50 VH_4GS3_VL)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ile Asn
Thr Ala Tyr Met Glu Leu Ser Ser Leu Thr Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
Gly Gly Gly Gly Ser Gly Gly Gly Gly Ser Gly Gly Gly Gly Ser
Asp Ile Val Met Thr Gln Thr Pro Leu Ser Leu Ser Val Thr Pro Gly Gln Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Thr Asp Phe Thr Leu
Lys Ile Ser Arg Val Glu Ala Glu Asp Val Gly Ile Tyr Tyr Cys Ser Gln Ser Ser Ile Tyr Pro Trp Thr Phe Gly Gln
Gly Thr Lys Leu Glu Ile Lys
SEQ ID NO: 57 (scFv BCMA_BCMA50 VH)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ile Asn
Thr Ala Tyr Met Glu Leu Ser Ser Leu Thr Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
SEQ ID NO: 58 (scFv BCMA_BCMA50 VL)
Asp Ile Val Met Thr Gln Thr Pro Leu Ser Leu Ser Val Thr Pro Gly Gln Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Thr Asp Phe Thr Leu
Lys Ile Ser Arg Val Glu Ala Glu Asp Val Gly Ile Tyr Tyr Cys Ser Gln Ser Ser Ile Tyr Pro Trp Thr Phe Gly Gln
Gly Thr Lys Leu Glu Ile Lys
SEQ ID NO: 19 (anti MiH repeats heavy chain variable region)
Gln Val Gln Leu Leu Glu Ser Gly Pro Gly Leu Val Ala Pro Ser Gln Thr Leu Ser Ile Thr Cys Thr Val Ser Gly
Phe Ser Phe Thr Asn Tyr Asp Leu His Trp Val Arg Gln Pro Pro Gly Lys Gly Leu Glu Trp Leu Gly Val Ile Trp
Ala Val Gly Ser Thr Asn Tyr Asn Ser Ala Leu Met Ser Arg Leu Ser Ile Ser Lys Asp Asn Ser Lys Ser Gln Val
Phe Leu Lys Met Asn Ser Leu Gln Thr Asp Asp Thr Ala Ile Tyr Tyr Cys Ala Arg Glu Glu Asp Tyr Arg Tyr
Gly Met Asp Tyr Trp Gly Gln Gly Thr Ser Val Thr Val Ser Ser
SEQ ID NO: 20 (anti MiH repeats heavy CDR1)
Gly Phe Ser Phe Thr Asn Tyr
SEQ ID NO: 21 (anti MiH repeats heavy CDR2)
Trp Ala Val Gly Ser
SEQ ID NO: 22 (anti MiH repeats heavy CDR3)
Glu Glu Asp Tyr Arg Tyr Gly Met Asp Tyr
SEQ ID NO: 23 (anti MiH repeats light chain variable region)
Glu Leu Val Met Thr Gln Ser Pro Leu Ser Leu Pro Val Ser Leu Gly Asp Gln Ala Ser Ile Ser Cys Arg Ser Ser
Gln Ser Leu Val His Ser Asn Gly Asn Thr Tyr Leu His Trp Tyr Leu Gln Lys Pro Gly Gln Ser Pro Lys Leu Leu
Ile Tyr Lys Val Ser Asn Arg Phe Ser Gly Val Pro Asp Arg Phe Ser Gly Ser Gly Ser Gly Thr Asp Phe Thr Leu
Lys Ile Ser Arg Val Glu Ala Glu Asp Leu Gly Val Tyr Phe Cys Ser Gln Ser Thr His Val Pro Tyr Thr Phe Gly
Gly Gly Thr Lys Leu Glu Ile Lys
SEQ ID NO: 24 (anti MiH repeats light CDR1)
Arg Ser Ser Gln Ser Leu Val His Ser Asn Gly Asn Thr Tyr Leu His
SEQ ID NO: 25 (anti MiH repeats light CDR2)
Lys Val Ser Asn Arg Phe Ser
SEQ ID NO: 26 (anti MiH repeats light CDR3)
Ser Gln Ser Thr His Val Pro Tyr Thr
SEQ ID NO: 59 (CPRCP)
CPRCP
SEQ ID NO: 60 (IgG3 lower hinge)
APELLGGP
SEQ ID NO: 61 (Lentiviral vector backbone 5′)
GTTAGACCAGATCTGAGCCTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGAGATCCCTCAGACCCTTTTA
GTCAGTGTGGAAAATCTCTAGCAGTGGCGCCCGAACAGGGACTTGAAAGCGAAAGGGAAACCAGAGGAGCT
CTCTCGACGCAGGACTCGGCTTGCTGAAGCGCGCACGGCAAGAGGCGAGGGGCGGCGACTGGTGAGTACGC
CAAAAATTTTGACTAGCGGAGGCTAGAAGGAGAGAGATGGGTGCGAGAGCGTCAGTATTAAGCGGGGGAGA
ATTAGATCGATGGGAAAAAATTCGGTTAAGGCCAGGGGGAAAGAAAAAATATAAATTAAAACATATAGTATG
GGCAAGCAGGGAGCTAGAACGATTCGCAGTTAATCCTGGCCTGTTAGAAACATCAGAAGGCTGTAGACAAAT
ACTGGGACAGCTACAACCATCCCTTCAGACAGGATCAGAAGAACTTAGATCATTATATAATACAGTAGCAACCC
TCTATTGTGTGCATCAAAGGATAGAGATAAAAGACACCAAGGAAGCTTTAGACAAGATAGAGGAAGAGCAAA
ACAAAAGTAAGAAAAAAGCACAGCAAGCAGCAGCTGACACAGGACACAGCAATCAGGTCAGCCAAAATTACC
CTATAGTGCAGAACATCCAGGGGCAAATGGTACATCAGGCCATATCACCTAGAACTTTAAATGCATGGGTAAA
AGTAGTAGAAGAGAAGGCTTTCAGCCCAGAAGTGATACCCATGTTTTCAGCATTATCAGAAGGAGCCACCCCA
CAAGATTTAAACACCATGCTAAACACAGTGGGGGGACATCAAGCAGCCATGCAAATGTTAAAAGAGACCATCA
ATGAGGAAGCTGCAGGCAAAGAGAAGAGTGGTGCAGAGAGAAAAAAGAGCAGTGGGAATAGGAGCTTTGTT
CCTTGGGTTCTTGGGAGCAGCAGGAAGCACTATGGGCGCAGCGTCAATGACGCTGACGGTACAGGCCAGACA
ATTATTGTCTGGTATAGTGCAGCAGCAGAACAATTTGCTGAGGGCTATTGAGGCGCAACAGCATCTGTTGCAA
CTCACAGTCTGGGGCATCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGGATCTACAAATGGCAGTA
TTCATCCACAATTTTAAAAGAAAAGGGGGGATTGGGGGGTACAGTGCAGGGGAAAGAATAGTAGACATAATA
GCAACAGACATACAAACTAAAGAATTACAAAAACAAATTACAAAAATTCAAAATTTTCGGGTTTATTACAGGG
ACAGCAGAGATCCAGTTTGGGGATCAATTGCATGAAGAATCTGCTTAGGGTTAGGCGTTTTGCGCTGCTTCGC
GAGGATCTGCGATCGCTCCGGTGCCCGTCAGTGGGCAGAGCGCACATCGCCCACAGTCCCCGAGAAGTTGGG
GGGAGGGGTCGGCAATTGAACCGGTGCCTAGAGAAGGTGGCGCGGGGTAAACTGGGAAAGTGATGTCGTGT
ACTGGCTCCGCCTTTTTCCGAGGGTGGGGGAGAACCGTATATAAGTGCAGTAGTCGCCGTGAACGTTCTTTTT
CGCAACGGGTTTGCCGCCAGAACACAGCTGAAGCTTCGAGGGGCTCGCATCTCTCCTTCACGCGCCCGCCGCC
CTACCTGAGGCCGCCATCCACGCCGGTTGAGTCGCGTTCTGCCGCCTCCCGCCTGTGGTGCCTCCTGAACTGCG
TCCGCCGTCTAGGTAAGTTTAAAGCTCAGGTCGAGACCGGGCCTTTGTCCGGCGCTCCCTTGGAGCCTACCTAG
ACTCAGCCGGCTCTCCACGCTTTGCCTGACCCTGCTTGCTCAACTCTACGTCTTTGTTTCGTTTTCTGTTCTGCGC
CGTTACAGATCCAAGCTGTGACCGGCGCCTACGGCTAGCGCCGCCACCATGCTGCTGCTCGTGACATCTCTGCT
GCTGTGCGAGCTGCCCCACCCCGCCTTTCTGCTGATCCCC
SEQ ID NO: 62 (Lentiviral vector backbone 3′)
CTCGAGGGCGGAGGCGAAGGCAGAGGCAGCCTGCTGACATGTGGCGACGTGGAAGAGAACCCAGGCCCCAG
AATGCTGCTGCTCGTGACCAGCCTGCTGCTGTGTGAACTGCCTCATCCTGCTTTTCTGCTGATTCCTCGGAAAGT
GTGCAACGGCATCGGCATCGGAGAGTTCAAGGACTCCCTGAGCATCAACGCCACCAACATCAAGCACTTCAAG
AACTGCACCAGCATCAGCGGCGACCTGCACATCCTGCCTGTGGCCTTTAGAGGCGACAGCTTCACCCACACAC
CCCCCCTGGATCCACAGGAACTGGATATTCTGAAAACCGTAAAGGAAATCACAGGGTTTTTGCTGATTCAGGC
TTGGCCTGAAAACAGGACGGACCTCCATGCCTTTGAGAACCTAGAAATCATACGCGGCAGGACCAAGCAACAT
GGTCAGTTTTCTCTTGCAGTCGTCAGCCTGAACATAACATCCTTGGGATTACGCTCCCTCAAGGAGATAAGTGA
TGGAGATGTGATAATTTCAGGAAACAAAAATTTGTGCTATGCAAATACAATAAACTGGAAAAAACTGTTTGGG
ACCTCCGGTCAGAAAACCAAAATTATAAGCAACAGAGGTGAAAACAGCTGCAAGGCCACAGGCCAGGTCTGC
CATGCCTTGTGCTCCCCCGAGGGCTGCTGGGGCCCGGAGCCCAGGGACTGCGTCTCTTGCCGGAATGTCAGCC
GAGGCAGGGAATGCGTGGACAAGTGCAACCTTCTGGAGGGTGAGCCAAGGGAGTTTGTGGAGAACTCTGAG
TGCATACAGTGCCACCCAGAGTGCCTGCCTCAGGCCATGAACATCACCTGCACAGGACGGGGACCAGACAACT
GTATCCAGTGTGCCCACTACATTGACGGCCCCCACTGCGTCAAGACCTGCCCGGCAGGAGTCATGGGAGAAAA
CAACACCCTGGTCTGGAAGTACGCAGACGCCGGCCATGTGTGCCACCTGTGCCATCCAAACTGCACCTACGGA
TGCACTGGGCCAGGTCTTGAAGGCTGTCCAACGAATGGGCCTAAGATCCCGTCCATCGCCACTGGGATGGTGG
GGGCCCTCCTCTTGCTGCTGGTGGTGGCCCTGGGGATCGGCCTCTTCATGTGAGCGGCCGCTCTAGACCCGGG
CTGCAGGAATTCGATATCAAGCTTATCGATAATCAACCTCTGGATTACAAAATTTGTGAAAGATTGACTGGTAT
TCTTAACTATGTTGCTCCTTTTACGCTATGTGGATACGCTGCTTTAATGCCTTTGTATCATGCTATTGCTTCCCGT
ATGGCTTTCATTTTCTCCTCCTTGTATAAATCCTGGTTGCTGTCTCTTTATGAGGAGTTGTGGCCCGTTGTCAGG
CAACGTGGCGTGGTGTGCACTGTGTTTGCTGACGCAACCCCCACTGGTTGGGGCATTGCCACCACCTGTCAGC
TCCTTTCCGGGACTTTCGCTTTCCCCCTCCCTATTGCCACGGCGGAACTCATCGCCGCCTGCCTTGCCCGCTGCT
GGACAGGGGCTCGGCTGTTGGGCACTGACAATTCCGTGGTGTTGTCGGGGAAATCATCGTCCTTTCCTTGGCT
GCTCGCCTGTGTTGCCACCTGGATTCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTTCGCCTTCGCCCTCAGACGAGTCGG
ATCTCCCTTTGGGCCGCCTCCCCGCATCGATACCGTCGACTAGCCGTACCTTTAAGACCAATGACTTACAAGGC
AGCTGTAGATCTTAGCCACTTTTTAAAAGAAAAGGGGGGACTGGAAGGGCTAATTCACTCCCAAAGAAGACAA
GATCTGCTTTTTGCCTGTACTGGGTCTCTCTGGTTAGACCAGATCTGAGCCTGGGAGCTCTCTGGCTAACTAGG
GAACCCACTGCTTAAGCCTCAATAAAGCTTGCCTTGAGTGCTTCAAGTAGTGTGTGCCCGTCTGTTGTGTGACT
CTGGTAACTAGAGATCCCTCAGACCCTTTTAGTCAGTGTGGAAAATCTCTAGCAGAATTCGATATCAAGCTTAT
CGATACCGTCGACCTCGAGGGGGGGCCCGGTACCCAATTCGCCCTATAGTGAGTCGTATTACAATTCACTGGC
CGTCGTTTTACAACGTCGTGACTGGGAAAACCCTGGCGTTACCCAACTTAATCGCCTTGCAGCACATCCCCCTTT
CGCCAGCTGGCGTAATAGCGAAGAGGCCCGCACCGATCGCCCTTCCCAACAGTTGCGCAGCCTGAATGGCGA
ATGGAAATTGTAAGCGTTAATATTTTGTTAAAATTCGCGTTAAATTTTTGTTAAATCAGCTCATTTTTTAACCAAT
AGGCCGAAATCGGCAAAATCCCTTATAAATCAAAAGAATAGACCGAGATAGGGTTGAGTGTTGTTCCAGTTTG
GAACAAGAGTCCACTATTAAAGAACGTGGACTCCAACGTCAAAGGGCGAAAAACCGTCTATCAGGGCGATGG
CCCACTACGTGAACCATCACCCTAATCAAGTTTTTTGGGGTCGAGGTGCCGTAAAGCACTAAATCGGAACCCTA
AAGGGAGCCCCCGATTTAGAGCTTGACGGGGAAAGCCGGCGAACGTGGCGAGAAAGGAAGGGAAGAAAGC
GAAAGGAGCGGGCGCTAGGGCGCTGGCAAGTGTAGCGGTCACGCTGCGCGTAACCACCACACCCGCCGCGCT
TAATGCGCCGCTACAGGGCGCGTCAGGTGGCACTTTTCGGGGAAATGTGCGCGGAACCCCTATTTGTTTATTTT
TCTAAATACATTCAAATATGTATCCGCTCATGAGACAATAACCCTGATAAATGCTTCAATAATATTGAAAAAGG
AAGAGTATGAGTATTCAACATTTCCGTGTCGCCCTTATTCCCTTTTTTGCGGCATTTTGCCTTCCTGTTTTTGCTC
ACCCAGAAACGCTGGTGAAAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAATGATGAGCACTTTTAAAGTT
CTGCTATGTGGCGCGGTATTATCCCGTATTGACGCCGGGCAAGAGCAACTCGGTCGCCGCATACACTATTCTCA
GAATGACTTGGTTGAGTACTCACCAGTCACAGAAAAGCATCTTACGGATGGCATGACAGTAAGAGAATTATGC
AGTGCTGCCATAACCATGAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAACCGGAGCTGAATGAAGC
CATACCAAACGACGAGCGTGACACCACGATGCCTGTAGCAATGGCAACAACGTTGCGCAAACTATTAACTGGC
GAACTACTTACTCTAGCTTCCCGGCAACAATTAATAGACTGGATGGAGGCGGATAAAGTTGCAGGACCACTTC
TGCGCTCGGCCCTTCCGGCTGGCTGGTTTATTGCTGATAAATCTGGAGCCGGTGAGCGTGGGTCTCGCGGTAT
CATTGCAGCACTGGGGCCAGATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAA
TACTGTTCTTCTAGTGTAGCCGTAGTTAGGCCACCACTTCAAGAACTCTGTAGCACCGCCTACATACCTCGCTCT
GCTAATCCTGTTACCAGTGGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCCAGCTTGGAGCGAACGAC
CTACACCGAACTGAGATACCTACAGCGTGAGCTATGAGAAAGCGCCACGCTTCCCGAAGGGAGAAAGGCGGA
CAGGTATCCGGTAAGCGGCAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGAGTGAGCTGATACCGCTCGCCGCAGC
CGAACGACCGAGCGCAGCGAGTCAGTGAGCGAGGAAGCGGAAGAGCGCCCAATACGCAAACCGCCTCTCCCC
GCGCGTTGGCCGATTCATTAATGCAGCTGGCACGACAGGTTTCCCGACTGGAAAGCGGGCAGTGAGCGCAAC
GCAATTAATGTGAGTTAGCTCACTCATTAGGCACCCCAGGCTTTACACTTTATGCTTCCGGCTCGTATGTTGTGT
GGAATTGTGAGCGGATAACAATTTCACACAGGAAACAGCTATGACCATGATTACGCCAAGCTCGAAATTAACC
CTCACTAAAGGGAACAAAAGCTGGAGCTCCACCGCGGTGGCGGCCTCGAGGTCGAGATCCGGTCGACCAGCA
ACCATAGTCCCGCCCCTAACTCCGCCCATCCCGCCCCTAACTCCGCCCAGTTCCGCCCATTCTCCGCCCCATGGC
TGACTAATTTTTTTTTTTATGCAGAGGCCGAGGCCGCCTCGGCCTCTGAGCTATTCCAGAAGTAGTGAGGAGG
CTTTTTTGGAGGCCTAGGCTTTTGCAAAAAGCTTCGACGGTATCGATTGGCTCATGTCCAACATTACCGCCATG
TTGACATTGATTATTGACTAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACATCTACGTATTAGTCATCGC
TATTACCATGGTGATGCGGTTTTGGCAGTACATCAATGGGCGTGGATAGCGGTTTGACTCACGGGGATTTCCA
AGTCTCCACCCCATTGACGTCAATGGGAGTTTGTTTTGGCACCAAAATCAACGGGACTTTCCAAAATGTCGTAA
CAACTCCGCCCCATTGACGCAAATGGGCGGTAGGCGTGTACGGAATTCGGAGTGGCGAGCCCTCAGATCCTG
CATATAAGCAGCTGCTTTTTGCCTGTACTGGGTCTCTCTG
SEQ ID NO: 63 (Sleeping Beauty vector backbone 5′)
CAAGGCGATTAAGTTGGGTAACGCCAGGGTTTTCCCAGTCACGACGTTGTAAAACGACGGCCAGTGAGCGCG
CGTAATACGACTCACTATAGGGCGAATTGGAGCTCGGGTCCCTATACAGTTGAAGTCGGAAGTTTACATACAC
TTAAGTTGGAGTCATTAAAACTCG TTTTTCAACTACTCCACAAATTTCTTGTTAACAAACAATAGTTTTGGCAAG
TCAGTTAGGACATCTACTTTGTGCATGACACAAGTCATTTTTCCAACAATTGTTTACAGACAGATTATTTCACTT
ATAATTCACTGTATCACAATTCCAGTGGGTCAGAAGTTTACATACACTAAGTTGACTGTGCCTTTAAACAGCTTG
GAAAATTCCAGAAAATGATGTCATGGCTTTAGAAGCTTGATATCCATGGAATTCGGATCTGCGATCGCTCCGGT
GCCCGTCAGTGGGCAGAGCGCACATCGCCCACAGTCCCCGAGAAGTTGGGGGGAGGGGTCGGCAATTGAAC
CGGTGCCTAGAGAAGGTGGCGCGGGGTAAACTGGGAAAGTGATGTCGTGTACTGGCTCCGCCTTTTT CCCGA
GGGTGGGGGAGAACCGTATATAAGTGCAGTAGTCGCCGTGAACGTTCTTTTTCGCAACGGGTTTGCCGCCAGA
ACACAGCTGAAGCTTCGAGGGGCTCGCATCTCTCCTTCACGCGCCCGCCGCCCTACCTGAGGCCGCCATCCACG
CCGGTTGAGTCGCGTTCTGCCGCCTCCCGCCTGTGGTGCCTCCTGAACTGCGTCCGCCGTCTAGGTAAGTTTAA
AGCTCAGGTCGAGACCGGGCCTTTGTCCGGCGCTCCCTTGGAGCCTACCTAGACTCAGCCGGCTCTCCACGCTT
TGCCTGACCCTGCTTGCTCAACTCTACGTCTTTGTTTCGTTTTCTGTTCTGCGCCGTTACAGATCCAAGCTGTGAC
CGGCGCCTACGGCTAGCGCCGCCACCATGCTGCTGCTCGTGACATCTCTGCTGCTGTGCGAGCTGCCCCACCCC
GCCTTTCTGCTGATCCCC
SEQ ID NO: 64 (Sleeping Beauty vector backbone 3′)
CTCGAGGGCGGAGGCGAAGGCAGAGGCAGCCTGCTGACATGTGGCGACGTGGAAGAGAACCCAGGCCCCAG
AATGCTGCTGCTCGTGACCAGCCTGCTGCTGTGTGAACTGCCTCATCCTGCTTTTCTGCTGATTCCTCGGAAAGT
GTGCAACGGCATCGGCATCGGAGAGTTCAAGGACTCCCTGAGCATCAACGCCACCAACATCAAGCACTTCAAG
AACTGCACCAGCATCAGCGGCGACCTGCACATCCTGCCTGTGGCCTTTAGAGGCGACAGCTTCACCCACACAC
CCCCCCTGGATCCACAGGAACTGGATATTCTGAAAACCGTAAAGGAAATCACAGGGTTTTT GCTGATTCAGGC
TTGGCCTGAAAACAGGACGGACCTCCATGCCTTTGAGAACCTAGAAATCATACGCGGCAGGACCAAGCAACAT
GGTCAGTTTTCTCTTGCAGTCGTCAGCCTGAACATAACATCCTTGGGATTACGCTCCCTCAAGGAGATAAGTGA
TGGAGATGTGATAATTTCAGGAAACAAAAATTTGTGCTATGCAAATACAATAAACTGGAAAAAACTGTTTGGG
ACCTCCGGTCAGAAAACCAAAATTATAAGCAACAGAGGTGAAAACAGCTGCAAGGCCACAGGCCAGGTCTGC
CATGCCTTGTGCTCCCCCGAGGGCTGCTGGGGCCCGGAGCCAAGGGACTGCGTCTCTTGCCGGAATGTCAGCC
GAGGCAGGGAATGCGTGGACAAGTGCAACCTTCTGGAGGGTGAGCCAAGGGAGTTTGTGGAGAACTCTGAG
TGCATACAGTGCCACCCAGAGTGCCTGCCTCAGGCCATGAACATCACCTGCACAGGACGGGGACCAGACAACT
GTATCCAGTGTGCCCACTACATTGACGGCCCCCACTGCGTCAAGACCTGCCCGGCAGGAGTCATGGGAGAAAA
CAACACCCTGGTCTGGAAGTACGCAGACGCCGGCCATGTGTGCCACCTGTGCCATCCAAACTGCACCTACGGA
TGCACTGGGCCAGGTCTTGAAGGCTGTCCAACGAATGGGCCTAAGATCCCGTCCATCGCCACTGGGATGGTGG
GGGCCCTCCTCTTGCTGCTGGTGGTGGCCCTAGGGATCGGCCTCTTCATGTGAGCGGCCGCTCTAGATGGCCA
GATCTAGCTTGTGGAAGGCTACTCGAAATGTTTGACCCAAGTTAAACAATTTAAAGGCAATGCTACCAAATACT
AATTGAGTGTATGTAAACTTCTGACCCACTGGGAATGTGATGAAAGAAATAAAAGCTGAAATGAATCATTCTC
TCTACTATTATTCTGATATTTCACATTCTTAAAATAAAGTGGTGATCCTAACTGACCTAAGACAGGGAATTTTTA
CTAGGATTAAATGTCAGGAATTGTGAAAAAGTGAGTTTAAATGTATTTGGCTAAGGTGTATGTAAACTTCCGA
CTTCAACTGTATAGGGGTCCTCTAGCTAGAGTCGACCTCGAGGGGGGGCCCGGTACCCAGCTTTTGTTCCCTTT
AGTGAGGGTTAATTGCGCGCTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTAATGAGTGAGCTAACTCACAT
TAATTGCGTTGCGCTCACTGCCCGCTTTCCAGTCGGGAAACCTGTCGTGCCAGCTGCATTAATGAATCGGCCAA
CGCGCGGGGAGAGGCGGTTTGCGTATTGGGCGCTCTTCCGCTTCCTCGCTCACTGACTCGCTGCGCTCGGTCG
TTCGGCTGCGGCGAGCGGTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGGCCGCGTTGCTGGCGTTTT
TCCATAGGCTCCGCCCCCCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAGCCACTGGTAACAGGAT
TAGCAGAGCGAGGTATGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGTAGCTCTTGATCCGGCA
AACAAACCACCGCTGGTAGCGGTGGTTTTTTTT GTTTGCAAGCAGCAGATTACGCGCAGAAAAAAAGGATCTCA
AGAAGATCCTTTGATCTTTTCTACGGGGTCTGACGCTCAGTGGAACGAAAACTCACGTTAAGGGATTTTGGTCA
TGAGATTATCAAAAAGGATCTTCACCTAGATCCTTTTAAATTAAAAATGAAGTTTTAAATCAATCTAAAGTATAT
ATGAGTAAACTTGGTCTGACAGTTACCAATGCTTAATCAGTGAGGCACCTATCTCAGCGATCTGTCTATTTCGTT
CATCCATAGTTGCCTGACTCCCCGTCGTGTAGATAACTACGATACGGGAGGGCTTACCATCTGGCCCCAGTGCT
GCAATGATACCGCGAGACCCACGCTCACCGGCTCCAGATTTATCAGCAATAAACCAGCCAGCCGGAAGGGCCG
AGCGCAGAAGTGGTCCTGCAACTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTGTTGAGA
TCCAGTTCGATGTAACCCACTCGTGCACCCAACTGATCTTCAGCATCTTTTACTTTCACCAGCGTTTCTGGGTGA
GCAAAAACAGGAAGGCAAAATGCCGCAAAAAAGGGAATAAGGGCGACACGGAAATGTTGAATACTCATACTC
TTCCTTTTTCAATATTATTGAAGCATTTATCAGGGTTATTGTCTCATGAGCGGATACATATTTGAATGTATTTAG
AAAAATAAACAAATAGGGGTTCCGCGCACATTTCCCCGAAAAGTGCCACCTGACGCGCCCTGTAGCGGCGCAT
TAAGCGCGGCGGGTGTGGTGGTTACGCGCAGCGTGACCGCTACACTTGCCAGCGCCCTAGCGCCCGCTCCTTT
CGCTTTCTTCCCTTCCTTTCTCGCCACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTAGGGTGATGGTTCACGTAGTGGGCCAT
CGCCCTGATAGACGGTTTTTCGCCCTTTGACGTTGGAGTCCACGTTCTTTAATAGTGGACTCTTGTTCCAAACTG
GAACAACACTCAACCCTATCTCGGTCTATTCTTTTGATTTATAAGGGATTTTGCCGATTTCGGCCTATTGGTTAA
AAAATGAGCTGATTTAACAAAAATTTAACGCGAATTTTAACAAAATATTAACGCTTACAATTTCCATTCGCCATT
CAGGCTGCGCAACTGTTGGGAAGGGCGATCGGTGCGGGCCTCTTCGCTATTACGCCAGCTGGCGAAAGGGGG
ATGTGCTG
SEQ ID NO: 65 (CAR transmembrane domain)
Met Phe Trp Val Leu Val Val Val Gly Gly Val Leu Ala Cys Tyr Ser Leu Leu Val Thr Val Ala Phe Ile Ile Phe
Trp Val
SEQ ID NO: 66 (CAR 4-1BB domain)
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
SEQ ID NO: 67 (CAR CD3 zeta domain)
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 68 (CD19 CAR)
Asp Ile Gln Met Thr Gln Thr Thr Ser Ser Leu Ser Ala Ser Leu Gly Asp Arg Val Thr Ile Ser Cys Arg Ala Ser
Gln Asp Ile Ser Lys Tyr Leu Asn Trp Tyr Gln Gln Lys Pro Asp Gly Thr Val Lys Leu Leu Ile Tyr His Thr Ser
Arg Leu His Ser Gly Val Pro Ser Arg Phe Ser Gly Ser Gly Ser Gly Thr Asp Tyr Ser Leu Thr Ile Ser Asn Leu
Glu Gln Glu Asp Ile Ala Thr Tyr Phe Cys Gln Gln Gly Asn Thr Leu Pro Tyr Thr Phe Gly Gly Gly Thr Lys Leu
Glu Ile Thr
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Val Lys Leu Gln Glu Ser Gly Pro Gly Leu Val Ala Pro Ser Gln Ser Leu Ser Val Thr Cys Thr Val Ser Gly
Val Ser Leu Pro Asp Tyr Gly Val Ser Trp Ile Arg Gln Pro Pro Arg Lys Gly Leu Glu Trp Leu Gly Val Ile Trp
Gly Ser Glu Thr Thr Tyr Tyr Asn Ser Ala Leu Lys Ser Arg Leu Thr Ile Ile Lys Asp Asn Ser Lys Ser Gln Val
Phe Leu Lys Met Asn Ser Leu Gln Thr Asp Asp Thr Ala Ile Tyr Tyr Cys Ala Lys His Tyr Tyr Tyr Gly Gly Ser
Tyr Ala Met Asp Tyr Trp Gly Gln Gly Thr Ser Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 69 (CD19 CAR SB vector, CAR insert underlined)
CAAGGCGATTAAGTTGGGTAACGCCAGGGTTTTCCCAGTCACGACGTTGTAAAACGACGGCCAGTGAGCGCG
CGTAATACGACTCACTATAGGGCGAATTGGAGCTCGGGTCCCTATACAGTTGAAGTCGGAAGTTTACATACAC
TTAAGTTGGAGTCATTAAAACTCGTTTTTCAACTACTCCACAAATTTCTTGTTAACAAACAATAGTTTTGGCAAG
TCAGTTAGGACATCTACTTTGTGCATGACACAAGTCATTTTTCCAACAATTGTTTACAGACAGATTATTTCACTT
ATAATTCACTGTATCACAATTCCAGTGGGTCAGAAGTTTACATACACTAAGTTGACTGTGCCTTTAAACAGCTTG
GAAAATTCCAGAAAATGATGTCATGGCTTTAGAAGCTTGATATCCATGGAATTCGGATCTGCGATCGCTCCGGT
GCCCGTCAGTGGGCAGAGCGCACATCGCCCACAGTCCCCGAGAAGTTGGGGGGAGGGGTCGGCAATTGAAC
CGGTGCCTAGAGAAGGTGGCGCGGGGTAAACTGGGAAAGTGATGTCGTGTACTGGCTCCGCCTTTTTCCCGA
GGGTGGGGGAGAACCGTATATAAGTGCAGTAGTCGCCGTGAACGTTCTTTTTCGCAACGGGTTTGCCGCCAGA
ACACAGCTGAAGCTTCGAGGGGCTCGCATCTCTCCTTCACGCGCCCGCCGCCCTACCTGAGGCCGCCATCCACG
CCGGTTGAGTCGCGTTCTGCCGCCTCCCGCCTGTGGTGCCTCCTGAACTGCGTCCGCCGTCTAGGTAAGTTTAA
AGCTCAGGTCGAGACCGGGCCTTTGTCCGGCGCTCCCTTGGAGCCTACCTAGACTCAGCCGGCTCTCCACGCTT
TGCCTGACCCTGCTTGCTCAACTCTACGTCTTTGTTTCGTTTTCTGTTCTGCGCCGTTACAGATCCAAGCTGTGAC
CGGCGCCTACGGCTAGCGCCGCCACCATGCTGCTGCTCGTGACATCTCTGCTGCTGTGCGAGCTGCCCCACCCC
GCCTTTCTGCTGATCCCCGACATCCAGATGACCCAGACCACCAGCAGCCTGAGCGCCAGCCTGGGCGATAGAG
TGACCATCAGCTGCAGAGCCAGCCAGGACATCAGCAAGTACCTGAACTGGTATCAGCAGAAACCCGACGGCA
CCGTGAAGCTGCTGATCTACCACACCAGCAGACTGCACAGCGGCGTGCCCAGCAGATTTTCTGGCAGCGGCTC
CGGCACCGACTACAGCCTGACCATCTCCAACCTGGAACAGGAAGATATTGCTACCTACTTCTGTCAGCAAGGC
AACACCCTGCCCTACACCTTCGGCGGAGGCACCAAGCTGGAAATCACCGAACTGAAAACCCCGCTTGGCGACA
CCACCCACACCTGTCCTAGATGTCCCGAACCCAAGAGCTGCGATACCCCCCCACCTTGCCCTAGATGCCCCGAG
CCTAAGTCCTGCGACACCCCTCCTCCATGCCCTCGGTGTCCTGAGCCTAAGAGCTGTGACACACCACCCCCCTG
CCCCAGATGTCCAGAGCCAAAATCTTGTGATACCCCTCCCCCCTGTCCCCGCTGCCCAGAACCCAAGTCCTGTG
ATACTCCACCTCCTTGTCCACGGTGCCCCGAAGTGAAACTGCAGGAAAGCGGCCCTGGACTGGTGGCCCCAAG
CCAGTCTCTGAGCGTGACCTGTACCGTGTCCGGCGTGTCCCTGCCTGACTATGGCGTGTCCTGGATCAGACAG
CCCCCCAGAAAGGGCCTGGAATGGCTGGGAGTGATCTGGGGCAGCGAGACAACCTACTACAACAGCGCCCTG
AAGTCCCGGCTGACCATCATCAAGGACAACTCCAAGAGCCAGGTGTTCCTGAAGATGAACAGCCTGCAGACCG
ACGACACCGCCATCTACTACTGCGCCAAGCACTACTACTACGGCGGCAGCTACGCTATGGACTACTGGGGCCA
GGGCACCAGCGTGACCGTGTCTAGCGAACTGAAAACCCCCCTGGGCGACACCACCCACACCTGTCCTAGATGT
CCGGAACCCAAGAGCTGCGATACCCCCCCACCTTGCCCCAGATGCCCCATGTTTTGGGTGCTGGTGGTCGTGG
GCGGAGTGCTGGCCTGTTACAGCCTGCTCGTGACCGTGGCCTTCATCATCTTTTGGGTCAAGCGGGGCAGAAA
GAAGCTGCTGTACATCTTTAAGCAGCCCTTCATGCGGCCCGTGCAGACCACCCAGGAAGAGGACGGCTGCTCC
TGCAGATTCCCCGAGGAAGAAGAAGGCGGCTGCGAGCTGAGAGTGAAGTTCAGCAGATCCGCCGACGCCCCT
GCCTATCAGCAGGGCCAGAACCAGCTATACAACGAGCTGAACCTGGGCAGACGGGAAGAGTACGACGTGCTG
GACAAGAGAAGAGGCCGGGACCCTGAGATGGGCGGAAAGCCCAGAAGAAAGAACCCCCAGGAAGGCCTGT
ATAACGAACTGCAGAAAGACAAGATGGCCGAGGCCTACAGCGAGATCGGAATGAAGGGCGAGCGGCGGAG
AGGCAAGGGCCACGATGGACTGTATCAGGGCCTGAGCACCGCCACCAAGGACACCTATGACGCCCTGCACAT
GCAGGCCCTGCCCCCTAGACTCGAGGGCGGAGGCGAAGGCAGAGGCAGCCTGCTGACATGTGGCGACGTGG
AAGAGAACCCAGGCCCCAGAATGCTGCTGCTCGTGACCAGCCTGCTGCTGTGTGAACTGCCTCATCCTGCTTTT
CTGCTGATTCCTCGGAAAGTGTGCAACGGCATCGGCATCGGAGAGTTCAAGGACTCCCTGAGCATCAACGCCA
CCAACATCAAGCACTTCAAGAACTGCACCAGCATCAGCGGCGACCTGCACATCCTGCCTGTGGCCTTTAGAGG
CGACAGCTTCACCCACACACCCCCCCTGGATCCACAGGAACTGGATATTCTGAAAACCGTAAAGGAAATCACA
GGGTTTTTGCTGATTCAGGCTTGGCCTGAAAACAGGACGGACCTCCATGCCTTTGAGAACCTAGAAATCATAC
GCGGCAGGACCAAGCAACATGGTCAGTTTTCTCTTGCAGTCGTCAGCCTGAACATAACATCCTTGGGATTACGC
TCCCTCAAGGAGATAAGTGATGGAGATGTGATAATTTCAGGAAACAAAAATTTGTGCTATGCAAATACAATAA
ACTGGAAAAAACTGTTTGGGACCTCCGGTCAGAAAACCAAAATTATAAGCAACAGAGGTGAAAACAGCTGCA
AGGCCACAGGCCAGGTCTGCCATGCCTTGTGCTCCCCCGAGGGCTGCTGGGGCCCGGAGCCAAGGGACTGCG
TCTCTTGCCGGAATGTCAGCCGAGGCAGGGAATGCGTGGACAAGTGCAACCTTCTGGAGGGTGAGCCAAGGG
AGTTTGTGGAGAACTCTGAGTGCATACAGTGCCACCCAGAGTGCCTGCCTCAGGCCATGAACATCACCTGCAC
AGGACGGGGACCAGACAACTGTATCCAGTGTGCCCACTACATTGACGGCCCCCACTGCGTCAAGACCTGCCCG
GCAGGAGTCATGGGAGAAAACAACACCCTGGTCTGGAAGTACGCAGACGCCGGCCATGTGTGCCACCTGTGC
CATCCAAACTGCACCTACGGATGCACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCCCTAGGGATCGGCCTCTTCATGTG
AGCGGCCGCTCTAGATGGCCAGATCTAGCTTGTGGAAGGCTACTCGAAATGTTTGACCCAAGTTAAACAATTT
AAAGGCAATGCTACCAAATACTAATTGAGTGTATGTAAACTTCTGACCCACTGGGAATGTGATGAAAGAAATA
AAAGCTGAAATGAATCATTCTCTCTACTATTATTCTGATATTTCACATTCTTAAAATAAAGTGGTGATCCTAACT
GACCTAAGACAGGGAATTTTTACTAGGATTAAATGTCAGGAATTGTGAAAAAGTGAGTTTAAATGTATTTGGC
TAAGGTGTATGTAAACTTCCGACTTCAACTGTATAGGGGTCCTCTAGCTAGAGTCGACCTCGAGGGGGGGCCC
GGTACCCAGCTTTTGTTCCCTTTAGTGAGGGTTAATTGCGCGCTTGGCGTAATCATGGTCATAGCTGTTTCCTGT
GTGAAATTGTTATCCGCTCACAATTCCACACAACATACGAGCCGGAAGCATAAAGTGTAAAGCCTGGGGTGCC
TAATGAGTGAGCTAACTCACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCGCTCTTCCGCTTCCTCGCTC
ACTGACTCGCTGCGCTCGGTCGTTCGGCTGCGGCGAGCGGTATCAGCTCACTCAAAGGCGGTAATACGGTTAT
CCACAGAATCAGGGGATAACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGGTGGCCTA
ACTACGGCTACACTAGAAGGACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAACGAAAACTC
ACGTTAAGGGATTTTGGTCATGAGATTATCAAAAAGGATCTTCACCTAGATCCTTTTAAATTAAAAATGAAGTT
TTAAATCAATCTAAAGTATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATCAGCAATAA
ACCAGCCAGCCGGAAGGGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGCCATTGCTACAGGCATC
GTGGTGTCACGCTCGTCGTTTGGTATGGCTTCATTCAGCTCCGGTTCCCAACGATCAAGGCGAGTTACATGATC
CCCCATGTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTGCCCGGCGTCAATACG
GGATAATACCGCGCCACATAGCAGAACTTTAAAAGTGCTCATCATTGGAAAACGTTCTTCGGGGCGAAAACTC
TCAAGGATCTTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGGGCGACA
CGGAAATGTTGAATACTCATACTCTTCCTTTTTCAATATTATTGAAGCATTTATCAGGGTTATTGTCTCATGAGC
GGATACATATTTGAATGTATTTAGAAAAATAAACAAATAGGGGTTCCGCGCACATTTCCCCGAAAAGTGCCACC
TGACGCGCCCTGTAGCGGCGCATTAAGCGCGGCGGGTGTGGTGGTTACGCGCAGCGTGACCGCTACACTTGC
CAGCGCCCTAGCGCCCGCTCCTTTCGCTTTCTTCCCTTCCTTTCTCGCCACGTTCGCCGGCTTTCCCCGTCAAGCT
CTAAATCGGGGGCTCCCTTTAGGGTTCCGATTTAGTGCTTTACGGCACCTCGACCCCAAAAAACTTGATTAGGG
TGATGGTTCACGTAGTGGGCCATCGCCCTGATAGACGGTTTTTCGCCCTTTGACGTTGGAGTCCACGTTCTTTA
ATAGTGGACTCTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAACAAAATATTA
ACGCTTACAATTTCCATTCGCCATTCAGGCTGCGCAACTGTTGGGAAGGGCGATCGGTGCGGGCCTCTTCGCTA
TTACGCCAGCTGGCGAAAGGGGGATGTGCTG
SEQ ID NO: 70 (CD19 CAR LV vector, CAR inserted underlined)
GTTAGACCAGATCTGAGCCTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGAGATCCCTCAGACCCTTTTA
GTCAGTGTGGAAAATCTCTAGCAGTGGCGCCCGAACAGGGACTTGAAAGCGAAAGGGAAACCAGAGGAG CT
CTCTCGACGCAGGACTCGGCTTGCTGAAGCGCGCACGGCAAGAGGCGAGGGGCGGCGACTGGTGAGTACGC
CAAAAATTTTGACTAGCGGAGGCTAGAAGGAGAGAGATGGGTGCGAGAGCGTCAGTATTAAGCGGGGGAGA
ATTAGATCGATGGGAAAAAATTCGGTTAAGGCCAGGGGGAAAGAAAAAATATAAATTAAAACATATAGTATG
GGCAAGCAGGGAGCTAGAACGATTCGCAGTTAATCCTGGCCTGTTAGAAACATCAGAAGGCTGTAGACAAAT
ACTGGGACAGCTACAACCATCCCTTCAGACAGGATCAGAAGAACTTAGATCATTATATAATACAGTAGCAACCC
TCTATTGTGTGCATCAAAGGATAGAGATAAAAGACACCAAGGAAGCTTTAGACAAGATAGAGGAAGAGCAAA
ACAAAAGTAAGAAAAAAGCACAGCAAGCAGCAGCTGACACAGGACACAGCAATCAGGTCAGCCAAAATTACC
CTATAGTGCAGAACATCCAGGGGCAAATGGTACATCAGGCCATATCACCTAGAACTTTAAATGCATGGGTAAA
AGTAGTAGAAGAGAAGGCTTTCAGCCCAGAAGTGATACCCATGTTTTCAGCATTATCAGAAGGAGCCACCCCA
CAAGATTTAAACACCATGCTAAACACAGTGGGGGGACATCAAGCAGCCATGCAAATGTTAAAAGAGACCATCA
ATGAGGAAGCTGCAGGCAAAGAGAAGAGTGGTGCAGAGAGAAAAAAGAGCAGTGGGAATAGGAGCTTTGTT
CCTTGGGTTCTTGGGAGCAGCAGGAAGCACTATGGGCGCAGCGTCAATGACGCTGACGGTACAGGCCAGACA
ATTATTGTCTGGTATAGTGCAGCAGCAGAACAATTTGCTGAGGGCTATTGAGGCGCAACAGCATCTGTTGCAA
CTCACAGTCTGGGGCATCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGGATCTACAAATGGCAGTA
TTCATCCACAATTTTAAAAGAAAAGGGGGGATTGGGGGGTACAGTGCAGGGGAAAGAATAGTAGACATAATA
GCAACAGACATACAAACTAAAGAATTACAAAAACAAATTACAAAAATTCAAAATTTTCGGGTTTATTACAGGG
ACAGCAGAGATCCAGTTTGGGGATCAATTGCATGAAGAATCTGCTTAGGGTTAGGCGTTTTGCGCTGCTTCGC
GAGGATCTGCGATCGCTCCGGTGCCCGTCAGTGGGCAGAGCGCACATCGCCCACAGTCCCCGAGAAGTTGGG
GGGAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTTTTT
CGCAACGGGTTTGCCGCCAGAACACAGCTGAAGCTTCGAGGGGCTCGCATCTCTCCTTCACGCGCCCGCCGCC
CTACCTGAGGCCGCCATCCACGCCGGTTGAGTCGCGTTCTGCCGCCTCCCGCCTGTGGTGCCTCCTGAACTGCG
TCCGCCGTCTAGGTAAGTTTAAAGCTCAGGTCGAGACCGGGCCTTTGTCCGGCGCTCCCTTGGAGCCTACCTAG
ACTCAGCCGGCTCTCCACGCTTTGCCTGACCCTGCTTGCTCAACTCTACGTCTTTGTTTCGTTTTCTGTTCTGCGC
CGTTACAGATCCAAGCTGTGACCGGCGCCTACGGCTAGCGCCGCCACCATGCTGCTGCTCGTGACATCTCTGCT
GCTGTGCGAGCTGCCCCACCCCGCCTTTCTGCTGATCCCCGACATCCAGATGACCCAGACCACCAGCAGCCTGA
GCGCCAGCCTGGGCGATAGAGTGACCATCAGCTGCAGAGCCAGCCAGGACATCAGCAAGTACCTGAACTGGT
ATCAGCAGAAACCCGACGGCACCGTGAAGCTGCTGATCTACCACACCAGCAGACTGCACAGCGGCGTGCCCA
GCAGATTTTCTGGCAGCGGCTCCGGCACCGACTACAGCCTGACCATCTCCAACCTGGAACAGGAAGATATTGC
TACCTACTTCTGTCAGCAAGGCAACACCCTGCCCTACACCTTCGGCGGAGGCACCAAGCTGGAAATCACCGAA
CTGAAAACCCCGCTTGGCGACACCACCCACACCTGTCCTAGATGTCCCGAACCCAAGAGCTGCGATACCCCCCC
ACCTTGCCCTAGATGCCCCGAGCCTAAGTCCTGCGACACCCCTCCTCCATGCCCTCGGTGTCCTGAGCCTAAGA
GCTGTGACACACCACCCCCCTGCCCCAGATGTCCAGAGCCAAAATCTTGTGATACCCCTCCCCCCTGTCCCCGCT
GCCCAGAACCCAAGTCCTGTGATACTCCACCTCCTTGTCCACGGTGCCCCGAAGTGAAACTGCAGGAAAGCGG
CCCTGGACTGGTGGCCCCAAGCCAGTCTCTGAGCGTGACCTGTACCGTGTCCGGCGTGTCCCTGCCTGACTAT
GGCGTGTCCTGGATCAGACAGCCCCCCAGAAAGGGCCTGGAATGGCTGGGAGTGATCTGGGGCAGCGAGAC
AACCTACTACAACAGCGCCCTGAAGTCCCGGCTGACCATCATCAAGGACAACTCCAAGAGCCAGGTGTTCCTG
AAGATGAACAGCCTGCAGACCGACGACACCGCCATCTACTACTGCGCCAAGCACTACTACTACGGCGGCAGCT
ACGCTATGGACTACTGGGGCCAGGGCACCAGCGTGACCGTGTCTAGCGAACTGAAAACCCCCCTGGGCGACA
CCACCCACACCTGTCCTAGATGTCCGGAACCCAAGAGCTGCGACACCCCTCCACCTTGCCCAAGATGCCCCATG
TTCTGGGTGCTGGTGGTCGTGGGCGGAGTGCTGGCCTGTTATAGCCTGCTCGTGACCGTGGCCTTCATCATCTT
TTGGGTCAAGCGGGGCAGAAAGAAACTGCTGTACATCTTTAAGCAGCCCTTCATGCGGCCCGTGCAGACCACC
CAGGAAGAGGACGGCTGCTCCTGCAGATTCCCCGAGGAAGAAGAAGGCGGCTGCGAGCTGAGAGTGAAGTT
CAGCAGATCCGCCGACGCCCCTGCCTATCAGCAGGGCCAGAACCAGCTGTACAACGAGCTGAACCTGGGCAG
ACGGGAAGAGTACGACGTGCTGGACAAGAGAAGAGGCCGGGACCCTGAGATGGGCGGAAAGCCCAGAAGA
AAGAACCCCCAGGAAGGCCTGTATAACGAACTGCAGAAAGACAAGATGGCCGAGGCCTACAGCGAGATCGG
AATGAAGGGCGAGCGGCGGAGAGGCAAGGGCCACGATGGACTGTATCAGGGCCTGAGCACCGCCACCAAGG
ACACCTATGACGCCCTGCACATGCAGGCCCTGCCCCCTAGACTCGAGGGCGGAGGCGAAGGCAGAGGCAGCC
TGCTGACATGTGGCGACGTGGAAGAGAACCCAGGCCCCAGAATGCTGCTGCTCGTGACCAGCCTGCTGCTGT
GTGAACTGCCTCATCCTGCTTTTCTGCTGATTCCTCGGAAAGTGTGCAACGGCATCGGCATCGGAGAGTTCAAG
GACTCCCTGAGCATCAACGCCACCAACATCAAGCACTTCAAGAACTGCACCAGCATCAGCGGCGACCTGCACA
TCCTGCCTGTGGCCTTTAGAGGCGACAGCTTCACCCACACACCCCCCCTGGATCCACAGGAACTGGATATTCTG
AAAACCGTAAAGGAAATCACAGGGTTTTTGCTGATTCAGGCTTGGCCTGAAAACAGGACGGACCTCCATGCCT
TTGAGAACCTAGAAATCATACGCGGCAGGACCAAGCAACATGGTCAGTTTTCTCTTGCAGTCGTCAGCCTGAA
CATAACATCCTTGGGATTACGCTCCCTCAAGGAGATAAGTGATGGAGATGTGATAATTTCAGGAAACAAAAAT
TTGTGCTATGCAAATACAATAAACTGGAAAAAACTGTTTGGGACCTCCGGTCAGAAAACCAAAATTATAAGCA
ACAGAGGTGAAAACAGCTGCAAGGCCACAGGCCAGGTCTGCCATGCCTTGTGCTCCCCCGAGGGCTGCTGGG
GCCCGGAGCCCAGGGACTGCGTCTCTTGCCGGAATGTCAGCCGAGGCAGGGAATGCGTGGACAAGTGCAACC
TTCTGGAGGGTGAGCCAAGGGAGTTTGTGGAGAACTCTGAGTGCATACAGTGCCACCCAGAGTGCCTGCCTC
AGGCCATGAACATCACCTGCACAGGACGGGGACCAGACAACTGTATCCAGTGTGCCCACTACATTGACGGCCC
CCACTGCGTCAAGACCTGCCCGGCAGGAGTCATGGGAGAAAACAACACCCTGGTCTGGAAGTACGCAGACGC
CGGCCATGTGTGCCACCTGTGCCATCCAAACTGCACCTACGGATGCACTGGGCCAGGTCTTGAAGGCTGTCCA
ACGAATGGGCCTAAGATCCCGTCCATCGCCACTGGGATGGTGGGGGCCCTCCTCTTGCTGCTGGTGGTGGCCC
TGGGGATCGGCCTCTTCATGTGAGCGGCCGCTCTAGACCCGGGCTGCAGGAATTCGATATCAAGCTTATCGAT
AATCAACCTCTGGATTACAAAATTTGTGAAAGATTGACTGGTATTCTTAACTATGTTGCTCCTTTTACGCTATGT
GGATACGCTGCTTTAATGCCTTTGTATCATGCTATTGCTTCCCGTATGGCTTTCATTTTCTCCTCCTTGTATAAAT
CCTGGTTGCTGTCTCTTTATGAGGAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGGGGCTCGGCTGTTGGGCACTGACA
ATTCCGTGGTGTTGTCGGGGAAATCATCGTCCTTTCCTTGGCTGCTCGCCTGTGTTGCCACCTGGATTCTGCGC
GGGACGTCCTTCTGCTACGTCCCTTCGGCCCTCAATCCAGCGGACCTTCCTTCCCGCGGCCTGCTGCCGGCTCT
GCGGCCTCTTCCGCGTCTTCGCCTTCGCCCTCAGACGAGTCGGATCTCCCTTTGGGCCGCCTCCCCGCATCGAT
ACCGTCGACTAGCCGTACCTTTAAGACCAATGACTTACAAGGCAGCTGTAGATCTTAGCCACTTTTTAAAAGAA
AAGGGGGGACTGGAAGGGCTAATTCACTCCCAAAGAAGACAAGATCTGCTTTTTGCCTGTACTGGGTCTCTCT
GGTTAGACCAGATCTGAGCCTGGGAGCTCTCTGGCTAACTAGGGAACCCACTGCTTAAGCCTCAATAAAGCTT
GCCTTGAGTGCTTCAAGTAGTGTGTGCCCGTCTGTTGTGTGACTCTGGTAACTAGAGATCCCTCAGACCCTTTT
AGTCAGTGTGGAAAATCTCTAGCAGAATTCGATATCAAGCTTATCGATACCGTCGACCTCGAGGGGGGGCCCG
GTACCCAATTCGCCCTATAGTGAGTCGTATTACAATTCACTGGCCGTCGTTTTACAACGTCGTGACTGGGAAAA
CCCTGGCGTTACCCAACTTAATCGCCTTGCAGCACATCCCCCTTTCGCCAGCTGGCGTAATAGCGAAGAGGCCC
GCACCGATCGCCCTTCCCAACAGTTGCGCAGCCTGAATGGCGAATGGAAATTGTAAGCGTTAATATTTTGTTAA
AATTCGCGTTAAATTTTTGTTAAATCAGCTCATTTTTTAACCAATAGGCCGAAATCGGCAAAATCCCTTATAAAT
CAAAAGAATAGACCGAGATAGGGTTGAGTGTTGTTCCAGTTTGGAACAAGAGTCCACTATTAAAGAACGTGG
ACTCCAACGTCAAAGGGCGAAAAACCGTCTATCAGGGCGATGGCCCACTACGTGAACCATCACCCTAATCAAG
TTTTTTGGGGTCGAGGTGCCGTAAAGCACTAAATCGGAACCCTAAAGGGAGCCCCCGATTTAGAGCTTGACGG
GGAAAGCCGGCGAACGTGGCGAGAAAGGAAGGGAAGAAAGCGAAAGGAGCGGGCGCTAGGGCGCTGGCA
AGTGTAGCGGTCACGCTGCGCGTAACCACCACACCCGCCGCGCTTAATGCGCCGCTACAGGGCGCGTCAGGT
GGCACTTTTCGGGGAAATGTGCGCGGAACCCCTATTTGTTTATTTTTCTAAATACATTCAAATATGTATCCGCTC
ATGAGACAATAACCCTGATAAATGCTTCAATAATATTGAAAAAGGAAGAGTATGAGTATTCAACATTTCCGTGT
CGCCCTTATTCCCTTTTTTGCGGCATTTTGCCTTCCTGTTTTTGCTCACCCAGAAACGCTGGTGAAAGTAAAAGA
TGCTGAAGATCAGTTGGGTGCACGAGTGGGTTACATCGAACTGGATCTCAACAGCGGTAAGATCCTTGAGAGT
TTTCGCCCCGAAGAACGTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGAGTACTCACCAGTCA
CAGAAAAGCATCTTACGGATGGCATGACAGTAAGAGAATTATGCAGTGCTGCCATAACCATGAGTGATAACAC
TGCGGCCAACTTACTTCTGACAACGATCGGAGGACCGAAGGAGCTAACCGCTTTTTTGCACAACATGGGGGAT
CATGTAACTCGCCTTGATCGTTGGGAACCGGAGCTGAATGAAGCCATACCAAACGACGAGCGTGACACCACG
ATGCCTGTAGCAATGGCAACAACGTTGCGCAAACTATTAACTGGCGAACTACTTACTCTAGCTTCCCGGCAACA
ATTAATAGACTGGATGGAGGCGGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCAGCACTGGGGCCAGATGGTAAG
CCCTCCCGTATCGTAGTTATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATATACTTTAGATTGATTTAA
AACTTCATTTTTAATTTAAAAGGATCTAGGTGAAGATCCTTTTTGATAATCTCATGACCAAAATCCCTTAACGTG
AGTTTTCGTTCCACTGAGCGTCAGACCCCGTAGAAAAGATCAAAGGATCTTCTTGAGATCCTTTTTTTTCTGCGCG
TAATCTGCTGCTTGCAAACAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTTCTTCTAGTGTAGCCGTAGTTAGG
CCACCACTTCAAGAACTCTGTAGCACCGCCTACATACCTCGCTCTGCTAATCCTGTTACCAGTGGCTGCTGCCAG
TGGCGATAAGTCGTGTCTTACCGGGTTGGACTCAAGACGATAGTTACCGGATAAGGCGCAGCGGTCGGGCTG
AACGGGGGGTTCGTGCACACAGCCCAGCTTGGAGCGAACGACCTACACCGAACTGAGATACCTACAGCGTGA
GCTATGAGAAAGCGCCACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCTGTCGGGTTTCGCCACCT
CTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGAT
AACCGTATTACCGCCTTTGAGTGAGCTGATACCGCTCGCCGCAGCCGAACGACCGAGCGCAGCGAGTCAGTGA
GCGAGGAAGCGGAAGAGCGCCCAATACGCAAACCGCCTCTCCCCGCGCGTTGGCCGATTCATTAATGCAGCT
GGCACGACAGGTTTCCCGACTGGAAAGCGGGCAGTGAGCGCAACGCAATTAATGTGAGTTAGCTCACTCATTA
GGCACCCCAGGCTTTACACTTTATGCTTCCGGCTCGTATGTTGTGTGGAATTGTGAGCGGATAACAATTTCACA
CAGGAAACAGCTATGACCATGATTACGCCAAGCTCGAAATTAACCCTCACTAAAGGGAACAAAAGCTGGAGCT
CCACCGCGGTGGCGGCCTCGAGGTCGAGATCCGGTCGACCAGCAACCATAGTCCCGCCCCTAACTCCGCCCAT
CCCGCCCCTAACTCCGCCCAGTTCCGCCCATTCTCCGCCCCATGGCTGACTAATTTTTTTTTATTTATGCAGAGGC
CGAGGCCGCCTCGGCCTCTGAGCTATTCCAGAAGTAGTGAGGAGGCTTTTTTGGAGGCCTAGGCTTTTGCAAA
AAGCTTCGACGGTATCGATTGGCTCATGTCCAACATTACCGCCATGTTGACATTGATTATTGACTAGTTATTAAT
AGTAATCAATTACGGGGTCATTAGTTCATAGCCCATATATGGAGTTCCGCGTTACATAACTTACGGTAAATGGC
CCGCCTGGCTGACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAG
TACATCAATGGGCGTGGATAGCGGTTTGACTCACGGGGATTTCCAAGTCTCCACCCCATTGACGTCAATGGGA
GTTTGTTTTGGCACCAAAATCAACGGGACTTTCCAAAATGTCGTAACAACTCCGCCCCATTGACGCAAATGGGC
GGTAGGCGTGTACGGAATTCGGAGTGGCGAGCCCTCAGATCCTGCATATAAGCAGCTGCTTTTTGCCTGTACT
GGGTCTCTCTG
SEQ ID NO: 71 (scFv CD19_FMC63 VH_MiH5_VL)
Asp Ile Gln Met Thr Gln Thr Thr Ser Ser Leu Ser Ala Ser Leu Gly Asp Arg Val Thr Ile Ser Cys Arg Ala Ser
Gln Asp Ile Ser Lys Tyr Leu Asn Trp Tyr Gln Gln Lys Pro Asp Gly Thr Val Lys Leu Leu Ile Tyr His Thr Ser
Arg Leu His Ser Gly Val Pro Ser Arg Phe Ser Gly Ser Gly Ser Gly Thr Asp Tyr Ser Leu Thr Ile Ser Asn Leu
Glu Gln Glu Asp Ile Ala Thr Tyr Phe Cys Gln Gln Gly Asn Thr Leu Pro Tyr Thr Phe Gly Gly Gly Thr Lys Leu
Glu Ile Thr
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Val Lys Leu Gln Glu Ser Gly Pro Gly Leu Val Ala Pro Ser Gln Ser Leu Ser Val Thr Cys Thr Val Ser Gly
Val Ser Leu Pro Asp Tyr Gly Val Ser Trp Ile Arg Gln Pro Pro Arg Lys Gly Leu Glu Trp Leu Gly Val Ile Trp
Gly Ser Glu Thr Thr Tyr Tyr Asn Ser Ala Leu Lys Ser Arg Leu Thr Ile Ile Lys Asp Asn Ser Lys Ser Gln Val
Phe Leu Lys Met Asn Ser Leu Gln Thr Asp Asp Thr Ala Ile Tyr Tyr Cys Ala Lys His Tyr Tyr Tyr Gly Gly Ser
Tyr Ala Met Asp Tyr Trp Gly Gln Gly Thr Ser Val Thr Val Ser Ser
SEQ ID NO: 72 (scFv CD20_Leu16 VL_MiH5_VH)
Asp Ile Val Leu Thr Gln Ser Pro Ala Ile Leu Ser Ala Ser Pro Gly Glu Lys Val Thr Met Thr Cys Arg Ala Ser
Ser Ser Val Asn Tyr Met Asp Trp Tyr Gln Lys Lys Pro Gly Ser Ser Pro Lys Pro Trp Ile Tyr Ala Thr Ser Asn
Leu Ala Ser Gly Val Pro Ala Arg Phe Ser Gly Ser Gly Ser Gly Thr Ser Tyr Ser Leu Thr Ile Ser Arg Val Glu
Ala Glu Asp Ala Ala Thr Tyr Tyr Cys Gln Gln Trp Ser Phe Asn Pro Pro Thr Phe Gly Gly Gly Thr Lys Leu
Glu Ile Lys
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Val Gln Leu Gln Gln Ser Gly Ala Glu Leu Val Lys Pro Gly Ala Ser Val Lys Met Ser Cys Lys Ala Ser Gly
Tyr Thr Phe Thr Ser Tyr Asn Met His Trp Val Lys Gln Thr Pro Gly Gln Gly Leu Glu Trp Ile Gly Ala Ile Tyr
Pro Gly Asn Gly Asp Thr Ser Tyr Asn Gln Lys Phe Lys Gly Lys Ala Thr Leu Thr Ala Asp Lys Ser Ser Ser Thr
Ala Tyr Met Gln Leu Ser Ser Leu Thr Ser Glu Asp Ser Ala Asp Tyr Tyr Cys Ala Arg Ser Asn Tyr Tyr Gly Ser
Ser Tyr Trp Phe Phe Asp Val Trp Gly Ala Gly Thr Thr Val Thr Val Ser Ser
SEQ ID NO: 73 (scFv ROR1_2A2 VH_MiH5_VL)
Gln Val Gln Leu Gln Gln Ser Gly Ala Glu Leu Val Arg Pro Gly Ala Ser Val Thr Leu Ser Cys Lys Ala Ser Gly
Tyr Thr Phe Ser Asp Tyr Glu Met His Trp Val Ile Gln Thr Pro Val His Gly Leu Glu Trp Ile Gly Ala Ile Asp
Pro Glu Thr Gly Gly Thr Ala Tyr Asn Gln Lys Phe Lys Gly Lys Ala Ile Leu Thr Ala Asp Lys Ser Ser Ser Thr
Ala Tyr Met Glu Leu Arg Ser Leu Thr Ser Glu Asp Ser Ala Val Tyr Tyr Cys Thr Gly Tyr Tyr Asp Tyr Asp Ser
Phe Thr Tyr Trp Gly Gln Gly Thr Leu Val Thr Val Ser Ala
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Met Thr Gln Ser Gln Lys Ile Met Ser Thr Thr Val Gly Asp Arg Val Ser Ile Thr Cys Lys Ala Ser
Gln Asn Val Asp Ala Ala Val Ala Trp Tyr Gln Gln Lys Pro Gly Gln Ser Pro Lys Leu Leu Ile Tyr Ser Ala Ser
Asn Arg Tyr Thr Gly Val Pro Asp Arg Phe Thr Gly Ser Gly Ser Gly Thr Asp Phe Thr Leu Thr Ile Ser Asn
Met Gln Ser Glu Asp Leu Ala Asp Tyr Phe Cys Gln Gln Tyr Asp Ile Tyr Pro Tyr Thr Phe Gly Gly Gly Thr
Lys Leu Glu Ile Lys
SEQ ID NO: 74 (scFv ROR1_4-2 VH_MiH5_VL)
Gln Glu Gln Gln Lys Glu Ser Gly Gly Gly Leu Phe Lys Pro Thr Asp Thr Leu Thr Leu Thr Cys Thr Ala Ser Gly
Phe Asp Ile Ser Ser Tyr Tyr Met Ser Trp Val Arg Gln Ala Pro Gly Asn Gly Leu Glu Trp Ile Gly Ala Ile Gly
Ile Ser Gly Asn Ala Tyr Tyr Ala Ser Trp Ala Lys Ser Arg Ser Thr Ile Thr Arg Asn Thr Asn Leu Asn Thr Val
Thr Leu Lys Met Thr Ser Leu Thr Ala Ala Asp Thr Ala Thr Tyr Phe Cys Ala Arg Asp His Pro Thr Tyr Gly
Met Asp Leu Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Ser Tyr Glu Leu Thr Gln Leu Pro Ser Val Ser Val Ser Leu Gly Gln Thr Ala Arg Ile Thr Cys Glu Gly Asn Asn
Ile Gly Ser Lys Ala Val His Trp Tyr Gln Gln Lys Pro Gly Leu Ala Pro Gly Leu Leu Ile Tyr Asp Asp Asp Glu
Arg Pro Ser Gly Val Pro Asp Arg Phe Ser Gly Ser Asn Ser Gly Asp Thr Ala Thr Leu Thr Ile Ser Gly Ala Gln
Ala Gly Asp Glu Ala Asp Tyr Tyr Cys Gln Val Trp Asp Ser Ser Ala Tyr Val Phe Gly Gly Gly Thr Gln Leu Thr
Val Thr Gly
SEQ ID NO: 75 (scFv ROR1_R11 VH_MiH5_VL)
Gln Ser Val Lys Glu Ser Glu Gly Asp Leu Val Thr Pro Ala Gly Asn Leu Thr Leu Thr Cys Thr Ala Ser Gly Ser
Asp Ile Asn Asp Tyr Pro Ile Ser Trp Val Arg Gln Ala Pro Gly Lys Gly Leu Glu Trp Ile Gly Phe Ile Asn Ser
Gly Gly Ser Thr Trp Tyr Ala Ser Trp Val Lys Gly Arg Phe Thr Ile Ser Arg Thr Ser Thr Thr Val Asp Leu Lys
Met Thr Ser Leu Thr Thr Asp Asp Thr Ala Thr Tyr Phe Cys Ala Arg Gly Tyr Ser Thr Tyr Tyr Gly Asp Phe
Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Met Thr Gln Thr Pro Ser Ser Thr Ser Gly Ala Val Gly Gly Thr Val Thr Ile Asn Cys Gln Ala Ser
Gln Ser Ile Asp Ser Asn Leu Ala Trp Phe Gln Gln Lys Pro Gly Gln Pro Pro Thr Leu Leu Ile Tyr Arg Ala Ser
Asn Leu Ala Ser Gly Val Pro Ser Arg Phe Ser Gly Ser Arg Ser Gly Thr Glu Tyr Thr Leu Thr Ile Ser Gly Val
Gln Arg Glu Asp Ala Ala Thr Tyr Tyr Cys Leu Gly Gly Val Gly Asn Val Ser Tyr Arg Thr Ser Phe Gly Gly Gly
Thr Glu Val Val Val Lys
SEQ ID NO: 76 (scFv ROR1_R12 VH_MiH5_VL)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
SEQ ID NO: 77 (scFv ROR2_4-1 VH_MiH5_VL)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 78 (scFv SLAM F7_ERCS409 VH_MiH5_VL)
Gln Ser Val Lys Glu Ser Glu Gly Gly Leu Phe Lys Pro Thr Asp Thr Leu Thr Leu Thr Cys Thr Val Ser Gly Phe
Ser Leu Asn Ser Tyr Gly Val Ile Trp Val Arg Gln Ala Pro Gly Asn Gly Leu Glu Tyr Ile Gly Ile Ile Gly Ser Ser
Gly Asn Thr Tyr Tyr Ala Ser Trp Ala Lys Ser Arg Ser Thr Ile Thr Arg Asn Thr Arg Leu Asn Thr Val Thr Leu
Lys Met Thr Ser Leu Thr Ala Ala Asp Thr Ala Thr Tyr Phe Cys Ala Arg Tyr Tyr Gly Asp Ser Gly Phe Asp
Ser Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Ala Gln Val Leu Thr Gln Thr Pro Ser Ser Thr Ser Val Ala Val Gly Gly Thr Val Thr Ile Lys Cys Gln Ala Ser
Gln Ser Ile Gly Ser Trp Leu Ser Trp Tyr Gln Gln Lys Pro Gly Gln Pro Pro Lys Leu Leu Ile Tyr Gly Ala Ser
Asn Leu Ala Ser Gly Val Pro Ser Arg Phe Lys Gly Gly Arg Ser Gly Thr Glu Tyr Ser Leu Thr Ile Ser Gly Val
Gln Arg Glu Asp Ala Ala Thr Tyr Tyr Cys Leu Gly Ala Ser Pro Asn Gly Trp Ala Phe Gly Ala Gly Thr Asn Val
Glu Ile Lys
SEQ ID NO: 79 (scFv SLAM F7_huLuc63 VH_MiH5_VL)
Glu Val Gln Leu Val Glu Ser Gly Gly Gly Leu Val Gln Pro Gly Gly Ser Leu Arg Leu Ser Cys Ala Ala Ser Gly
Phe Asp Phe Ser Arg Tyr Trp Met Ser Trp Val Arg Gln Ala Pro Gly Lys Gly Leu Glu Trp Ile Gly Glu Ile Asn
Pro Asp Ser Ser Thr Ile Asn Tyr Ala Pro Ser Leu Lys Asp Lys Phe Ile Ile Ser Arg Asp Asn Ala Lys Asn Ser
Leu Tyr Leu Gln Met Asn Ser Leu Arg Ala Glu Asp Thr Ala Val Tyr Tyr Cys Ala Arg Pro Asp Gly Asn Tyr
Trp Tyr Phe Asp Val Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Gln Met Thr Gln Ser Pro Ser Ser Leu Ser Ala Ser Val Gly Asp Arg Val Thr Ile Thr Cys Lys Ala Ser
Gln Asp Val Gly Ile Ala Val Ala Trp Tyr Gln Gln Lys Pro Gly Lys Val Pro Lys Leu Leu Ile Tyr Trp Ala Ser
Thr Arg His Thr Gly Val Pro Asp Arg Phe Ser Gly Ser Gly Ser Gly Thr Asp Phe Thr Leu Thr Ile Ser Ser Leu
Gln Pro Glu Asp Val Ala Thr Tyr Tyr Cys Gln Gln Tyr Ser Ser Tyr Pro Tyr Thr Phe Gly Gln Gly Thr Lys Val
Glu Ile Lys
SEQ ID NO: 80 (scFv FLT3_BV10 VH_MiH5_VL)
Gln Val Gln Leu Lys Gln Ser Gly Pro Gly Leu Val Gln Pro Ser Gln Ser Leu Ser Ile Thr Cys Thr Val Ser Gly
Phe Ser Leu Thr Asn Tyr Gly Leu His Trp Val Arg Gln Ser Pro Gly Lys Gly Leu Glu Trp Leu Gly Val Ile Trp
Ser Gly Gly Ser Thr Asp Tyr Asn Ala Ala Phe Ile Ser Arg Leu Ser Ile Ser Lys Asp Asn Ser Lys Ser Gln Val
Phe Phe Lys Met Asn Ser Leu Gln Ala Asp Asp Thr Ala Ile Tyr Tyr Cys Ala Arg Lys Gly Gly Ile Tyr Tyr Ala
Asn His Tyr Tyr Ala Met Asp Tyr Trp Gly Gln Gly Thr Ser Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Met Thr Gln Ser Pro Ser Ser Leu Ser Val Ser Ala Gly Glu Lys Val Thr Met Ser Cys Lys Ser Ser
Gln Ser Leu Leu Asn Ser Gly Asn Gln Lys Asn Tyr Met Ala Trp Tyr Gln Gln Lys Pro Gly Gln Pro Pro Lys
Leu Leu Ile Tyr Gly Ala Ser Thr Arg Glu Ser Gly Val Pro Asp Arg Phe Thr Gly Ser Gly Ser Gly Thr Asp Phe
Thr Leu Thr Ile Ser Ser Val Gln Ala Glu Asp Leu Ala Val Tyr Tyr Cys Gln Asn Asp His Ser Tyr Pro Leu Thr
Phe Gly Ala Gly Thr Lys Leu Glu Leu Lys Arg
SEQ ID NO: 81 (scFv FLT3_4G8 VH_MiH5_VL)
Gln Val Gln Leu Gln Gln Pro Gly Ala Glu Leu Val Lys Pro Gly Ala Ser Leu Lys Leu Ser Cys Lys Ser Ser Gly
Tyr Thr Phe Thr Ser Tyr Trp Met His Trp Val Arg Gln Arg Pro Gly His Gly Leu Glu Trp Ile Gly Glu Ile Asp
Pro Ser Asp Ser Tyr Lys Asp Tyr Asn Gln Lys Phe Lys Asp Lys Ala Thr Leu Thr Val Asp Arg Ser Ser Asn
Thr Ala Tyr Met His Leu Ser Ser Leu Thr Ser Asp Asp Ser Ala Val Tyr Tyr Cys Ala Arg Ala Ile Thr Thr Thr
Pro Phe Asp Phe Trp Gly Gln Gly Thr Thr Leu Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Leu Thr Gln Ser Pro Ala Thr Leu Ser Val Thr Pro Gly Asp Ser Val Ser Leu Ser Cys Arg Ala Ser
Gln Ser Ile Ser Asn Asn Leu His Trp Tyr Gln Gln Lys Ser His Glu Ser Pro Arg Leu Leu Ile Lys Tyr Ala Ser
Gln Ser Ile Ser Gly Ile Pro Ser Arg Phe Ser Gly Ser Gly Ser Gly Thr Asp Phe Thr Leu Ser Ile Asn Ser Val
Glu Thr Glu Asp Phe Gly Val Tyr Phe Cys Gln Gln Ser Asn Thr Trp Pro Tyr Thr Phe Gly Gly Gly Thr Lys
Leu Glu Ile Lys Arg
SEQ ID NO: 82 (scFv Siglec-6_JML-1 VH_MiH5_VL)
Lys Val Gln Leu Leu Glu Ser Gly Gly Gly Leu Val Gln Pro Gly Arg Ser Leu Arg Leu Ser Cys Ala Ala Ser Gly
Phe Thr Phe Asp Asp Tyr Gly Met His Trp Val Arg Gln Ala Pro Gly Lys Gly Leu Glu Trp Val Ser Gly Ile Ser
Trp Asn Ser Gly Ser Ile Gly Tyr Ala Asp Ser Val Lys Gly Arg Phe Thr Ile Ser Arg Asp Asn Ser Lys Asn Thr
Leu Tyr Leu Gln Met Asn Ser Leu Arg Ala Glu Asp Thr Ala Val Tyr Tyr Cys Ala Arg Gly Gly Gln Thr Ile Asp
Ile Trp Gly Gln Gly Thr Met Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Gln Met Thr Gln Ser Pro Ser Ser Leu Ser Ala Ser Val Gly Asp Arg Val Thr Ile Thr Cys Arg Ala Ser
Gln Ser Ile Ser Ser Tyr Leu Asn Trp Tyr Gln Gln Lys Pro Gly Lys Ala Pro Lys Leu Leu Ile Tyr Ala Ala Ser
Ser Leu Gln Ser Gly Val Pro Ser Arg Phe Ser Gly Ser Gly Ser Gly Thr Asp Phe Thr Leu Thr Ile Ser Ser Leu
Gln Pro Glu Asp Phe Ala Thr Tyr Tyr Cys Gln Gln Ser Tyr Ser Thr Pro Phe Thr Phe Gly Pro Gly Thr Lys
Val Asp Ile Lys
SEQ ID NO: 83 (scFv avb3_LM609v7 VH_MiH5_VL)
Gln Val Gln Leu Gln Glu Ser Gly Pro Gly Leu Val Lys Pro Ser Gln Thr Leu Ser Leu Thr Cys Thr Val Ser Gly
Ala Ser Ile Ser Arg Gly Gly Tyr Tyr Trp Ser Trp Ile Arg Gln Tyr Pro Gly Lys Gly Leu Glu Trp Ile Gly Tyr Ile
His His Ser Gly Ser Thr Tyr Tyr Asn Pro Ser Leu Lys Ser Arg Val Thr Ile Ala Ile Asp Thr Ser Lys Asn Gln
Leu Ser Leu Arg Leu Thr Ser Val Thr Ala Ala Asp Thr Ala Val Tyr Tyr Cys Ala Arg His Asn Tyr Gly Ser Phe
Ala Tyr Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Met Thr Gln Ser Pro Glu Phe Gln Ser Val Thr Pro Lys Glu Thr Val Thr Ile Thr Cys Arg Ala Ser
Gln Asp Ile Gly Asn Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Ser Arg Leu
Glu Pro Glu Asp Phe Ala Val Tyr Tyr Cys Gln Gln Ser Asn Ser Trp Pro His Thr Phe Gly Gln Gly Thr Lys Leu
Glu Ile Lys
SEQ ID NO: 84 (scFv avb3_LM609v11 VH_MiH5_VL)
Gln Val Gln Leu Val Gln Ser Gly Ala Glu Val Arg Lys Pro Gly Ser Ser Val Arg Val Ser Cys Lys Ala Ser Gly
Gly Thr Phe Ser Gly Phe Ala Val Ser Trp Val Arg Gln Ala Pro Gly Gln Arg Phe Glu Trp Leu Gly Gly Ile Val
Ala Ser Leu Gly Ser Thr Asp Tyr Ala Gln Lys Phe Gln Asp Lys Leu Thr Ile Thr Val Asp Glu Ser Thr Ala Thr
Val Tyr Met Glu Met Arg Asn Leu Arg Ser Asp Asp Thr Ala Val Tyr Tyr Cys Ala Arg His Asn Tyr Gly Ser
Phe Ala Tyr Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Met Thr Gln Ser Pro Glu Phe Gln Ser Val Thr Pro Lys Glu Thr Val Thr Ile Thr Cys Arg Ala Ser
Gln Asp Ile Gly Thr Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Tyr Ser Leu
Glu Ala Glu Asp Phe Ala Val Tyr Tyr Cys Gln Gln Ser Asn Ser Trp Pro His Thr Phe Gly Gln Gly Thr Lys Leu
Glu Ile Lys
SEQ ID NO: 85 (scFv BCMA_BCMA30 VH_MiH5_VL)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ser Ser
Thr Ala Tyr Met Glu Leu Ser Ser Leu Arg Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Met Thr Gln Thr Pro Leu Ser Leu Ser Val Thr Pro Gly Glu Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Ala Asp Phe Thr Leu
Lys Ile Ser Arg Val Glu Ala Glu Asp Val Gly Val Tyr Tyr Cys Ala Glu Thr Ser His Val Pro Trp Thr Phe Gly
Gln Gly Thr Lys Leu Glu Ile Lys
SEQ ID NO: 86 (scFv BCMA_BCMA50 VH_MiH5_VL)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ile Asn
Thr Ala Tyr Met Glu Leu Ser Ser Leu Thr Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Met Thr Gln Thr Pro Leu Ser Leu Ser Val Thr Pro Gly Gln Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Thr Asp Phe Thr Leu
Lys Ile Ser Arg Val Glu Ala Glu Asp Val Gly Ile Tyr Tyr Cys Ser Gln Ser Ser Ile Tyr Pro Trp Thr Phe Gly Gln
Gly Thr Lys Leu Glu Ile Lys
SEQ ID NO: 87 (scFv ROR1_huR12 VH_Linker_VL)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Ala Asp
Asp Gly Ala Leu Phe Asn Ile Trp Gly Gln Gly Thr Leu Val Thr Val Ser Ser
Gly Gly Gly Gly Ser Gly Gly Gly Gly Ser Gly Gly Gly Gly Ser
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Gly
SEQ ID NO: 88 (scFv ROR1_huR12 VH_MiH5_VL)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Ala Asp
Asp Gly Ala Leu Phe Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Gly
SEQ ID NO: 89 (scFv ROR1_R12/V16 VH_Linker_VL)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Thr Gly
SEQ ID NO: 90 (scFv ROR1_R12/V16 VH_MiH5_VL)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Thr Gly
SEQ ID NO: 91 (scFv ROR1_R12/V20 VH_Linker_VL)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Gly Glu
Asp Leu Gly Leu Phe Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
SEQ ID NO: 92 (scFv ROR1_R12/V20 VH_MiH5_VL)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Gly Glu
Asp Leu Gly Leu Phe Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
SEQ ID NO: 93 (scFv ROR1_R12/V16-20 VH_Linker_VL)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Gly Glu
Asp Leu Gly Leu Phe Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Thr Gly
SEQ ID NO: 94 (scFv ROR1_R12/V16-20 VH_MiH5_VL)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Gly Glu
Asp Leu Gly Leu Phe Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Thr Gly
SEQ ID NO: 95 (scFv ROR1_huR12/V16 VH_Linker_VL)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Ala Asp
Asp Gly Ala Leu Phe Asn Ile Trp Gly Gln Gly Thr Leu Val Thr Val Ser Ser
Gly Gly Gly Gly Ser Gly Gly Gly Gly Ser Gly Gly Gly Gly Ser
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Gly
SEQ ID NO: 96 (scFv ROR1_huR12/V16 VH_MiH5_VL)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Ala Asp
Asp Gly Ala Leu Phe Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln AlaAsp Asp Glu Ala Asp Tyr Tyr Cys Gly Ala Glu Ser Arg Gly Tyr Val Phe Gly Gly Gly
Thr Gln Leu Thr Val Gly
SEQ ID NO: 97 (scFv ROR1_huR12/V20 VH_Linker_VL)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Gly Glu
Asp Leu Gly Leu Phe Asn Ile Trp Gly Gln Gly Thr Leu Val Thr Val Ser Ser
Gly Gly Gly Gly Ser Gly Gly Gly Gly Ser Gly Gly Gly Gly Ser
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Gly
SEQ ID NO: 98 (scFv ROR1_huR12/V20 VH_MiH5_VL)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Gly Glu
Asp Leu Gly Leu Phe Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Asp Tyr Ile Gly Tyr Val Phe Gly Gly Gly
Thr Gln Leu Thr Val Gly
SEQ ID NO: 99 (scFv ROR1_huR12/V16-20 VH_Linker_VL)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Gly Glu
Asp Leu Gly Leu Phe Asn Ile Trp Gly Gln Gly Thr Leu Val Thr Val Ser Ser
Gly Gly Gly Gly Ser Gly Gly Gly Gly Ser Gly Gly Gly Gly Ser
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Gly
SEQ ID NO: 100 (scFv ROR1_huR12/V16-20 VH_MiH5_VL)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Gly Glu
Asp Leu Gly Leu Phe Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Glu Ser Arg Gly Tyr Val Phe Gly Gly Gly
Thr Gln Leu Thr Val Gly
SEQ ID NO: 101 (scFv ROR2_X3.12 VH_4GS3_VL)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Asp Arg Trp Ser Leu Asn
Ile Trp Gly Pro Gly Thr Leu Val Thr Val Ser Ser
Gly Gly Gly Gly Ser Gly Gly Gly Gly Ser Gly Gly Gly Gly Ser
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 102 (scFv ROR2_X3.12 VH_MiH5_VL)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Asp Arg Trp Ser Leu Asn
Ile Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 103 (scFv ROR2_XBR2-401-DM VH_4GS3_VL)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Gly
Arg Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
Gly Gly Gly Gly Ser Gly Gly Gly Gly Ser Gly Gly Gly Gly Ser
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 104 (scFv ROR2_XBR2-401-DM VH_MiH5_VL)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Gly
Arg Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 105 (scFv ROR2_huX3.12.5 VH_4GS3_VL)
Glu Val Gln Leu Val Glu Ser Gly Gly Gly Leu Ile Gln Pro Gly Gly Ser Leu Arg Leu Ser Cys Ala Ala Ser Gly
Phe Thr Val Ser Ser Tyr Gly Val Thr Trp Val Arg Gln Ala Pro Gly Lys Gly Leu Glu Trp Val Ser Tyr Ile Asn
Thr Ala Gly Asn Thr Tyr Tyr Ala Ser Trp Ala Lys Ser Arg Phe Thr Ile Ser Arg Asp Asn Ser Lys Asn Thr Leu
Tyr Leu Gln Met Asn Ser Leu Arg Ala Glu Asp Thr Ala Val Tyr Tyr Cys Ala Arg Asp Asp Arg Trp Ser Leu
Asn Ile Trp Gly Gln Gly Thr Leu Val Thr Val Ser Ser
Gly Gly Gly Gly Ser Gly Gly Gly Gly Ser Gly Gly Gly Gly Ser
Asp Pro Met Leu Thr Gln Ser Pro Ser Ser Leu Ser Ala Ser Val Gly Asp Arg Val Thr Ile Thr Cys Gln Ala Ser
Gln Ser Ile Ser Ser Asp Leu Ser Trp Tyr Gln Gln Lys Pro Gly Lys Ala Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Ser Leu
Gln Pro Glu Asp Phe Ala Thr Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 106 (scFv ROR2_huX3.12.5 VH_MiH5_VL)
Glu Val Gln Leu Val Glu Ser Gly Gly Gly Leu Ile Gln Pro Gly Gly Ser Leu Arg Leu Ser Cys Ala Ala Ser Gly
Phe Thr Val Ser Ser Tyr Gly Val Thr Trp Val Arg Gln Ala Pro Gly Lys Gly Leu Glu Trp Val Ser Tyr Ile Asn
Thr Ala Gly Asn Thr Tyr Tyr Ala Ser Trp Ala Lys Ser Arg Phe Thr Ile Ser Arg Asp Asn Ser Lys Asn Thr Leu
Tyr Leu Gln Met Asn Ser Leu Arg Ala Glu Asp Thr Ala Val Tyr Tyr Cys Ala Arg Asp Asp Arg Trp Ser Leu
Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Ser Pro Ser Ser Leu Ser Ala Ser Val Gly Asp Arg Val Thr Ile Thr Cys Gln Ala Ser
Gln Ser Ile Ser Ser Asp Leu Ser Trp Tyr Gln Gln Lys Pro Gly Lys Ala Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Ser Leu
Gln Pro Glu Asp Phe Ala Thr Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 107(scFv ROR2_huX3.12.6 VH_4GS3_VL)
Glu Val Gln Leu Val Glu Ser Gly Gly Gly Leu Val Gln Pro Gly Gly Ser Leu Arg Leu Ser Cys Ala Ala Ser Gly
Phe Thr Phe Ser Ser Tyr Gly Val Thr Trp Val Arg Gln Ala Pro Gly Lys Gly Leu Glu Trp Val Ser Tyr Ile Asn
Thr Ala Gly Asn Thr Tyr Tyr Ala Ser Trp Ala Lys Ser Arg Phe Thr Ile Ser Arg Asp Asn Ala Lys Asn Ser Leu
Tyr Leu Gln Met Asn Ser Leu Arg Ala Glu Asp Thr Ala Val Tyr Tyr Cys Ala Arg Asp Asp Arg Trp Ser Leu
Asn Ile Trp Gly Gln Gly Thr Leu Val Thr Val Ser Ser
Gly Gly Gly Gly Ser Gly Gly Gly Gly Ser Gly Gly Gly Gly Ser
Asp Pro Met Leu Thr Gln Ser Pro Ser Ser Leu Ser Ala Ser Val Gly Asp Arg Val Thr Ile Thr Cys Gln Ala Ser
Gln Ser Ile Ser Ser Asp Leu Ser Trp Tyr Gln Gln Lys Pro Gly Lys Ala Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Ser Leu
Gln Pro Glu Asp Phe Ala Thr Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 108 (scFv ROR2_huX3.12.6 VH_MiH5_VL)
Glu Val Gln Leu Val Glu Ser Gly Gly Gly Leu Val Gln Pro Gly Gly Ser Leu Arg Leu Ser Cys Ala Ala Ser Gly
Phe Thr Phe Ser Ser Tyr Gly Val Thr Trp Val Arg Gln Ala Pro Gly Lys Gly Leu Glu Trp Val Ser Tyr Ile Asn
Thr Ala Gly Asn Thr Tyr Tyr Ala Ser Trp Ala Lys Ser Arg Phe Thr Ile Ser Arg Asp Asn Ala Lys Asn Ser Leu
Tyr Leu Gln Met Asn Ser Leu Arg Ala Glu Asp Thr Ala Val Tyr Tyr Cys Ala Arg Asp Asp Arg Trp Ser Leu
Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Ser Pro Ser Ser Leu Ser Ala Ser Val Gly Asp Arg Val Thr Ile Thr Cys Gln Ala Ser
Gln Ser Ile Ser Ser Asp Leu Ser Trp Tyr Gln Gln Lys Pro Gly Lys Ala Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Ser Leu
Gln Pro Glu Asp Phe Ala Thr Tyr Tyr Cys Leu Gly Gly Tyr Ala Asp Ala Ser Tyr Arg Thr Ala Phe Gly Gly Gly
Thr Lys Leu Glu Ile Lys
SEQ ID NO: 109 (CD28tm + CD28/zeta)
Met Phe Trp Val Leu Val Val Val Gly Gly Val Leu Ala Cys Tyr Ser Leu Leu Val Thr Val Ala Phe Ile Ile Phe
Trp Val
Arg Ser Lys Arg Ser Arg Gly Gly His Ser Asp Tyr Met Asn Met Thr Pro Arg Arg Pro Gly Pro Thr Arg Lys
His Tyr Gln Pro Tyr Ala Pro Pro Arg Asp Phe Ala Ala Tyr Arg Ser
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 110 (CD28tm + 4-1BB/zeta)
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 111 (CD28tm + CD28/4-1BB/zeta)
Met Phe Trp Val Leu Val Val Val Gly Gly Val Leu Ala Cys Tyr Ser Leu Leu Val Thr Val Ala Phe Ile Ile Phe
Trp Val
Arg Ser Lys Arg Ser Arg Gly Gly His Ser Asp Tyr Met Asn Met Thr Pro Arg Arg Pro Gly Pro Thr Arg Lys
His Tyr Gln Pro Tyr Ala Pro Pro Arg Asp Phe Ala Ala Tyr Arg Ser
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 112 (CD28tm + 4-1BB/CD28/zeta)
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Ser Lys Arg Ser Arg Gly Gly His Ser Asp Tyr Met Asn Met Thr Pro Arg Arg Pro Gly Pro Thr Arg Lys
His Tyr Gln Pro Tyr Ala Pro Pro Arg Asp Phe Ala Ala Tyr Arg Ser
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 113 (CD28tm + zeta)
Met Phe Trp Val Leu Val Val Val Gly Gly Val Leu Ala Cys Tyr Ser Leu Leu Val Thr Val Ala Phe Ile Ile Phe
Trp Val
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 114 (ICOStm + zeta)
Phe Trp Leu Pro Ile Gly Cys Ala Ala Phe Val Val Val Cys Ile Leu Gly Cys Ile Leu Ile
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 115 (OX40tm + OX40/zeta)
Val Ala Ala Ile Leu Gly Leu Gly Leu Val Leu Gly Leu Leu Gly Pro Leu Ala Ile Leu Leu Ala Leu Tyr Leu Leu
Arg Arg Asp Gln Arg Leu Pro Pro Asp Ala His Lys Pro Pro Gly Gly Gly Ser Phe Arg Thr Pro Ile Gln Glu Glu
Gln Ala Asp Ala His Ser Thr Leu Ala Lys Ile
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 174 (CD4tm + intracellular)
Met Ala Leu Ile Val Leu Gly Gly Val Ala Gly Leu Leu Leu Phe Ile Gly Leu Gly Ile Phe Phe Cys Val Arg Cys
Arg His Arg Arg Arg Gln Ala Glu Arg Met Ser Gln Ile Lys Arg Leu Leu Ser Glu Lys Lys Thr Cys Gln Cys Pro
His Arg Phe Gln Lys Thr Cys Ser Pro Ile
SEQ ID NO: 116 (scFv CD19_FMC63 VH_MiH5_VL_MiH0_CD28tm + 4-113B/zeta)
Asp Ile Gln Met Thr Gln Thr Thr Ser Ser Leu Ser Ala Ser Leu Gly Asp Arg Val Thr Ile Ser Cys Arg Ala Ser
Gln Asp Ile Ser Lys Tyr Leu Asn Trp Tyr Gln Gln Lys Pro Asp Gly Thr Val Lys Leu Leu Ile Tyr His Thr Ser
Arg Leu His Ser Gly Val Pro Ser Arg Phe Ser Gly Ser Gly Ser Gly Thr Asp Tyr Ser Leu Thr Ile Ser Asn Leu
Glu Gln Glu Asp Ile Ala Thr Tyr Phe Cys Gln Gln Gly Asn Thr Leu Pro Tyr Thr Phe Gly Gly Gly Thr Lys Leu
Glu Ile Thr
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Val Lys Leu Gln Glu Ser Gly Pro Gly Leu Val Ala Pro Ser Gln Ser Leu Ser Val Thr Cys Thr Val Ser Gly
Val Ser Leu Pro Asp Tyr Gly Val Ser Trp Ile Arg Gln Pro Pro Arg Lys Gly Leu Glu Trp Leu Gly Val Ile Trp
Gly Ser Glu Thr Thr Tyr Tyr Asn Ser Ala Leu Lys Ser Arg Leu Thr Ile Ile Lys Asp Asn Ser Lys Ser Gln Val
Phe Leu Lys Met Asn Ser Leu Gln Thr Asp Asp Thr Ala Ile Tyr Tyr Cys Ala Lys His Tyr Tyr Tyr Gly Gly Ser
Tyr Ala Met Asp Tyr Trp Gly Gln Gly Thr Ser Val Thr Val Ser Ser
Glu Leu Lys Thr Pro Leu Gly Asp Thr Thr His Thr Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 117 (scFv CD19_FMC63 VH_MiH5_VL_MiH1_CD28tm + 4-113B/zeta)
Asp Ile Gln Met Thr Gln Thr Thr Ser Ser Leu Ser Ala Ser Leu Gly Asp Arg Val Thr Ile Ser Cys Arg Ala Ser
Gln Asp Ile Ser Lys Tyr Leu Asn Trp Tyr Gln Gln Lys Pro Asp Gly Thr Val Lys Leu Leu Ile Tyr His Thr Ser
Arg Leu His Ser Gly Val Pro Ser Arg Phe Ser Gly Ser Gly Ser Gly Thr Asp Tyr Ser Leu Thr Ile Ser Asn Leu
Glu Gln Glu Asp Ile Ala Thr Tyr Phe Cys Gln Gln Gly Asn Thr Leu Pro Tyr Thr Phe Gly Gly Gly Thr Lys Leu
Glu Ile Thr
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Val Lys Leu Gln Glu Ser Gly Pro Gly Leu Val Ala Pro Ser Gln Ser Leu Ser Val Thr Cys Thr Val Ser Gly
Val Ser Leu Pro Asp Tyr Gly Val Ser Trp Ile Arg Gln Pro Pro Arg Lys Gly Leu Glu Trp Leu Gly Val Ile Trp
Gly Ser Glu Thr Thr Tyr Tyr Asn Ser Ala Leu Lys Ser Arg Leu Thr Ile Ile Lys Asp Asn Ser Lys Ser Gln Val
Phe Leu Lys Met Asn Ser Leu Gln Thr Asp Asp Thr Ala Ile Tyr Tyr Cys Ala Lys His Tyr Tyr Tyr Gly Gly Ser
Tyr Ala Met Asp Tyr Trp Gly Gln Gly Thr Ser Val Thr Val Ser Ser
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 118 (scFv CD20_Leu16 VL_MiH5_VH_MiH3_CD28tm + 4-113B/zeta)
Asp Ile Val Leu Thr Gln Ser Pro Ala Ile Leu Ser Ala Ser Pro Gly Glu Lys Val Thr Met Thr Cys Arg Ala Ser
Ser Ser Val Asn Tyr Met Asp Trp Tyr Gln Lys Lys Pro Gly Ser Ser Pro Lys Pro Trp Ile Tyr Ala Thr Ser Asn
Leu Ala Ser Gly Val Pro Ala Arg Phe Ser Gly Ser Gly Ser Gly Thr Ser Tyr Ser Leu Thr Ile Ser Arg Val Glu
Ala Glu Asp Ala Ala Thr Tyr Tyr Cys Gln Gln Trp Ser Phe Asn Pro Pro Thr Phe Gly Gly Gly Thr Lys Leu
Glu Ile Lys
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Val Gln Leu Gln Gln Ser Gly Ala Glu Leu Val Lys Pro Gly Ala Ser Val Lys Met Ser Cys Lys Ala Ser Gly
Tyr Thr Phe Thr Ser Tyr Asn Met His Trp Val Lys Gln Thr Pro Gly Gln Gly Leu Glu Trp Ile Gly Ala Ile Tyr
Pro Gly Asn Gly Asp Thr Ser Tyr Asn Gln Lys Phe Lys Gly Lys Ala Thr Leu Thr Ala Asp Lys Ser Ser Ser Thr
Ala Tyr Met Gln Leu Ser Ser Leu Thr Ser Glu Asp Ser Ala Asp Tyr Tyr Cys Ala Arg Ser Asn Tyr Tyr Gly Ser
Ser Tyr Trp Phe Phe Asp Val Trp Gly Ala Gly Thr Thr Val Thr Val Ser Ser
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 119 (scFv ROR1_2A2 VH_MiH5_VL_MiH1_CD28tm + 4-1BB/zeta)
Gln Val Gln Leu Gln Gln Ser Gly Ala Glu Leu Val Arg Pro Gly Ala Ser Val Thr Leu Ser Cys Lys Ala Ser Gly
Tyr Thr Phe Ser Asp Tyr Glu Met His Trp Val Ile Gln Thr Pro Val His Gly Leu Glu Trp Ile Gly Ala Ile Asp
Pro Glu Thr Gly Gly Thr Ala Tyr Asn Gln Lys Phe Lys Gly Lys Ala Ile Leu Thr Ala Asp Lys Ser Ser Ser Thr
Ala Tyr Met Glu Leu Arg Ser Leu Thr Ser Glu Asp Ser Ala Val Tyr Tyr Cys Thr Gly Tyr Tyr Asp Tyr Asp Ser
Phe Thr Tyr Trp Gly Gln Gly Thr Leu Val Thr Val Ser Ala
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Met Thr Gln Ser Gln Lys Ile Met Ser Thr Thr Val Gly Asp Arg Val Ser Ile Thr Cys Lys Ala Ser
Gln Asn Val Asp Ala Ala Val Ala Trp Tyr Gln Gln Lys Pro Gly Gln Ser Pro Lys Leu Leu Ile Tyr Ser Ala Ser
Asn Arg Tyr Thr Gly Val Pro Asp Arg Phe Thr Gly Ser Gly Ser Gly Thr Asp Phe Thr Leu Thr Ile Ser Asn
Met Gln Ser Glu Asp Leu Ala Asp Tyr Phe Cys Gln Gln Tyr Asp Ile Tyr Pro Tyr Thr Phe Gly Gly Gly Thr
Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 120 (scFv ROR1_4-2 VH_MiH5_VL_MiH1_CD28tm + 4-1BB/zeta)
Gln Glu Gln Gln Lys Glu Ser Gly Gly Gly Leu Phe Lys Pro Thr Asp Thr Leu Thr Leu Thr Cys Thr Ala Ser Gly
Phe Asp Ile Ser Ser Tyr Tyr Met Ser Trp Val Arg Gln Ala Pro Gly Asn Gly Leu Glu Trp Ile Gly Ala Ile Gly
Ile Ser Gly Asn Ala Tyr Tyr Ala Ser Trp Ala Lys Ser Arg Ser Thr Ile Thr Arg Asn Thr Asn Leu Asn Thr Val
Thr Leu Lys Met Thr Ser Leu Thr Ala Ala Asp Thr Ala Thr Tyr Phe Cys Ala Arg Asp His Pro Thr Tyr Gly
Met Asp Leu Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Ser Tyr Glu Leu Thr Gln Leu Pro Ser Val Ser Val Ser Leu Gly Gln Thr Ala Arg Ile Thr Cys Glu Gly Asn Asn
Ile Gly Ser Lys Ala Val His Trp Tyr Gln Gln Lys Pro Gly Leu Ala Pro Gly Leu Leu Ile Tyr Asp Asp Asp Glu
Arg Pro Ser Gly Val Pro Asp Arg Phe Ser Gly Ser Asn Ser Gly Asp Thr Ala Thr Leu Thr Ile Ser Gly Ala Gln
Ala Gly Asp Glu Ala Asp Tyr Tyr Cys Gln Val Trp Asp Ser Ser Ala Tyr Val Phe Gly Gly Gly Thr Gln Leu Thr
Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 121 (scFv ROR1_R11 VH_MiH5_VL_MiH3_CD28tm + 4-1BB/zeta)
Gln Ser Val Lys Glu Ser Glu Gly Asp Leu Val Thr Pro Ala Gly Asn Leu Thr Leu Thr Cys Thr Ala Ser Gly Ser
Asp Ile Asn Asp Tyr Pro Ile Ser Trp Val Arg Gln Ala Pro Gly Lys Gly Leu Glu Trp Ile Gly Phe Ile Asn Ser
Gly Gly Ser Thr Trp Tyr Ala Ser Trp Val Lys Gly Arg Phe Thr Ile Ser Arg Thr Ser Thr Thr Val Asp Leu Lys
Met Thr Ser Leu Thr Thr Asp Asp Thr Ala Thr Tyr Phe Cys Ala Arg Gly Tyr Ser Thr Tyr Tyr Gly Asp Phe
Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Met Thr Gln Thr Pro Ser Ser Thr Ser Gly Ala Val Gly Gly Thr Val Thr Ile Asn Cys Gln Ala Ser
Gln Ser Ile Asp Ser Asn Leu Ala Trp Phe Gln Gln Lys Pro Gly Gln Pro Pro Thr Leu Leu Ile Tyr Arg Ala Ser
Asn Leu Ala Ser Gly Val Pro Ser Arg Phe Ser Gly Ser Arg Ser Gly Thr Glu Tyr Thr Leu Thr Ile Ser Gly Val
Gln Arg Glu Asp Ala Ala Thr Tyr Tyr Cys Leu Gly Gly Val Gly Asn Val Ser Tyr Arg Thr Ser Phe Gly Gly Gly
Thr Glu Val Val Val Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 122 (scFv ROR1_R12 VH_MiH5_VL_MiH1_CD28tm + 4-1BB/zeta)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 123 (scFv ROR1_huR12 VH_MiH5_VL_MiH1_CD28tm + 4-1BB/zeta)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Ala Asp
Asp Gly Ala Leu Phe Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 124 (scFv ROR1_R12/V16 VH_MiH5_VL_MiH1_CD28tm + 4-1BB/zeta)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 125 (scFv ROR1_R12/V20 VH_MiH5_VL_MiH1_CD28tm + 4-1BB/zeta)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Gly Glu
Asp Leu Gly Leu Phe Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 126 (scFv ROR1_R12/V16-20 VH_MiH5_VL_MiH1_CD28tm + 4-1BB/zeta)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Gly Glu
Asp Leu Gly Leu Phe Asn Ile Trp Gly Pro Gly Thr Leu Val Thr Ile Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 127 (scFv ROR1_huR12/V16 VH_MiH5_VL_MiH1_CD28tm + 4-1BB/zeta)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Ala Asp
Asp Gly Ala Leu Phe Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Glu Ser Arg Gly Tyr Val Phe Gly Gly Gly
Thr Gln Leu Thr Val Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 128 (scFv ROR1_huR12/V20 VH_MiH5_VL_MiH1_CD28tm + 4-1BB/zeta)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Gly Glu
Asp Leu Gly Leu Phe Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Asp Tyr Ile Gly Tyr Val Phe Gly Gly Gly
Thr Gln Leu Thr Val Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 129 (scFv ROR1_huR12/V16-20 VH_MiH5_VL_MiH1_CD28tm + 4-1BB/zeta)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Gly Glu
Asp Leu Gly Leu Phe Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Glu Ser Arg Gly Tyr Val Phe Gly Gly Gly
Thr Gln Leu Thr Val Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 130 (scFv ROR2_4-2 VH_MiH5_VL_MiH1_CD28tm + 4-1BB/zeta)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 131 (scFv ROR2_X3.12 VH_MiH5_VL_MiH1_CD28tm + 4-1BB/zeta)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Asp Arg Trp Ser Leu Asn
Ile Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 132 (scFv ROR2_XBR2-401-DM VH_MiH5_VL_MiH1_CD28tm + 4-1BB/zeta)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Gly
Arg Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 133 (scFv ROR2_huX3.12.5 VH_MiH5_VL_MiH1_CD28tm + 4-1BB/zeta)
Glu Val Gln Leu Val Glu Ser Gly Gly Gly Leu Ile Gln Pro Gly Gly Ser Leu Arg Leu Ser Cys Ala Ala Ser Gly
Phe Thr Val Ser Ser Tyr Gly Val Thr Trp Val Arg Gln Ala Pro Gly Lys Gly Leu Glu Trp Val Ser Tyr Ile Asn
Thr Ala Gly Asn Thr Tyr Tyr Ala Ser Trp Ala Lys Ser Arg Phe Thr Ile Ser Arg Asp Asn Ser Lys Asn Thr Leu
Tyr Leu Gln Met Asn Ser Leu Arg Ala Glu Asp Thr Ala Val Tyr Tyr Cys Ala Arg Asp Asp Arg Trp Ser Leu
Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Ser Pro Ser Ser Leu Ser Ala Ser Val Gly Asp Arg Val Thr Ile Thr Cys Gln Ala Ser
Gln Ser Ile Ser Ser Asp Leu Ser Trp Tyr Gln Gln Lys Pro Gly Lys Ala Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Ser Leu
Gln Pro Glu Asp Phe Ala Thr Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 134 (scFv ROR2_huX3.12.6 VH_MiH5_VL_MiH1_CD28tm + 4-11313/zeta)
Glu Val Gln Leu Val Glu Ser Gly Gly Gly Leu Val Gln Pro Gly Gly Ser Leu Arg Leu Ser Cys Ala Ala Ser Gly
Phe Thr Phe Ser Ser Tyr Gly Val Thr Trp Val Arg Gln Ala Pro Gly Lys Gly Leu Glu Trp Val Ser Tyr Ile Asn
Thr Ala Gly Asn Thr Tyr Tyr Ala Ser Trp Ala Lys Ser Arg Phe Thr Ile Ser Arg Asp Asn Ala Lys Asn Ser Leu
Tyr Leu Gln Met Asn Ser Leu Arg Ala Glu Asp Thr Ala Val Tyr Tyr Cys Ala Arg Asp Asp Arg Trp Ser Leu
Asn Ile Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Pro Met Leu Thr Gln Ser Pro Ser Ser Leu Ser Ala Ser Val Gly Asp Arg Val Thr Ile Thr Cys Gln Ala Ser
Gln Ser Ile Ser Ser Asp Leu Ser Trp Tyr Gln Gln Lys Pro Gly Lys Ala Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Ser Leu
Gln Pro Glu Asp Phe Ala Thr Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 135 (scFv SLAMF7_ERCS409 VH_MiH5_VL_MiH1_CD28tm + 4-11313/zeta)
Gln Ser Val Lys Glu Ser Glu Gly Gly Leu Phe Lys Pro Thr Asp Thr Leu Thr Leu Thr Cys Thr Val Ser Gly Phe
Ser Leu Asn Ser Tyr Gly Val Ile Trp Val Arg Gln Ala Pro Gly Asn Gly Leu Glu Tyr Ile Gly Ile Ile Gly Ser Ser
Gly Asn Thr Tyr Tyr Ala Ser Trp Ala Lys Ser Arg Ser Thr Ile Thr Arg Asn Thr Arg Leu Asn Thr Val Thr Leu
Lys Met Thr Ser Leu Thr Ala Ala Asp Thr Ala Thr Tyr Phe Cys Ala Arg Tyr Tyr Gly Asp Ser Gly Phe Asp
Ser Trp Gly Pro Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Ala Gln Val Leu Thr Gln Thr Pro Ser Ser Thr Ser Val Ala Val Gly Gly Thr Val Thr Ile Lys Cys Gln Ala Ser
Gln Ser Ile Gly Ser Trp Leu Ser Trp Tyr Gln Gln Lys Pro Gly Gln Pro Pro Lys Leu Leu Ile Tyr Gly Ala Ser
Asn Leu Ala Ser Gly Val Pro Ser Arg Phe Lys Gly Gly Arg Ser Gly Thr Glu Tyr Ser Leu Thr Ile Ser Gly Val
Gln Arg Glu Asp Ala Ala Thr Tyr Tyr Cys Leu Gly Ala Ser Pro Asn Gly Trp Ala Phe Gly Ala Gly Thr Asn Val
Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 136 (scFv SLAMF7_huLuc63 VH_MiR5_VL_MiH1_CD28tm + 4-1BB/zeta)
Glu Val Gln Leu Val Glu Ser Gly Gly Gly Leu Val Gln Pro Gly Gly Ser Leu Arg Leu Ser Cys Ala Ala Ser Gly
Phe Asp Phe Ser Arg Tyr Trp Met Ser Trp Val Arg Gln Ala Pro Gly Lys Gly Leu Glu Trp Ile Gly Glu Ile Asn
Pro Asp Ser Ser Thr Ile Asn Tyr Ala Pro Ser Leu Lys Asp Lys Phe Ile Ile Ser Arg Asp Asn Ala Lys Asn Ser
Leu Tyr Leu Gln Met Asn Ser Leu Arg Ala Glu Asp Thr Ala Val Tyr Tyr Cys Ala Arg Pro Asp Gly Asn Tyr
Trp Tyr Phe Asp Val Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Gln Met Thr Gln Ser Pro Ser Ser Leu Ser Ala Ser Val Gly Asp Arg Val Thr Ile Thr Cys Lys Ala Ser
Gln Asp Val Gly Ile Ala Val Ala Trp Tyr Gln Gln Lys Pro Gly Lys Val Pro Lys Leu Leu Ile Tyr Trp Ala Ser
Thr Arg His Thr Gly Val Pro Asp Arg Phe Ser Gly Ser Gly Ser Gly Thr Asp Phe Thr Leu Thr Ile Ser Ser Leu
Gln Pro Glu Asp Val Ala Thr Tyr Tyr Cys Gln Gln Tyr Ser Ser Tyr Pro Tyr Thr Phe Gly Gln Gly Thr Lys Val
Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 137 (scFv FLT3_13V10 VH_MiR5_VL_MiH1_CD28tm + 4-1BB/zeta)
Gln Val Gln Leu Lys Gln Ser Gly Pro Gly Leu Val Gln Pro Ser Gln Ser Leu Ser Ile Thr Cys Thr Val Ser Gly
Phe Ser Leu Thr Asn Tyr Gly Leu His Trp Val Arg Gln Ser Pro Gly Lys Gly Leu Glu Trp Leu Gly Val Ile Trp
Ser Gly Gly Ser Thr Asp Tyr Asn Ala Ala Phe Ile Ser Arg Leu Ser Ile Ser Lys Asp Asn Ser Lys Ser Gln Val
Phe Phe Lys Met Asn Ser Leu Gln Ala Asp Asp Thr Ala Ile Tyr Tyr Cys Ala Arg Lys Gly Gly Ile Tyr Tyr Ala
Asn His Tyr Tyr Ala Met Asp Tyr Trp Gly Gln Gly Thr Ser Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Met Thr Gln Ser Pro Ser Ser Leu Ser Val Ser Ala Gly Glu Lys Val Thr Met Ser Cys Lys Ser Ser
Gln Ser Leu Leu Asn Ser Gly Asn Gln Lys Asn Tyr Met Ala Trp Tyr Gln Gln Lys Pro Gly Gln Pro Pro Lys
Leu Leu Ile Tyr Gly Ala Ser Thr Arg Glu Ser Gly Val Pro Asp Arg Phe Thr Gly Ser Gly Ser Gly Thr Asp Phe
Thr Leu Thr Ile Ser Ser Val Gln Ala Glu Asp Leu Ala Val Tyr Tyr Cys Gln Asn Asp His Ser Tyr Pro Leu Thr
Phe Gly Ala Gly Thr Lys Leu Glu Leu Lys Arg
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 138 (scFv FLT3_4G8 VH_MiR5_VL_MiH1_CD28tm + 4-1BB/zeta)
Gln Val Gln Leu Gln Gln Pro Gly Ala Glu Leu Val Lys Pro Gly Ala Ser Leu Lys Leu Ser Cys Lys Ser Ser Gly
Tyr Thr Phe Thr Ser Tyr Trp Met His Trp Val Arg Gln Arg Pro Gly His Gly Leu Glu Trp Ile Gly Glu Ile Asp
Pro Ser Asp Ser Tyr Lys Asp Tyr Asn Gln Lys Phe Lys Asp Lys Ala Thr Leu Thr Val Asp Arg Ser Ser Asn
Thr Ala Tyr Met His Leu Ser Ser Leu Thr Ser Asp Asp Ser Ala Val Tyr Tyr Cys Ala Arg Ala Ile Thr Thr Thr
Pro Phe Asp Phe Trp Gly Gln Gly Thr Thr Leu Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Leu Thr Gln Ser Pro Ala Thr Leu Ser Val Thr Pro Gly Asp Ser Val Ser Leu Ser Cys Arg Ala Ser
Gln Ser Ile Ser Asn Asn Leu His Trp Tyr Gln Gln Lys Ser His Glu Ser Pro Arg Leu Leu Ile Lys Tyr Ala Ser
Gln Ser Ile Ser Gly Ile Pro Ser Arg Phe Ser Gly Ser Gly Ser Gly Thr Asp Phe Thr Leu Ser Ile Asn Ser Val
Glu Thr Glu Asp Phe Gly Val Tyr Phe Cys Gln Gln Ser Asn Thr Trp Pro Tyr Thr Phe Gly Gly Gly Thr Lys
Leu Glu Ile Lys Arg
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 139 (scFv Siglec-6_JML-1 VH_MiH5_VL_MiH1_CD28tm + 4-1BB/zeta)
Lys Val Gln Leu Leu Glu Ser Gly Gly Gly Leu Val Gln Pro Gly Arg Ser Leu Arg Leu Ser Cys Ala Ala Ser Gly
Phe Thr Phe Asp Asp Tyr Gly Met His Trp Val Arg Gln Ala Pro Gly Lys Gly Leu Glu Trp Val Ser Gly Ile Ser
Trp Asn Ser Gly Ser Ile Gly Tyr Ala Asp Ser Val Lys Gly Arg Phe Thr Ile Ser Arg Asp Asn Ser Lys Asn Thr
Leu Tyr Leu Gln Met Asn Ser Leu Arg Ala Glu Asp Thr Ala Val Tyr Tyr Cys Ala Arg Gly Gly Gln Thr Ile Asp
Ile Trp Gly Gln Gly Thr Met Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Gln Met Thr Gln Ser Pro Ser Ser Leu Ser Ala Ser Val Gly Asp Arg Val Thr Ile Thr Cys Arg Ala Ser
Gln Ser Ile Ser Ser Tyr Leu Asn Trp Tyr Gln Gln Lys Pro Gly Lys Ala Pro Lys Leu Leu Ile Tyr Ala Ala Ser
Ser Leu Gln Ser Gly Val Pro Ser Arg Phe Ser Gly Ser Gly Ser Gly Thr Asp Phe Thr Leu Thr Ile Ser Ser Leu
Gln Pro Glu Asp Phe Ala Thr Tyr Tyr Cys Gln Gln Ser Tyr Ser Thr Pro Phe Thr Phe Gly Pro Gly Thr Lys
Val Asp Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 140 (scFv avb3_LM609v7 VH_MiH5_VL_MiH1_CD28tm + 4-1BB/zeta)
Gln Val Gln Leu Gln Glu Ser Gly Pro Gly Leu Val Lys Pro Ser Gln Thr Leu Ser Leu Thr Cys Thr Val Ser Gly
Ala Ser Ile Ser Arg Gly Gly Tyr Tyr Trp Ser Trp Ile Arg Gln Tyr Pro Gly Lys Gly Leu Glu Trp Ile Gly Tyr Ile
His His Ser Gly Ser Thr Tyr Tyr Asn Pro Ser Leu Lys Ser Arg Val Thr Ile Ala Ile Asp Thr Ser Lys Asn Gln
Leu Ser Leu Arg Leu Thr Ser Val Thr Ala Ala Asp Thr Ala Val Tyr Tyr Cys Ala Arg His Asn Tyr Gly Ser Phe
Ala Tyr Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Met Thr Gln Ser Pro Glu Phe Gln Ser Val Thr Pro Lys Glu Thr Val Thr Ile Thr Cys Arg Ala Ser
Gln Asp Ile Gly Asn Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Ser Arg Leu
Glu Pro Glu Asp Phe Ala Val Tyr Tyr Cys Gln Gln Ser Asn Ser Trp Pro His Thr Phe Gly Gln Gly Thr Lys Leu
Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 141 (scFv avb3_LM609v11 VH_MiH5_VL_MiH1_CD28tm + 4-1BB/zeta)
Gln Val Gln Leu Val Gln Ser Gly Ala Glu Val Arg Lys Pro Gly Ser Ser Val Arg Val Ser Cys Lys Ala Ser Gly
Gly Thr Phe Ser Gly Phe Ala Val Ser Trp Val Arg Gln Ala Pro Gly Gln Arg Phe Glu Trp Leu Gly Gly Ile Val
Ala Ser Leu Gly Ser Thr Asp Tyr Ala Gln Lys Phe Gln Asp Lys Leu Thr Ile Thr Val Asp Glu Ser Thr Ala Thr
Val Tyr Met Glu Met Arg Asn Leu Arg Ser Asp Asp Thr Ala Val Tyr Tyr Cys Ala Arg His Asn Tyr Gly Ser
Phe Ala Tyr Trp Gly Gln Gly Thr Leu Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Glu Leu Val Met Thr Gln Ser Pro Glu Phe Gln Ser Val Thr Pro Lys Glu Thr Val Thr Ile Thr Cys Arg Ala Ser
Gln Asp Ile Gly Thr Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Tyr Ser Leu
Glu Ala Glu Asp Phe Ala Val Tyr Tyr Cys Gln Gln Ser Asn Ser Trp Pro His Thr Phe Gly Gln Gly Thr Lys Leu
Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 142 (scFvBCMA _BCMA30 VH_MiH5_VL_MiH1_CD28tm + 4-11313/zeta)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ser Ser
Thr Ala Tyr Met Glu Leu Ser Ser Leu Arg Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Met Thr Gln Thr Pro Leu Ser Leu Ser Val Thr Pro Gly Glu Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Ala Asp Phe Thr Leu
Lys Ile Ser Arg Val Glu Ala Glu Asp Val Gly Val Tyr Tyr Cys Ala Glu Thr Ser His Val Pro Trp Thr Phe Gly
Gln Gly Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 143 (scFv BCMA_BCMA50 VH_MiH5_VL_MiH1_CD28tm + 4-1BB/zeta)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ile Asn
Thr Ala Tyr Met Glu Leu Ser Ser Leu Thr Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
Glu Leu Lys Thr Pro Leu Gly Asp Thr Thr His Thr Cys Pro Arg Cys Pro Glu Pro Lys Ser Cys Asp Thr Pro
Pro Pro Cys Pro Arg Cys Pro Glu Pro Lys Ser Cys Asp Thr Pro Pro Pro Cys Pro Arg Cys Pro Glu Pro Lys
Ser Cys Asp Thr Pro Pro Pro Cys Pro Arg Cys Pro Glu Pro Lys Ser Cys Asp Thr Pro Pro Pro Cys Pro Arg
Cys Pro Glu Pro Lys Ser Cys Asp Thr Pro Pro Pro Cys Pro Arg Cys Pro
Asp Ile Val Met Thr Gln Thr Pro Leu Ser Leu Ser Val Thr Pro Gly Gln Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Thr Asp Phe Thr Leu
Lys Ile Ser Arg Val Glu Ala Glu Asp Val Gly Ile Tyr Tyr Cys Ser Gln Ser Ser Ile Tyr Pro Trp Thr Phe Gly Gln
Gly Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 144 (scFv CD19_FMC63 VH_Linker_VL_MiH0_CD28tm + 4-1BB/zeta)
Asp Ile Gln Met Thr Gln Thr Thr Ser Ser Leu Ser Ala Ser Leu Gly Asp Arg Val Thr Ile Ser Cys Arg Ala Ser
Gln Asp Ile Ser Lys Tyr Leu Asn Trp Tyr Gln Gln Lys Pro Asp Gly Thr Val Lys Leu Leu Ile Tyr His Thr Ser
Arg Leu His Ser Gly Val Pro Ser Arg Phe Ser Gly Ser Gly Ser Gly Thr Asp Tyr Ser Leu Thr Ile Ser Asn Leu
Glu Gln Glu Asp Ile Ala Thr Tyr Phe Cys Gln Gln Gly Asn Thr Leu Pro Tyr Thr Phe Gly Gly Gly Thr Lys Leu
Glu Ile Thr
Gly Ser Thr Ser Gly Ser Gly Lys Pro Gly Ser Gly Glu Gly Ser Thr Lys Gly
Glu Val Lys Leu Gln Glu Ser Gly Pro Gly Leu Val Ala Pro Ser Gln Ser Leu Ser Val Thr Cys Thr Val Ser Gly
Val Ser Leu Pro Asp Tyr Gly Val Ser Trp Ile Arg Gln Pro Pro Arg Lys Gly Leu Glu Trp Leu Gly Val Ile Trp
Gly Ser Glu Thr Thr Tyr Tyr Asn Ser Ala Leu Lys Ser Arg Leu Thr Ile Ile Lys Asp Asn Ser Lys Ser Gln Val
Phe Leu Lys Met Asn Ser Leu Gln Thr Asp Asp Thr Ala Ile Tyr Tyr Cys Ala Lys His Tyr Tyr Tyr Gly Gly Ser
Tyr Ala Met Asp Tyr Trp Gly Gln Gly Thr Ser Val Thr Val Ser Ser
Glu Leu Lys Thr Pro Leu Gly Asp Thr Thr His Thr Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 145 (scFv CD19_FMC63 VH_Linker_VL_MiH1_CD28tm + 4-1BB/zeta)
Asp Ile Gln Met Thr Gln Thr Thr Ser Ser Leu Ser Ala Ser Leu Gly Asp Arg Val Thr Ile Ser Cys Arg Ala Ser
Gln Asp Ile Ser Lys Tyr Leu Asn Trp Tyr Gln Gln Lys Pro Asp Gly Thr Val Lys Leu Leu Ile Tyr His Thr Ser
Arg Leu His Ser Gly Val Pro Ser Arg Phe Ser Gly Ser Gly Ser Gly Thr Asp Tyr Ser Leu Thr Ile Ser Asn Leu
Glu Gln Glu Asp Ile Ala Thr Tyr Phe Cys Gln Gln Gly Asn Thr Leu Pro Tyr Thr Phe Gly Gly Gly Thr Lys Leu
Glu Ile Thr
Gly Ser Thr Ser Gly Ser Gly Lys Pro Gly Ser Gly Glu Gly Ser Thr Lys Gly
Glu Val Lys Leu Gln Glu Ser Gly Pro Gly Leu Val Ala Pro Ser Gln Ser Leu Ser Val Thr Cys Thr Val Ser Gly
Val Ser Leu Pro Asp Tyr Gly Val Ser Trp Ile Arg Gln Pro Pro Arg Lys Gly Leu Glu Trp Leu Gly Val Ile Trp
Gly Ser Glu Thr Thr Tyr Tyr Asn Ser Ala Leu Lys Ser Arg Leu Thr Ile Ile Lys Asp Asn Ser Lys Ser Gln Val
Phe Leu Lys Met Asn Ser Leu Gln Thr Asp Asp Thr Ala Ile Tyr Tyr Cys Ala Lys His Tyr Tyr Tyr Gly Gly Ser
Tyr Ala Met Asp Tyr Trp Gly Gln Gly Thr Ser Val Thr Val Ser Ser
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 146 (scFv CD20_Leu16 VL_Linker_VH_MiH3_CD28tm + 4-1BB/zeta)
Asp Ile Val Leu Thr Gln Ser Pro Ala Ile Leu Ser Ala Ser Pro Gly Glu Lys Val Thr Met Thr Cys Arg Ala Ser
Ser Ser Val Asn Tyr Met Asp Trp Tyr Gln Lys Lys Pro Gly Ser Ser Pro Lys Pro Trp Ile Tyr Ala Thr Ser Asn
Leu Ala Ser Gly Val Pro Ala Arg Phe Ser Gly Ser Gly Ser Gly Thr Ser Tyr Ser Leu Thr Ile Ser Arg Val Glu
Ala Glu Asp Ala Ala Thr Tyr Tyr Cys Gln Gln Trp Ser Phe Asn Pro Pro Thr Phe Gly Gly Gly Thr Lys Leu
Glu Ile Lys
Gly Ser Thr Ser Gly Gly Gly Ser Gly Gly Gly Ser Gly Gly Gly Gly Ser Ser
Glu Val Gln Leu Gln Gln Ser Gly Ala Glu Leu Val Lys Pro Gly Ala Ser Val Lys Met Ser Cys Lys Ala Ser Gly
Tyr Thr Phe Thr Ser Tyr Asn Met His Trp Val Lys Gln Thr Pro Gly Gln Gly Leu Glu Trp Ile Gly Ala Ile Tyr
Pro Gly Asn Gly Asp Thr Ser Tyr Asn Gln Lys Phe Lys Gly Lys Ala Thr Leu Thr Ala Asp Lys Ser Ser Ser Thr
Ala Tyr Met Gln Leu Ser Ser Leu Thr Ser Glu Asp Ser Ala Asp Tyr Tyr Cys Ala Arg Ser Asn Tyr Tyr Gly Ser
Ser Tyr Trp Phe Phe Asp Val Trp Gly Ala Gly Thr Thr Val Thr Val Ser Ser
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 147 (scFv ROR1_2A2 VH_4GS3_VL_MiH1_CD28tm + 4-113B/zeta)
Gln Val Gln Leu Gln Gln Ser Gly Ala Glu Leu Val Arg Pro Gly Ala Ser Val Thr Leu Ser Cys Lys Ala Ser Gly
Tyr Thr Phe Ser Asp Tyr Glu Met His Trp Val Ile Gln Thr Pro Val His Gly Leu Glu Trp Ile Gly Ala Ile Asp
Pro Glu Thr Gly Gly Thr Ala Tyr Asn Gln Lys Phe Lys Gly Lys Ala Ile Leu Thr Ala Asp Lys Ser Ser Ser Thr
Ala Tyr Met Glu Leu Arg Ser Leu Thr Ser Glu Asp Ser Ala Val Tyr Tyr Cys Thr Gly Tyr Tyr Asp Tyr Asp Ser
Phe Thr Tyr Trp Gly Gln Gly Thr Leu Val Thr Val Ser Ala
Gly Gly Gly Gly Ser Gly Gly Gly Gly Ser Gly Gly Gly Gly Ser
Asp Ile Val Met Thr Gln Ser Gln Lys Ile Met Ser Thr Thr Val Gly Asp Arg Val Ser Ile Thr Cys Lys Ala Ser
Gln Asn Val Asp Ala Ala Val Ala Trp Tyr Gln Gln Lys Pro Gly Gln Ser Pro Lys Leu Leu Ile Tyr Ser Ala Ser
Asn Arg Tyr Thr Gly Val Pro Asp Arg Phe Thr Gly Ser Gly Ser Gly Thr Asp Phe Thr Leu Thr Ile Ser Asn
Met Gln Ser Glu Asp Leu Ala Asp Tyr Phe Cys Gln Gln Tyr Asp Ile Tyr Pro Tyr Thr Phe Gly Gly Gly Thr
Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 148 (scFv ROR1_4-2 VH_4GS3_VL_MiH1_CD28tm + 4-1BB/zeta)
Gln Glu Gln Gln Lys Glu Ser Gly Gly Gly Leu Phe Lys Pro Thr Asp Thr Leu Thr Leu Thr Cys Thr Ala Ser Gly
Phe Asp Ile Ser Ser Tyr Tyr Met Ser Trp Val Arg Gln Ala Pro Gly Asn Gly Leu Glu Trp Ile Gly Ala Ile Gly
Ile Ser Gly Asn Ala Tyr Tyr Ala Ser Trp Ala Lys Ser Arg Ser Thr Ile Thr Arg Asn Thr Asn Leu Asn Thr Val
Thr Leu Lys Met Thr Ser Leu Thr Ala Ala Asp Thr Ala Thr Tyr Phe Cys Ala Arg Asp His Pro Thr Tyr Gly
Met Asp Leu Trp Gly Pro Gly Thr Leu Val Thr Val Ser Ser
Gly Gly Gly Gly Ser Gly Gly Gly Gly Ser Gly Gly Gly Gly Ser
Ser Tyr Glu Leu Thr Gln Leu Pro Ser Val Ser Val Ser Leu Gly Gln Thr Ala Arg Ile Thr Cys Glu Gly Asn Asn
Ile Gly Ser Lys Ala Val His Trp Tyr Gln Gln Lys Pro Gly Leu Ala Pro Gly Leu Leu Ile Tyr Asp Asp Asp Glu
Arg Pro Ser Gly Val Pro Asp Arg Phe Ser Gly Ser Asn Ser Gly Asp Thr Ala Thr Leu Thr Ile Ser Gly Ala Gln
Ala Gly Asp Glu Ala Asp Tyr Tyr Cys Gln Val Trp Asp Ser Ser Ala Tyr Val Phe Gly Gly Gly Thr Gln Leu Thr
Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 149 (scFv ROR1_R11 VH_4GS3_VL_MiH3_CD28tm + 4-1BB/zeta)
Gln Ser Val Lys Glu Ser Glu Gly Asp Leu Val Thr Pro Ala Gly Asn Leu Thr Leu Thr Cys Thr Ala Ser Gly Ser
Asp Ile Asn Asp Tyr Pro Ile Ser Trp Val Arg Gln Ala Pro Gly Lys Gly Leu Glu Trp Ile Gly Phe Ile Asn Ser
Gly Gly Ser Thr Trp Tyr Ala Ser Trp Val Lys Gly Arg Phe Thr Ile Ser Arg Thr Ser Thr Thr Val Asp Leu Lys
Met Thr Ser Leu Thr Thr Asp Asp Thr Ala Thr Tyr Phe Cys Ala Arg Gly Tyr Ser Thr Tyr Tyr Gly Asp Phe
Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Met Thr Gln Thr Pro Ser Ser Thr Ser Gly Ala Val Gly Gly Thr Val Thr Ile Asn Cys Gln Ala Ser
Gln Ser Ile Asp Ser Asn Leu Ala Trp Phe Gln Gln Lys Pro Gly Gln Pro Pro Thr Leu Leu Ile Tyr Arg Ala Ser
Asn Leu Ala Ser Gly Val Pro Ser Arg Phe Ser Gly Ser Arg Ser Gly Thr Glu Tyr Thr Leu Thr Ile Ser Gly Val
Gln Arg Glu Asp Ala Ala Thr Tyr Tyr Cys Leu Gly Gly Val Gly Asn Val Ser Tyr Arg Thr Ser Phe Gly Gly Gly
Thr Glu Val Val Val Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 150 (scFv ROR1_R12 VH_Linker_VL_MiH1_CD28tm + 4-1BB/zeta)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 151 (scFv ROR1_huR12 VH_Linker_VL_MiH1_CD28tm + 4-1BB/zeta)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Ala Asp
Asp Gly Ala Leu Phe Asn Ile Trp Gly Gln Gly Thr Leu Val Thr Val Ser Ser
Gly Gly Gly Gly Ser Gly Gly Gly Gly Ser Gly Gly Gly Gly Ser
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 152 (scFv ROR1_R12/V16 VH_Linker_VL_MiH1_CD28tm + 4-1BB/zeta)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Ala Asp
Asp Gly Ala Leu Phe Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 153 (scFv ROR1_R12/V20 VH_Linker_VL_MiH1_CD28tm + 4-1BB/zeta)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Gly Glu
Asp Leu Gly Leu Phe Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Asp Tyr Ile Gly Gly Tyr Val Phe Gly Gly
Gly Thr Gln Leu Thr Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 154 (scFv ROR1_R12/V16-20 VH_Linker_VL_MiH1_CD28tm + 4-1BB/zeta)
Gln Glu Gln Leu Val Glu Ser Gly Gly Arg Leu Val Thr Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Thr Trp Val Asn Gly Arg Phe Thr Ile Ser Ser Asp Asn Ala Gln Asn Thr
Val Asp Leu Gln Met Asn Ser Leu Thr Ala Ala Asp Arg Ala Thr Tyr Phe Cys Ala Arg Asp Ser Tyr Gly Glu
Asp Leu Gly Leu Phe Asn Ile Trp Gly Pro Gly Thr Leu Val Thr Ile Ser Ser
Gly Gly Gly Gly Ser Gly Gly Gly Gly Ser Gly Gly Gly Gly Ser
Glu Leu Val Leu Thr Gln Ser Pro Ser Val Ser Ala Ala Leu Gly Ser Pro Ala Lys Ile Thr Cys Thr Leu Ser Ser
Ala His Lys Thr Asp Thr Ile Asp Trp Tyr Gln Gln Leu Gln Gly Glu Ala Pro Arg Tyr Leu Met Gln Val Gln Ser
Asp Gly Ser Tyr Thr Lys Arg Pro Gly Val Pro Asp Arg Phe Ser Gly Ser Ser Ser Gly Ala Asp Arg Tyr Leu Ile
Ile Pro Ser Val Gln Ala Asp Asp Glu Ala Asp Tyr Tyr Cys Gly Ala Glu Ser Arg Gly Gly Tyr Val Phe Gly Gly
Gly Thr Gln Leu Thr Val Thr Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 155 (scFv ROR1_huR12/V16 VH_Linker_VL_MiH1_CD28tm + 4-1BB/zeta)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Ala Asp
Asp Gly Ala Leu Phe Asn Ile Trp Gly Gln Gly Thr Leu Val Thr Val Ser Ser
Gly Gly Gly Gly Ser Gly Gly Gly Gly Ser Gly Gly Gly Gly Ser
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Glu Ser Arg Gly Tyr Val Phe Gly Gly Gly
Thr Gln Leu Thr Val Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 156 (scFv ROR1_huR12/V20 VH_Linker_VL_MiH1_CD28tm + 4-1BB/zeta)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Gly Glu
Asp Leu Gly Leu Phe Asn Ile Trp Gly Gln Gly Thr Leu Val Thr Val Ser Ser
Gly Gly Gly Gly Ser Gly Gly Gly Gly Ser Gly Gly Gly Gly Ser
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Asp Tyr Ile Gly Tyr Val Phe Gly Gly Gly
Thr Gln Leu Thr Val Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 157 (scFv ROR1_huR12/V16-20 VH_Linker_VL_MiH1_CD28tm + 4-1BB/zeta)
Gln Val Gln Leu Val Glu Ser Gly Gly Ala Leu Val Gln Pro Gly Gly Ser Leu Thr Leu Ser Cys Lys Ala Ser Gly
Phe Asp Phe Ser Ala Tyr Tyr Met Ser Trp Val Arg Gln Ala Pro Gly Lys Gly Leu Glu Trp Ile Ala Thr Ile Tyr
Pro Ser Ser Gly Lys Thr Tyr Tyr Ala Ala Ser Val Gln Gly Arg Phe Thr Ile Ser Ala Asp Asn Ala Lys Asn Thr
Val Tyr Leu Gln Met Asn Ser Leu Thr Ala Ala Asp Thr Ala Thr Tyr Phe Cys Ala Arg Asp Ser Tyr Gly Glu
Asp Leu Gly Leu Phe Asn Ile Trp Gly Gln Gly Thr Leu Val Thr Val Ser Ser
Gly Gly Gly Gly Ser Gly Gly Gly Gly Ser Gly Gly Gly Gly Ser
Gln Leu Val Leu Thr Gln Ser Pro Ser Val Ser Ala Ala Leu Gly Ser Ser Ala Lys Ile Thr Cys Thr Leu Ser Ser
Ala His Lys Thr Asp Thr Ile Asp Trp Tyr Gln Gln Leu Ala Gly Gln Ala Pro Arg Tyr Leu Met Tyr Val Gln Ser
Asp Gly Ser Tyr Glu Lys Arg Ser Gly Val Pro Asp Arg Phe Ser Gly Ser Ser Ser Gly Ala Asp Arg Tyr Leu Ile
Ile Ser Ser Val Gln Ala Asp Asp Glu Ala Asp Tyr Tyr Cys Gly Ala Glu Ser Arg Gly Tyr Val Phe Gly Gly Gly
Thr Gln Leu Thr Val Gly
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 158 (scFv R0R2_4-2 VH_4GS3_VL_MiH1_CD28tm + 4-1BB/zeta)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
Gly Gly Gly Gly Ser Gly Gly Gly Gly Ser Gly Gly Gly Gly Ser
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 159 (scFv ROR2_X3.12 VH_4G53_VL_MiH1_CD28tm + 4-1BB/zeta)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Thr
Ala Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Asp Arg Trp Ser Leu Asn
Ile Trp Gly Pro Gly Thr Leu Val Thr Val Ser Ser
Gly Gly Gly Gly Ser Gly Gly Gly Gly Ser Gly Gly Gly Gly Ser
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 160 (scFv ROR2_XBR2-401-DM VH_4GS3_VL_MiH1_CD28tm + 4-1BB/zeta)
Gln Ser Val Lys Glu Ser Glu Gly Gly Leu Phe Lys Pro Thr Asp Thr Leu Thr Leu Thr Cys Thr Val Ser Gly Phe
Ser Leu Ser Ser Tyr Gly Val Thr Trp Val Arg Gln Ala Pro Gly Ser Gly Leu Glu Trp Ile Gly Tyr Ile Asn Gly
Arg Gly Asn Thr Tyr Tyr Ala Ser Trp Ala Lys Ser Arg Ser Thr Ile Thr Arg Asn Thr Asn Glu Asn Thr Val Thr
Leu Lys Met Thr Ser Leu Thr Ala Ala Asp Thr Ala Thr Tyr Phe Cys Ala Arg Asp Trp Thr Ser Leu Asn Ile
Trp Gly Pro Gly Thr Leu Val Thr Val Ser Ser
Gly Gly Gly Gly Ser Gly Gly Gly Gly Ser Gly Gly Gly Gly Ser
Asp Pro Met Leu Thr Gln Thr Pro Ser Ser Thr Ser Thr Ala Val Gly Asp Thr Val Thr Ile Lys Cys Gln Ala Ser
Gln Ser Ile Ser Ser Asp Leu Ser Trp Tyr Gln Gln Lys Pro Gly Gln Arg Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Gly Val
Gln Arg Glu Asp Ala Ala Ile Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 161 (scFv ROR2_huX3.12.5 VH_4GS3_VL_MiH1_CD28tm + 4-1BB/zeta)
Glu Val Gln Leu Val Glu Ser Gly Gly Gly Leu Ile Gln Pro Gly Gly Ser Leu Arg Leu Ser Cys Ala Ala Ser Gly
Phe Thr Val Ser Ser Tyr Gly Val Thr Trp Val Arg Gln Ala Pro Gly Lys Gly Leu Glu Trp Val Ser Tyr Ile Asn
Thr Ala Gly Asn Thr Tyr Tyr Ala Ser Trp Ala Lys Ser Arg Phe Thr Ile Ser Arg Asp Asn Ser Lys Asn Thr Leu
Tyr Leu Gln Met Asn Ser Leu Arg Ala Glu Asp Thr Ala Val Tyr Tyr Cys Ala Arg Asp Asp Arg Trp Ser Leu
Asn Ile Trp Gly Gln Gly Thr Leu Val Thr Val Ser Ser
Gly Gly Gly Gly Ser Gly Gly Gly Gly Ser Gly Gly Gly Gly Ser
Asp Pro Met Leu Thr Gln Ser Pro Ser Ser Leu Ser Ala Ser Val Gly Asp Arg Val Thr Ile Thr Cys Gln Ala Ser
Gln Ser Ile Ser Ser Asp Leu Ser Trp Tyr Gln Gln Lys Pro Gly Lys Ala Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Ser Leu
Gln Pro Glu Asp Phe Ala Thr Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 162 (scFv ROR2_huX3.12.6 VH_4GS3_VL_MiH1_CD28tm + 4-1BB/zeta)
Glu Val Gln Leu Val Glu Ser Gly Gly Gly Leu Val Gln Pro Gly Gly Ser Leu Arg Leu Ser Cys Ala Ala Ser Gly
Phe Thr Phe Ser Ser Tyr Gly Val Thr Trp Val Arg Gln Ala Pro Gly Lys Gly Leu Glu Trp Val Ser Tyr Ile Asn
Thr Ala Gly Asn Thr Tyr Tyr Ala Ser Trp Ala Lys Ser Arg Phe Thr Ile Ser Arg Asp Asn Ala Lys Asn Ser Leu
Tyr Leu Gln Met Asn Ser Leu Arg Ala Glu Asp Thr Ala Val Tyr Tyr Cys Ala Arg Asp Asp Arg Trp Ser Leu
Asn Ile Trp Gly Gln Gly Thr Leu Val Thr Val Ser Ser
Gly Gly Gly Gly Ser Gly Gly Gly Gly Ser Gly Gly Gly Gly Ser
Asp Pro Met Leu Thr Gln Ser Pro Ser Ser Leu Ser Ala Ser Val Gly Asp Arg Val Thr Ile Thr Cys Gln Ala Ser
Gln Ser Ile Ser Ser Asp Leu Ser Trp Tyr Gln Gln Lys Pro Gly Lys Ala Pro Lys Leu Leu Ile Tyr Gln Ala Ser
Thr Leu Ala Ser Gly Val Pro Ser Arg Phe Lys Gly Ser Gly Tyr Gly Thr Glu Tyr Thr Leu Thr Ile Ser Ser Leu
Gln Pro Glu Asp Phe Ala Thr Tyr Tyr Cys Leu Gly Gly Tyr Ala Asp Ala Ser Tyr Arg Thr Ala Phe Gly Gly Gly
Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 163 (scFv SLAMF7_ERCS409 VH_4GS3_VL_MiH1_CD28tm + 4-1BB/zeta)
Gln Ser Val Lys Glu Ser Glu Gly Gly Leu Phe Lys Pro Thr Asp Thr Leu Thr Leu Thr Cys Thr Val Ser Gly Phe
Ser Leu Asn Ser Tyr Gly Val Ile Trp Val Arg Gln Ala Pro Gly Asn Gly Leu Glu Tyr Ile Gly Ile Ile Gly Ser Ser
Gly Asn Thr Tyr Tyr Ala Ser Trp Ala Lys Ser Arg Ser Thr Ile Thr Arg Asn Thr Arg Leu Asn Thr Val Thr Leu
Lys Met Thr Ser Leu Thr Ala Ala Asp Thr Ala Thr Tyr Phe Cys Ala Arg Tyr Tyr Gly Asp Ser Gly Phe Asp
Ser Trp Gly Pro Gly Thr Leu Val Thr Val Ser Ser
Gly Gly Gly Gly Ser Gly Gly Gly Gly Ser Gly Gly Gly Gly Ser
Ala Gln Val Leu Thr Gln Thr Pro Ser Ser Thr Ser Val Ala Val Gly Gly Thr Val Thr Ile Lys Cys Gln Ala Ser
Gln Ser Ile Gly Ser Trp Leu Ser Trp Tyr Gln Gln Lys Pro Gly Gln Pro Pro Lys Leu Leu Ile Tyr Gly Ala Ser
Asn Leu Ala Ser Gly Val Pro Ser Arg Phe Lys Gly Gly Arg Ser Gly Thr Glu Tyr Ser Leu Thr Ile Ser Gly Val
Gln Arg Glu Asp Ala Ala Thr Tyr Tyr Cys Leu Gly Ala Ser Pro Asn Gly Trp Ala Phe Gly Ala Gly Thr Asn Val
Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 164 (scFv SLAMF7_huLuc63 VH_4GS3_VL_MiH1_CD28tm + 4-1BB/zeta)
Glu Val Gln Leu Val Glu Ser Gly Gly Gly Leu Val Gln Pro Gly Gly Ser Leu Arg Leu Ser Cys Ala Ala Ser Gly
Phe Asp Phe Ser Arg Tyr Trp Met Ser Trp Val Arg Gln Ala Pro Gly Lys Gly Leu Glu Trp Ile Gly Glu Ile Asn
Pro Asp Ser Ser Thr Ile Asn Tyr Ala Pro Ser Leu Lys Asp Lys Phe Ile Ile Ser Arg Asp Asn Ala Lys Asn Ser
Leu Tyr Leu Gln Met Asn Ser Leu Arg Ala Glu Asp Thr Ala Val Tyr Tyr Cys Ala Arg Pro Asp Gly Asn Tyr
Trp Tyr Phe Asp Val Trp Gly Gln Gly Thr Leu Val Thr Val Ser Ser
Gly Gly Gly Gly Ser Gly Gly Gly Gly Ser Gly Gly Gly Gly Ser
Asp Ile Gln Met Thr Gln Ser Pro Ser Ser Leu Ser Ala Ser Val Gly Asp Arg Val Thr Ile Thr Cys Lys Ala Ser
Gln Asp Val Gly Ile Ala Val Ala Trp Tyr Gln Gln Lys Pro Gly Lys Val Pro Lys Leu Leu Ile Tyr Trp Ala Ser
Thr Arg His Thr Gly Val Pro Asp Arg Phe Ser Gly Ser Gly Ser Gly Thr Asp Phe Thr Leu Thr Ile Ser Ser Leu
Gln Pro Glu Asp Val Ala Thr Tyr Tyr Cys Gln Gln Tyr Ser Ser Tyr Pro Tyr Thr Phe Gly Gln Gly Thr Lys Val
Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 165 (scFv FLT3_BV10 VH_4G53_VL_MiH1_CD28tm + 4-1BB/zeta)
Gln Val Gln Leu Lys Gln Ser Gly Pro Gly Leu Val Gln Pro Ser Gln Ser Leu Ser Ile Thr Cys Thr Val Ser Gly
Phe Ser Leu Thr Asn Tyr Gly Leu His Trp Val Arg Gln Ser Pro Gly Lys Gly Leu Glu Trp Leu Gly Val Ile Trp
Ser Gly Gly Ser Thr Asp Tyr Asn Ala Ala Phe Ile Ser Arg Leu Ser Ile Ser Lys Asp Asn Ser Lys Ser Gln Val
Phe Phe Lys Met Asn Ser Leu Gln Ala Asp Asp Thr Ala Ile Tyr Tyr Cys Ala Arg Lys Gly Gly Ile Tyr Tyr Ala
Asn His Tyr Tyr Ala Met Asp Tyr Trp Gly Gln Gly Thr Ser Val Thr Val Ser Ser
Gly Gly Gly Gly Ser Gly Gly Gly Gly Ser Gly Gly Gly Gly Ser
Asp Ile Val Met Thr Gln Ser Pro Ser Ser Leu Ser Val Ser Ala Gly Glu Lys Val Thr Met Ser Cys Lys Ser Ser
Gln Ser Leu Leu Asn Ser Gly Asn Gln Lys Asn Tyr Met Ala Trp Tyr Gln Gln Lys Pro Gly Gln Pro Pro Lys
Leu Leu Ile Tyr Gly Ala Ser Thr Arg Glu Ser Gly Val Pro Asp Arg Phe Thr Gly Ser Gly Ser Gly Thr Asp Phe
Thr Leu Thr Ile Ser Ser Val Gln Ala Glu Asp Leu Ala Val Tyr Tyr Cys Gln Asn Asp His Ser Tyr Pro Leu Thr
Phe Gly Ala Gly Thr Lys Leu Glu Leu Lys Arg
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 166 (scFv FLT3_4G8 VH_4GS3_VL_MiH1_CD28tm + 4-1BB/zeta)
Gln Val Gln Leu Gln Gln Pro Gly Ala Glu Leu Val Lys Pro Gly Ala Ser Leu Lys Leu Ser Cys Lys Ser Ser Gly
Tyr Thr Phe Thr Ser Tyr Trp Met His Trp Val Arg Gln Arg Pro Gly His Gly Leu Glu Trp Ile Gly Glu Ile Asp
Pro Ser Asp Ser Tyr Lys Asp Tyr Asn Gln Lys Phe Lys Asp Lys Ala Thr Leu Thr Val Asp Arg Ser Ser Asn
Thr Ala Tyr Met His Leu Ser Ser Leu Thr Ser Asp Asp Ser Ala Val Tyr Tyr Cys Ala Arg Ala Ile Thr Thr Thr
Pro Phe Asp Phe Trp Gly Gln Gly Thr Thr Leu Thr Val Ser Ser
Gly Gly Gly Gly Ser Gly Gly Gly Gly Ser Gly Gly Gly Gly Ser
Asp Ile Val Leu Thr Gln Ser Pro Ala Thr Leu Ser Val Thr Pro Gly Asp Ser Val Ser Leu Ser Cys Arg Ala Ser
Gln Ser Ile Ser Asn Asn Leu His Trp Tyr Gln Gln Lys Ser His Glu Ser Pro Arg Leu Leu Ile Lys Tyr Ala Ser
Gln Ser Ile Ser Gly Ile Pro Ser Arg Phe Ser Gly Ser Gly Ser Gly Thr Asp Phe Thr Leu Ser Ile Asn Ser Val
Glu Thr Glu Asp Phe Gly Val Tyr Phe Cys Gln Gln Ser Asn Thr Trp Pro Tyr Thr Phe Gly Gly Gly Thr Lys
Leu Glu Ile Lys Arg
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 167 (scFv Siglec-6_JML-1 VH_4GS3_VL_MiH1_CD28tm + 4-1BB/zeta)
Lys Val Gln Leu Leu Glu Ser Gly Gly Gly Leu Val Gln Pro Gly Arg Ser Leu Arg Leu Ser Cys Ala Ala Ser Gly
Phe Thr Phe Asp Asp Tyr Gly Met His Trp Val Arg Gln Ala Pro Gly Lys Gly Leu Glu Trp Val Ser Gly Ile Ser
Trp Asn Ser Gly Ser Ile Gly Tyr Ala Asp Ser Val Lys Gly Arg Phe Thr Ile Ser Arg Asp Asn Ser Lys Asn Thr
Leu Tyr Leu Gln Met Asn Ser Leu Arg Ala Glu Asp Thr Ala Val Tyr Tyr Cys Ala Arg Gly Gly Gln Thr Ile Asp
Ile Trp Gly Gln Gly Thr Met Val Thr Val Ser Ser
Gly Gly Gly Gly Ser Gly Gly Gly Gly Ser Gly Gly Gly Gly Ser
Asp Ile Gln Met Thr Gln Ser Pro Ser Ser Leu Ser Ala Ser Val Gly Asp Arg Val Thr Ile Thr Cys Arg Ala Ser
Gln Ser Ile Ser Ser Tyr Leu Asn Trp Tyr Gln Gln Lys Pro Gly Lys Ala Pro Lys Leu Leu Ile Tyr Ala Ala Ser
Ser Leu Gln Ser Gly Val Pro Ser Arg Phe Ser Gly Ser Gly Ser Gly Thr Asp Phe Thr Leu Thr Ile Ser Ser Leu
Gln Pro Glu Asp Phe Ala Thr Tyr Tyr Cys Gln Gln Ser Tyr Ser Thr Pro Phe Thr Phe Gly Pro Gly Thr Lys
Val Asp Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 168 (scFv avb3_LM609v7 VH_4GS3_VL_MiH1_CD28tm + 4-1BB/zeta)
Gln Val Gln Leu Gln Glu Ser Gly Pro Gly Leu Val Lys Pro Ser Gln Thr Leu Ser Leu Thr Cys Thr Val Ser Gly
Ala Ser Ile Ser Arg Gly Gly Tyr Tyr Trp Ser Trp Ile Arg Gln Tyr Pro Gly Lys Gly Leu Glu Trp Ile Gly Tyr Ile
His His Ser Gly Ser Thr Tyr Tyr Asn Pro Ser Leu Lys Ser Arg Val Thr Ile Ala Ile Asp Thr Ser Lys Asn Gln
Leu Ser Leu Arg Leu Thr Ser Val Thr Ala Ala Asp Thr Ala Val Tyr Tyr Cys Ala Arg His Asn Tyr Gly Ser Phe
Ala Tyr Trp Gly Gln Gly Thr Leu Val Thr Val Ser Ser
Gly Gly Gly Gly Ser Gly Gly Gly Gly Ser Gly Gly Gly Gly Ser
Glu Leu Val Met Thr Gln Ser Pro Glu Phe Gln Ser Val Thr Pro Lys Glu Thr Val Thr Ile Thr Cys Arg Ala Ser
Gln Asp Ile Gly Asn Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Ser Arg Leu
Glu Pro Glu Asp Phe Ala Val Tyr Tyr Cys Gln Gln Ser Asn Ser Trp Pro His Thr Phe Gly Gln Gly Thr Lys Leu
Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 169 (scFv avb3_LM609v11 VH_4GS3_VL_MiH1_CD28tm + 4-1BB/zeta)
Gln Val Gln Leu Val Gln Ser Gly Ala Glu Val Arg Lys Pro Gly Ser Ser Val Arg Val Ser Cys Lys Ala Ser Gly
Gly Thr Phe Ser Gly Phe Ala Val Ser Trp Val Arg Gln Ala Pro Gly Gln Arg Phe Glu Trp Leu Gly Gly Ile Val
Ala Ser Leu Gly Ser Thr Asp Tyr Ala Gln Lys Phe Gln Asp Lys Leu Thr Ile Thr Val Asp Glu Ser Thr Ala Thr
Val Tyr Met Glu Met Arg Asn Leu Arg Ser Asp Asp Thr Ala Val Tyr Tyr Cys Ala Arg His Asn Tyr Gly Ser
Phe Ala Tyr Trp Gly Gln Gly Thr Leu Val Thr Val Ser Ser
Gly Gly Gly Gly Ser Gly Gly Gly Gly Ser Gly Gly Gly Gly Ser
Glu Leu Val Met Thr Gln Ser Pro Glu Phe Gln Ser Val Thr Pro Lys Glu Thr Val Thr Ile Thr Cys Arg Ala Ser
Gln Asp Ile Gly Thr Ser Leu His Trp Tyr Gln Gln Lys Pro Gly Gln Ser Pro Lys Leu Leu Ile Lys Tyr Ala Ser
Gln Pro Val Phe Gly Val Pro Ser Arg Phe Arg Gly Ser Gly Ser Gly Thr Asp Phe Thr Leu Thr Ile Tyr Ser Leu
Glu Ala Glu Asp Phe Ala Val Tyr Tyr Cys Gln Gln Ser Asn Ser Trp Pro His Thr Phe Gly Gln Gly Thr Lys Leu
Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 170 (scFv BCMA_BCMA30 VH_4GS3_VL_MiH1_CD28tm + 4-1BB/zeta)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ser Ser
Thr Ala Tyr Met Glu Leu Ser Ser Leu Arg Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
Gly Gly Gly Gly Ser Gly Gly Gly Gly Ser Gly Gly Gly Gly Ser
Asp Ile Val Met Thr Gln Thr Pro Leu Ser Leu Ser Val Thr Pro Gly Glu Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Ala Asp Phe Thr Leu
Lys Ile Ser Arg Val Glu Ala Glu Asp Val Gly Val Tyr Tyr Cys Ala Glu Thr Ser His Val Pro Trp Thr Phe Gly
Gln Gly Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 171 (scFv BCMA_BCMA50 VH_4GS3_VL_MiH1_CD28tm + 4-1BB/zeta)
Gln Val Gln Leu Val Gln Ser Gly Ala Glu Val Lys Lys Pro Gly Ala Ser Val Lys Val Ser Cys Lys Ala Ser Gly
Tyr Ser Phe Pro Asp Tyr Tyr Ile Asn Trp Val Arg Gln Ala Pro Gly Gln Gly Leu Glu Trp Met Gly Trp Ile Tyr
Phe Ala Ser Gly Asn Ser Glu Tyr Asn Gln Lys Phe Thr Gly Arg Val Thr Met Thr Arg Asp Thr Ser Ile Asn
Thr Ala Tyr Met Glu Leu Ser Ser Leu Thr Ser Glu Asp Thr Ala Val Tyr Phe Cys Ala Ser Leu Tyr Asp Tyr
Asp Trp Tyr Phe Asp Val Trp Gly Gln Gly Thr Met Val Thr Val Ser Ser
Gly Gly Gly Gly Ser Gly Gly Gly Gly Ser Gly Gly Gly Gly Ser
Asp Ile Val Met Thr Gln Thr Pro Leu Ser Leu Ser Val Thr Pro Gly Gln Pro Ala Ser Ile Ser Cys Lys Ser Ser
Gln Ser Leu Val His Ser Asn Gly Asn Thr Tyr Leu His Trp Tyr Leu Gln Lys Pro Gly Gln Ser Pro Gln Leu Leu
Ile Tyr Lys Val Ser Asn Arg Phe Ser Gly Val Pro Asp Arg Phe Ser Gly Ser Gly Ser Gly Thr Asp Phe Thr Leu
Lys Ile Ser Arg Val Glu Ala Glu Asp Val Gly Ile Tyr Tyr Cys Ser Gln Ser Ser Ile Tyr Pro Trp Thr Phe Gly Gln
Gly Thr Lys Leu Glu Ile Lys
Glu Leu Lys Thr Pro Leu Gly Asp Thr Thr His Thr Cys Pro Arg Cys Pro
Glu Pro Lys Ser Cys Asp Thr Pro Pro Pro Cys Pro Arg Cys Pro
Met Phe Trp Val Leu Val Val Val Gly Gly Val Leu Ala Cys Tyr Ser Leu Leu Val Thr Val Ala Phe Ile Ile Phe
Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro Gln Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr Tyr
Asp Ala Leu His Met Gln Ala Leu Pro Pro Arg
SEQ ID NO: 172 (CH2 domain of human IgG3)
SVFLFPPKPKDTLMISRTPEVTCVVVDVSHEDPEVQFKWYVDGVEVHNAKTKPREEQYNSTFRVVSVLTVLHQDWL
NGKEYKCKVSNKALPAPIEKTIS
SEQ ID NO: 173 (CH3 domain of human IgG3)
PQVYTLPPSREEMTKNQVSLTCLVKGFYPSDIAVEWESSGQPENNYNTTPPMLDSDGSFFLYSKLTVDKSRWQQG
NIFSCSVMHEALHNRFTQKSLSLS

### EXAMPLES

Additional aspects and details of the invention are exemplified by the following non-limiting examples. In particular, the Examples were carried out as follows:

Human Subjects

T cells for CAR-modification were isolated from the peripheral blood of healthy donors. All participants provided written informed consent to participate in research protocols approved by the institutional review board of the University of Würzburg.

Cell Lines and cell Culture Media

Jeko-1, K562, MDA-MB231, Raji, MM.1S, T-47D and U266 (all ATCC, Manassas, Va., USA) and OPM-2 (DSMZ, Braunschweig, Germany) cells were maintained in RPMI-1640 medium containing 8% fetal calf serum (FCS), 2 mM L-glutamine, and 100 U/mL penicillin/streptomycin (all components from Gibco, Thermo Scientific, Schwerte, Germany). K562_CD19, K562_CD20, K562_SLAMF7 and K562_ROR1 cells were generated by lentiviral transduction with full-length human CD19, CD20, SLAMF7 or ROR1, respectively. K562_IgG3_MiH5 were generated by lentiviral transduction with the CD19 CAR construct CD19_IgG3_MiH5 (described in ‘Generation of T cell section’). K562_ROR1/E3AK cells were generated by lentiviral transduction with a truncated form of human ROR1 protein (UniProtKB-Q01973, aa 312-440) carrying an inflexible linker (AEAAAKA)¹⁶ introduced between aa 391 and 392. MDA-MB231_hROR2 cells were generated by lentiviral transduction with full-length human ROR2. All tumor cell lines were transduced with a lentiviral vector encoding a firefly luciferase (ffluc)/green fluorescent protein (GFP) transgene to enable detection by flow cytometry (GFP) and bioluminescence imaging (ffLuc) in mice, and to use it for bioluminescence-based cytotoxicity assays. T cells were maintained in RPMI-1640 medium containing 8% human serum, 2 mM Glutamax, 0,1% β-mercaptoethanol and 100 U/mL penicillin/streptomycin (T cell medium; all components from Gibco), or, where stated, in X-VIVO™ 15 serum-free medium (Lonza, Basel, Switzerland, containing 2 mM Glutamax, 0,1% β-mercaptoethanol and 100 U/mL penicillin/streptomycin (Serum-free medium). T cell cultures were supplemented with 50 U/ml IL-2 (Proleukin, Novartis, Basel, Switzerland).

Generation of CAR T Cells

The vector design and experimental procedure have been described in a previous study¹⁷. In brief, peripheral blood mononuclear cells (PBMCs) of healthy donors were purified using Ficoll-hypaque density centrifugation in 50 mL LeukoSep tubes (Greiner Bio One), and CD4⁺ and CD8⁺ T cells were isolated using negative magnetic sorting (CD4⁺ and CD8⁺ T cell Isolation Kits, human, Miltenyi). T cells were stimulated with anti-CD3/CD28 magnetic beads (Dynabeads® Human T-Activator CD3/CD28, ThermoScientific) and genetically modified either by lentiviral transduction (epHIV7 lentivirus) or by non-viral Sleeping Beauty gene transfer. The CAR constructs used comprise the following: an antigen-specific single chain variable fragment derived from monoclonal antibodies; an IgG4 or IgG3 hinge-derived spacer; a CD28 transmembrane region; a 4-1BB_CD3ζ signaling module; and a truncated epidermal growth factor receptor (EGFR) transduction marker¹⁸. T cells were enriched for EGFRt⁺ using the anti-EGFR monoclonal antibody (mAb) Cetuximab (Merck, Darmstadt, Germany), that had been biotinylated in-house (EZ-Link™Sulfo-NHS-SS-Biotin, ThermoFisher Scientific, IL) according to the manufacturer's instructions) and anti-Biotin Microbeads (Miltenyi). Purified CAR T and non-transduced control T cells were expanded using a rapid expansion protocol^{7, 19} or—for CD19,CD20 and SLAMF7-CAR T cells—using antigen-specific stimulation with irradiated (80Gy) CD19⁺/CD20/SLAMF7⁺ feeder cells^{7, 19}.

In a preferred embodiment of the invention, the chimeric antigen receptor is a CD19 CAR having the amino acid sequence of SEQ ID NO: 68. In a more preferred embodiment, the CD19 CAR having the amino acid sequence of SEQ ID NO: 68 can be expressed using the lentiviral vector having the nucleotide sequence of SEQ ID NO: 70, or using the Sleeping Beauty vector having the nucleotide sequence of SEQ ID NO: 69.

SEQ ID NO: 61 and 62 (CAR lentiviral backbone, 5′ and 3′ sequences before and after CAR insert, respectively)

SEQ ID NO: 63 and 64 (CAR Sleeping Beauty backbone, 5′ and 3′ sequences before and after CAR insert, respectively)

scFvs Used for CAR Generation

Codon optimized targeting domains comprising VH and VL segments of the following antibodies were synthesized (GeneArt ThermoFisher, Regensburg, Germany) and used as targeting domain for CAR constructs: CD19: FMC63²⁰; CD20: Leu16²¹; SLAMF7: huLuc63²²; ROR1: R11²³ and 4-2²⁴; ROR2: 4-1²⁴; IgG3 Hinge: anti-MiH antibody #1 (this invention).

CD19 (FMC63) scFv: heavy chain variable domain having the amino acid sequence of SEQ ID NO: 27, light chain chain variable domain having the amino acid sequence of SEQ ID NO: 28. CD20 (Leu16) scFv: heavy chain variable domain having the amino acid sequence of SEQ ID NO: 30, light chain variable domain having the amino acid sequence of SEQ ID NO: 29. SLAMF7 (huLuc63) scFv: heavy chain variable domain having the amino acid sequence of SEQ ID NO: 43, light chain variable domain having the amino acid sequence of SEQ ID NO: 44. ROR1 (R11) scFv: heavy chain variable domain having the amino acid sequence of SEQ ID NO: 35, light chain variable domain having the amino acid sequence of SEQ ID NO: 36. ROR1 (4-2) scFv: heavy chain variable domain having the amino acid sequence of SEQ ID NO: 33, light chain variable domain having the amino acid sequence of SEQ ID NO: 34. ROR2 (4-1) scFv: heavy chain variable domain having the amino acid sequence of SEQ ID NO: 39, light chain variable domain having the amino acid sequence of SEQ ID NO: 40. anti-MiH #1 scFv: heavy chain variable domain having the amino acid sequence of SEQ ID NO: 19, light chain variable domain having the amino acid sequence of SEQ ID NO: 23.

Antibodies and Flow Cytometry

CAR-transduced (i.e. EGFRt⁺) T cells were detected by staining with the anti-EGFR monoclonal antibody Cetuximab (Merck, Darmstadt, Germany), or the anti-Her2 monoclonal antibody Trastuzumab (Roche, Penzberg, Germany) that have been conjugated to AF647 using the Alexa Fluor™ 647 Protein Labeling Kit (ThermoFisher).

Antibodies against CD19 (clone HIB19; AF647), CD20 (clone 2H7; PE, AF647, APC), SLAMF7/CD319 (clone 162.1; PE) from BioLegend (London, United Kingdom); CD4 (clone M-T466; VioBlue & PE-Vio770), CD8 (clone BW135/80; VioBlue & PE-Vio770), ROR1 (clone 2A2; PE & APC) from Miltenyi, ROR2 (polyclonal goat; BioTeche, Minneapolis, Minn., USA) as well as 7-AAD (BD Biosciences, Heidelberg, Germany) to exclude dead cells from analysis were used. The anti-MiH antibody #1 (characterized by a heavy chain variable domain having the amino acid sequence of SEQ ID NO: 19, and a light chain variable domain having the amino acid sequence of by SEQ ID NO: 23, and having a mouse IgG1 backbone) was synthesized by evitria (Zürich-Schlieren, Switzerland). Flow cytometric analyses were performed with a FACS Canto II (BD) machine and analyzed using FlowJo software (TreeStar, Ashland, Oreg.).

### Analysis of CAR T Cell Function In Vitro

Functional analyses were performed as previously described^{5, 7, 25-27}. In brief, target cells expressing firefly luciferase (ffLuc) were incubated in triplicate at 5×10³ cells/well with effector T cells at various effector to target (E:T) ratios. Luciferin substrate was added to the co-culture and the decrease in luminescence signal in wells that contained target cells and T cells was measured using a luminometer (Tecan, Männedorf, Switzerland) and compared to target cells alone. Specific lysis was calculated using the standard formula. For analysis of cytokine secretion, 5×10⁴ T cells were plated in triplicate wells with target cells at a ratio of 4:1 and IFNγ and IL-2 production were measured by ELISA (Biolegend) in supernatant removed after 24-hour incubation. For analysis of proliferation, 5×10⁴ T cells were labeled with 0.2 μM carboxyfluorescein succinimidyl ester (CFSE, ThermoFisher), washed and plated in triplicate wells with target cells at a ratio of 4:1 in medium without exogenous cytokines. After 72-hour incubation, cells were stained with anti-CD8/CD4 mAb and 7-AAD to exclude dead cells from analysis. Samples were analyzed by flow cytometry and division of live T cells assessed by CFSE dilution.

Analysis of CAR T Cell Function In Vivo

All experiments were approved by the competent Institutional Animal Care and Use Committees. NOD.Cg-Prkdcscid Il2rgtm1Wjl/SzJ (NSG) mice (female, 6-8 week old) were purchased from Charles River (Sulzfeld, Germany) or bred in-house. Mice were inoculated with 1×10⁶ ffluc_GFP⁺ tumor cells by tail vein injection on day 0 and randomly allocated to treatment and control groups. On day 7, mice received a single dose of 5×10⁶ T cells (i.e., 2.5×10⁶ CD4⁺ and 2.5×10⁶ CD8⁺ in 200 μL of PBS/0.5% FCS) by tail vein injection. Tumor progression/regression was assessed by serial bioluminescence imaging following i.p. administration of D-luciferin substrate (0.3 mg/g body weight) (Biosynth, Staad, Switzerland) using an IVIS Lumina imaging system (PerkinElmer, Waltham, Mass., USA). The data were analyzed using LivingImage software (PerkinElmer).

Targeting the Multi-Function Site In Vitro

Comparison of sorting efficiency was conducted by mixing 1×10⁶ CAR T cells and 1×10⁶ untransduced control T cells and labelling with anti-MiH antibody #1 or anti-EGFR antibody (Cetuximab, Merck, Darmstadt, Germany), that have been biotinylated in-house (EZ-Link™Sulfo-NHS-SS-Biotin, ThermoFisher Scientific, IL) according to the manufacturer's instructions, and anti-Biotin Microbeads (Miltenyi), followed by purification via the MACS system using LS columns (Miltenyi). Negative and positive fractions were stained with antibodies against CD4, CD8 and EGFRt; 123Count eBeads (ThermoFisher), were added directly before the measurement. In the following flow cytometric analysis, per sample, 1000 123count eBeads were taken up to allow a quantitative comparison of the yield.

For antigen-independent, but CAR-specific activation and expansion using plate-bound antibody, 5×10⁴ T cells were plated in triplicate wells on 96 well plates precoated with 5 μg/mlanti-MiH antibody #1 and cultured in Serum-free medium either for 24 h followed by flow cytometric analysis of CD25 and CD69 expression, or for 7 days for expansion assays, followed by counting of the cells.

For analysis of proliferation in response to anti-MiH antibody #1-coupled Beads or K562 carrying the Anti-CAR, 5×10⁴ T cells were labeled with 0.2 μM carboxyfluorescein succinimidyl ester (CFSE, ThermoFisher), washed and plated in triplicate wells with DynaBeads® (coupled with anti-CD3/anti-CD28, anti-MiH antibody #1, anti-MiH antibody #1+anti-CD28, anti-MiH antibody #1+anti-4-1BB) at a Bead:T cell ratio of 1.6:1 or target cells at a ratio of 4:1 in Serum-free medium without exogenous cytokines. After 72-hour incubation, cells were labeled with anti-CD8/CD4 mAb and 7-AAD to exclude dead cells from analysis. Samples were analyzed by flow cytometry and division of live T cells assessed by CFSE dilution.

For assessing the potential of depleting cells using a anti-MiH antibody #1-derived antibody drug-conjugate (ADC), 5×10⁴ T cells were plated in triplicate wells and treated with different concentrations of anti-MiH antibody #1 that was conjugated to an anthracycline-based cytotoxic payload (NBE Therapeutics, Basel, Switzerland). Cells were cultivated in Serum-free medium in the presence of 50 IU IL-2 for 72 h, washed and stained with antibodies against CD4, CD8 and EGFRt as well as 7AAD; 123Count eBeads (ThermoFisher), were added directly before the measurement. In the following flow cytometric analysis, per sample, 1000 123count eBeads were taken up to allow a quantitative comparison of cytotoxic effects.

Targeting the Multi-Function Sites In Vivo

For in vivo tracking, CD4⁺ T cells were transduced with the advanced version of the IgG3-based CD19 CAR (CD19_IgG3_MiH5/MiH1) as well as with a ffluc_GFP fusion protein, enriched and expanded as above.

NSG mice (female, 6-8 week old, purchased from Charles River (Sulzfeld, Germany) were inoculated with 4.5×10⁶ ffluc_GFP⁺ CART cells by tail vein injection on day 0. At day 8, half of the mice were treated with 100 μg of anti-MiH antibody #1 ADC (approximately 4.5 mg/kg bodyweight). At d11, T cells were restimulated with irradiated K562 cells equipped with an anti-MiH antibody #1-based Anti-CAR (1×10⁶ irradiated K562 cells per mice). Kinetics of T cell persistence was assessed by serial bioluminescence imaging following i.p. administration of D—luciferin substrate (0.3 mg/g body weight) (Biosynth, Staad, Switzerland) using an IVIS Lumina imaging system (PerkinElmer, Waltham, Mass., USA). The data were analyzed using LivingImage software (PerkinElmer).

For analysis of in vivo proliferation, CD4⁺ T cells were transduced with the advanced version of the IgG3-based CD19 CAR (CD19_IgG3_MiH5/MiH1), enriched and expanded as above and labeled with 5 μM of the proliferation dye eFluor 670 (ThermoFisher) according to the manufacturer's instruction.

NSG mice (female, 6-8 week old, purchased from Charles River, Sulzfeld, Germany) were inoculated with 4.5×10⁶ CAR T cells by tail vein injection on day 0. Groups of n=5 mice received irradiated stimulatory cells (either K562 or K562_Anti-CAR) subsequently at different time points per tail vein injection as indicated. At d4 after T cell transfer, mice were sacrificed, bone marrow cells were isolated, stained with antibodies against CD4, CD45 and EGFRt and subjected to flow cytometric analysis as above. CD45⁺/CD4⁺/EGFR⁺ bone-marrow derived T cells were analyzed for eFluor 670 dilution.

### Example 1

### Construction of an IgG3 Hinge Library

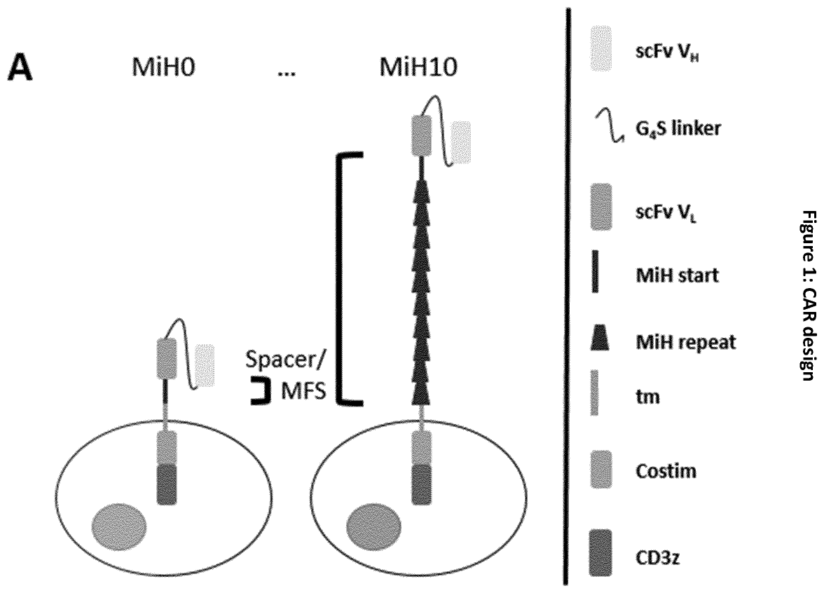

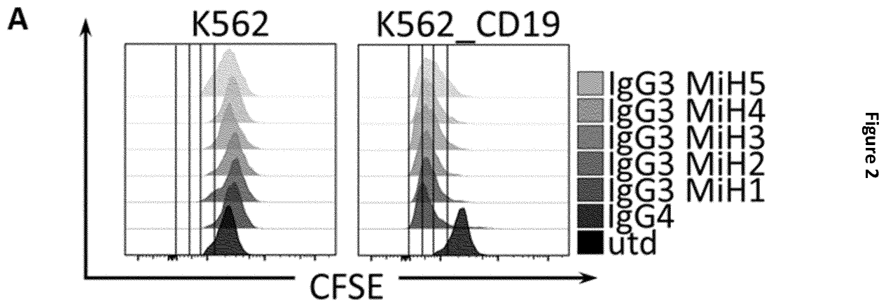

The inventors generated an IgG3 Hinge-based CAR spacer library, in which scFv and transmembrane domain are connected by variants of the human IgG3 Hinge domain. This naturally consists of upper hinge (12 aa, ELKTPLGDTTHT, SEQ ID NO: 2), middle hinge (50 aa, CPRCP, SEQ ID NO: 59+3 repeats of EPKSCDTPPPCPRCP, SEQ ID NO: 1) and lower hinge (8 aa, APELLGGP, SEQ ID NO: 60), leading to a total spacer size of 70 aa for this wild-type spacer termed IgG3_UMLH (upper, middle and lower hinge). From that the inventors constructed variants consisting of upper hinge, the start of the middle hinge (CPRCP, SEQ ID NO: 59) and 0-10 copies of the EPKSCDTPPPCPRCP motif (SEQ ID NO: 1) leading to spacer domains spanning 17 to 167 aa in 15 aa steps named IgG3_MiHO to IgG3_MiH10 (FIG. 1).

### Example 2

### In Vitro Function of CD19-Specific CAR T Cells Carrying IgG3-Derived Spacers

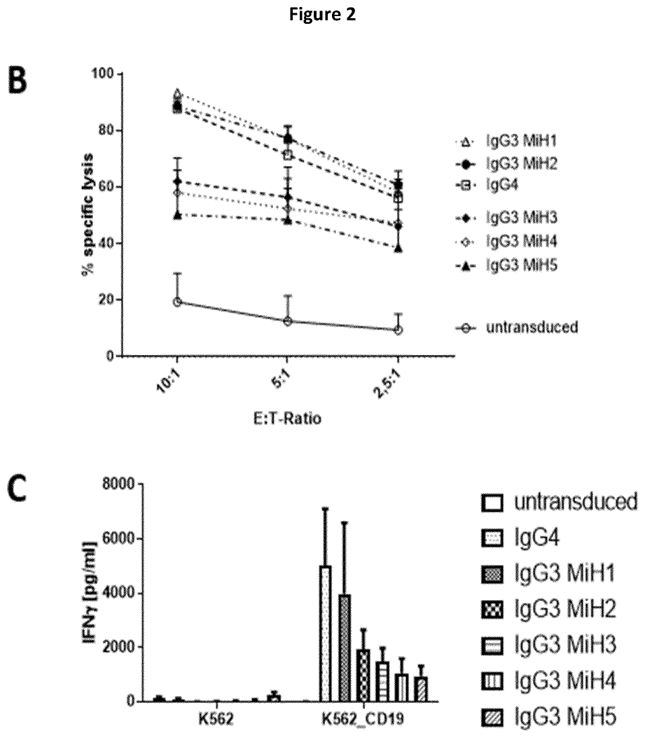

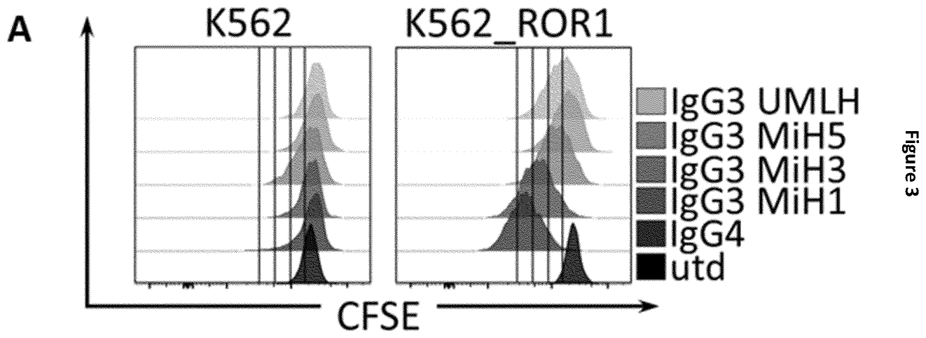

A first set of experiments was conducted using the well-characterized CD19 scFv FMC63⁷. Five IgG3 Hinge variants (IgG3_MiH1, IgG3_MiH2, IgG3_MiH3, IgG3_MiH4 and IgG3_MiH5) were compared to the optimized IgG4-based construct pJ02459 containing a short spacer from IgG4 (12 aa) in CD8⁺ bulk T cells. All other parts of the CARs were constructed in the same way (same scFV, CD28 transmembrane domain, 4-1BB and CD3 signaling domains). In functional in vitro assays, all variants showed a comparably strong specific proliferation upon encounter of CD19-expressing target cells. In contrast to that, variants IgG3_MiH1 and IgG3_MiH2 displayed a pronounced cytotoxic effect similar to that of the IgG4 CAR, while cytolysis was reduced for longer IgG3 variants. A similar outcome was observed for cytokine production: IgG3_MiH1 and the IgG4 variant led to highest secretion of IFNγ, all longer IgG3 variants secreted less (FIG. 2).

### Example 3

### In Vitro Function of ROR1-Specific CAR T Cells Carrying IgG3-Derived Spacers

A second set of experiments compared IgG3 variants of the ROR1 CARs R11 and 4-2 to their best-working IgG4 version (long IgG4 spacer for R11, short IgG4 spacer for 4-2).

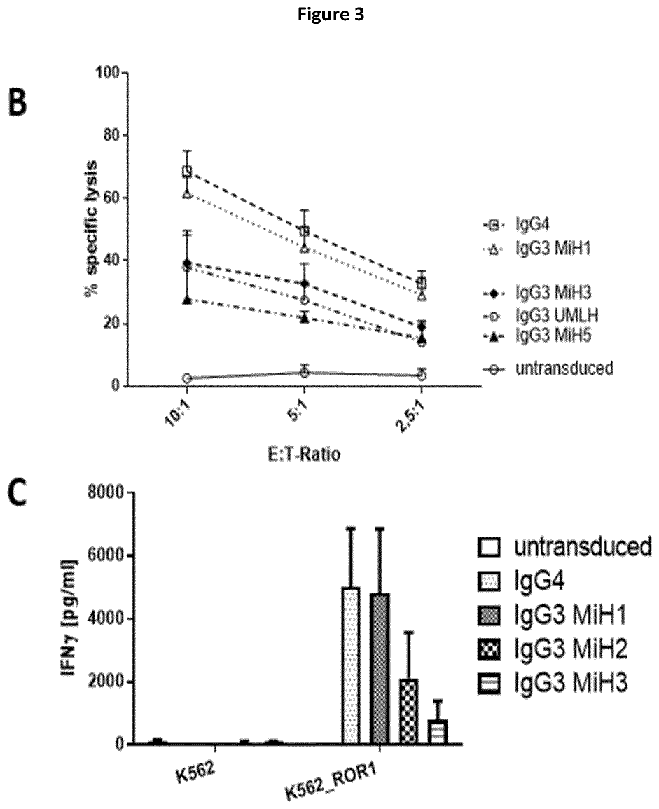

In case of the 4-2 scFv which is targeting a membrane-distal epitope of ROR1²⁴, the IgG3_MiH1 variant and IgG4 showed comparable proliferation, cytotoxicity and cytokine secretion upon antigen encounter, while IgG3_MiH3, IgG3_MiH5 and IgG3_UMLH exhibited reduced antitumor responses (FIG. 3A-C).

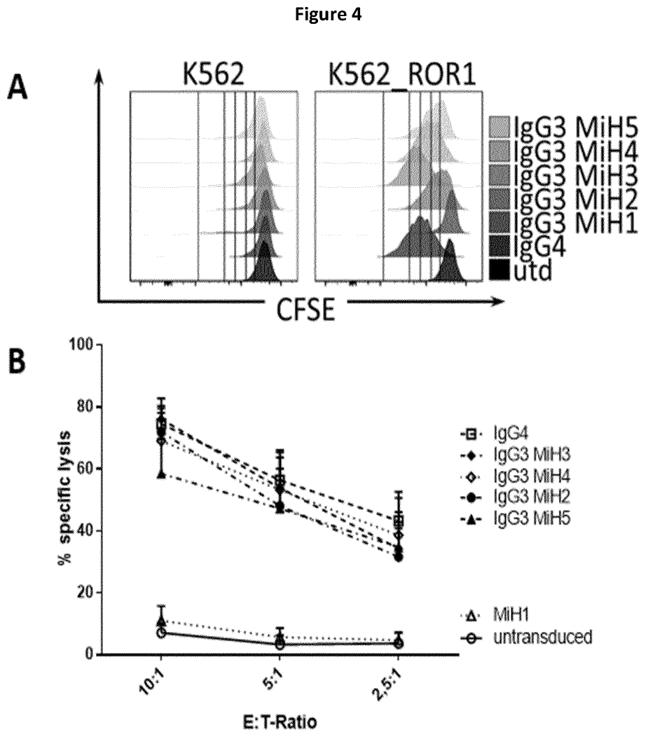

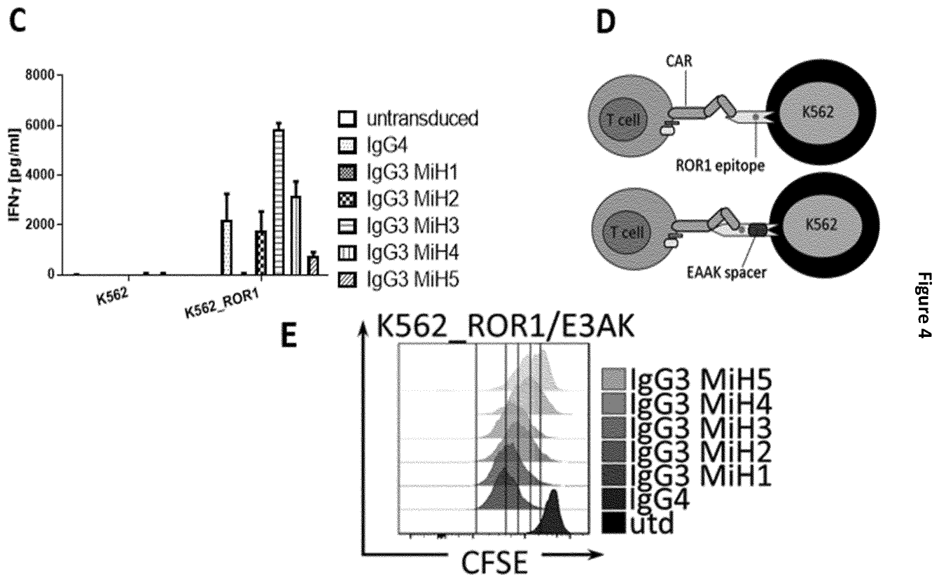

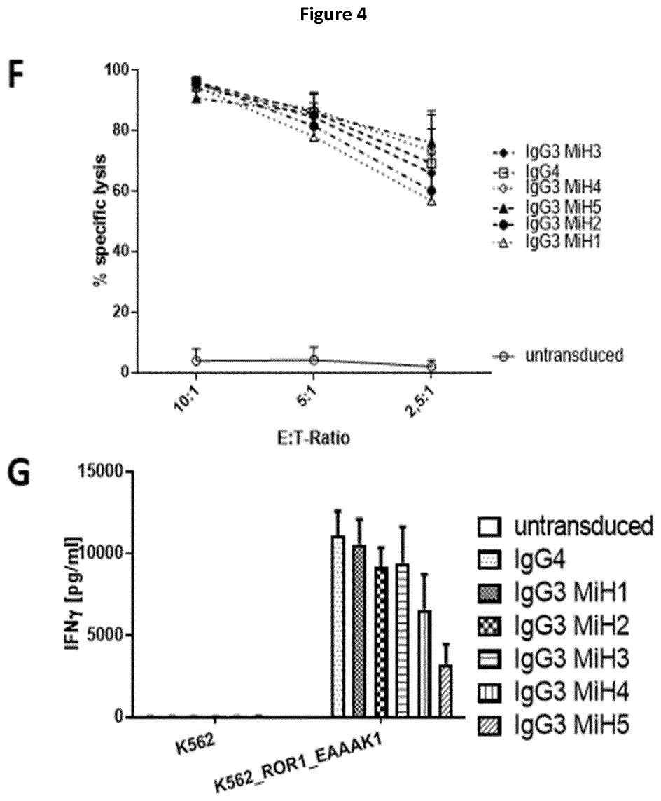

In contrast, the IgG3_MiH1 variant of the R11 scFv, which is targeting a membrane-proximal epitope of ROR1⁷, does not induce antigen-specific proliferation upon encounter of ROR1⁺ target cells. While IgG3_MiH2, IgG3_MiH4 and IgG3_MiH5 display specific proliferation, the optimum seems to be induced by IgG3_MiH3, in a similar manner as the IgG4 variant, suggesting that the sweet spot for IgG3 spacer length of this scFV is located at three repeats (FIG. 4A). Consequently, IgG3_MiH1 does not display any cytotoxic response, while all other variants lead to effective tumor cell lysis with IgG3_MiH2, IgG3_MiH3 and IgG3_MiH4 being as effective as the IgG4 variant (FIG. 4B). In regard of cytokine production, IgG3_MiH3 significantly surpasses the IgG4 variant in secreting IFNγ; IgG3_MiH2 and IgG3_MiH4 secreted similar amounts as the IgG4 variant, while IgG3_MiH5 was less effective and IgG3_MiH1 did not secrete any IFNγ (FIG. 4C).

Interestingly, the inability of IgG3_MiH1 to induce antigen-dependent T cell effector functions is not caused by steric inability to bind the epitope in the target molecule but is caused by a spacer length insufficient to reach the epitope: when the kringle domain, bearing the targeting epitope of R11⁷, is moved further away from the tumor cell membrane by introducing a small, inflexible A(EAAAK)A linker²⁸ between transmembrane and kringle domain (FIG. 4D), IgG3_MiH1 exhibits similar proliferation, cytotoxicity and cytokine secretion as all other variants (FIG. 4E-G).

These results prove in general that the hinge domain of IgG3 is an effective option for the use as flexible spacer in CAR T cells offering a greater variability to optimize the interaction of scFV and target molecule.

### Example 4

### In Vitro Function of CD20-Specific CAR T Cells Carrying IgG3-Derived Spacers

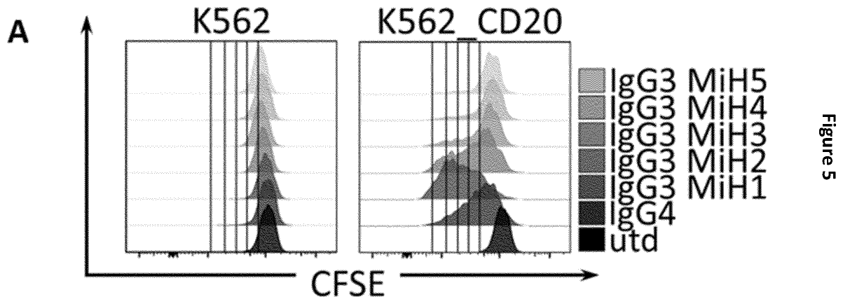

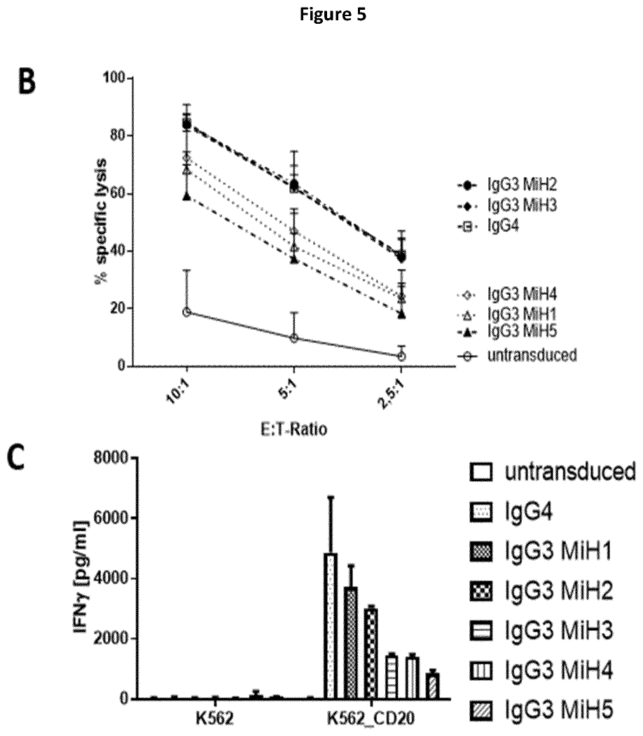

To extend the proof of function to other targets, the inventors investigated IgG3 variants (IgG3_MiH1-IgG3_MiH5) of CARs equipped with the CD20-specific scFv Leu16. As this was reported to target a membrane-proximal epitope of CD20²⁹, consequently, a longer IgG4-based spacer (Hinge-CH2-CH3) proved to be the optimal IgG4 format. Surprisingly, this does not translate to IgG3-based spacers one-to-one. Interestingly, the shortest IgG3 variant (IgG3_MiH1) showed the best proliferation upon antigen encounter, thereby surpassing the IgG4 variant by a wide margin, while longer IgG3 variants proliferated much less (FIG. 5A). In contrast, variants IgG3_MiH2 and IgG3_MiH3 led (together with the IgG4 variant) to best cytotoxic effects, while IgG3_MiH1, IgG3_MiH4 and IgG3_MiH5 exhibited far less cytotoxicity (FIG. 5B). IgG3_MiH1 and IgG3_MiH2 showed comparable amounts of IFNγ to be released while the longer IgG3 variants secreted less (FIG. 5C).

This example illustrates the great flexibility of the IgG3 spacer, as even the shortest version (32aa) seems to be able to bind to relatively membrane-proximal epitopes, whereas a short IgG4 spacer (12 aa) was found to be inferior to a longer one (228 aa)²⁹.

### Example 5

### In Vitro Function of SLAMF7-Specific CAR T Cells Carrying IgG3-Derived Spacers

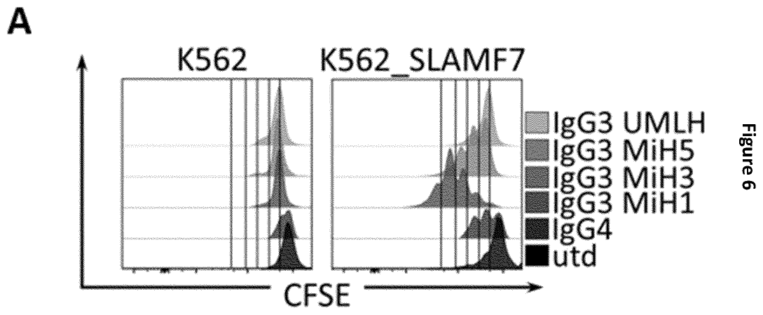

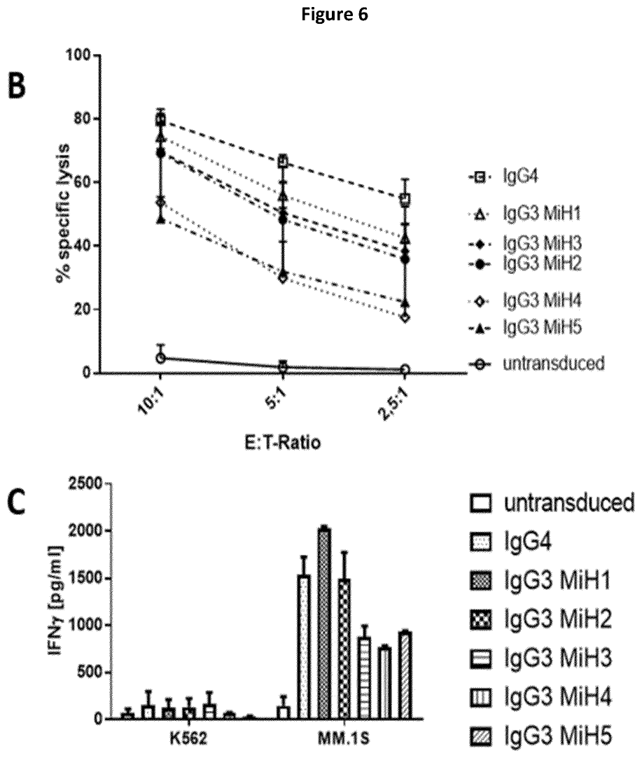

Next, the investigators examined IgG3 variants (IgG3_MiH1-IgG3_MiH5, IgG3_UMLH) of CARs based on the SLAMF7-specific scFv huLuc63. As the inventors previously reported, huLuc63 IgG4 CARs work best when engineered to have a long IgG4 spacer (Hinge-CH2-CH3)³⁰. Surprisingly, the shortest spacer variant investigated (IgG3_MiH1) showed the highest level of antigen-specific proliferation, outperforming the IgG4 variant equipped with a long IgG4-based spacer (Hinge-CH2-CH3). All CAR variants led to profound antigen-specific cytotoxicity and cytokine secretion. Even though none of the IgG3 variants could reach the level of IgG4 for killing of the SLAMF7 expressing myeloma cell line MM.1S, IgG3 variants equipped with 1, 2 or 3 IgG3_MiH repeats led to profound cytolysis. In regard of IFNy secretion, the IgG3_MiH1 IgG3 variant led to the highest secretion with IgG3_MiH2 equaling the IgG4 variant right behind (FIG. 6).

### Example 6

### In Vitro Function of ROR2-Specific CAR T Cells Carrying IgG3-Derived Spacers

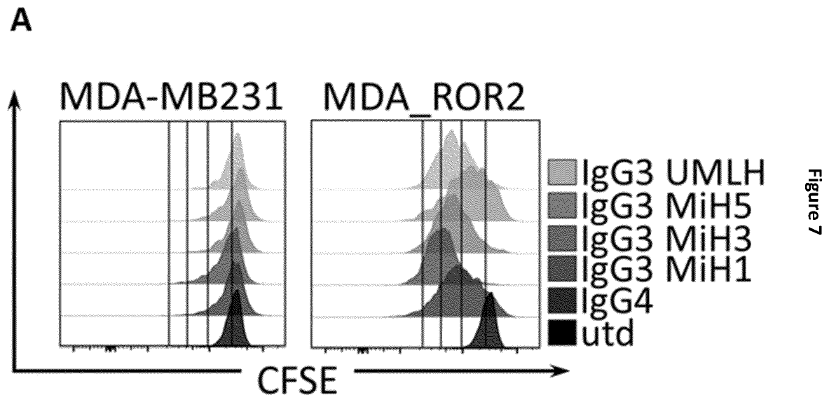

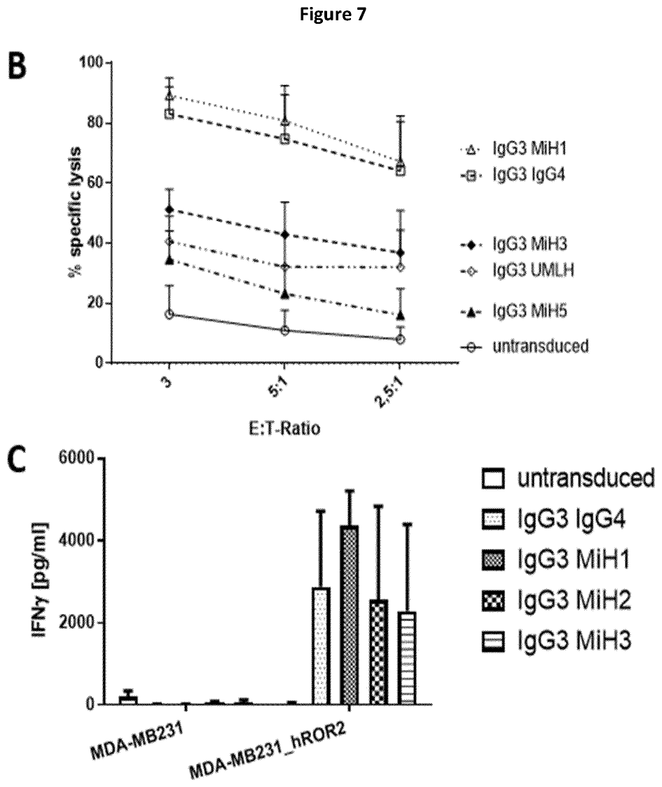

In another example, the inventors constructed IgG3-based spacer variants (IgG3_MiH1, IgG3_MiH3, IgG3_MiH5, IgG3_UMLH) of CARs carrying the ROR2-trageting scFv 4-1, which the inventors previously reported to work better when quipped with a longer IgG4 spacer (Hinge-CH2-CH3) as compared to the shorter one (Hinge only)²⁴. IgG3_MiH1 outperforms the IgG4 variant (IgG4 long) in specific proliferation and cytokine secretion (IFNγ) upon encounter of the antigen, while both variants display equal cytotoxic capacity. In contrast, the longer IgG3 variants (IgG3_MiH3, IgG3_MiH5, IgG3_UMLH) show reduced levels of proliferation, cytokine secretion and especially cytotoxicity with IgG3_MiH5 being the least functional one investigated (FIG. 7A-C).

### Example 7

### In Vivo Functionality and Persistence

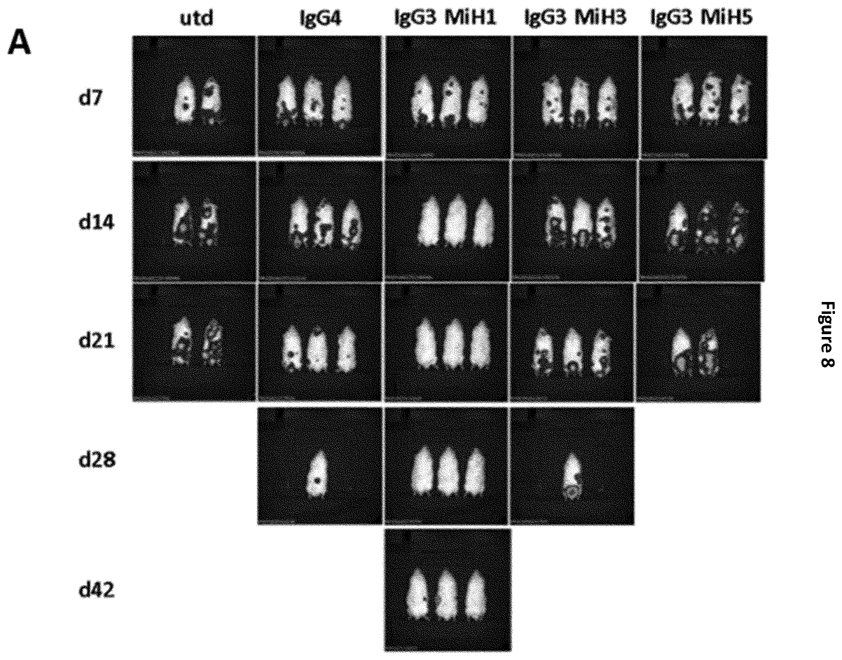

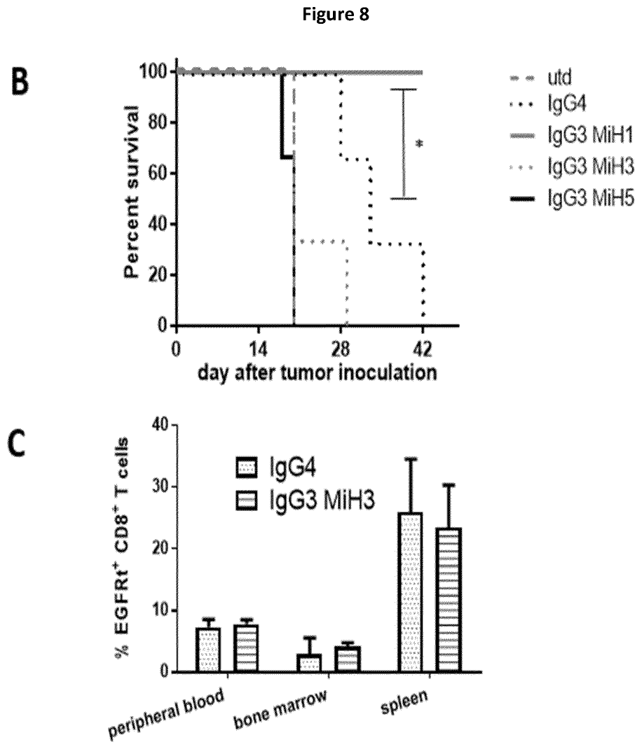

To translate the investigator's in vitro results to an in vivo model, 1×10⁶ CD19⁺ Raji tumor cells were engrafted in NSG mice that were treated 7d after tumor engraftment with 5×10⁶ CD8⁺ bulk T cells. T cells comprising the IgG3_MiH5 variant exhibited no beneficial effect on tumor growth and survival as compared to unmodified control T cells. While the IgG3_MiH3 variant slightly slowed down the increase in tumor burden and led to a not significant increase in survival, the IgG3_MiH1 variant and the IgG4 CAR led to complete eradication of the tumor. Though tumor cells eventually grew out in all mice, the IgG3_MiH1 variant delayed this outgrow and led to a significantly prolonged survival rate as compared to the IgG4 variant (FIG. 8A-B).

No immunogenicity against the IgG3 hinge was observed in mice (similar counts of T cells equipped with either IgG4 or IgG3-based spacers were detectable until the end of the experiment 35 days after T cell infusion), making it possible to study in vivo function of IgG3 Hinge variants of CAR T cells without the need for further modifications (e.g. removal of FcRγ binding sites, as for IgG4²⁶) (FIG. 8C).

Another mouse experiment was performed applying ROR1-specific CAR T cells equipped with the R11 scFV in mice engrafted with Jeko-1 for 7d. While neither the IgG4 spacer variant, nor IgG3 variants IgG3_MiH1 and IgG3_MiH4 influenced Jeko-1 tumor growth and survival of the treated animals, IgG3_MiH3 and especially IgG3_MiH2 led to attenuated tumor growth and prolonged animal survival (FIG. 9).

In summary, these in vivo data confirm the suitability and functionality of CARs with IgG3 Hinge-based spacer domains elaborated in in vitro experiments previously.

### Example 8

### Detection of the CAR and Exploiting Additional CAR-Intrinsic Functions Using IgG3 Hinge-Based Multi-Function Sites

The inventors identified an antibody (termed anti-MiH antibody #1, characterized by a heavy chain variable domain having the amino acid sequence of SEQ ID NO: 19, and a light chain variable domain having the amino acid sequence of SEQ ID NO: 23) specifically targeting the IgG3_MiH repeats of human IgG3 and aimed to use this to utilize additional antigen-independent though CAR-specific functions.

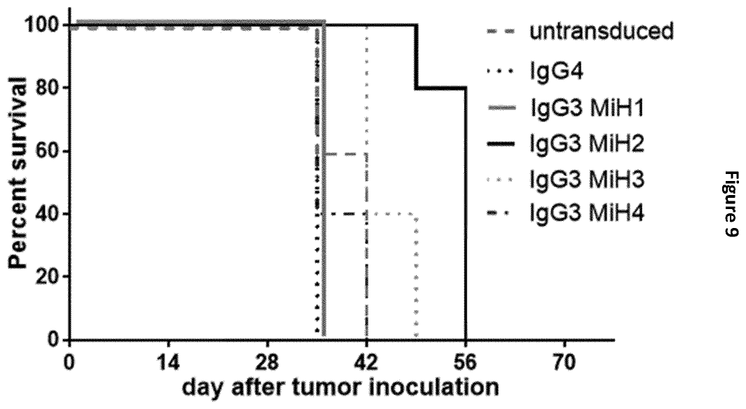

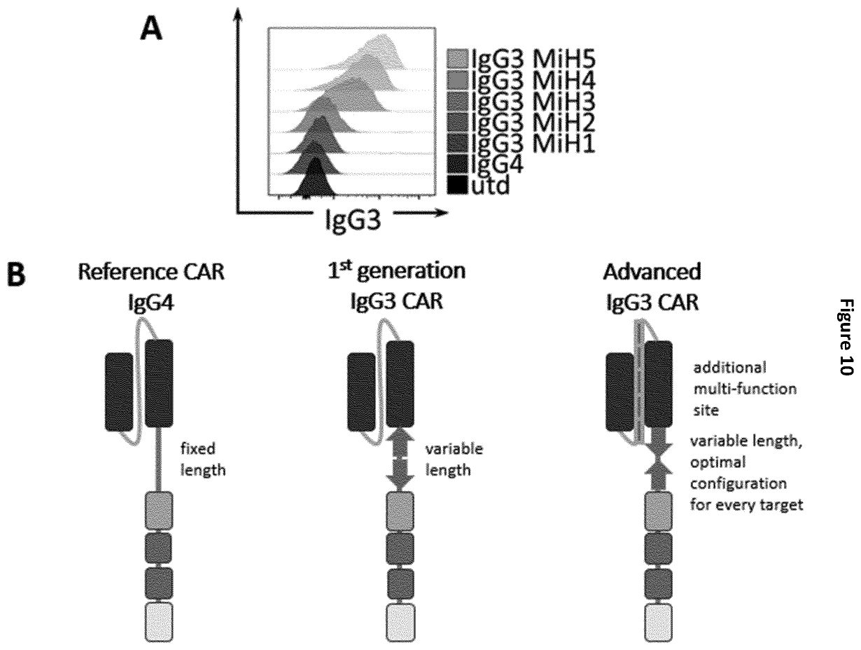

Since the inventors found proper binding of the antibody only from 3 or more IgG3_MiH repeats (FIG. 10A), and most scFvs tested show better function with relatively short IgG3-based spacers, the inventors introduced additional 5 IgG3_MiH repeats between scFv heavy and light chains replacing the commonly used (G₄S)₃ linker (“advanced format”, FIG. 10B).

### Example 9

### Proof of Functionality of Advanced Format In Vitro/In Vivo

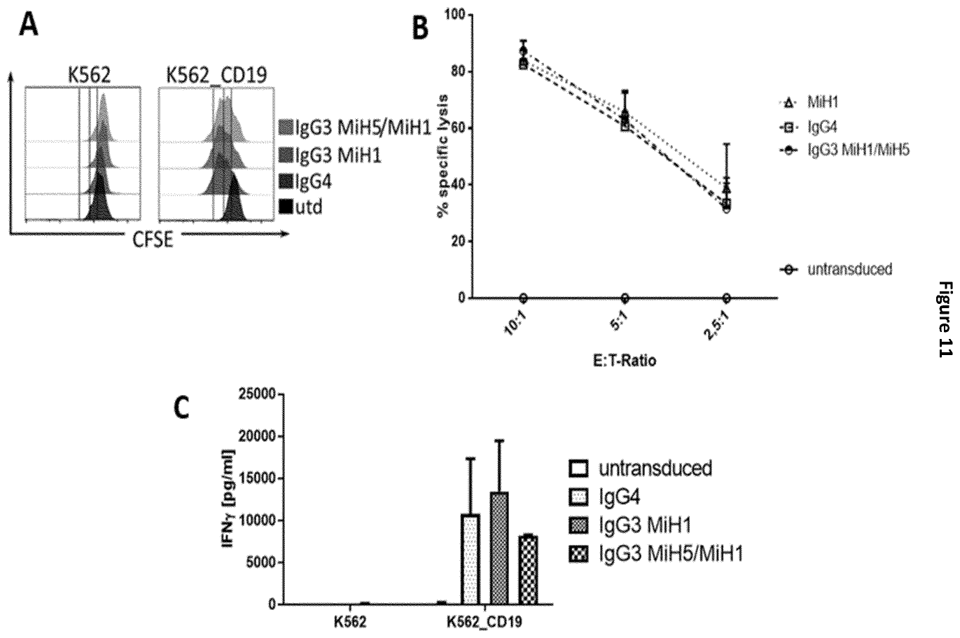

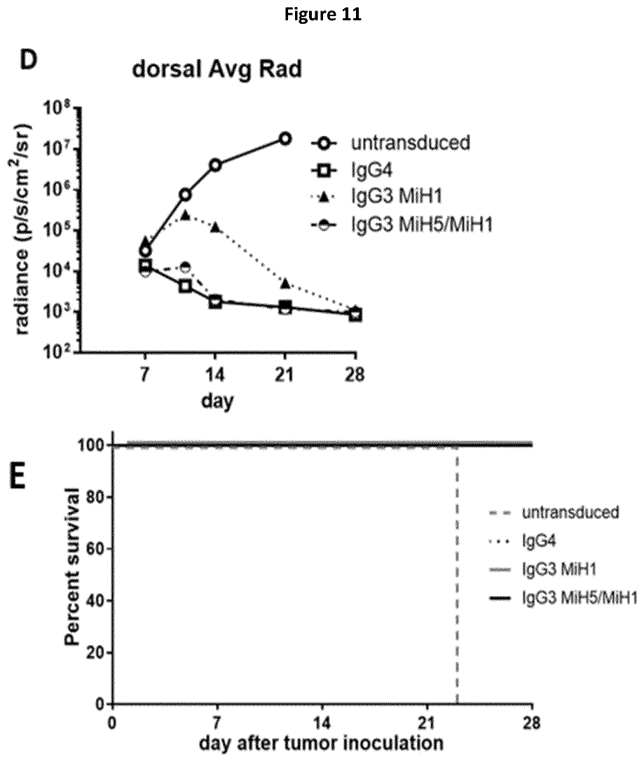

To exclude that the introduction of this multi-function site between scFv heavy and light chain (“advanced format”) impairs the antigen binding and thereby functionality of the CAR, the inventors compared CD19 CAR T cells engineered in the advanced IgG3 format to the optimal first generation IgG3 variant and the IgG4 reference CAR. No obvious differences occurred for in vitro proliferation, cytotoxicity and cytokine production between any of the variants (FIG. 11A-C). Similarly, all variants were equally capable of eradicating Raji tumor cells in vivo in NSG mice leading to enhanced survival of the animals (FIG. 11D-E). These results suggest that the introduction of the multifunction site between scFv VH and VL does not impair the CAR functionality, allowing the investigators to exploit it for additional functions.

### Example 10

### Multifunction-Site-Directed CAR T Purification

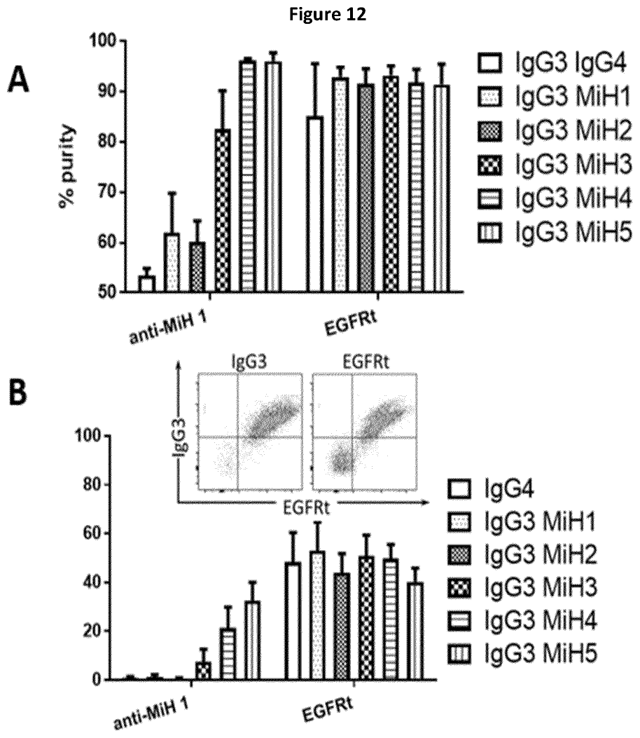

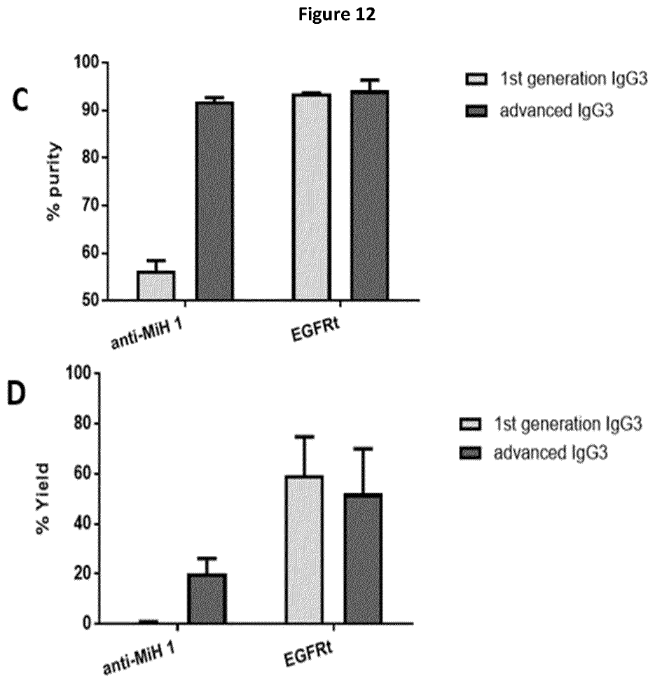

First, the inventors attempted to use the multi-function sites for purification of CAR-positive T cells. Therefore, the inventors compared their IgG3_MiH-specific antibody to an antibody targeting the well-established EGFRt (truncated epidermal growth factor receptor; included in the CAR transgene cassette, separated from the CAR by a T2A cleavage site) in the ability to purify CAR T cells from a 1:1 mixture of CART and untransduced T cells. While purification via EGFRt worked equally well for IgG4 and all IgG3_MiH variants (IgG3_MiH1-IgG3_MiH5), leading to purities of ˜90%, purification via the IgG3 Hinge achieved good purity only for 3 or more IgG3_MiH repeats. While for the longer IgG3_MiH variants, the cell products were comparable in purity, purification via IgG3_MiH lead to reduced yield of cells after purification (FIG. 12A-B).

These reduced levels in yield as compared to purification via EGFRt persisted, even after introduction of a second multifunction site between scFv VH and VL in the advanced format: while allowing to receive a highly pure cell population after sorting, the yield still falls behind EGFRt, also for the advanced IgG3-based CARs (FIG. 12C-D). Nonetheless, the investigator's data demonstrate that efficient sorting via the spacer domain or multi-function sites is feasible, leading to enrichment of a highly pure cell population.

### Example 11

### Multifunction-Site-Directed CAR T Cell Activation and Expansion

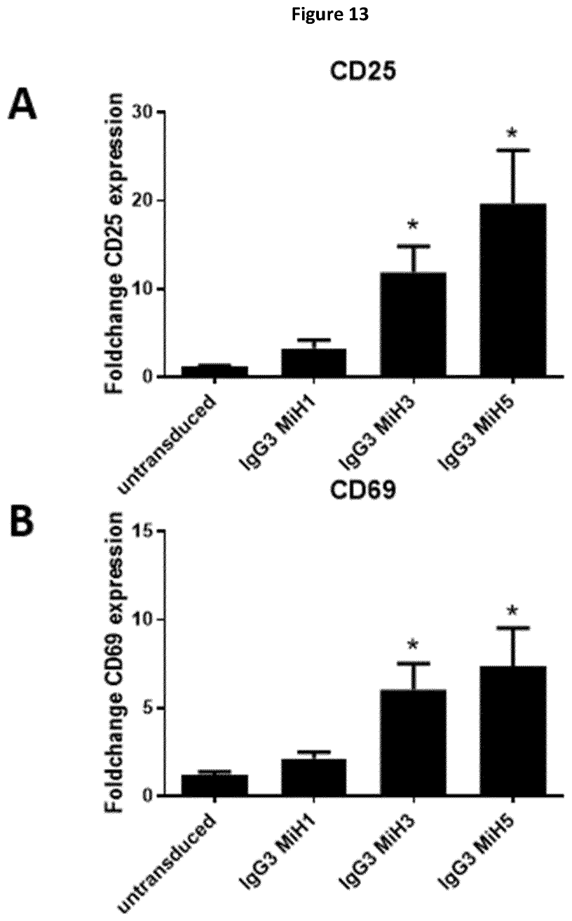

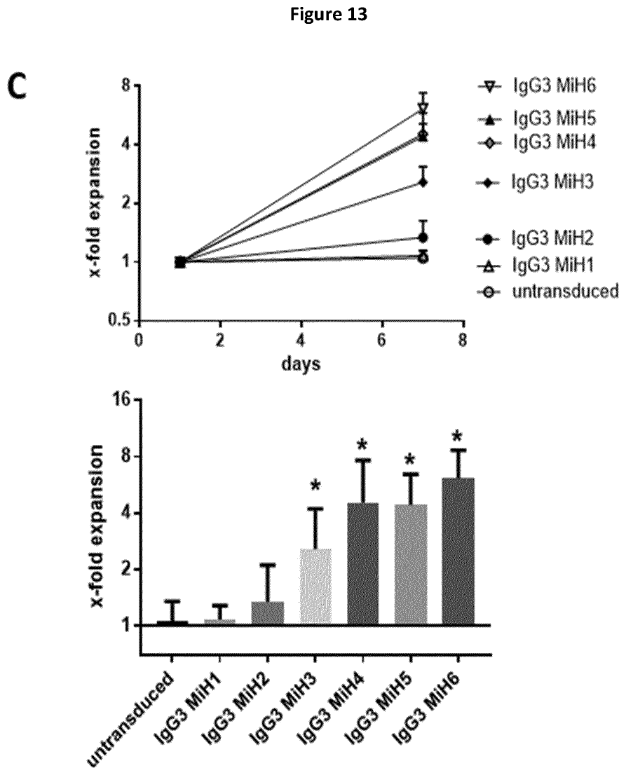

Activating CAR-modified T cells antigen-independently but CAR specifically offers the opportunity to expand these to large numbers in vitro without the need for irradiated feeder cells or bulk T cell activation by targeting CD3 and CD28. An additive beneficial effect is that the purity of the transgenic cell product is thereby increased without the need to manually enrich the cells. Therefore, the inventors investigated the ability of plate-bound IgG3 Hinge-specific anti-MiH antibody #1 to activate CAR T cells with IgG3-derived spacer domains. In good concordance with results obtained for purification, the antibody failed to induce upregulation of the T cell activation markers CD25 and CD69 for the IgG3_MiH1 variant. In contrast, both molecules were upregulated significantly in the IgG3_MiH3 and IgG3_MiH5 variants, with the 5 repeat variant being even more responsive (FIG. 13A-B).

These findings also correlated with the ability of the antibody to induce proliferation and expansion in CAR T cells equipped with spacers carrying 3 or more IgG3_MiH repeats. The presence of IgG3_MiH3 lead to a more than two-fold increase in CAR T cell numbers after 7 days of stimulation, 4 or more repeats resulted in a 4-fold expansion after one week (FIG. 13C).

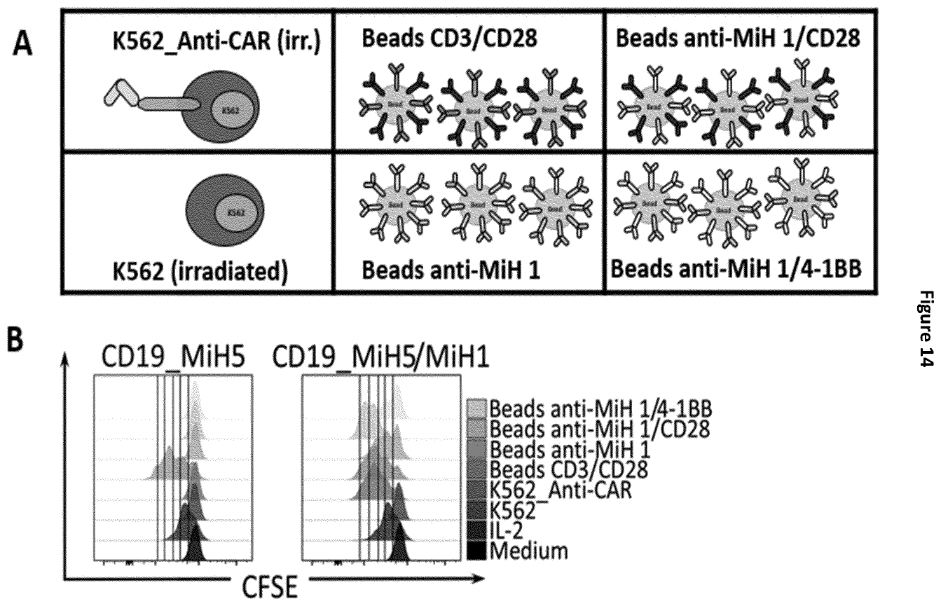

In search for methods allowing specific stimulation that are more feasible than using precoated antibody, the inventors conjugated their IgG3 Hinge-specific antibody to magnetic beads (ThermoFisher)Dynabeads®, alone or in combination with a-CD28 or α-4-1BB costimulatory antibodies and compared these to the well-established α-CD3/α-CD28 Dynabeads®(ThermoFisher Dynabeads®Human T Activator). In addition, the inventors generated a CAR with IgG4-derived spacer equipped with anti-MiH antibody #1 scFv as targeting domain and stably introduced this ‘Anti-CAR’ in K562 cells (FIG. 14A).

While α-CD3/α-CD28 Dynabeads®were able to induce proliferation in CAR T cells carrying a IgG3_MiH1 spacer, Beads coupled with anti-MiH antibody #1 or irradiated K562 with Anti-CAR had no stimulatory effect (FIG. 14B). In contrast, K562_Anti-CAR as well as all variants of anti-MiH antibody #1-coupled Dynabeads®induced proliferation in CAR T cells carrying the advanced IgG3 spacer format. This effect was most pronounced with anti-MiH antibody #1/α-CD28 Beads, which outperformed the established CD3/CD28 Dynabeads®in (FIG. 14C).

These results prove that especially CAR T cells carrying the advanced IgG3 format can be efficiently activated and expanded to large numbers antigen-independent but CAR-specific.

### Example 12

### Depletion In Vitro Using an ADC

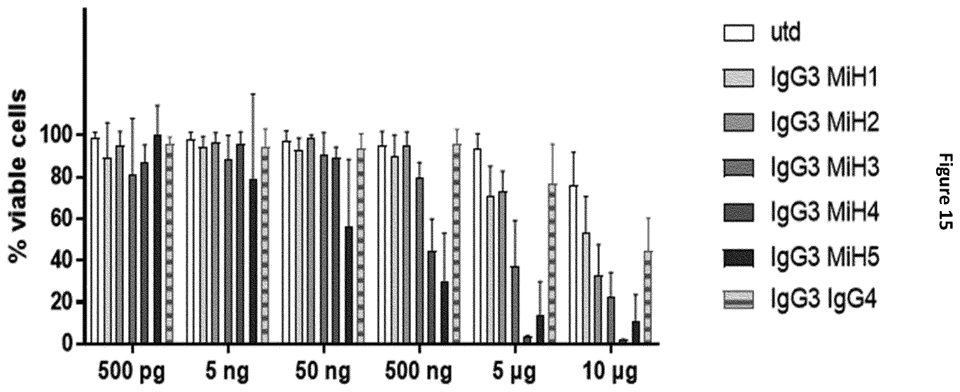

Even though an EGFRt safety switch is included in all CAR transgene cassettes described in this invention, having the possibility of a second option of intervention is highly wanted for the management of potential life-threatening toxicities that may occur upon CAR T cell treatment. Therefore, the inventors conjugated their IgG3 Hinge-specific antibody to a cytotoxic payload to obtain an antibody-drug-conjugate (ADC) that is capable of directly targeting the CAR itself. While already a concentration of 50 ng/ml shows a slight cytotoxic effect on CAR T cells with the IgG3_MiH5 variant, only 5 μg/ml led to a near-complete elimination of all cells equipped with a IgG3_MiH4 or IgG3_MiH5 variant after 3 days of culture. The IgG3_MiH3 variant showed at least a more than half reduction at 5 μg/ml. The highest concentration investigated (10 μg/ml) seems to mediate also unspecific effects, as the number of viable IgG4 Spacer CART cells did also decrease (FIG. 15).

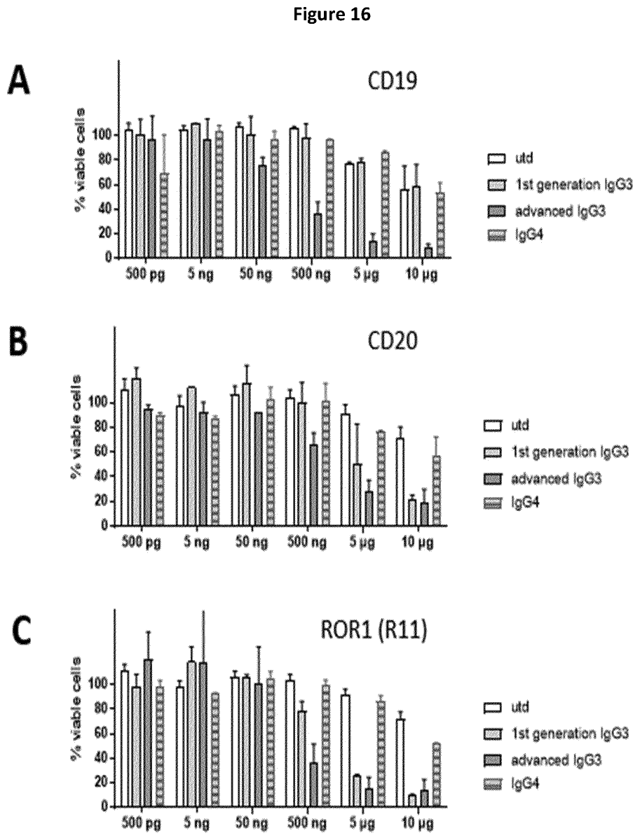

Similar results were obtained when investigating the effects on CAR T cells carrying the advanced IgG3 spacer format: while the first generation IgG3 spacer variant for CD19 (1 IgG3_MiH repeat) was not susceptible to specific ADC effects even at 10 μg/ml, the advanced version showed a 60% reduction at 500 ng/ml and a near-complete elimination at 5 μg/ml (FIG. 16A).

The CD20-specific CAR Leu16 (carrying 3 IgG3_MiH repeats in its spacer domain) showed overall a slightly weaker response to the ADC, with the advanced IgG3 version responding to 500 ng/ml while the majority of cells was eliminated at f.c. 5 μg/ml (FIG. 16B).

For CARs with the ROR1-specific scFV R11, equipped with 3 IgG3_MiH repeats, already 500 ng/ml showed a strong effect, that was further pronounced at 5 μg/ml and led to near-complete elimination of all cells (FIG. 16C).

These results prove the potency of an ADC-based way of CAR T cell elimination.

### Example 13

### Depletion In Vitro Using an Anti-CAR

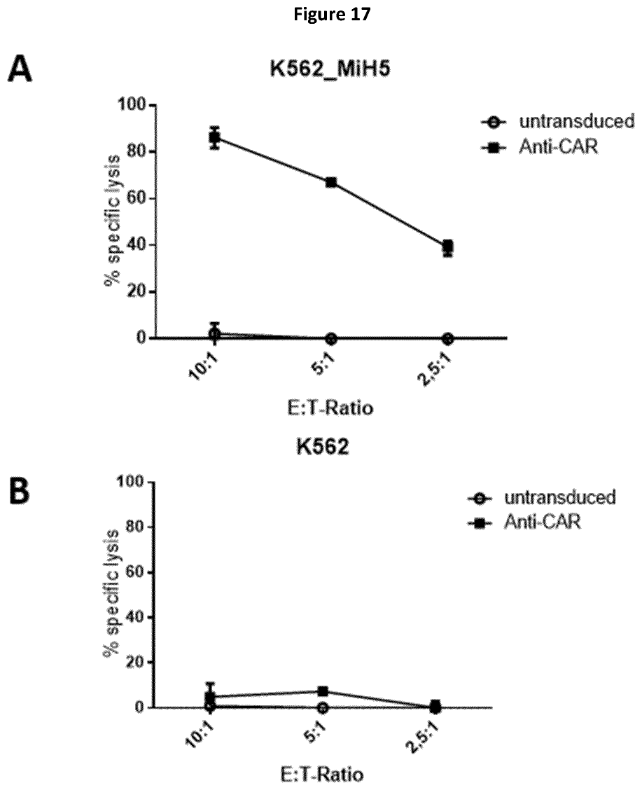

Another potential option for CAR T cell depletion would be to target unwanted IgG3 Hinge-based CAR t cells with other T cells equipped with the before-mentioned Anti-CAR (spacer derived from IgG4 Hinge). In cytotoxicity experiments targeting K562 cells transduced with a IgG3_MiH5 IgG3 CAR, specific recognition and elimination of these target cells was mediated by T cells carrying the Anti-CAR (FIG. 17), suggesting that CAR T cells could also be targeted and eliminated by other CAR Ts.

### Example 14

### In Vivo Depletion

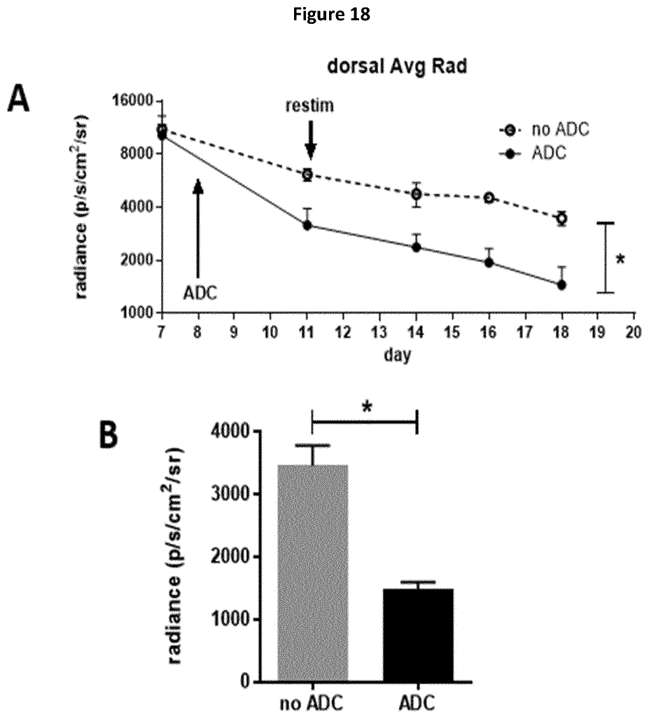

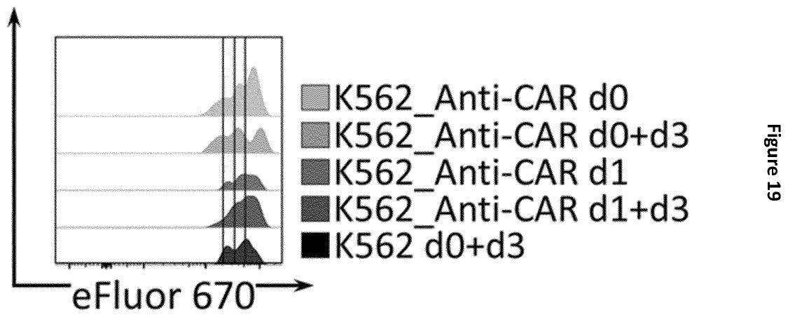

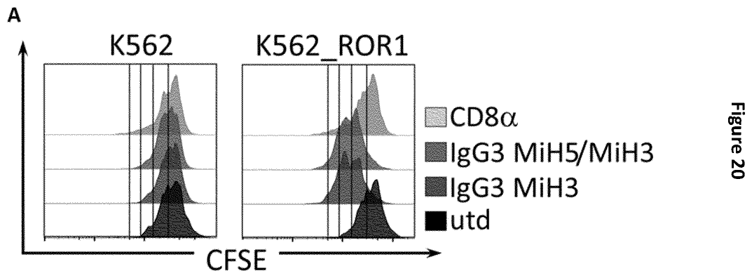

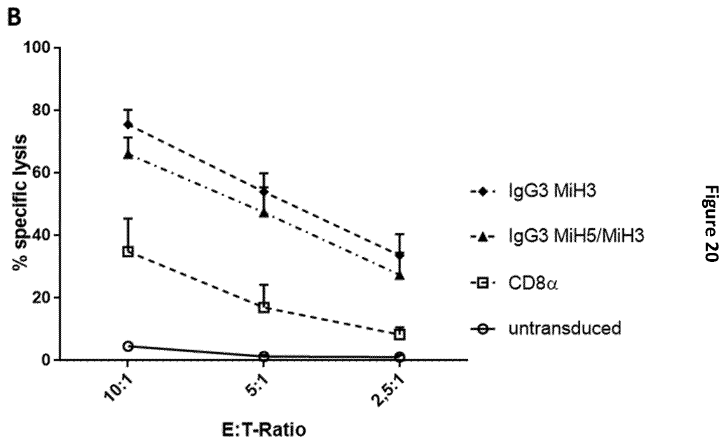

Next, the inventors checked whether depletion would be also possible in vivo. Therefore, the inventors used CD4⁺ T cells transduced with the advanced IgG3 format version of the CD19 CAR (CD19_IgG3_MiH5/MiH1) together with a firefly luciferase/GFP fusion protein, allowing bioluminescent imaging of the T cells in mice. T cells were inoculated and had engrafted by day 7 mainly in the bone marrow. At day 8, half of the mice were treated with 100 μg of anti-MiH antibody #1 ADC (approximately 4.5 mg/kg bodyweight). While the overall luminescence signal was slowly reducing, the mice in the ADC-treated group showed significantly lower radiance. The difference between the two groups was further increased when all mice were subjected to restimulation using irradiated K562 cells equipped with the before mentioned anti-MiH antibody #1-based Anti-CAR (1×10\hat{ }6 irradiated cells per mice) at day 11. This finally led to a significant 2.4-fold reduction in bioluminescence signal (and thereby T cell count) at the end of the experiment at day 18 (FIG. 18), thereby proving the option of significantly reducing the number of CAR T cells in a therapeutic setting if needed.

### Example 15

### In Vivo Proliferation

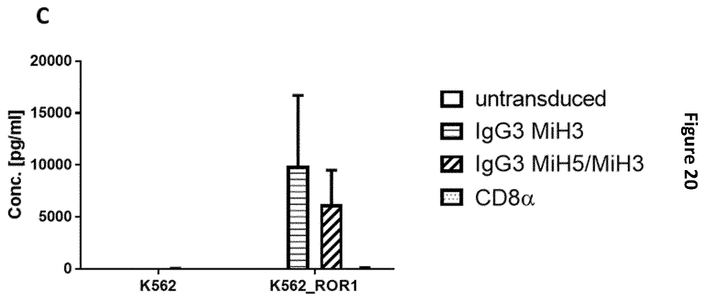

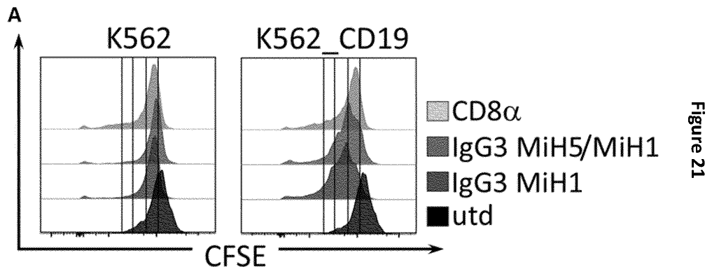

Next, the inventors examined whether induction of proliferation can be achieved in vivo. Therefore, the inventors used CD4⁺ T cells transduced with the advanced IgG3 format version of the CD19 CAR (CD19_IgG3_MiH5/MiH1) and labeled with the proliferation dye eFluor 670. T cells were inoculated and animals were additionally treated subsequently with 3×10\hat{ }6 irradiated K562 or K562_Anti-CAR cells at different time points. One group of mice (n=5 animals per group) received K562_Anti-CAR cells at the day of T cell injection (d0), 3 h after T transfer. A second group received an additional dose of irradiated K526_Anti-CAR cells at d3 post T cell injection (d0+d3), two other groups were treated with irradiated K562_Anti-CAR cells at day 1 post T cell transfer (d1) or at d1+d3, respectively. A control group received irradiated K562 cells at d0+d3. At day 4 post T cell transfer, mice were sacrificed and T cells from the bone marrow cells were collected and analyzed for eFluor 670 dilution. T cells from all groups showed proliferation to some extent. While mice treated with K562_Anti-CAR at d1 or d1+d3 or treated with K562 exhibited a lower proliferation rate, mice that received K562_Anti-CAR cells at d0 showed a much more pronounced rate of eFluor 670 dilution. Best proliferation was achieved after treatment with K562_Anti-CAR cells at d0+d3 (FIG. 19). These results demonstrate that CAR T cells equipped with an IgG3-based spacer can be successfully and specifically stimulated and activated in vivo.

The following additional Examples were carried out in the same way as the previous examples, with the following additions:

Cell Lines and Cell Culture Media

MV4-11, MOLM-13 (all ATCC, Manassas, Va., USA), as well as TM-EBV-LCL³⁵ (a kind gift from Fred Hutchinson Cancer Research Center, Seattle, Wash., US) cells were maintained in RPMI-1640 medium containing 8% fetal calf serum (FCS), 2 mM L-glutamine, and 100 U/mL penicillin/streptomycin (all components from Gibco, Thermo Scientific, Schwerte, Germany).

scFvs Used for CAR Generation

Codon optimized targeting domains comprising VH and VL segments of the following antibodies were synthesized (GeneArt ThermoFisher, Regensburg, Germany) and used as targeting domain for CAR constructs: FLT3: 4G8³², BV10³⁴, Siglec-6: JML-1³¹.

FLT3 (BV10) scFv: heavy chain variable domain having the amino acid sequence of SEQ ID NO: 45, light chain variable domain having the amino acid sequence of SEQ ID NO: 46.

FLT3 (4G8) scFv: heavy chain variable domain having the amino acid sequence of SEQ ID NO: 47, light chain variable domain having the amino acid sequence of SEQ ID NO: 48.

Siglec-6 (JML-1) scFv: heavy chain variable domain having the amino acid sequence of SEQ ID NO: 49, light chain variable domain having the amino acid sequence of SEQ ID NO: 50.

Antibodies and Flow Cytometry

Antibodies against Siglec-6 (clone REA852; APC) from Miltenyi, FLT3 (clone 4G8; AF647) from BD Biosciences (Heidelberg, Germany), and Siglec-6 (767329; PE) from BioTeche, Minneapolis, Minn., USA) were used.

Targeting the Multi-Function Site In Vitro

For antigen-independent though CAR-specific expansion, 5×10⁵ CAR T cells were co-cultured together with 5×10⁶ TM-EBV-LCL or K562_Anti-CAR cells, that have been irradiated to 80 Gy using a gamma irradiator, in X-VIVO™ 15 serum-free medium in the presence of 50 IU IL-2 for 14 days.

Targeting the Multi-Function Sites In Vivo

For in vivo tracking, CD8⁺ T cells were transduced with the advanced version of the IgG3-based CD19 CAR (CD19_IgG3_MiH5/MiH1) as well as with a ffluc_GFP fusion protein, enriched and expanded as above.

For ADC-Depletion, NSG mice (female, 6-8 week old, purchased from Charles River (Sulzfeld, Germany) were inoculated with 4.5×10⁶ ffluc_GFP⁺ CAR T cells by tail vein injection on day 0. At day 8, half of the mice were treated with 100 μg of anti-MiH antibody #1 ADC (approximately 4.5 mg/kg bodyweight). At d11, T cells were restimulated with irradiated K562 cells equipped with an anti-MiH antibody #1-based Anti-CAR (1×10⁶ irradiated K562 cells per mice). Kinetics of T cell persistence was assessed by serial bioluminescence imaging following i.p. administration of D-luciferin substrate (0.3 mg/g body weight) (Biosynth, Staad, Switzerland) using an IVIS Lumina imaging system (PerkinElmer, Waltham, Mass., USA). The data were analyzed using LivingImage software (PerkinElmer).

For Anti-CAR-T cell mediated depletion, NSG mice (female, 6-8 week old, purchased from Charles River, Sulzfeld, Germany) per group were inoculated with 2.2×10⁶ Target T cells (ffluc⁺GFP⁺+anti-CD19-CAR CD19_MiH5/MiH1; (CD4⁺:CD8⁺ ratio 1:1) and treated after 24 h with 4×10⁶ CD8⁺ Anti-CAR-CAR T cells or untransduced control T cells from the same donor. Serial bioluminescence imaging was conducted to assess T cell persistence/depletion in each treatment group following i.p. administration of D-luciferin substrate using an IVIS Lumina imaging system. The data were analyzed using LivingImage software.

For analysis of in vivo proliferation, CD4⁺ and CD8⁺ T cells were transduced with the advanced version of the IgG3-based CD19 CAR (CD19_IgG3_MiH5/MiH1), enriched and expanded as above and labeled with 5 μM of the proliferation dye eFluor 670 (ThermoFisher) according to the manufacturer's instruction or left unlabeled.

NSG mice (female, 6-8 week old, purchased from Charles River, Sulzfeld, Germany) were inoculated with indicated amounts of CAR T cells by tail vein injection on day 0. Groups of n=4-5 mice received irradiated stimulatory cells (either K562 or K562_Anti-CAR) subsequently at different time points per tail vein injection as indicated. Kinetics of T cell persistence/expansion was assessed by serial bioluminescence imaging following i.p. administration of D-luciferin substrate using an IVIS Lumina imaging system. The data were analyzed using LivingImage software. In some experiments, mice were sacrificed at d4 after T cell transfer, bone marrow cells were isolated, stained with antibodies against CD4, CD45 and EGFRt and subjected to flow cytometric analysis as above. CD45⁺/CD4⁺/EGFR⁺ bone-marrow derived T cells were analyzed for eFluor 670 dilution.

### Example 16

### In Vitro Function of Advanced IgG3 Format ROR1-Specific CAR T Cells and Comparison to CD8α Format

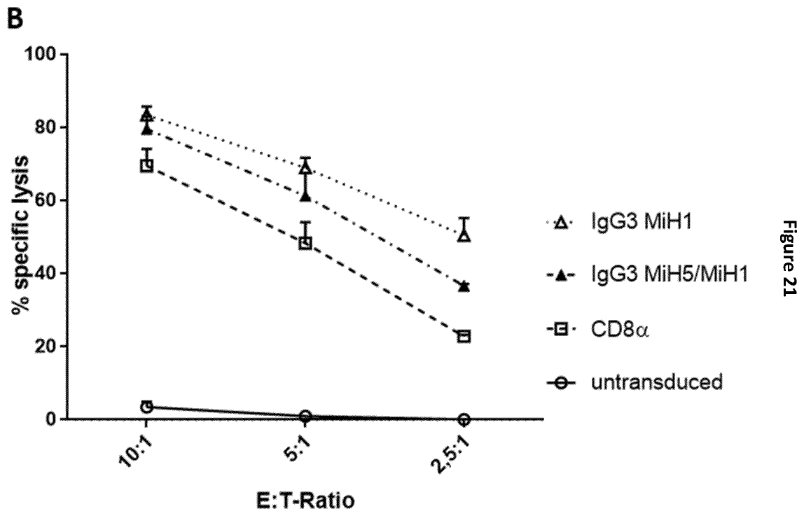

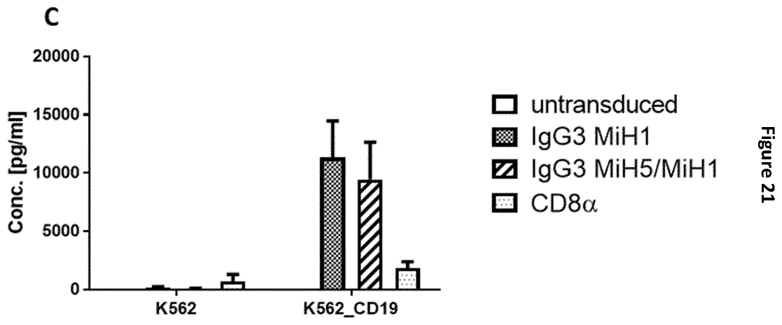

In an additional set of experiments, the inventors compared ROR1-specific CAR T cells (R11 scFv) engineered in the advanced IgG3 format to the optimal first generation IgG3 variant and a reference CAR in the widely applied CD8α setup (CD8α hinge and transmembrane domains)³³. While advanced and first generation IgG3 variants showed a comparably good antigen-specific proliferation, the CD8α variant revealed only minor proliferative capacity. This weaker response of the latter also translated to a significantly reduced cytotoxicity and cytokine secretion while first generation and advanced IgG3 variants behaved similarly effective (FIG. 20A-C).

### Example 17

### In Vitro Function of Advanced IgG3 Format CD19-Specific CAR T Cells and Comparison to CD8α Format

In an additional set of experiments, the inventors compared CD19-specific CAR T cells (FMC63 scFv) engineered in the advanced IgG3 format to the optimal first generation IgG3 variant and a reference CAR in the widely applied CD8α setup (CD8α hinge and transmembrane domains)³³.

No obvious differences occurred for in vitro proliferation, cytotoxicity and cytokine production between first generation and advanced IgG3 variants of the CD19-specific CAR while the CD8α control variant revealed weaker responses (FIG. 21A-C).

### Example 18

### In Vitro Cytotoxic Function of Additional Advanced IgG3 Format CAR T Cells and Comparison to CD8α Format

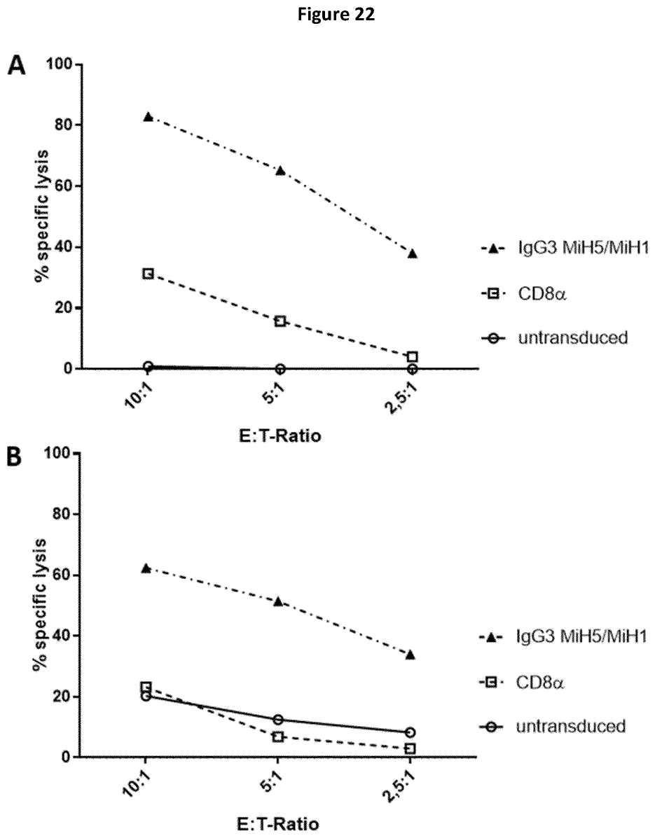

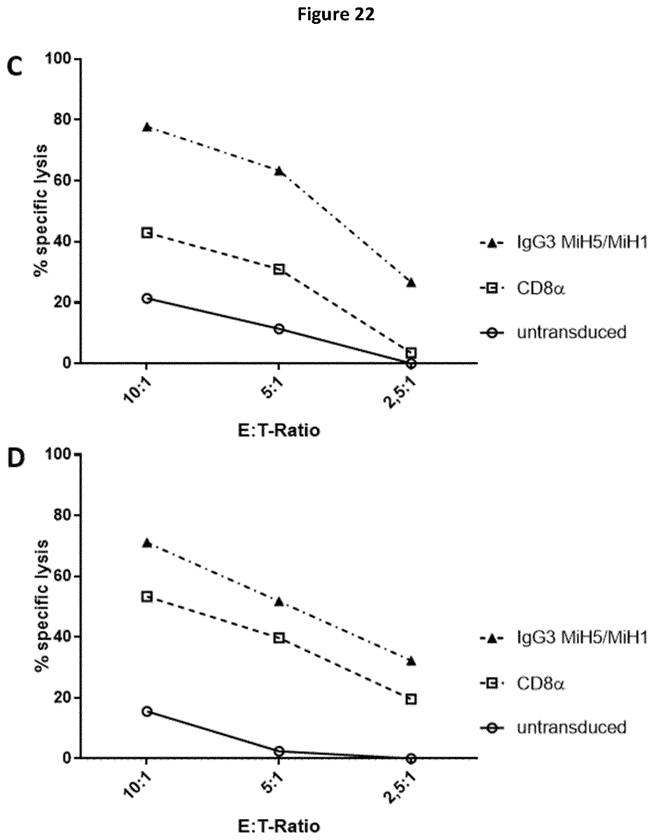

The inventors investigated the cytotoxic capacity of T cells equipped with optimized IgG3 variants of additional CARs targeting ROR1 (4-2 scFv), FLT3 (4G8 and BV10 scFv) and Siglec-6 (JML-1 scFv) and compared them to CARs with the same scFvs constructed in the widely applied CD8α setup (CD8α hinge and transmembrane domains)³³. All advanced IgG3 versions exhibited a significantly enhanced cytotoxic potential as compared to CD8α versions (FIG. 22A-D).

### Example 19

### In Vivo Function of Advanced IgG3 Format CAR T Cells and Comparison to CD8α Format

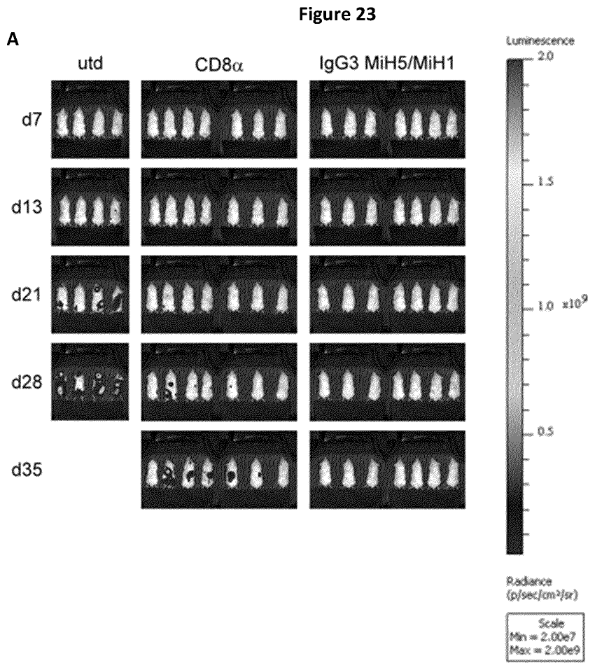

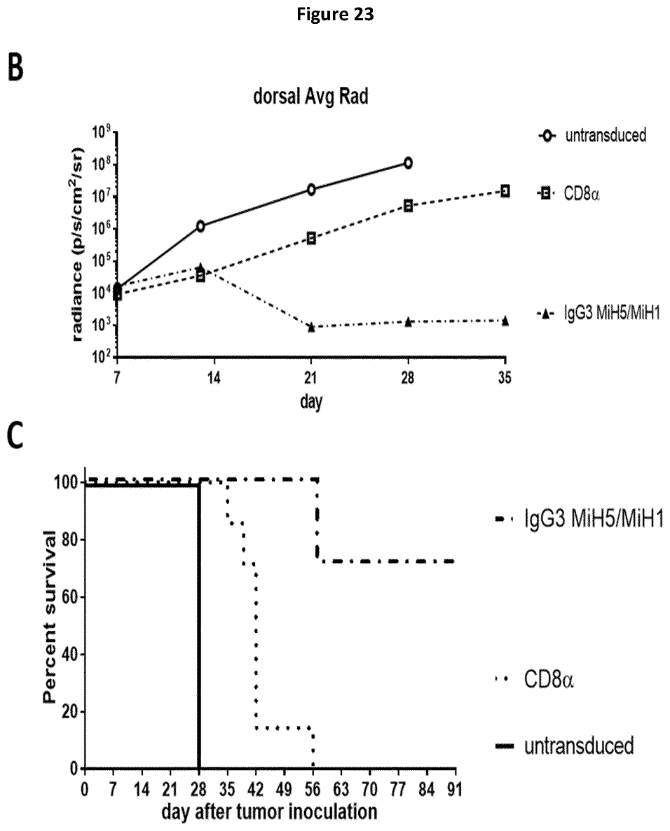

Next, the investigators aimed to examine whether the superiority of their advanced IgG3 CAR format over the CD8a control translated to a better antitumor efficacy also in vivo. Therefore, NSG mice were engrafted with 1×10\hat{ }6 ffluc/GFP⁺ Raji tumor cells and treated at d7 with 5×10⁶ (1:1 CD8⁺:CD4⁺ ratio) control or CD19-specific CAR T cells. While treatment with the clinically used CD8α CAR led only to a slowdown in tumor growth and a moderately prolonged survival of the treated mice in comparison to the control T cell group, application of the advanced IgG3 format CAR T cells led to complete tumor eradication associated with significantly enhanced survival (FIG. 23A-C).

### Example 20

### Spacer-Directed CAR T Cell Activation and Expansion In Vitro and In Vivo

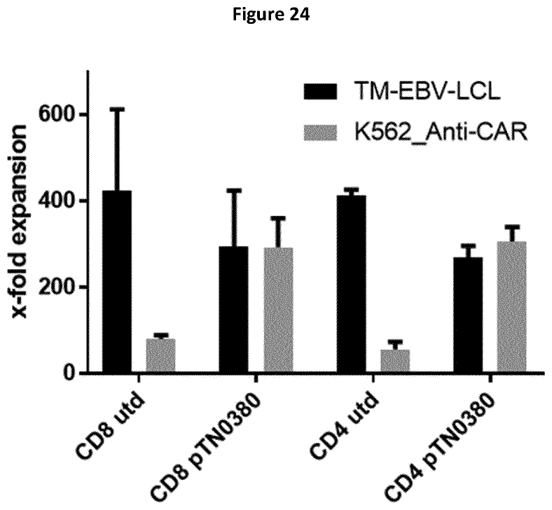

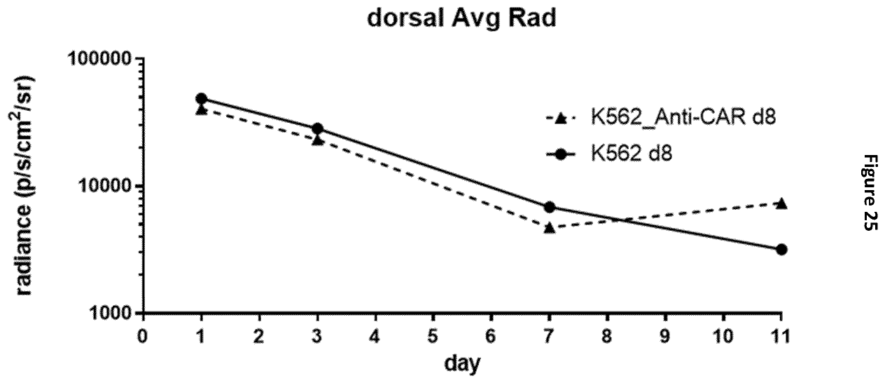

In search of methods for specific stimulation and expansion that are more feasible than precoated antibody, the investigators generated a CAR with IgG4-derived spacer equipped with anti-MiH1 scFv as targeting domains and stably introduced this ‘Anti-CAR’ in K562 cells. The inventors used irradiated K562 with Anti-CAR for T cell expansion and compared this to a well-established expansion protocol using irradiated TM-EBV-LCL feeder cells. Both, CD4⁺ and CD8⁺ T cells equipped with an advanced IgG3 version of the CD19-specific CAR exhibited similar expansion kinetics in the range of 250-fold expansion after 14 days with both protocols. In contrast, untransduced control T cells successfully expanded only when the TM-EBV-LCL feeder cell protocol+OKT3 was applied (FIG. 24).

Next, the inventors tested, whether T cells can be activated in vivo. Therefore, NSG mice were inoculated with 1×10⁷ GFP/ffluc⁺ CAR T cells (advanced IgG3 format), and after 8 days, mice were injected with 1×10⁷ K562 or K562 with Anti-CAR. While BLI signal further decreased in the K562 treated mice, BLI signal was enhanced in the Anti-CAR treated mice (FIG. 25). These results suggest that CAR T cells carrying advanced format IgG3-based CARs can be efficiently activated and expanded to large numbers antigen-independent but CAR-specifically in vitro as well as in vivo.

### Example 21

### Depletion In Vitro

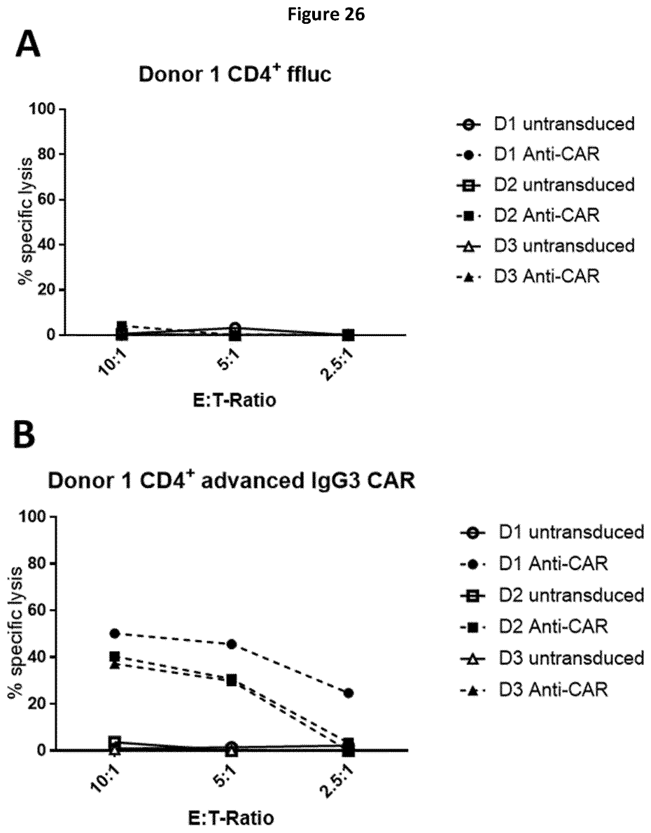

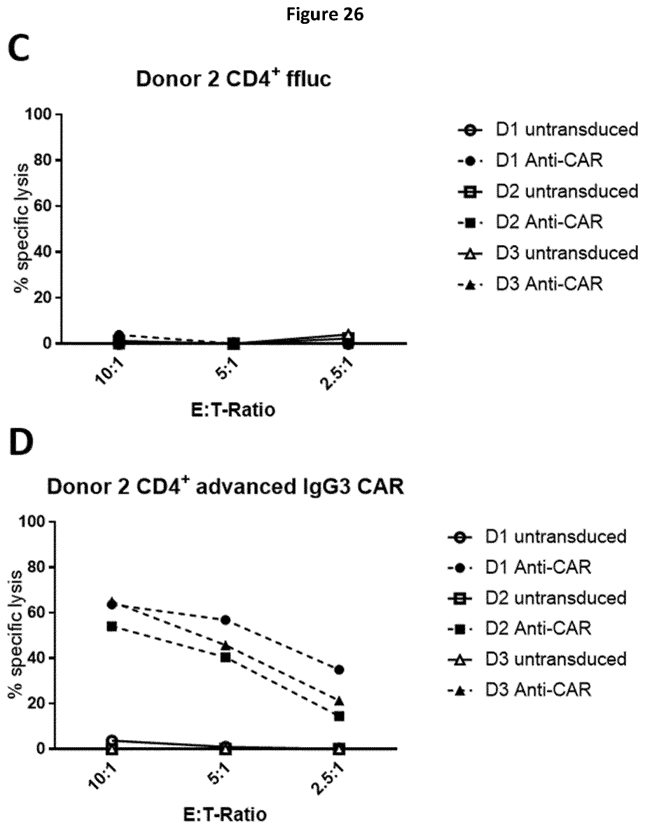

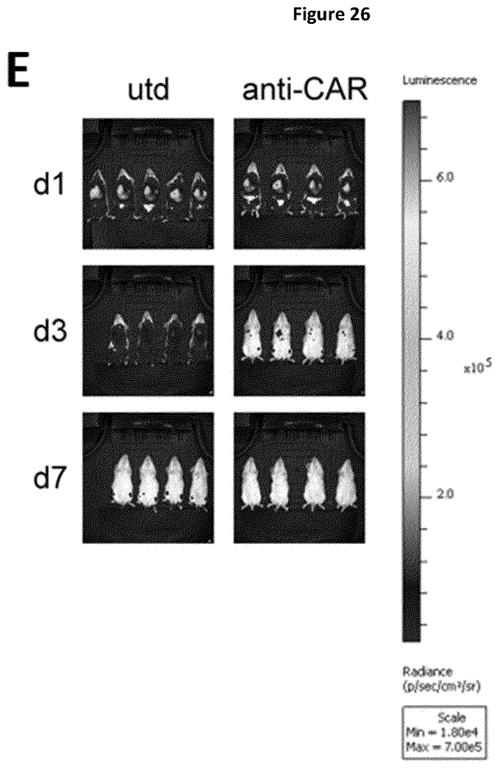

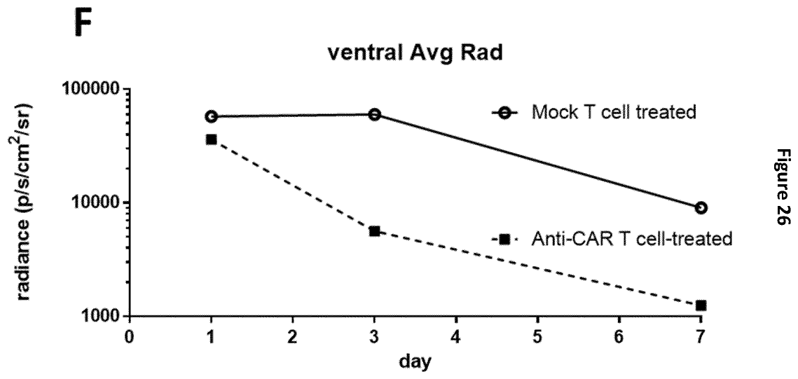

The inventors equipped T cells with the before-mentioned Anti-CAR (spacer derived from IgG4 Hinge). In cytotoxicity experiments with T cells transduced with an advanced format IgG3 CAR as target cells, these were specifically recognized and eliminated by T cells carrying the Anti-CAR in an auto—as well as in an allogeneic setting (FIG. 26A-D), suggesting that CAR T cells could also be targeted and eliminated by other CARTs, potentially even ‘off the shelf’.

### Example 22

### In Vivo Depletion

Next, the investigators checked whether depletion would be also possible in vivo. Therefore, they used CD4⁺ and CD8⁺ T cells transduced with the advanced IgG3 format version of the CD19 CAR together with a firefly luciferase/GFP fusion protein, allowing bioluminescent imaging of the T cells in mice. Target T cells were inoculated, and 24 h later mice were treated at a 2:1 E:T ratio with either Mock or Anti-CAR CD8⁺ T cells. While overall luminescence signal was slowly reducing, the mice in the anti-CAR-treated group showed significantly lower radiance, thereby proving the significant reduction of the number of CAR T cells in vivo that could be used in a therapeutic setting if needed (FIG. 26E-F).

### INDUSTRIAL APPLICABILITY

The immune cells for the uses according to the invention, as well as materials used for the methods of the invention, may be industrially manufactured and sold as products for the claimed methods and uses (e.g. for treating a cancer as defined herein), in accordance with known standards for the manufacture of pharmaceutical and diagnostic products. Accordingly, the present invention is industrially applicable.

### REFERENCES

1. Eshhar, Z., Waks, T., Gross, G. & Schindler, D. G. Specific activation and targeting of cytotoxic lymphocytes through chimeric single chains consisting of antibody-binding domains and the gamma or zeta subunits of the immunoglobulin and T-cell receptors. Proceedings of the National Academy of Sciences of the United States of America 90, 720-724 (1993).
2. Moritz, D. & Groner, B. A spacer region between the single chain antibody— and the CD3 zeta-chain domain of chimeric T cell receptor components is required for efficient ligand binding and signaling activity. Gene therapy 2, 539-546 (1995).
3. Guest, R. D. et al. The role of extracellular spacer regions in the optimal design of chimeric immune receptors: evaluation of four different scFvs and antigens. Journal of immunotherapy 28, 203-211 (2005).
4. Wilkie, S. et al. Retargeting of human T cells to tumor-associated MUC1: the evolution of a chimeric antigen receptor. Journal of immunology 180, 4901-4909 (2008).
5. Hudecek, M. et al. The B-cell tumor-associated antigen ROR1 can be targeted with T cells modified to express a ROR1-specific chimeric antigen receptor. Blood 116, 4532-4541 (2010).
6. Dotti, G., Gottschalk, S., Savoldo, B. & Brenner, M. K. Design and development of therapies using chimeric antigen receptor-expressing T cells. Immunological reviews 257, 107-126 (2014).
7. Hudecek, M. et al. The nonsignaling extracellular spacer domain of chimeric antigen receptors is decisive for in vivo antitumor activity. Cancer immunology research 3, 125-135 (2015).
8. Roux, K. H., Strelets, L., Brekke, O. H., Sandlie, I. & Michaelsen, T.E. Comparisons of the ability of human IgG3 hinge mutants, IgM, IgE, and IgA2, to form small immune complexes: A role for flexibility and geometry. Journal of immunology 161, 4083-4090 (1998).
9. Roux, K. H., Strelets, L. & Michaelsen, T.E. Flexibility of human IgG subclasses. Journal of immunology 159, 3372-3382 (1997).
10. Lu, Y. et al. Solution conformation of wild-type and mutant IgG3 and IgG4 immunoglobulins using crystallohydrodynamics: possible implications for complement activation. Biophysical journal 93, 3733-3744 (2007).
11. Lu, Y. et al. Crystallohydrodynamics of protein assemblies: Combining sedimentation, viscometry, and x-ray scattering. Biophysical journal 91, 1688-1697 (2006).
12. Iri-Sofia, F. J., Rahbarizadeh, F., Ahmadvand, D. & Rasaee, M. J. Nanobody-based chimeric receptor gene integration in Jurkat cells mediated by PhiC31 integrase. Experimental cell research 317, 2630-2641 (2011).
13. Jamnani, F. R. et al. T cells expressing VHH-directed oligoclonal chimeric HER2 antigen receptors: Towards tumor-directed oligoclonal T cell therapy. Bba-Gen Subjects 1840, 378-386 (2014).
14. Liu, L. F. et al. Inclusion of Strep-tag II in design of antigen receptors for T-cell immunotherapy. Nature biotechnology 34, 430-+ (2016).
15. Chu, J. et al. CS1-specific chimeric antigen receptor (CAR)-engineered natural killer cells enhance in vitro and in vivo antitumor activity against human multiple myeloma. Leukemia 28, 917-927 (2014).
16. Klein, J. S., Jiang, S., Galimidi, R. P., Keeffe, J. R. & Bjorkman, P. J. Design and characterization of structured protein linkers with differing flexibilities. Protein Eng Des Sel 27, 325-330 (2014).
17. Sommermeyer, D. et al. Chimeric antigen receptor-modified T cells derived from defined CD8+ and CD4+ subsets confer superior antitumor reactivity in vivo. Leukemia 30, 492-500 (2016).
18. Wang, X. et al. A transgene-encoded cell surface polypeptide for selection, in vivo tracking, and ablation of engineered cells. Blood 118, 1255-1263 (2011).
19. Riddell, S. R. & Greenberg, P. D. The use of anti-CD3 and anti-CD28 monoclonal antibodies to clone and expand human antigen-specific T cells. Journal of immunological methods 128, 189-201 (1990).
20. Zola, H. et al. Preparation and Characterization of a Chimeric-Cd19 Monoclonal-Antibody. Immunol Cell Biol 69, 411-422 (1991).
21. Ling N R, M. I., Mason D Y in Leucocyte Typing III White Cell Differentiation Antigens. (ed. C. S. McMichael A J, Crumpton M J, Gilka W, Peter C) 302-335 (Oxford University Press, Oxford; 1987).
22. Tai, Y. T. et al. Anti-CS1 humanized monoclonal antibody HuLuc63 inhibits myeloma cell adhesion and induces antibody-dependent cellular cytotoxicity in the bone marrow milieu. Blood 112, 1329-1337 (2008).
23. Yang, J. et al. Therapeutic potential and challenges of targeting receptor tyrosine kinase ROR1 with monoclonal antibodies in B-cell malignancies. PloS one 6, e21018(2011).
24. Peng, H. et al. Mining Naive Rabbit Antibody Repertoires by Phage Display for Monoclonal Antibodies of Therapeutic Utility. J Mol Biol 429, 2954-2973 (2017).
25. Brown, C.E. et al. Recognition and killing of brain tumor stem-like initiating cells by CD8+ cytolytic T cells. Cancer research 69, 8886-8893 (2009).
26. Hudecek, M. et al. Receptor affinity and extracellular domain modifications affect tumor recognition by ROR1-specific chimeric antigen receptor T cells. Clinical cancer research: an official journal of the American Association for Cancer Research 19, 3153-3164 (2013).
27. Monjezi, R. et al. Enhanced CAR T-cell engineering using non-viral Sleeping Beauty transposition from minicircle vectors. Leukemia 31, 186-194 (2017).
28. Chen, X. Y., Zaro, J. L. & Shen, W. C. Fusion protein linkers: Property, design and functionality. Adv Drug Deliver Rev 65, 1357-1369 (2013).
29. Zah, E., Lin, M. Y., Silva-Benedict, A., Jensen, M. C. & Chen, Y. Y. T Cells Expressing CD19/CD20 Bispecific Chimeric Antigen Receptors Prevent Antigen Escape by Malignant B Cells. Cancer immunology research 4, 498-508 (2016).
30. Gogishvili, T. et al. SLAMF7-CAR T cells eliminate myeloma and confer selective fratricide of SLAMF7(+) normal lymphocytes. Blood 130, 2838-2847 (2017).
31. Baskar S. et al. A human monoclonal antibody drug and target discovery platform for B-cell chronic lymphocytic leukemia based on allogeneic hematopoietic stem cell transplantation and phage display. Blood 114, 4494-502 (2009).
32. Hofmann M. et al. Generation, selection and preclinical characterization of an Fc-optimized FLT3 antibody for the treatment of myeloid leukemia. Leukemia 26, 1228-37 (2012).
33. Porter, D L., Levine, B L., Kalos, M., Bagg, A. & June, CH. Chimeric antigen receptor-modified T cells in chronic lymphoid leukemia. N Engl J Med 365, 725-33 (2011).
34. Rappold I. et al. Functional and phenotypic characterization of cord blood and bone marrow subsets expressing FLT3 (CD135) receptor tyrosine kinase. Blood 90, 111-125 (1997).
35. Terakura, S. et al. Generation of CD19-chimeric antigen receptor modified CD8+ T cells derived from virus-specific central memory T cells. Blood 119, 72-82 (2012).

1. An immunoreceptor, comprising one or more IgG3 middle hinge repeat domain motifs, wherein the immunoreceptor does not comprise an IgG3 CH2 and/or CH3 domain.

2. The immunoreceptor according to claim 1, wherein the immunoreceptor comprises an amino acid sequence which has at least 80% sequence identity, preferably at least 90% sequence identity, or most preferably 100% sequence identity with the amino acid sequence of [A-Bₙ],
wherein
A is the amino acid sequence of SEQ ID NO: 2;
B is said IgG3 middle hinge domain repeat motif, wherein said motif has the amino acid sequence of SEQ ID NO: 1; and
n is selected from the group consisting of 0, 1, 2, 3, 4, 5, 6, 7, 8, 9, 10, 11, 12, 13, 14 and 15 and is preferably an integer between 1 and 15, more preferably an integer between 1 and 10, even more preferably an integer between 1 and 5, most preferably an integer between 3 and 5.

3. The immunoreceptor according to claim 2, wherein the immunoreceptor comprises an amino acid sequence which has 100% sequence identity with the amino acid sequence of [A-Bₙ].

4. The immunoreceptor according to claim 2 or 3, wherein n is an integer between 1 and 10.

5. The immunoreceptor according to claim 2 or 3, wherein n is an integer between 1 and 5.

6. The immunoreceptor according to claim 2 or 3, wherein n is an integer between 3 and 5.

7. The immunoreceptor according to any one of the preceding claims, comprising:
an extracellular antigen-binding domain,
a spacer domain,
a transmembrane domain, and
an intracellular signaling domain;
wherein the spacer domain is located between the extracellular antigen-binding domain and the transmembrane domain,
and wherein optionally the spacer domain comprises one or more IgG3 middle hinge domain repeat motifs.

8. The immunoreceptor according to claim 7, wherein the transmembrane domain and the intracellular domain together consist of a sequence selected from the group consisting of SEQ ID NO: 109, 110, 111, 112, 113, 114, 115 and 174.

9. The immunoreceptor according to any one of the preceding claims, comprising an extracellular antigen-binding domain comprising:
a first domain,
a linker, and, optionally,
a second domain;
optionally wherein the linker is located between the first domain and the second domain,
and wherein optionally the linker comprises one or more IgG3 middle hinge domain repeat motifs.

10. The immunoreceptor according to claim 7, 8 or 9,
wherein the spacer domain comprises one or more IgG3 middle hinge domain repeat motifs,
and/or
wherein the linker comprised in the extracellular antigen-binding domain comprises one or more IgG3 middle hinge domain repeat motifs.

11. The immunoreceptor according to any one of the preceding claims, wherein the immunoreceptor is selected from the group consisting of a T-cell receptor (TCR), preferably a recombinant TCR; a B-cell receptor (BCR), preferably a recombinant BCR; and a chimeric antigen receptor (CAR).

12. The immunoreceptor according to any one of claims 9 to 11, wherein the immunoreceptor comprises the antigen-binding domain, wherein
I) the first domain comprises a heavy chain variable domain;
II) the first domain comprises a light chain variable domain;
III) the first domain comprises a heavy chain variable domain, and the second domain comprises a light chain variable domain;
IV) the first domain comprises a heavy chain variable domain, and the second domain comprises a heavy chain variable domain; or
V) the first domain comprises a light chain variable domain, and the second domain comprises a light chain variable domain.

13. The immunoreceptor according to any one of claims 9 to 12, wherein the immunoreceptor comprises the antigen-binding domain, said antigen-binding domain comprising the first domain, linker, and second domain, which are part of a single chain variable fragment (scFv),
wherein the scFv optionally comprises, as heavy/light chain variable sequences comprised in the first/second domain, heavy/light chain variable sequences of scFvs specific for one of the following antigens:
A) CD19, optionally wherein
i) the heavy chain variable domain has an amino acid sequence having at least 80% sequence identity, preferably at least 90% sequence identity, to SEQ ID NO: 27,
the light chain variable domain has an amino acid sequence having at least 80% sequence identity, preferably at least 90% sequence identity, to SEQ ID NO: 28,
and the scFv is capable of specifically binding to CD19; or
ii) the heavy chain variable domain has the amino acid sequence of SEQ ID NO: 27 and the light chain variable domain has the amino acid sequence of SEQ ID NO: 28;
B) CD20, optionally wherein
i) the heavy chain variable domain has an amino acid sequence having at least 80% sequence identity, preferably at least 90% sequence identity, to SEQ ID NO: 30,
the light chain variable domain has an amino acid sequence having at least 80% sequence identity, preferably at least 90% sequence identity, to SEQ ID NO: 29,
and the scFv is capable of specifically binding to CD20; or
ii) the heavy chain variable domain has the amino acid sequence of SEQ ID NO: 30 and the light chain variable domain has the amino acid sequence of SEQ ID NO: 29;
C) ROR1, optionally wherein
i) the heavy chain variable domain has an amino acid sequence having at least 80% sequence identity, preferably at least 90% sequence identity, to SEQ ID NO: 31, 33, 35, or 37,
the light chain variable domain has an amino acid sequence having at least 80% sequence identity, preferably at least 90% sequence identity, to SEQ ID NO: 32, 34, 36, or 38, respectively, and the scFv is capable of specifically binding to ROR1; or
ii) the heavy chain variable domain has the amino acid sequence of SEQ ID NO: 31, 33, 35, or 37 and the light chain variable domain has the amino acid sequence of SEQ ID NO: 32, 34, 36, 38, respectively;
D) ROR2, optionally wherein
i) the heavy chain variable domain has an amino acid sequence having at least 80% sequence identity, preferably at least 90% sequence identity, to SEQ ID NO: 39,
the light chain variable domain has an amino acid sequence having at least 80% sequence identity, preferably at least 90% sequence identity, to SEQ ID NO: 40,
and the scFv is capable of specifically binding to ROR2; or
ii) the heavy chain variable domain has the amino acid sequence of SEQ ID NO: 39 and the light chain variable domain has the amino acid sequence of SEQ ID NO: 40;
E) SLAMF7, optionally wherein
i) the heavy chain variable domain has an amino acid sequence having at least 80% sequence identity, preferably at least 90% sequence identity, to SEQ ID NO: 41 or 43,
the light chain variable domain has an amino acid sequence having at least 80% sequence identity, preferably at least 90% sequence identity, to SEQ ID NO: 42 or 44, respectively,
and the scFv is capable of specifically binding to SLAMF7; or
ii) the heavy chain variable domain has the amino acid sequence of SEQ ID NO: 41 or 43 and the light chain variable domain has the amino acid sequence of SEQ ID NO: 42 or 44, respectively;
F) FLT3, optionally wherein
i) the heavy chain variable domain has an amino acid sequence having at least 80% sequence identity, preferably at least 90% sequence identity, to SEQ ID NO: 45 or 47,
the light chain variable domain has an amino acid sequence having at least 80% sequence identity, preferably at least 90% sequence identity, to SEQ ID NO: 46 or 48, respectively,
and the scFv is capable of specifically binding to FLT3; or
ii) the heavy chain variable domain has the amino acid sequence of SEQ ID NO: 45 or 47 and the light chain variable domain has the amino acid sequence of SEQ ID NO: 46 or 48, respectively;
G) Siglec-6, optionally wherein
i) the heavy chain variable domain has an amino acid sequence having at least 80% sequence identity, preferably at least 90% sequence identity, to SEQ ID NO: 49,
the light chain variable domain has an amino acid sequence having at least 80% sequence identity, preferably at least 90% sequence identity, to SEQ ID NO: 50,
and the scFv is capable of specifically binding to Siglec-6; or
ii) the heavy chain variable domain has the amino acid sequence of SEQ ID NO: 49 and the light chain variable domain has the amino acid sequence of SEQ ID NO: 50;
H) αᵥβ₃ integrin, optionally wherein
i) the heavy chain variable domain has an amino acid sequence having at least 80% sequence identity, preferably at least 90% sequence identity, to SEQ ID NO: 51 or 53,
the light chain variable domain has an amino acid sequence having at least 80% sequence identity, preferably at least 90% sequence identity, to SEQ ID NO: 52 or 54, respectively,
and the scFv is capable of specifically binding to αᵥβ₃integrin; or
ii) the heavy chain variable domain has the amino acid sequence of SEQ ID NO: 51 or 53 and the light chain variable domain has the amino acid sequence of SEQ ID NO: 52 or 54, respectively;
or
I) BCMA, optionally wherein
i) the heavy chain variable domain has an amino acid sequence having at least 80% sequence identity, preferably at least 90% sequence identity, to SEQ ID NO: 55 or 57,
the light chain variable domain has an amino acid sequence having at least 80% sequence identity, preferably at least 90% sequence identity, to SEQ ID NO: 56 or 58, respectively,
and the scFv is capable of specifically binding to BCMA; or
ii) the heavy chain variable domain has the amino acid sequence of SEQ ID NO: 55 or 57 and the light chain variable domain has the amino acid sequence of SEQ ID NO: 56 or 58, respectively.

14. The immunoreceptor according to any one of claims 9 to 13, wherein the immunoreceptor comprises the antigen-binding domain, said antigen-binding domain comprising an scFv:
I) specific to CD19, optionally wherein said scFv comprises an amino acid sequence having at least 80% sequence identity, preferably at least 90% sequence identity, to SEQ ID NO: 3 or 71 and is capable of specifically binding to CD19, or wherein said scFv has the amino acid sequence of SEQ ID NO: 3 or 71;
II) specific to CD20 optionally wherein said scFv comprises an amino acid sequence having at least 80% sequence identity, preferably at least 90% sequence identity, to SEQ ID NO: 4 or 72 and is capable of specifically binding to CD20, or wherein said scFv has the amino acid sequence of SEQ ID NO: 4 or 72;
III) specific to ROR1, optionally wherein said scFv comprises an amino acid sequence having at least 80% sequence identity, preferably at least 90% sequence identity, to SEQ ID NO: 5, 6, 7, 8, 73, 74, 75, 76, 87, 88, 89, 90, 91, 92, 93, 94, 95, 96, 97, 98, 99 or 100 and is capable of specifically binding to ROR1, or wherein said scFv has the amino acid sequence of SEQ ID NO: 5, 6, 7, 8, 73, 74, 75, 76, 87, 88, 89, 90, 91, 92, 93, 94, 95, 96, 97, 98, 99 or 100;
IV) specific to ROR2, optionally wherein said scFv comprises an amino acid sequence having at least 80% sequence identity, preferably at least 90% sequence identity, to SEQ ID NO: 9, 77, 101, 102, 103, 104, 105, 106, 107 or 108 and is capable of specifically binding to ROR2, or wherein said scFv has the amino acid sequence of SEQ ID NO: 9, 77, 101, 102, 103, 104, 105, 106, 107 or 108;
V) specific to SLAMF7, optionally wherein said scFv comprises an amino acid sequence having at least 80% sequence identity, preferably at least 90% sequence identity, to SEQ ID NO: 10, 11, 78 or 79 and is capable of specifically binding to SLAMF7, or wherein said scFv has the amino acid sequence of SEQ ID NO: 10, 11, 78 or 79;
VI) specific to FLT3, optionally wherein said scFv comprises an amino acid sequence having at least 80% sequence identity, preferably at least 90% sequence identity, to SEQ ID NO: 12, 13, 80 or 81 and is capable of specifically binding to FLT3, or wherein said scFv has the amino acid sequence of SEQ ID NO: 12, 13, 80 or 81;
VII) specific to Siglec-6, optionally wherein said scFv comprises an amino acid sequence having at least 80% sequence identity, preferably at least 90% sequence identity, to SEQ ID NO: 14 or 82 and is capable of specifically binding to Siglec-6, or wherein said scFv has the amino acid sequence of SEQ ID NO: 14 or 82;
VIII) specific to αᵥβ₃ integrin, optionally wherein said scFv comprises an amino acid sequence having at least 80% sequence identity, preferably at least 90% sequence identity, to SEQ ID NO: 15, 16, 83 or 84 and is capable of specifically binding to αᵥβ₃integrin, or wherein said scFv has the amino acid sequence of SEQ ID NO: 15, 16, 83 or 84;
IX) specific to BCMA, optionally wherein said scFv comprises an amino acid sequence having at least 80% sequence identity, preferably at least 90% sequence identity, to SEQ ID NO: 17, 18, 85 or 86 and is capable of specifically binding to BCMA, or wherein said scFv has the amino acid sequence of SEQ ID NO: 17, 18, 85 or 86.

15. The immunoreceptor according to any one claims 1 to 14, wherein the immunoreceptor is a chimeric antigen receptor (CAR).

16. The immunoreceptor or CAR according to any one of claims 1 to 15, wherein the one or more IgG3 middle hinge domain repeat motifs
I) Are from a human IgG3 middle hinge; and/or
II) Consist of the amino acid sequence of SEQ ID NO: 1; and/or
III) Have reduced immunogenicity compared to repeats of an IgG1 hinge domain and/or an IgG4 hinge domain.

17. The immunoreceptor or CAR according to any one of claims 1 to 16, wherein the immunoreceptor or CAR:
I) Does not comprise all or part of the sequence of the lower hinge domain of a human IgG3 hinge domain;
II) Comprises an amino acid sequence which has at least 80% sequence identity, preferably at least 90% sequence identity, or most preferably 100% sequence identity
with the amino acid sequence of [A-Bₙ],
wherein
A is the amino acid sequence of SEQ ID NO: 2;
B is said IgG3 middle hinge domain repeat motif, wherein said motif has the amino acid sequence of SEQ ID NO: 1; and
n is selected from the group consisting of 0, 1, 2, 3, 4, 5, 6, 7, 8, 9, 10, 11, 12, 13, 14 and 15 and is preferably an integer between 1 and 15, more preferably an integer between 1 and 10, even more preferably an integer between 1 and 5, most preferably an integer between 3 and 5;
III) Comprises the IgG3 middle hinge domain repeat motif 1, 2, 3, 4, 5, 6, 7, 8, 9 or 10 times; and/or
IV) has reduced immunogenicity compared to a second CAR which differs from the first CAR in that it does not comprise said one or more IgG3 middle hinge domain repeat motifs.

18. The immunoreceptor or CAR according to any one of claims 1 to 17, wherein the immunoreceptor or CAR comprises at least two, preferably at least three of said IgG3 middle hinge domain repeat motifs which are adjacent to each other.

19. A CAR according to any one of the preceding claims, comprising an amino acid sequence selected from the group consisting of SEQ ID NO: 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, and 171.

20. A nucleic acid encoding the immunoreceptor or CAR according to any one of claims 1 to 19.

21. A cell, comprising the nucleic acid according to claim 20.

22. The cell according to claim 21, wherein:
I) The cell is an immune cell, preferably a B cell, macrophage, NK cell or T cell, more preferably T cell, and even more preferably a CD4⁺ and/or CD8⁺ T cell;
II) The cell expresses the immunoreceptor or CAR according to any one of claims 1 to 19;
III) The cell comprises the nucleic acid stably integrated into the genome; and/or
IV) The nucleic acid comprised in the cell is comprised in an episomal vector.

23. The nucleic acid, cell comprising the nucleic acid, immunoreceptor, or CAR, according to any one of claims 1 to 22 for use in a method of treating a cancer, an autoimmune disease, an infectious disease or a degenerative disease.

24. The immunoreceptor, CAR, nucleic acid or cell comprising the nucleic acid for use of claim 23, wherein the disease is a cancer, wherein the cancer is is a hematological cancer or a solid cancer,
optionally wherein the hematological cancer is leukemia or lymphoma, preferably acute myeloid leukemia, multiple myeloma, non-Hodgkin-lymphoma, Burkitt's lymphoma, mantle cell lymphoma, acute lymphoblastic leukemia, chronic lymphocytic leukemia, or diffuse large B cell lymphoma;
optionally wherein the solid cancer is breast cancer, colon carcinoma, lung cancer, pancreatic or prostate cancer or glioblastoma.

25. An antigen-binding protein, streptamer or aptamer which is capable of binding to an epitope comprised by a sequence consisting of at least one, preferably at least two, more preferably at least three repeats of the amino acid sequence of SEQ NO: 1, optionally wherein at least two repeats are adjacent to each other.

26. The antigen-binding protein, streptamer or aptamer of claim 25, wherein the antigen-binding protein, streptamer or aptamer is capable of binding to the immunoreceptor or CAR according to any one of claims 1 to 19.

27. The antigen-binding protein, streptamer or aptamer of claim 26, wherein the antigen-binding protein, streptamer or aptamer is capable of stimulating the immunoreceptor or CAR according to any one of claims 1 to 19.

28. The antigen-binding protein, streptamer or aptamer according to any one of claims 25 to 27, wherein the antigen-binding protein, streptamer or aptamer is an antigen-binding protein which is an antibody or fragment thereof, preferably a monoclonal antibody or fragment thereof.

29. The antigen-binding protein of any one of claims 25 to 28, wherein the antigen-binding protein comprises
a) a heavy chain variable region having at least 80%, preferably at least 90% sequence identity with the amino acid sequence of SEQ ID NO: 19, and wherein the heavy chain variable region preferably contains a CDR1 having the amino acid sequence of SEQ ID NO: 20, a CDR2 having the amino acid sequence of SEQ ID NO: 21, and a CDR3 having the amino acid sequence of SEQ ID NO: 22; and
b) a light chain variable region having at least 80%, preferably at least 90% sequence identity with the amino acid sequence of SEQ ID NO: 23, wherein the light chain variable region preferably contains a CDR1 having the amino acid sequence of SEQ ID NO: 24, a CDR2 having the amino acid sequence of SEQ ID NO: 25, and a CDR3 having the amino acid sequence of SEQ ID NO: 26.

30. Use of the antigen-binding protein, streptamer or aptamer according to any one of claims 25 to 29 for purification, detection, depletion, stimulation, expansion, or enrichment of cells expressing the immunoreceptor or CAR as defined in any one of claims 1 to 19.

31. A method, comprising the step of:
Binding an antigen-binding protein, streptamer or aptamer to cells expressing the immunoreceptor or CAR as defined in any one of claims 1 to 19, preferably wherein the binding is binding specifically to the IgG3 middle hinge repeat domain comprised in said immunoreceptor or CAR, and/or wherein the antigen-binding protein, streptamer or aptamer is an antigen-binding protein, streptamer or aptamer as defined in any one of claims 25 to 29.

32. The method of claim 31, wherein the method is a method of purification of cells expressing the immunoreceptor or CAR as defined in any one of claims 1 to 19, comprising the steps of:
A) Optionally obtaining the cells expressing the chimeric antigen receptor;
B) Incubating said cells with a primary antibody, streptamer or aptamer, wherein the primary antibody, streptamer or aptamer is said antigen-binding protein, streptamer or aptamer as defined in any one of claims 25 to 29, under conditions which allow the antibody, streptamer or aptamer to bind to the immunoreceptor or CAR expressed by the cells;
C) Separating the antibody-, streptamer- or aptamer-bound cells from the non-bound cells in order to obtain the purified cells.

33. The purification method of claim 32, wherein step C comprises incubating the cells of step B with an entity capable of binding to the antibody, streptamer or aptamer; and wherein
I) The entity is preferably a secondary antibody, more preferably labelled with a fluorescent marker; or a bead, more preferably a magnetic bead;
II) The primary antibody, streptamer or aptamer is labelled, wherein the label is preferably a tag or a fluorescent dye;
III) The separation of step C is carried out by means of MACS or FACS; and/or
IV) Wherein the separation is carried out using a Streptamer or an Aptamer.

34. The method of claim 31, wherein the method is a method of depletion of cells expressing the immunoreceptor or CAR as defined in any one of claims 1 to 19, comprising the steps of:
A) Optionally obtaining the cells expressing the immunoreceptor or CAR; and
B) Incubating said cells with an antigen-binding protein, streptamer or aptamer as defined in any one of claims 25 to 29 coupled to a cytotoxic molecule.

35. The method of claim 31, wherein the method is a method of a) stimulation and/or b) expansion of cells expressing the immunoreceptor or CAR as defined in any one of claims 1 to 19, comprising the steps of:
A) Optionally obtaining the cells expressing the immunoreceptor or CAR; and
B) Incubating said cells with an antigen-binding protein, streptamer or aptamer as defined in any one of claims 25 to 29, optionally wherein the antigen-binding protein, streptamer or aptamer is coupled to a solid phase, or wherein the antigen-binding protein, streptamer or aptamer is expressed on the surface of a cell.

36. The stimulation or expansion method of claim 35, wherein:
I) The solid phase is a tissue culture surface or a bead, preferably a magnetic bead; and/or
II) The solid phase is a scaffold consisting of polymers, preferably starch or sugar.

37. The method of claim 31, wherein the method is a method of enrichment of cells expressing the immunoreceptor or CAR as defined in any one of claims 1 to 19, comprising the steps of:
A) Stimulating and/or expanding the cells according to the method of claim 35 or 36; and
B) Purifying the cells of step A according to the method of claim 32 or 33.

38. The method or use of any one of claims 30 to 37, wherein said method or use is an in vitro method or use.

39. The method or use of any one of claims 30 to 38, wherein said method or use does not comprise a method for treatment of the human or animal body by surgery or therapy or a diagnostic method practised on the human or animal body.

40. A pharmaceutical composition, comprising the antigen-binding protein, streptamer or aptamer according to any one of claims 25 to 29 or a cell expressing a chimeric antigen receptor comprising all or part of said antigen-binding protein, streptamer or aptamer, the composition optionally further comprising a pharmaceutically acceptable carrier and/or excipient.

41. The antigen-binding protein, streptamer or aptamer according to any one of claims 25 to 29 or a cell expressing a chimeric antigen receptor comprising all or part of said antigen-binding protein, streptamer or aptamer, or the pharmaceutical composition of claim 40, for use in a therapeutic method of depletion of cells expressing the immunoreceptor or CAR as defined in any one of claims 1 to 19, comprising administering to a subject in need thereof said antigen-binding protein, streptamer or aptamer coupled to a cytotoxic molecule or cells expressing said chimeric antigen receptor comprising said all or part of said antigen-binding protein, streptamer or aptamer.

42. A kit, comprising the immunoreceptor or CAR as defined in any one of claims 1 to 19 and the antigen-binding protein, streptamer or aptamer as defined in any one of claims 25 to 27.

43. A bispecific antibody, comprising one or more IgG3 middle hinge repeat domain motifs.

44. The bispecific antibody according to claim 43, wherein the one or more IgG3 middle hinge domain repeat motifs
I) Are from a human IgG3 middle hinge; and/or
II) Consist of the amino acid sequence of SEQ ID NO: 1; and/or
III) Have reduced immunogenicity compared to repeats of an IgG1 hinge domain and/or an IgG4 hinge domain.

45. The bispecific antibody according to claim 43 or 44, wherein the bispecific antibody:
I) Does not comprise all or part of the sequence of the lower hinge domain of a human IgG3 hinge domain;
II) Comprises an amino acid sequence which has at least 80% sequence identity, preferably at least 90% sequence identity, or most preferably 100% sequence identity
with the amino acid sequence of [A-Bₙ],
wherein
A is the amino acid sequence of SEQ ID NO: 2;
B is said IgG3 middle hinge domain repeat motif, wherein said motif has the amino acid sequence of SEQ ID NO: 1; and
n is selected from the group consisting of 0, 1, 2, 3, 4, 5, 6, 7, 8, 9, 10, 11, 12, 13, 14 and 15 and is preferably an integer between 1 and 15, more preferably an integer between 1 and 10, even more preferably an integer between 1 and 5, most preferably an integer between 3 and 5;
III) Comprises the IgG3 middle hinge domain repeat motif 1, 2, 3, 4, 5, 6, 7, 8, 9 or 10 times; and/or
IV) has reduced immunogenicity compared to a second bispecific antibody which differs from the first bispecific antibody in that it does not comprise said one or more IgG3 middle hinge domain repeat motifs.

46. The bispecific antibody according to any one of claims 43 to 45, wherein the immunoreceptor comprises an amino acid sequence which has 100% sequence identity with the amino acid sequence of [A-Bₙ].

47. The bispecific antibody according to claim 45 or 46, wherein n is an integer between 1 and 10.

48. The immunoreceptor according to claim 45 or 46, wherein n is an integer between 1 and 5.

49. The immunoreceptor according to claim 45 or 46, wherein n is an integer between 3 and 5.

50. The bispecific antibody according to any one of claims 43 to 49, comprising at least two, preferably at least three IgG3 middle hinge repeat domain motifs, optionally wherein at least two of said IgG3 middle hinge repeat domain motifs are adjacent to each other.

51. The immunoreceptor, CAR, nucleic acid, cell, method, pharmaceutical composition, kit or bispecific antibody according to any one of claims 1 to 24 or 31 to 50, wherein the IgG3 middle hinge repeat domain motif is not a mouse IgG3 middle hinge repeat domain.


SEQUENCE LISTING (174 sequences)

SEQ ID NO: 1 - protein, 15 aa, Artificial Sequence
  15 aa MiH repeat sequence
EPKSCDTPPPCPRCP

SEQ ID NO: 2 - protein, 17 aa, Artificial Sequence
  Upper Hinge
ELKTPLGDTTHTCPRCP

SEQ ID NO: 3 - protein, 245 aa, Artificial Sequence
  scFv CD19_FMC63 VH_Linker_VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 4 - protein, 246 aa, Artificial Sequence
  scFv CD20_Leu16 VL_Linker_VH
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 5 - protein, 240 aa, Artificial Sequence
  scFv ROR1_2A2 VH_4GS3_VL
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAGGGGSGGGGSGGGGSDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 6 - protein, 239 aa, Artificial Sequence
  scFv ROR1_4-2 VH_4GS3_VL
QEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSGGGGSGGGGSGGGGSSYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTG

SEQ ID NO: 7 - protein, 241 aa, Artificial Sequence
  scFv ROR1_ R11 VH_4GS3_VL
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 8 - protein, 248 aa, Artificial Sequence
  scFv ROR1_ R12 VH_Linker_VL
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 9 - protein, 239 aa, Artificial Sequence
  scFv ROR2_4-1 VH_4GS3_VL
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDWTSLNIWGPGTLVTVSSGGGGSGGGGSGGGGSDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIK

SEQ ID NO: 10 - protein, 238 aa, Artificial Sequence
  scFv SLAMF7_ERCS409 VH_4GS3_VL
QSVKESEGGLFKPTDTLTLTCTVSGFSLNSYGVIWVRQAPGNGLEYIGIIGSSGNTYYASWAKSRSTITRNTRLNTVTLKMTSLTAADTATYFCARYYGDSGFDSWGPGTLVTVSSGGGGSGGGGSGGGGSAQVLTQTPSSTSVAVGGTVTIKCQASQSIGSWLSWYQQKPGQPPKLLIYGASNLASGVPSRFKGGRSGTEYSLTISGVQREDAATYYCLGASPNGWAFGAGTNVEIK

SEQ ID NO: 11 - protein, 241 aa, Artificial Sequence
  scFv SLAMF7_huLuc63 VH_4GS3_VL
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSGGGGSGGGGSDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 12 - protein, 252 aa, Artificial Sequence
  scFv FLT3_BV10 VH_4GS3_VL
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSSGGGGSGGGGSGGGGSDIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 13 - protein, 241 aa, Artificial Sequence
  scFv FLT3_4G8 VH_4GS3_VL
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSGGGGSGGGGSGGGGSDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 14 - protein, 238 aa, Artificial Sequence
  scFv Siglec-6_JML-1 VH_4GS3_VL
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 15 - protein, 240 aa, Artificial Sequence
  scFv avb3_LM609v7 VH_4GS3_VL
QVQLQESGPGLVKPSQTLSLTCTVSGASISRGGYYWSWIRQYPGKGLEWIGYIHHSGSTYYNPSLKSRVTIAIDTSKNQLSLRLTSVTAADTAVYYCARHNYGSFAYWGQGTLVTVSSGGGGSGGGGSGGGGSELVMTQSPEFQSVTPKETVTITCRASQDIGNSLHWYQQKPGQSPKLLIKYASQPVFGVPSRFRGSGSGTDFTLTISRLEPEDFAVYYCQQSNSWPHTFGQGTKLEIK

SEQ ID NO: 16 - protein, 239 aa, Artificial Sequence
  scFv avb3_LM609v11 VH_4GS3_VL
QVQLVQSGAEVRKPGSSVRVSCKASGGTFSGFAVSWVRQAPGQRFEWLGGIVASLGSTDYAQKFQDKLTITVDESTATVYMEMRNLRSDDTAVYYCARHNYGSFAYWGQGTLVTVSSGGGGSGGGGSGGGGSELVMTQSPEFQSVTPKETVTITCRASQDIGTSLHWYQQKPGQSPKLLIKYASQPVFGVPSRFRGSGSGTDFTLTIYSLEAEDFAVYYCQQSNSWPHTFGQGTKLEIK

SEQ ID NO: 17 - protein, 246 aa, Artificial Sequence
  scFv BCMA_BCMA30 VH_4GS3_VL
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 18 - protein, 246 aa, Artificial Sequence
  scFv BCMA_BCMA50 VH_4GS3_VL
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 19 - protein, 118 aa, Artificial Sequence
  anti MiH repeats heavy chain variable region
QVQLLESGPGLVAPSQTLSITCTVSGFSFTNYDLHWVRQPPGKGLEWLGVIWAVGSTNYNSALMSRLSISKDNSKSQVFLKMNSLQTDDTAIYYCAREEDYRYGMDYWGQGTSVTVSS

SEQ ID NO: 20 - protein, 7 aa, Artificial Sequence
  anti MiH repeats heavy CDR1
GFSFTNY

SEQ ID NO: 21 - protein, 5 aa, Artificial Sequence
  anti MiH repeats heavy CDR2
WAVGS

SEQ ID NO: 22 - protein, 10 aa, Artificial Sequence
  anti MiH repeats heavy CDR3
EEDYRYGMDY

SEQ ID NO: 23 - protein, 112 aa, Artificial Sequence
  anti MiH repeats light chain variable region
ELVMTQSPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 24 - protein, 16 aa, Artificial Sequence
  anti MiH repeats light CDR1
RSSQSLVHSNGNTYLH

SEQ ID NO: 25 - protein, 7 aa, Artificial Sequence
  anti MiH repeats light CDR2
KVSNRFS

SEQ ID NO: 26 - protein, 9 aa, Artificial Sequence
  anti MiH repeats light CDR3
SQSTHVPYT

SEQ ID NO: 27 - protein, 107 aa, Artificial Sequence
  scFv CD19_FMC63 VH
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 28 - protein, 120 aa, Artificial Sequence
  scFv CD19_FMC63 VL
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 29 - protein, 106 aa, Artificial Sequence
  scFv CD20_Leu16 VL
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 30 - protein, 122 aa, Artificial Sequence
  scFv CD20_Leu16 VH
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 31 - protein, 118 aa, Artificial Sequence
  scFv ROR1_2A2 VH
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 32 - protein, 107 aa, Artificial Sequence
  scFv ROR1_2A2 VL
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 33 - protein, 117 aa, Artificial Sequence
  scFv ROR1_4-2 VH
QEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSS

SEQ ID NO: 34 - protein, 107 aa, Artificial Sequence
  scFv ROR1_4-2 VL
SYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTG

SEQ ID NO: 35 - protein, 116 aa, Artificial Sequence
  scFv ROR1_ R11 VH
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISS

SEQ ID NO: 36 - protein, 110 aa, Artificial Sequence
  scFv ROR1_ R11 VL
ELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 37 - protein, 121 aa, Artificial Sequence
  scFv ROR1_ R12 VH
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 38 - protein, 112 aa, Artificial Sequence
  scFv ROR1_ R12 VL
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 39 - protein, 114 aa, Artificial Sequence
  scFv ROR2_4-1 VH
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDWTSLNIWGPGTLVTVSS

SEQ ID NO: 40 - protein, 110 aa, Artificial Sequence
  scFv ROR2_4-1 VL
DPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIK

SEQ ID NO: 41 - protein, 116 aa, Artificial Sequence
  scFv SLAMF7_ERCS409 VH
QSVKESEGGLFKPTDTLTLTCTVSGFSLNSYGVIWVRQAPGNGLEYIGIIGSSGNTYYASWAKSRSTITRNTRLNTVTLKMTSLTAADTATYFCARYYGDSGFDSWGPGTLVTVSS

SEQ ID NO: 42 - protein, 107 aa, Artificial Sequence
  scFv SLAMF7_ERCS409 VL
AQVLTQTPSSTSVAVGGTVTIKCQASQSIGSWLSWYQQKPGQPPKLLIYGASNLASGVPSRFKGGRSGTEYSLTISGVQREDAATYYCLGASPNGWAFGAGTNVEIK

SEQ ID NO: 43 - protein, 119 aa, Artificial Sequence
  scFv SLAMF7_huLuc63 VH
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 44 - protein, 107 aa, Artificial Sequence
  scFv SLAMF7_huLuc63 VL
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 45 - protein, 123 aa, Artificial Sequence
  scFv FLT3_BV10 VH
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSS

SEQ ID NO: 46 - protein, 114 aa, Artificial Sequence
  scFv FLT3_BV10 VL
DIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 47 - protein, 118 aa, Artificial Sequence
  scFv FLT3_4G8 VH
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 48 - protein, 108 aa, Artificial Sequence
  scFv FLT3_4G8 VL
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 49 - protein, 116 aa, Artificial Sequence
  scFv Siglec-6_JML-1 VH
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSS

SEQ ID NO: 50 - protein, 107 aa, Artificial Sequence
  scFv Siglec-6_JML-1 VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 51 - protein, 118 aa, Artificial Sequence
  scFv avb3_LM609v7 VH
QVQLQESGPGLVKPSQTLSLTCTVSGASISRGGYYWSWIRQYPGKGLEWIGYIHHSGSTYYNPSLKSRVTIAIDTSKNQLSLRLTSVTAADTAVYYCARHNYGSFAYWGQGTLVTVSS

SEQ ID NO: 52 - protein, 107 aa, Artificial Sequence
  scFv avb3_LM609v7 VL
ELVMTQSPEFQSVTPKETVTITCRASQDIGNSLHWYQQKPGQSPKLLIKYASQPVFGVPSRFRGSGSGTDFTLTISRLEPEDFAVYYCQQSNSWPHTFGQGTKLEIK

SEQ ID NO: 53 - protein, 117 aa, Artificial Sequence
  scFv avb3_LM609v11 VH
QVQLVQSGAEVRKPGSSVRVSCKASGGTFSGFAVSWVRQAPGQRFEWLGGIVASLGSTDYAQKFQDKLTITVDESTATVYMEMRNLRSDDTAVYYCARHNYGSFAYWGQGTLVTVSS

SEQ ID NO: 54 - protein, 107 aa, Artificial Sequence
  scFv avb3_LM609v11 VL
ELVMTQSPEFQSVTPKETVTITCRASQDIGTSLHWYQQKPGQSPKLLIKYASQPVFGVPSRFRGSGSGTDFTLTIYSLEAEDFAVYYCQQSNSWPHTFGQGTKLEIK

SEQ ID NO: 55 - protein, 119 aa, Artificial Sequence
  scFv BCMA_BCMA30 VH
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 56 - protein, 112 aa, Artificial Sequence
  scFv BCMA_BCMA30 VL
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 57 - protein, 119 aa, Artificial Sequence
  scFv BCMA_BCMA50 VH
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 58 - protein, 112 aa, Artificial Sequence
  scFv BCMA_BCMA50 VL
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 59 - protein, 5 aa, Artificial Sequence
  CPRCP
CPRCP

SEQ ID NO: 60 - protein, 8 aa, Artificial Sequence
  IgG3 lower hinge
APELLGGP

SEQ ID NO: 61 - DNA, 2149 bp, Artificial Sequence
  Lentiviral vector backbone 5
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctcgtgacatctctgctgctgtgcgagctgccccaccccgcctttctgctgatcccc

SEQ ID NO: 62 - DNA, 5916 bp, Artificial Sequence
  Lentiviral vector backbone 3
ctcgagggcggaggcgaaggcagaggcagcctgctgacatgtggcgacgtggaagagaacccaggccccagaatgctgctgctcgtgaccagcctgctgctgtgtgaactgcctcatcctgcttttctgctgattcctcggaaagtgtgcaacggcatcggcatcggagagttcaaggactccctgagcatcaacgccaccaacatcaagcacttcaagaactgcaccagcatcagcggcgacctgcacatcctgcctgtggcctttagaggcgacagcttcacccacacaccccccctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 63 - DNA, 1050 bp, Artificial Sequence
  Sleeping Beauty vector backbone 5
caaggcgattaagttgggtaacgccagggttttcccagtcacgacgttgtaaaacgacggccagtgagcgcgcgtaatacgactcactatagggcgaattggagctcgggtccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttgatatccatggaattcggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctcgtgacatctctgctgctgtgcgagctgccccaccccgcctttctgctgatcccc

SEQ ID NO: 64 - DNA, 4270 bp, Artificial Sequence
  Sleeping Beauty vector backbone 3
ctcgagggcggaggcgaaggcagaggcagcctgctgacatgtggcgacgtggaagagaacccaggccccagaatgctgctgctcgtgaccagcctgctgctgtgtgaactgcctcatcctgcttttctgctgattcctcggaaagtgtgcaacggcatcggcatcggagagttcaaggactccctgagcatcaacgccaccaacatcaagcacttcaagaactgcaccagcatcagcggcgacctgcacatcctgcctgtggcctttagaggcgacagcttcacccacacaccccccctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagccaagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctagggatcggcctcttcatgtgagcggccgctctagatggccagatctagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtataggggtcctctagctagagtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 65 - protein, 28 aa, Artificial Sequence
  CAR transmembrane domain
MFWVLVVVGGVLACYSLLVTVAFIIFWV

SEQ ID NO: 66 - protein, 42 aa, Artificial Sequence
  CAR 4-1BB domain
KRGRKKLLYIFKQPFMRPVQTTQEEDGCSCRFPEEEEGGCEL

SEQ ID NO: 67 - protein, 112 aa, Artificial Sequence
  CAR CD3 zeta domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 533 aa, Artificial Sequence
  CD19 CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - DNA, 6919 bp, Artificial Sequence
  CD19 CAR SB vector
caaggcgattaagttgggtaacgccagggttttcccagtcacgacgttgtaaaacgacggccagtgagcgcgcgtaatacgactcactatagggcgaattggagctcgggtccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttgatatccatggaattcggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatattgctacctacttctgtcagcaaggcaacaccctgccctacaccttcggcggaggcaccaagctggaaatcaccgaactgaaaaccccgcttggcgacaccacccacacctgtcctagatgtcccgaacccaagagctgcgatacccccccaccttgccctagatgccccgagcctaagtcctgcgacacccctcctccatgccctcggtgtcctgagcctaagagctgtgacacaccacccccctgccccagatgtccagagccaaaatcttgtgatacccctcccccctgtccccgctgcccagaacccaagtcctgtgatactccacctccttgtccacggtgccccgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtctagcgaactgaaaacccccctgggcgacaccacccacacctgtcctagatgtccggaacccaagagctgcgatacccccccaccttgccccagatgccccatgttttgggtgctggtggtcgtgggcggagtgctggcctgttacagcctgctcgtgaccgtggccttcatcatcttttgggtcaagcggggcagaaagaagctgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctatcagcagggccagaaccagctatacaacgagctgaacctgggcagacgggaagagtacgacgtgctggacaagagaagaggccgggaccctgagatgggcggaaagcccagaagaaagaacccccaggaaggcctgtataacgaactgcagaaagacaagatggccgaggcctacagcgagatcggaatgaagggcgagcggcggagaggcaagggccacgatggactgtatcagggcctgagcaccgccaccaaggacacctatgacgccctgcacatgcaggccctgccccctagactcgagggcggaggcgaaggcagaggcagcctgctgacatgtggcgacgtggaagagaacccaggccccagaatgctgctgctcgtgaccagcctgctgctgtgtgaactgcctcatcctgcttttctgctgattcctcggaaagtgtgcaacggcatcggcatcggagagttcaaggactccctgagcatcaacgccaccaacatcaagcacttcaagaactgcaccagcatcagcggcgacctgcacatcctgcctgtggcctttagaggcgacagcttcacccacacaccccccctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagccaagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctagggatcggcctcttcatgtgagcggccgctctagatggccagatctagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtataggggtcctctagctagagtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 70 - DNA, 9664 bp, Artificial Sequence
  CD19 CAR LV vector
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatattgctacctacttctgtcagcaaggcaacaccctgccctacaccttcggcggaggcaccaagctggaaatcaccgaactgaaaaccccgcttggcgacaccacccacacctgtcctagatgtcccgaacccaagagctgcgatacccccccaccttgccctagatgccccgagcctaagtcctgcgacacccctcctccatgccctcggtgtcctgagcctaagagctgtgacacaccacccccctgccccagatgtccagagccaaaatcttgtgatacccctcccccctgtccccgctgcccagaacccaagtcctgtgatactccacctccttgtccacggtgccccgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtctagcgaactgaaaacccccctgggcgacaccacccacacctgtcctagatgtccggaacccaagagctgcgacacccctccaccttgcccaagatgccccatgttctgggtgctggtggtcgtgggcggagtgctggcctgttatagcctgctcgtgaccgtggccttcatcatcttttgggtcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctatcagcagggccagaaccagctgtacaacgagctgaacctgggcagacgggaagagtacgacgtgctggacaagagaagaggccgggaccctgagatgggcggaaagcccagaagaaagaacccccaggaaggcctgtataacgaactgcagaaagacaagatggccgaggcctacagcgagatcggaatgaagggcgagcggcggagaggcaagggccacgatggactgtatcagggcctgagcaccgccaccaaggacacctatgacgccctgcacatgcaggccctgccccctagactcgagggcggaggcgaaggcagaggcagcctgctgacatgtggcgacgtggaagagaacccaggccccagaatgctgctgctcgtgaccagcctgctgctgtgtgaactgcctcatcctgcttttctgctgattcctcggaaagtgtgcaacggcatcggcatcggagagttcaaggactccctgagcatcaacgccaccaacatcaagcacttcaagaactgcaccagcatcagcggcgacctgcacatcctgcctgtggcctttagaggcgacagcttcacccacacaccccccctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 71 - protein, 319 aa, Artificial Sequence
  scFv CD19_FMC63 VH_MiH5_VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 72 - protein, 320 aa, Artificial Sequence
  scFv CD20_Leu16 VL_MiH5_VH
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 73 - protein, 317 aa, Artificial Sequence
  scFv ROR1_2A2 VH_MiH5_VL
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 74 - protein, 316 aa, Artificial Sequence
  scFv ROR1_4-2 VH_MiH5_VL
QEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPSYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTG

SEQ ID NO: 75 - protein, 318 aa, Artificial Sequence
  scFv ROR1_ R11 VH_MiH5_VL
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 76 - protein, 325 aa, Artificial Sequence
  scFv ROR1_ R12 VH_MiH5_VL
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 77 - protein, 316 aa, Artificial Sequence
  scFv ROR2_4-1 VH_MiH5_VL
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDWTSLNIWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIK

SEQ ID NO: 78 - protein, 315 aa, Artificial Sequence
  scFv SLAMF7_ERCS409 VH_MiH5_VL
QSVKESEGGLFKPTDTLTLTCTVSGFSLNSYGVIWVRQAPGNGLEYIGIIGSSGNTYYASWAKSRSTITRNTRLNTVTLKMTSLTAADTATYFCARYYGDSGFDSWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPAQVLTQTPSSTSVAVGGTVTIKCQASQSIGSWLSWYQQKPGQPPKLLIYGASNLASGVPSRFKGGRSGTEYSLTISGVQREDAATYYCLGASPNGWAFGAGTNVEIK

SEQ ID NO: 79 - protein, 318 aa, Artificial Sequence
  scFv SLAMF7_huLuc63 VH_MiH5_VL
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 80 - protein, 329 aa, Artificial Sequence
  scFv FLT3_BV10 VH_MiH5_VL
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 81 - protein, 318 aa, Artificial Sequence
  scFv FLT3_4G8 VH_MiH5_VL
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 82 - protein, 315 aa, Artificial Sequence
  scFv Siglec-6_JML-1 VH_MiH5_VL
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 83 - protein, 317 aa, Artificial Sequence
  scFv avb3_LM609v7 VH_MiH5_VL
QVQLQESGPGLVKPSQTLSLTCTVSGASISRGGYYWSWIRQYPGKGLEWIGYIHHSGSTYYNPSLKSRVTIAIDTSKNQLSLRLTSVTAADTAVYYCARHNYGSFAYWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVMTQSPEFQSVTPKETVTITCRASQDIGNSLHWYQQKPGQSPKLLIKYASQPVFGVPSRFRGSGSGTDFTLTISRLEPEDFAVYYCQQSNSWPHTFGQGTKLEIK

SEQ ID NO: 84 - protein, 316 aa, Artificial Sequence
  scFv avb3_LM609v11 VH_MiH5_VL
QVQLVQSGAEVRKPGSSVRVSCKASGGTFSGFAVSWVRQAPGQRFEWLGGIVASLGSTDYAQKFQDKLTITVDESTATVYMEMRNLRSDDTAVYYCARHNYGSFAYWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVMTQSPEFQSVTPKETVTITCRASQDIGTSLHWYQQKPGQSPKLLIKYASQPVFGVPSRFRGSGSGTDFTLTIYSLEAEDFAVYYCQQSNSWPHTFGQGTKLEIK

SEQ ID NO: 85 - protein, 323 aa, Artificial Sequence
  scFv BCMA_BCMA30 VH_MiH5_VL
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 86 - protein, 323 aa, Artificial Sequence
  scFv BCMA_BCMA50 VH_MiH5_VL
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 87 - protein, 247 aa, Artificial Sequence
  scFv ROR1_ huR12 VH_Linker_VL
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSSGGGGSGGGGSGGGGSQLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGGYVFGGGTQLTVG

SEQ ID NO: 88 - protein, 324 aa, Artificial Sequence
  scFv ROR1_ huR12 VH_MiH5_VL
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPQLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGGYVFGGGTQLTVG

SEQ ID NO: 89 - protein, 248 aa, Artificial Sequence
  scFv ROR1_ R12/V16 VH_Linker_VL
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGAESRGGYVFGGGTQLTVTG

SEQ ID NO: 90 - protein, 325 aa, Artificial Sequence
  scFv ROR1_ R12/V16 VH_MiH5_VL
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVLTQSPSVSAALGSPAKITCTLSSAHKTDTIDWYQQLQGEAPRYLMQVQSDGSYTKRPGVPDRFSGSSSGADRYLIIPSVQADDEADYYCGAESRGGYVFGGGTQLTVTG

SEQ ID NO: 91 - protein, 248 aa, Artificial Sequence
  scFv ROR1_ R12/V20 VH_Linker_VL
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYGEDLGLFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 92 - protein, 325 aa, Artificial Sequence
  scFv ROR1_ R12/V20 VH_MiH5_VL
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYGEDLGLFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 93 - protein, 248 aa, Artificial Sequence
  scFv ROR1_ R12/V16-20 VH_Linker_VL
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYGEDLGLFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGAESRGGYVFGGGTQLTVTG

SEQ ID NO: 94 - protein, 325 aa, Artificial Sequence
  scFv ROR1_ R12/V16-20 VH_MiH5_VL
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYGEDLGLFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVLTQSPSVSAALGSPAKITCTLSSAHKTDTIDWYQQLQGEAPRYLMQVQSDGSYTKRPGVPDRFSGSSSGADRYLIIPSVQADDEADYYCGAESRGGYVFGGGTQLTVTG

SEQ ID NO: 95 - protein, 247 aa, Artificial Sequence
  scFv ROR1_ huR12/V16 VH_Linker_VL
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSSGGGGSGGGGSGGGGSQLVLTQSPSVSAALGSSAKITCTLSSAHKTDTIDWYQQLAGQAPRYLMYVQSDGSYEKRSGVPDRFSGSSSGADRYLIISSVQADDEADYYCGAESRGGYVFGGGTQLTVG

SEQ ID NO: 96 - protein, 323 aa, Artificial Sequence
  scFv ROR1_ huR12/V16 VH_MiH5_VL
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPQLVLTQSPSVSAALGSSAKITCTLSSAHKTDTIDWYQQLAGQAPRYLMYVQSDGSYEKRSGVPDRFSGSSSGADRYLIISSVQADDEADYYCGAESRGYVFGGGTQLTVG

SEQ ID NO: 97 - protein, 247 aa, Artificial Sequence
  scFv ROR1_ huR12/V20 VH_Linker_VL
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYGEDLGLFNIWGQGTLVTVSSGGGGSGGGGSGGGGSQLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGGYVFGGGTQLTVG

SEQ ID NO: 98 - protein, 323 aa, Artificial Sequence
  scFv ROR1_ huR12/V20 VH_MiH5_VL
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYGEDLGLFNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPQLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGYVFGGGTQLTVG

SEQ ID NO: 99 - protein, 247 aa, Artificial Sequence
  scFv ROR1_ huR12/V16-20 VH_Linker_VL
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYGEDLGLFNIWGQGTLVTVSSGGGGSGGGGSGGGGSQLVLTQSPSVSAALGSSAKITCTLSSAHKTDTIDWYQQLAGQAPRYLMYVQSDGSYEKRSGVPDRFSGSSSGADRYLIISSVQADDEADYYCGAESRGGYVFGGGTQLTVG

SEQ ID NO: 100 - protein, 323 aa, Artificial Sequence
  scFv ROR1_ huR12/V16-20 VH_MiH5_VL
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYGEDLGLFNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPQLVLTQSPSVSAALGSSAKITCTLSSAHKTDTIDWYQQLAGQAPRYLMYVQSDGSYEKRSGVPDRFSGSSSGADRYLIISSVQADDEADYYCGAESRGYVFGGGTQLTVG

SEQ ID NO: 101 - protein, 240 aa, Artificial Sequence
  scFv ROR2_X3.12 VH_4GS3_VL
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDDRWSLNIWGPGTLVTVSSGGGGSGGGGSGGGGSDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIK

SEQ ID NO: 102 - protein, 317 aa, Artificial Sequence
  scFv ROR2_X3.12 VH_MiH5_VL
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDDRWSLNIWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIK

SEQ ID NO: 103 - protein, 239 aa, Artificial Sequence
  scFv ROR2_XBR2-401-DM VH_4GS3_VL
QSVKESEGGLFKPTDTLTLTCTVSGFSLSSYGVTWVRQAPGSGLEWIGYINGRGNTYYASWAKSRSTITRNTNENTVTLKMTSLTAADTATYFCARDWTSLNIWGPGTLVTVSSGGGGSGGGGSGGGGSDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIK

SEQ ID NO: 104 - protein, 316 aa, Artificial Sequence
  scFv ROR2_XBR2-401-DM VH_MiH5_VL
QSVKESEGGLFKPTDTLTLTCTVSGFSLSSYGVTWVRQAPGSGLEWIGYINGRGNTYYASWAKSRSTITRNTNENTVTLKMTSLTAADTATYFCARDWTSLNIWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIK

SEQ ID NO: 105 - protein, 241 aa, Artificial Sequence
  scFv ROR2_huX3.12.5 VH_4GS3_VL
EVQLVESGGGLIQPGGSLRLSCAASGFTVSSYGVTWVRQAPGKGLEWVSYINTAGNTYYASWAKSRFTISRDNSKNTLYLQMNSLRAEDTAVYYCARDDRWSLNIWGQGTLVTVSSGGGGSGGGGSGGGGSDPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIK

SEQ ID NO: 106 - protein, 318 aa, Artificial Sequence
  scFv ROR2_huX3.12.5 VH_MiH5_VL
EVQLVESGGGLIQPGGSLRLSCAASGFTVSSYGVTWVRQAPGKGLEWVSYINTAGNTYYASWAKSRFTISRDNSKNTLYLQMNSLRAEDTAVYYCARDDRWSLNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIK

SEQ ID NO: 107 - protein, 241 aa, Artificial Sequence
  scFv ROR2_huX3.12.6 VH_4GS3_VL
EVQLVESGGGLVQPGGSLRLSCAASGFTFSSYGVTWVRQAPGKGLEWVSYINTAGNTYYASWAKSRFTISRDNAKNSLYLQMNSLRAEDTAVYYCARDDRWSLNIWGQGTLVTVSSGGGGSGGGGSGGGGSDPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIK

SEQ ID NO: 108 - protein, 318 aa, Artificial Sequence
  scFv ROR2_huX3.12.6 VH_MiH5_VL
EVQLVESGGGLVQPGGSLRLSCAASGFTFSSYGVTWVRQAPGKGLEWVSYINTAGNTYYASWAKSRFTISRDNAKNSLYLQMNSLRAEDTAVYYCARDDRWSLNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIK

SEQ ID NO: 109 - protein, 181 aa, Artificial Sequence
  CD28tm+CD28/zeta
MFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 182 aa, Artificial Sequence
  CD28tm+4-1BB/zeta
MFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 223 aa, Artificial Sequence
  CD28tm+CD28/4-1BB/zeta
MFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 223 aa, Artificial Sequence
  CD28tm+4-1BB/CD28/zeta
MFWVLVVVGGVLACYSLLVTVAFIIFWVKRGRKKLLYIFKQPFMRPVQTTQEEDGCSCRFPEEEEGGCELRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 140 aa, Artificial Sequence
  CD28tm+zeta
MFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 133 aa, Artificial Sequence
  ICOStm+zeta
FWLPIGCAAFVVVCILGCILIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 175 aa, Artificial Sequence
  OX40tm+OX40/zeta
VAAILGLGLVLGLLGPLAILLALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 518 aa, Artificial Sequence
  scFv CD19_FMC63 VH_MiH5_VL_MiH0_ CD28tm+4-1BB/zeta
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSELKTPLGDTTHTCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 533 aa, Artificial Sequence; identical to SEQ ID NO: 68
  scFv CD19_FMC63 VH_MiH5_VL_MiH1_ CD28tm+4-1BB/zeta

SEQ ID NO: 118 - protein, 564 aa, Artificial Sequence
  scFv CD20_Leu16 VL_MiH5_VH_MiH3_ CD28tm+4-1BB/zeta
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSELKTPLGDTTHTCPRCPEPKSCDTPPPCPRCPEPKSCDTPPPCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 531 aa, Artificial Sequence
  scFv ROR1_2A2 VH_MiH5_VL_MiH1_ CD28tm+4-1BB/zeta
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 530 aa, Artificial Sequence
  scFv ROR1_4-2 VH_MiH5_VL_MiH1_ CD28tm+4-1BB/zeta
QEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPSYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 562 aa, Artificial Sequence
  scFv ROR1_R11 VH_MiH5_VL_MiH3_ CD28tm+4-1BB/zeta
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVKELKTPLGDTTHTCPRCPEPKSCDTPPPCPRCPEPKSCDTPPPCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 539 aa, Artificial Sequence
  scFv ROR1_R12 VH_MiH5_VL_MiH1_ CD28tm+4-1BB/zeta
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 538 aa, Artificial Sequence
  scFv ROR1_huR12 VH_MiH5_VL_MiH1_ CD28tm+4-1BB/zeta
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPQLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGGYVFGGGTQLTVGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 539 aa, Artificial Sequence
  scFv ROR1_R12/V16 VH_MiH5_VL_MiH1_ CD28tm+4-1BB/ zeta
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVLTQSPSVSAALGSPAKITCTLSSAHKTDTIDWYQQLQGEAPRYLMQVQSDGSYTKRPGVPDRFSGSSSGADRYLIIPSVQADDEADYYCGAESRGGYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 539 aa, Artificial Sequence
  scFv ROR1_R12/V20 VH_MiH5_VL_MiH1_ CD28tm+4-1BB/ zeta
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYGEDLGLFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 539 aa, Artificial Sequence
  scFv ROR1_R12/V16-20 VH_MiH5_VL_MiH1_ CD28tm+4-1BB/zeta
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYGEDLGLFNIWGPGTLVTISSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVLTQSPSVSAALGSPAKITCTLSSAHKTDTIDWYQQLQGEAPRYLMQVQSDGSYTKRPGVPDRFSGSSSGADRYLIIPSVQADDEADYYCGAESRGGYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 537 aa, Artificial Sequence
  scFv ROR1_huR12/V16 VH_MiH5_VL_MiH1_ CD28tm+4-1BB/zeta
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPQLVLTQSPSVSAALGSSAKITCTLSSAHKTDTIDWYQQLAGQAPRYLMYVQSDGSYEKRSGVPDRFSGSSSGADRYLIISSVQADDEADYYCGAESRGYVFGGGTQLTVGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 537 aa, Artificial Sequence
  scFv ROR1_huR12/V20 VH_MiH5_VL_MiH1_ CD28tm+4-1BB/zeta
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYGEDLGLFNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPQLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGYVFGGGTQLTVGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 537 aa, Artificial Sequence
  scFv ROR1_huR12/V16-20 VH_MiH5_VL_MiH1_ CD28tm+4-1BB/zeta
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYGEDLGLFNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPQLVLTQSPSVSAALGSSAKITCTLSSAHKTDTIDWYQQLAGQAPRYLMYVQSDGSYEKRSGVPDRFSGSSSGADRYLIISSVQADDEADYYCGAESRGYVFGGGTQLTVGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 530 aa, Artificial Sequence
  scFv ROR2_4-2 VH_MiH5_VL_MiH1_ CD28tm+4-1BB/zeta
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDWTSLNIWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 531 aa, Artificial Sequence
  scFv ROR2_X3.12 VH_MiH5_VL_MiH1_ CD28tm+4-1BB/zeta
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDDRWSLNIWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 530 aa, Artificial Sequence
  scFv ROR2_ XBR2-401-DM VH_MiH5_VL_MiH1_ CD28tm+4-1BB/zeta
QSVKESEGGLFKPTDTLTLTCTVSGFSLSSYGVTWVRQAPGSGLEWIGYINGRGNTYYASWAKSRSTITRNTNENTVTLKMTSLTAADTATYFCARDWTSLNIWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 532 aa, Artificial Sequence
  scFv ROR2_huX3.12.5 VH_MiH5_VL_MiH1_ CD28tm+4-1BB/zeta
EVQLVESGGGLIQPGGSLRLSCAASGFTVSSYGVTWVRQAPGKGLEWVSYINTAGNTYYASWAKSRFTISRDNSKNTLYLQMNSLRAEDTAVYYCARDDRWSLNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 532 aa, Artificial Sequence
  scFv ROR2_huX3.12.6 VH_MiH5_VL_MiH1_ CD28tm+4-1BB/zeta
EVQLVESGGGLVQPGGSLRLSCAASGFTFSSYGVTWVRQAPGKGLEWVSYINTAGNTYYASWAKSRFTISRDNAKNSLYLQMNSLRAEDTAVYYCARDDRWSLNIWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 529 aa, Artificial Sequence
  scFv SLAMF7_ERCS409 VH_MiH5_VL_MiH1_ CD28tm+4-1BB/zeta
QSVKESEGGLFKPTDTLTLTCTVSGFSLNSYGVIWVRQAPGNGLEYIGIIGSSGNTYYASWAKSRSTITRNTRLNTVTLKMTSLTAADTATYFCARYYGDSGFDSWGPGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPAQVLTQTPSSTSVAVGGTVTIKCQASQSIGSWLSWYQQKPGQPPKLLIYGASNLASGVPSRFKGGRSGTEYSLTISGVQREDAATYYCLGASPNGWAFGAGTNVEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 532 aa, Artificial Sequence
  scFv SLAMF7_huLuc63 VH_MiH5_VL_MiH1_ CD28tm+4-1BB/zeta
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 543 aa, Artificial Sequence
  scFv FLT3_BV10 VH_MiH5_VL_MiH1_ CD28tm+4-1BB/zeta
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKRELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 532 aa, Artificial Sequence
  scFv FLT3_4G8 VH_MiH5_VL_MiH1_ CD28tm+4-1BB/zeta
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKRELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 529 aa, Artificial Sequence
  scFv Siglec-6_JML-1 VH_MiH5_VL_MiH1_ CD28tm+4-1BB/zeta
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 531 aa, Artificial Sequence
  scFv avb3_LM609v7 VH_MiH5_VL_MiH1_ CD28tm+4-1BB/zeta
QVQLQESGPGLVKPSQTLSLTCTVSGASISRGGYYWSWIRQYPGKGLEWIGYIHHSGSTYYNPSLKSRVTIAIDTSKNQLSLRLTSVTAADTAVYYCARHNYGSFAYWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVMTQSPEFQSVTPKETVTITCRASQDIGNSLHWYQQKPGQSPKLLIKYASQPVFGVPSRFRGSGSGTDFTLTISRLEPEDFAVYYCQQSNSWPHTFGQGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 530 aa, Artificial Sequence
  scFv avb3_LM609v11 VH_MiH5_VL_MiH1_ CD28tm+4-1BB/zeta
QVQLVQSGAEVRKPGSSVRVSCKASGGTFSGFAVSWVRQAPGQRFEWLGGIVASLGSTDYAQKFQDKLTITVDESTATVYMEMRNLRSDDTAVYYCARHNYGSFAYWGQGTLVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPELVMTQSPEFQSVTPKETVTITCRASQDIGTSLHWYQQKPGQSPKLLIKYASQPVFGVPSRFRGSGSGTDFTLTIYSLEAEDFAVYYCQQSNSWPHTFGQGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 537 aa, Artificial Sequence
  scFv BCMA_BCMA30 VH_MiH5_VL_MiH1_ CD28tm+4-1BB/ zeta
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 537 aa, Artificial Sequence
  scFv BCMA_BCMA50 VH_MiH5_VL_MiH1_ CD28tm+4-1BB/ zeta
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSELKTPLGDTTHTCPRCPEPKSCDTPPPCPRCPEPKSCDTPPPCPRCPEPKSCDTPPPCPRCPEPKSCDTPPPCPRCPEPKSCDTPPPCPRCPDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 444 aa, Artificial Sequence
  scFv CD19_FMC63 VH_Linker_VL_MiH0_ CD28tm+4-1BB/ zeta
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSELKTPLGDTTHTCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 459 aa, Artificial Sequence
  scFv CD19_FMC63 VH_Linker_VL_MiH1_ CD28tm+4-1BB/ zeta
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 490 aa, Artificial Sequence
  scFv CD20_Leu16 VL_Linker_VH_MiH3_ CD28tm+4-1BB/ zeta
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSELKTPLGDTTHTCPRCPEPKSCDTPPPCPRCPEPKSCDTPPPCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 454 aa, Artificial Sequence
  scFv ROR1_2A2 VH_4GS3_VL_MiH1_ CD28tm+4-1BB/zeta
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAGGGGSGGGGSGGGGSDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 453 aa, Artificial Sequence
  scFv ROR1_4-2 VH_4GS3_VL_MiH1_ CD28tm+4-1BB/zeta
QEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSGGGGSGGGGSGGGGSSYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 485 aa, Artificial Sequence
  scFv ROR1_R11 VH_4GS3_VL_MiH3_ CD28tm+4-1BB/zeta
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVKELKTPLGDTTHTCPRCPEPKSCDTPPPCPRCPEPKSCDTPPPCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 462 aa, Artificial Sequence
  scFv ROR1_R12 VH_Linker_VL_MiH1_ CD28tm+4-1BB/ zeta
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 461 aa, Artificial Sequence
  scFv ROR1_huR12 VH_Linker_VL_MiH1_ CD28tm+4-1BB/ zeta
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSSGGGGSGGGGSGGGGSQLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGGYVFGGGTQLTVGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 462 aa, Artificial Sequence
  scFv ROR1_R12/V16 VH_Linker_VL_MiH1_ CD28tm+4-1BB/ zeta
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGAESRGGYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 462 aa, Artificial Sequence
  scFv ROR1_R12/V20 VH_Linker_VL_MiH1_ CD28tm+4-1BB/ zeta
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYGEDLGLFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 462 aa, Artificial Sequence
  scFv ROR1_R12/V16-20 VH_Linker_VL_MiH1_ CD28tm+4-1BB/zeta
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYGEDLGLFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGAESRGGYVFGGGTQLTVTGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 460 aa, Artificial Sequence
  scFv ROR1_huR12/V16 VH_Linker_VL_MiH1_ CD28tm+4-1BB/zeta
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSSGGGGSGGGGSGGGGSQLVLTQSPSVSAALGSSAKITCTLSSAHKTDTIDWYQQLAGQAPRYLMYVQSDGSYEKRSGVPDRFSGSSSGADRYLIISSVQADDEADYYCGAESRGYVFGGGTQLTVGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 460 aa, Artificial Sequence
  scFv ROR1_huR12/V20 VH_Linker_VL_MiH1_ CD28tm+4-1BB/zeta
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYGEDLGLFNIWGQGTLVTVSSGGGGSGGGGSGGGGSQLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGYVFGGGTQLTVGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 460 aa, Artificial Sequence
  scFv ROR1_huR12/V16-20 VH_Linker_VL_MiH1_ CD28tm+4-1BB/zeta
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYGEDLGLFNIWGQGTLVTVSSGGGGSGGGGSGGGGSQLVLTQSPSVSAALGSSAKITCTLSSAHKTDTIDWYQQLAGQAPRYLMYVQSDGSYEKRSGVPDRFSGSSSGADRYLIISSVQADDEADYYCGAESRGYVFGGGTQLTVGELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 453 aa, Artificial Sequence
  scFv ROR2_4-2 VH_4GS3_VL_MiH1_ CD28tm+4-1BB/zeta
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDWTSLNIWGPGTLVTVSSGGGGSGGGGSGGGGSDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 454 aa, Artificial Sequence
  scFv ROR2_X3.12 VH_4GS3_VL_MiH1_ CD28tm+4-1BB/zeta
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDDRWSLNIWGPGTLVTVSSGGGGSGGGGSGGGGSDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 453 aa, Artificial Sequence
  scFv ROR2_ XBR2-401-DM VH_4GS3_VL_MiH1_ CD28tm+4-1BB/zeta
QSVKESEGGLFKPTDTLTLTCTVSGFSLSSYGVTWVRQAPGSGLEWIGYINGRGNTYYASWAKSRSTITRNTNENTVTLKMTSLTAADTATYFCARDWTSLNIWGPGTLVTVSSGGGGSGGGGSGGGGSDPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 455 aa, Artificial Sequence
  scFv ROR2_huX3.12.5 VH_4GS3_VL_MiH1_ CD28tm+4-1BB/zeta
EVQLVESGGGLIQPGGSLRLSCAASGFTVSSYGVTWVRQAPGKGLEWVSYINTAGNTYYASWAKSRFTISRDNSKNTLYLQMNSLRAEDTAVYYCARDDRWSLNIWGQGTLVTVSSGGGGSGGGGSGGGGSDPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 455 aa, Artificial Sequence
  scFv ROR2_huX3.12.6 VH_4GS3_VL_MiH1_ CD28tm+4-1BB/zeta
EVQLVESGGGLVQPGGSLRLSCAASGFTFSSYGVTWVRQAPGKGLEWVSYINTAGNTYYASWAKSRFTISRDNAKNSLYLQMNSLRAEDTAVYYCARDDRWSLNIWGQGTLVTVSSGGGGSGGGGSGGGGSDPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 452 aa, Artificial Sequence
  scFv SLAMF7_ERCS409 VH_4GS3_VL_MiH1_ CD28tm+4-1BB/zeta
QSVKESEGGLFKPTDTLTLTCTVSGFSLNSYGVIWVRQAPGNGLEYIGIIGSSGNTYYASWAKSRSTITRNTRLNTVTLKMTSLTAADTATYFCARYYGDSGFDSWGPGTLVTVSSGGGGSGGGGSGGGGSAQVLTQTPSSTSVAVGGTVTIKCQASQSIGSWLSWYQQKPGQPPKLLIYGASNLASGVPSRFKGGRSGTEYSLTISGVQREDAATYYCLGASPNGWAFGAGTNVEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 455 aa, Artificial Sequence
  scFv SLAMF7_huLuc63 VH_4GS3_VL_MiH1_ CD28tm+4-1BB/zeta
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSGGGGSGGGGSDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 466 aa, Artificial Sequence
  scFv FLT3_BV10 VH_4GS3_VL_MiH1_ CD28tm+4-1BB/zeta
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSSGGGGSGGGGSGGGGSDIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKRELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 455 aa, Artificial Sequence
  scFv FLT3_4G8 VH_4GS3_VL_MiH1_ CD28tm+4-1BB/zeta
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSGGGGSGGGGSGGGGSDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKRELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 452 aa, Artificial Sequence
  scFv Siglec-6_JML-1 VH_4GS3_VL_MiH1_ CD28tm+4-1BB/zeta
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 454 aa, Artificial Sequence
  scFv avb3_LM609v7 VH_4GS3_VL_MiH1_ CD28tm+4-1BB/zeta
QVQLQESGPGLVKPSQTLSLTCTVSGASISRGGYYWSWIRQYPGKGLEWIGYIHHSGSTYYNPSLKSRVTIAIDTSKNQLSLRLTSVTAADTAVYYCARHNYGSFAYWGQGTLVTVSSGGGGSGGGGSGGGGSELVMTQSPEFQSVTPKETVTITCRASQDIGNSLHWYQQKPGQSPKLLIKYASQPVFGVPSRFRGSGSGTDFTLTISRLEPEDFAVYYCQQSNSWPHTFGQGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 453 aa, Artificial Sequence
  scFv avb3_LM609v11 VH_4GS3_VL_MiH1_ CD28tm+4-1BB/zeta
QVQLVQSGAEVRKPGSSVRVSCKASGGTFSGFAVSWVRQAPGQRFEWLGGIVASLGSTDYAQKFQDKLTITVDESTATVYMEMRNLRSDDTAVYYCARHNYGSFAYWGQGTLVTVSSGGGGSGGGGSGGGGSELVMTQSPEFQSVTPKETVTITCRASQDIGTSLHWYQQKPGQSPKLLIKYASQPVFGVPSRFRGSGSGTDFTLTIYSLEAEDFAVYYCQQSNSWPHTFGQGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 460 aa, Artificial Sequence
  scFv BCMA_BCMA30 VH_4GS3_VL_MiH1_ CD28tm+4-1BB/zeta
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - protein, 460 aa, Artificial Sequence
  scFv BCMA_BCMA50 VH_4GS3_VL_MiH1_ CD28tm+4-1BB/zeta
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 99 aa, Artificial Sequence
  CH2 domain of human IgG3
SVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTIS

SEQ ID NO: 173 - protein, 99 aa, Artificial Sequence
  CH3 domain of human IgG3
PQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLS

SEQ ID NO: 174 - protein, 62 aa, Artificial Sequence
  CD4tm+intracellular
MALIVLGGVAGLLLFIGLGIFFCVRCRHRRRQAERMSQIKRLLSEKKTCQCPHRFQKTCSPI